(19) United States
(12) Patent Application Publication
(10) Pub. No.: US 2003/0073622 A1
(43) Pub. Date: Apr. 17, 2003

(54) Novel proteins and nucleic acids encoding same

(76) Inventors: Kumud Majumder, Stamford, CT (US); Kimberly A. Spytek, New Haven, CT (US); Velizar T. Tchernev, Branford, CT (US); Steven D. Colman, Gainesville, FL (US); Muralidhara Padigaru, Bronx, NY (US); Bryan Zerhusen, Branford, CT (US); Vladimir Gusev, Madison, CT (US); Catherine Burgess, Branford, CT (US); Li Li, Branford, CT (US); Uriel M. Malyankar, North Branford, CT (US); Esha Gangolli, Madison, CT (US); David Stone, Guilford, CT (US); John MacDougall, Hamden, CT (US); Glennda Smithson, Branford, CT (US); Karen Ellerman, Branford, CT (US)

Correspondence Address: Ivor R. Elrifi, Esq., MINTZ, LEVIN, COHN, FERRIS,, GLOVSKY and POPEO, P.C., One Financial Center, Boston, MA, 02111, US

(21) Appl. No.: 09/877,843

(22) Filed: Jun. 7, 2001

Related U.S. Application Data
(60) Provisional application No. 60/209,927, filed on Jun. 7, 2000.
(60) Provisional application No. 60/210,091, filed on Jun. 7, 2000.
(60) Provisional application No. 60/209,928, filed on Jun. 7, 2000.
(60) Provisional application No. 60/210,208, filed on Jun. 8, 2000.
(60) Provisional application No. 60/210,425, filed on Jun. 8, 2000.
(60) Provisional application No. 60/214,150, filed on Jun. 26, 2000.
(60) Provisional application No. 60/214,023, filed on Jun. 26, 2000.
(60) Provisional application No. 60/215,005, filed on Jun. 29, 2000.
(60) Provisional application No. 60/270,060, filed on Feb. 20, 2001.
(60) Provisional application No. 60/271,623, filed on Feb. 26, 2001.
(60) Provisional application No. 60/278,915, filed on Mar. 26, 2001.

(51) Int. Cl. A61K 38/17; G01N 33/574; C07H 21/04; C12P 21/02; C12N 5/06; C07K 14/435

(52) U.S. Cl. 514/12; 530/350; 536/23.5; 435/7.23; 435/69.1; 435/320.1; 435/325

(57) ABSTRACT

Disclosed herein are nucleic acid sequences that encode novel polypeptides. Also disclosed are polypeptides encoded by these nucleic acid sequences, and antibodies, which immunospecifically-bind to the polypeptide, as well as derivatives, variants, mutants, or fragments of the aforementioned polypeptide, polynucleotide, or antibody. The invention further discloses therapeutic, diagnostic and research methods for diagnosis, treatment, and prevention of disorders involving any one of these novel human nucleic acids and proteins.

### RELATED APPLICATIONS

This application claims priority from U.S. Ser. No. 60/209,927, filed Jun. 7, 2000; U.S. Ser. No. 60/210,091, filed Jun. 7, 2000; U.S. Ser. No. 60/209,928, filed Jun. 7, 2000; U.S. Ser. No. 60/210,208, filed Jun. 8, 2000; U.S. Ser. No. 60/210,425, filed Jun. 8, 2000; U.S. Ser. No. 60/214,150, filed Jun. 26, 2000; U.S. Ser. No. 60/214,023, filed Jun. 26, 2000; U.S. Ser. No. 60/215,005, filed Jun. 29, 2000; U.S. Ser. No. 60/270,060, filed Feb. 20, 2001; U.S. Ser. No. 60/271,623, filed Feb. 26, 2001; U.S. Ser. No. 60/278,915 filed Mar. 26, 2001, each of which is incorporated by reference in its entirety.

### FIELD OF THE INVENTION

The invention generally relates to nucleic acids and polypeptides encoded thereby.

### BACKGROUND OF THE INVENTION

The invention generally relates to nucleic acids and polypeptides encoded therefrom. More specifically, the invention relates to nucleic acids encoding cytoplasmic, nuclear, membrane bound, and secreted polypeptides, as well as vectors, host cells, antibodies, and recombinant methods for producing these nucleic acids and polypeptides.

### SUMMARY OF THE INVENTION

The invention is based in part upon the discovery of nucleic acid sequences encoding novel polypeptides. The novel nucleic acids and polypeptides are referred to herein as NOVX, or NOV1, NOV2, NOV3, NOV4, NOV5, NOV6, NOV7, and NOV8 nucleic acids and polypeptides. These nucleic acids and polypeptides, as well as derivatives, homologs, analogs and fragments thereof, will hereinafter be collectively designated as “NOVX” nucleic acid or polypeptide sequences.

In one aspect, the invention provides an isolated NOVX nucleic acid molecule encoding a NOVX polypeptide that includes a nucleic acid sequence that has identity to the nucleic acids disclosed in SEQ ID NOS: 1, 3, 5, 7, 9, 11, 13, 15, 17, 19, 21, and 23. In some embodiments, the NOVX nucleic acid molecule will hybridize under stringent conditions to a nucleic acid sequence complementary to a nucleic acid molecule that includes a protein-coding sequence of a NOVX nucleic acid sequence. The invention also includes an isolated nucleic acid that encodes a NOVX polypeptide, or a fragment, homolog, analog or derivative thereof. For example, the nucleic acid can encode a polypeptide at least 80% identical to a polypeptide comprising the amino acid sequences of SEQ ID NOS: 2, 4, 6, 8, 10, 12, 14, 16, 18, 20, 22, and 24. The nucleic acid can be, for example, a genomic DNA fragment or a cDNA molecule that includes the nucleic acid sequence of any of SEQ ID NOS: 1, 3, 5, 7, 9, 11, 13, 15, 17, 19, 21, and 23.

Also included in the invention is an oligonucleotide, e.g., an oligonucleotide which includes at least 6 contiguous nucleotides of a NOVX nucleic acid (e.g., SEQ ID NOS: 1, 3, 5, 7, 9, 11, 13, 15, 17, 19, 21, and 23) or a complement of said oligonucleotide.

Also included in the invention are substantially purified NOVX polypeptides (SEQ ID NOS:2, 4, 6, 8, 10, 12, 14, 16, 18, 20, 22, and 24). In certain embodiments, the NOVX polypeptides include an amino acid sequence that is substantially identical to the amino acid sequence of a human NOVX polypeptide.

The invention also features antibodies that immunoselectively bind to NOVX polypeptides, or fragments, homologs, analogs or derivatives thereof.

In another aspect, the invention includes pharmaceutical compositions that include therapeutically- or prophylactically-effective amounts of a therapeutic and a pharmaceutically-acceptable carrier. The therapeutic can be, e.g., a NOVX nucleic acid, a NOVX polypeptide, or an antibody specific for a NOVX polypeptide. In a further aspect, the invention includes, in one or more containers, a therapeutically- or prophylactically-effective amount of this pharmaceutical composition.

In a further aspect, the invention includes a method of producing a polypeptide by culturing a cell that includes a NOVX nucleic acid, under conditions allowing for expression of the NOVX polypeptide encoded by the DNA. If desired, the NOVX polypeptide can then be recovered.

In another aspect, the invention includes a method of detecting the presence of a NOVX polypeptide in a sample. In the method, a sample is contacted with a compound that selectively binds to the polypeptide under conditions allowing for formation of a complex between the polypeptide and the compound. The complex is detected, if present, thereby identifying the NOVX polypeptide within the sample.

The invention also includes methods to identify specific cell or tissue types based on their expression of a NOVX.

Also included in the invention is a method of detecting the presence of a NOVX nucleic acid molecule in a sample by contacting the sample with a NOVX nucleic acid probe or primer, and detecting whether the nucleic acid probe or primer bound to a NOVX nucleic acid molecule in the sample.

In a further aspect, the invention provides a method for modulating the activity of a NOVX polypeptide by contacting a cell sample that includes the NOVX polypeptide with a compound that binds to the NOVX polypeptide in an amount sufficient to modulate the activity of said polypeptide. The compound can be, e.g, a small molecule, such as a nucleic acid, peptide, polypeptide, peptidomimetic, carbohydrate, lipid or other organic (carbon containing) or inorganic molecule, as further described herein.

Also within the scope of the invention is the use of a therapeutic in the manufacture of a medicament for treating or preventing disorders or syndromes including, e.g., various tissue/organ inflammation, muscular dystrophy, neurological and neurodegenerative disorders, Duchenne muscular dystrophy (DMD), cardiovascular diseases and disorders, coagulation disorders, Mediterranean fever, various cancers including but not limited to meningiomas, breast, lung, colorectal cancers, adenocarcinoma, leukemia, lymphoma, melanoma, myeloma, sarcoma, infertility, reproductive disorders, birth control, developmental disorders, seizures, Alzheimer's disease, sleep disorders, appetite disorders, thermoregulation, pain perception, hormone secretion, sexual behavior, mental depression, migraine, epilepsy, obsessive-compulsive behavior (schizophrenia), vertex balding (hair loss), muscle fibre atrophy (motor neuron disease), Infantile neuronal ceroid lipofuscinosis (INCL), smooth muscle disorder, immunological disorder, Addison's disease, bronchitis, dermatomyositis, polymyositis, Crohn's disease, diabetes mellitus, lupus erythematosus, multiple sclerosis, ulcerative colitis, anaemia, osteoarthritis, rheumatoid arthritis, gout, hypertension, myocardial infarction, cardiovascular shock, angina, asthma, trauma, tissue regeneration (in vitro and in vivo), viral/bacterial/parasitic infections, respiratory disease, gastro-intestinal diseases, endocrine diseases, allergy and inflammation, nephrological disorders, muscle, bone disorders, hematopoietic disorders, urinary system disorders and/or other pathologies and disorders of the like. The therapeutic can be, e.g., a NOVX nucleic acid, a NOVX polypeptide, or a NOVX-specific antibody, or biologically-active derivatives or fragments thereof.

For example, the compositions of the present invention will have efficacy for treatment of patients suffering from the diseases and disorders disclosed above and/or other pathologies and disorders of the like. The polypeptides can be used as immunogens to produce antibodies specific for the invention, and as vaccines. They can also be used to screen for potential agonist and antagonist compounds. For example, a cDNA encoding NOVX may be useful in gene therapy, and NOVX may be useful when administered to a subject in need thereof. By way of non-limiting example, the compositions of the present invention will have efficacy for treatment of patients suffering from the diseases and disorders disclosed above and/or other pathologies and disorders of the like.

The invention further includes a method for screening for a modulator of disorders or syndromes including, e.g., the diseases and disorders disclosed above and/or other pathologies and disorders of the like. The method includes contacting a test compound with a NOVX polypeptide and determining if the test compound binds to said NOVX polypeptide. Binding of the test compound to the NOVX polypeptide indicates the test compound is a modulator of activity, or of latency or predisposition to the aforementioned disorders or syndromes.

Also within the scope of the invention is a method for screening for a modulator of activity, or of latency or predisposition to an disorders or syndromes including, e.g., the diseases and disorders disclosed above and/or other pathologies and disorders of the like by administering a test compound to a test animal at increased risk for the aforementioned disorders or syndromes. The test animal expresses a recombinant polypeptide encoded by a NOVX nucleic acid. Expression or activity of NOVX polypeptide is then measured in the test animal, as is expression or activity of the protein in a control animal which recombinantly-expresses NOVX polypeptide and is not at increased risk for the disorder or syndrome. Next, the expression of NOVX polypeptide in both the test animal and the control animal is compared. A change in the activity of NOVX polypeptide in the test animal relative to the control animal indicates the test compound is a modulator of latency of the disorder or syndrome.

In yet another aspect, the invention includes a method for determining the presence of or predisposition to a disease associated with altered levels of a NOVX polypeptide, a NOVX nucleic acid, or both, in a subject (e.g., a human subject). The method includes measuring the amount of the NOVX polypeptide in a test sample from the subject and comparing the amount of the polypeptide in the test sample to the amount of the NOVX polypeptide present in a control sample. An alteration in the level of the NOVX polypeptide in the test sample as compared to the control sample indicates the presence of or predisposition to a disease in the subject. Preferably, the predisposition includes, e.g., the diseases and disorders disclosed above and/or other pathologies and disorders of the like. Also, the expression levels of the new polypeptides of the invention can be used in a method to screen for various cancers as well as to determine the stage of cancers.

In a further aspect, the invention includes a method of treating or preventing a pathological condition associated with a disorder in a mammal by administering to the subject a NOVX polypeptide, a NOVX nucleic acid, or a NOVX-specific antibody to a subject (e.g., a human subject), in an amount sufficient to alleviate or prevent the pathological condition. In preferred embodiments, the disorder, includes, e.g., the diseases and disorders disclosed above and/or other pathologies and disorders of the like.

In yet another aspect, the invention can be used in a method to identity the cellular receptors and downstream effectors of the invention by any one of a number of techniques commonly employed in the art. These include but are not limited to the two-hybrid system, affinity purification, co-precipitation with antibodies or other specific-interacting molecules.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and not intended to be limiting.

Other features and advantages of the invention will be apparent from the following detailed description and claims.

### DETAILED DESCRIPTION OF THE INVENTION

The present invention provides novel nucleotides and polypeptides encoded thereby. Included in the invention are the novel nucleic acid sequences and their polypeptides. The sequences are collectively referred to as “NOVX nucleic acids” or “NOVX polynucleotides” and the corresponding encoded polypeptides are referred to as “NOVX polypeptides” or “NOVX proteins.” Unless indicated otherwise, “NOVX” is meant to refer to any of the novel sequences disclosed herein. Table A provides a summary of the NOVX nucleic acids and their encoded polypeptides.

TABLE A
Sequences and Corresponding SEQ ID Numbers

| | | SEQ ID | | |
|---|---|---|---|---|
| | | NO | | |
| NOVX | | (nucleic | SEQ ID NO | |
| Assignment | Internal Identification | acid) | (polypeptide) | Homology |
| 1a | GMAC068831_A | 1 | 2 | Calpactin-like |
| 1b | CG54593-02 | 3 | 4 | Calpactin-like |
| 1c | CG54593-03 | 5 | 6 | Calpactin-like |
| 2 | GM_AC069022_A | 7 | 8 | Spermadhesin-like |
| 3 | GM_AC036220_A | 9 | 10 | Disintegrin-like |
| 4 | GMAC023790_A | 11 | 12 | 5-Hydrozytryptamine-7 |
| | | | | Recetor-like |
| 5 | SC105318106_A | 13 | 14 | Insulin Growth Factor |
| | | | | Binding Protein-like |
| 6 | 117478122-A | 15 | 16 | Cell Cycle P38-2G4-like |
| 7a | SC117873416-A | 17 | 18 | Microsomal Signal Peptidase |
| | | | | 18KDa-like |
| 7b | CG57520-01 | 19 | 20 | Microsomal Signal Peptidase |
| | | | | 18KDa-like |
| 7c | CG57520-02 | 21 | 22 | Microsomal Signal Peptidase |
| | | | | 18KDa-like |
| 8 | GMAC006928_1 | 23 | 24 | Stromal Interaction Molecule-like |

NOVX nucleic acids and their encoded polypeptides are useful in a variety of applications and contexts. The various NOVX nucleic acids and polypeptides according to the invention are useful as novel members of the protein families according to the presence of domains and sequence relatedness to previously described proteins. Additionally, NOVX nucleic acids and polypeptides can also be used to identify proteins that are members of the family to which the NOVX polypeptides belong.

For example, NOV1a, 1b and 1c are homologous to a Calpactin-like family of proteins. Thus, the NOV1 nucleic acids, polypeptides, antibodies and related compounds according to the invention will be useful in therapeutic and diagnostic applications implicated in, for example; various cancers, tissue/organ inflammation, muscular dystrophy, neurodegenerative disorders, Duchenne muscular dystrophy (DMD), cardiovascular diseases and disorders, coagulation disorders and diseases, Mediterranean fever, and/or other pathologies/disorders.

Also, NOV2 is homologous to the Spermadhesin family of proteins. Thus NOV2 nucleic acids, polypeptides, antibodies and related compounds according to the invention will be useful in therapeutic and diagnostic applications implicated in, for example; infertility and other reproductive disorders, for development of a birth control composition and/or other pathologies/disorders.

Further, NOV3 is homologous to a family of connexin-like proteins. Thus, the NOV3 nucleic acids and polypeptides, antibodies and related compounds according to the invention will be useful in therapeutic and diagnostic applications implicated in, for example; various cancers, reproductive disorders, early development disorders and/or other pathologies/disorders.

Also, NOV4 is homologous to the 5-hydroxytryptamine-7 receptor family of proteins. Thus, NOV4 nucleic acids, polypeptides, antibodies and related compounds according to the invention will be useful in therapeutic and diagnostic applications implicated in, for example; seizures, Alzheimer's disease, sleep disorders, appetite disorders, thermoregulation, pain perception, hormone secretion, sexual behavior, mental depression, migraine, epilepsy, obsessive-compulsive behavior (schizophrenia), and affective disorder and/or other pathologies/disorders.

Additionally, NOV5 is homologous to the insulin growth factor binding protein family of proteins. Thus NOV5 nucleic acids, polypeptides, antibodies and related compounds according to the invention will be useful in therapeutic and diagnostic applications implicated in, for example; vertex balding (hair loss), cancer, meningioma, breast carcinoma, colorectal tumors, lung cancer, muscle fibre atrophy (motor neuron disease), Infantile neuronal ceroid lipofuscinosis (INCL) and/or other pathologies/disorders.

Also, NOV6 is homologous to the cell cycle P38-2G4-like family of proteins. Thus NOV6 nucleic acids, polypeptides, antibodies and related compounds according to the invention will be useful in therapeutic and diagnostic applications implicated in diseases or disorders related to aberrant expression, aberrant function or aberrant physiologic interactions of the cell cycle P38-2G4-like nucleic acid or protein.

Further, NOV7a, 7b and 7c are homologous to members of the microsomal signal peptidase 18KDa-like family of proteins which play an essential role in protein modification. Thus, the NOV7 nucleic acids, polypeptides, antibodies and related compounds according to the invention will be useful in therapeutic and diagnostic applications implicated in, for example; Addison's disease, bronchitis, dermatomyositis, polymyositis, Crohn's disease, diabetes mellitus, lupus erythematosus, multiple sclerosis, ulcerative colitis, anaemia, osteoarthritis, rheumatoid arthritis, gout, hypertension, myocardial infarction, cardiovascular shock, angina, asthma, migraine, adenocarcinoma, leukemia, lymphoma, melanoma, myeloma, sarcoma and/or other pathologies/disorders.

Still further, NOV8 is homologous to a stromal interaction molecule-like family of proteins that are important in cell adhesion. Thus, NOV8 nucleic acids and polypeptides, antibodies and related compounds according to the invention will be useful in therapeutic and diagnostic applications implicated in, for example; cancer, trauma, tissue regeneration (in vitro and in vivo), viral/bacterial/parasitic infections, immunological disease, respiratory disease, gastro-intestinal diseases, reproductive health, neurological and neurodegenerative diseases, endocrine diseases, allergy and inflammation, nephrological disorders, cardiovascular diseases, muscle, bone disorders, hematopoietic disorders, urinary system disorders, developmental disorders and/or other pathologies/disorders.

The NOVX nucleic acids and polypeptides can also be used to screen for molecules, which inhibit or enhance NOVX activity or function. Specifically, the nucleic acids and polypeptides according to the invention may be used as targets for the identification of small molecules that modulate or inhibit, e.g., neurogenesis, cell differentiation, cell proliferation, hematopoiesis, wound healing and angiogenesis.

Additional utilities for the NOVX nucleic acids and polypeptides according to the invention are disclosed herein.

NOV1

NOV1 includes three novel Calpactin-like proteins disclosed below. The disclosed proteins have been named NOV1a, NOV1b and NOV1c.

NOV1a

A NOV1a nucleic acid of 320 nucleotides (also referred to as GMAC068831_A) encoding a novel calpactin-like protein is shown in Table 1A. An open reading frame was identified beginning with an ATG initiation codon at nucleotides 4-6 and ending with a TGA codon at nucleotides 313-315. A putative untranslated region upstream from the initiation codon and downstream from the termination codon is underlined in Table 1A, and the start and stop codons are in bold letters.

TABLE 1A
NOV1a nucleotide sequence (SEQ ID NO:1).

CCAATGGAACACACCATGGAAACCGTGTTGTTTACGTTTCACAGATTTGCCGGGGATAAAGGCTACTTAATG
AAGGAGGGCCTGAAAGTACTCATGGGAAAGGAGTTCCTTGGATTTTTGGAGAATCAAAAAGACCCTCTGGCT
GCAGACATAACAATGAAGGACATGGACCAGTGCCAAGACAGCACACTGAACTTCCAGAACTTGTTTTCACTC
ACTGCGGCGCTCACCACTGTGGACAACAACTATTTTGTAGTACCTATGAAGCAGAAGGGAACGAAGCAGGCA
GAACTAAGCAATTACTCAGCCCTGTGAGAGTT

A NOV1a nucleic acid was identified on chromosome 15 by TblastN using CuraGen Corporation's sequence file for calpactin or homolog as run against the Genomic Daily Files made available by GenBank or from files downloaded from the individual sequencing centers. The nucleic acid sequence was predicted from the genomic file Genbank accession number: AC068831 by homology to a known calpactin or homolog. Exons were predicted by homology and the intron/exon boundaries were determined using standard genetic rules. Exons were further selected and refined by means of similarity determination using multiple BLAST (for example, tBlastN, BlastX, and BlastN) searches, and, in some instances, GeneScan and Grail. Expressed sequences from both public and proprietary databases were also added when available to further define and complete the gene sequence. The DNA sequence was then manually corrected for apparent inconsistencies thereby obtaining the sequences encoding the full-length protein.

In a search of public sequence databases, it was found, for example, that the NOV1a nucleic acid sequence disclosed in this invention has 267 of 322 bases (82%) identical to one region of a Homo Sapiens cellular ligand of annexin II (Calpactin I Light Chain) mRNA, with an E-value of 1.8e⁻⁴³ (GENBANK-ID:HUMCLANNII|acc:M38591). Public nucleotide databases include all GenBank databases and the GeneSeq patent database.

In all BLAST alignments herein, the “E-value” or “Expect” value is a numeric indication of the probability that the aligned sequences could have achieved their similarity to the BLAST query sequence by chance alone, within the database that was searched. For example, the probability that the subject (“Sbjct”) retrieved from the NOV1 BLAST analysis, e.g., Homo sapiens cellular ligand of annexin II (Calpactin I Light Chain) mRNA, matched the Query NOV1 sequence purely by chance is 1.8e⁻⁴³. The Expect value (E) is a parameter that describes the number of hits one can “expect” to see just by chance when searching a database of a particular size. It decreases exponentially with the Score (S) that is assigned to a match between two sequences. Essentially, the E value describes the random background noise that exists for matches between sequences.

The Expect value is used as a convenient way to create a significance threshold for reporting results. The default value used for blasting is typically set to 0.0001. In BLAST 2.0, the Expect value is also used instead of the P value (probability) to report the significance of matches. For example, an E value of one assigned to a hit can be interpreted as meaning that in a database of the current size one might expect to see one match with a similar score simply by chance. An E value of zero means that one would not expect to see any matches with a similar score simply by chance. See, e.g., http://www.ncbi.nlm.nih.gov/Education/BLASTinfo/. Occasionally, a string of X's or N's will result from a BLAST search. This is a result of automatic filtering of the query for low-complexity sequence that is performed to prevent artifactual hits. The filter substitutes any low-complexity sequence that it finds with the letter “N” in nucleotide sequence (e.g., “NNNNNNNNNNNN”) or the letter “X” in protein sequences (e.g., “XXXXXXXXX”). Low-complexity regions can result in high scores that reflect compositional bias rather than significant position-by-position alignment. Wootton and Federhen, Methods Enzymol 266:554-571, 1996.

A disclosed encoded NOV1a protein has 103 amino acid residues, referred to as the NOV1a protein. The NOV1a protein was analyzed for signal peptide prediction and cellular localization. The SignalP and Psort results predict that NOV1a does not have a signal peptide and is likely to be localized to the microbody (peroxisome) with a certainty of 0.4770 and to the cytoplasm, with a certainty of 0.4500. The disclosed NOV1a polypeptide sequence is presented in Table 1B using the one-letter amino acid code. NOV1a has a molecular weight of 11682.3 Daltons.

TABLE 1B
Encoded NOV1a protein sequence (SEQ ID NO:2).

MEHTMETVLFTFHRFAGDKGYLMKEGLKVLMGKEFLGFLENQKDPLAADITMKDMDQCQDSTLN
FQNLFSLTAGLTTVDNNYFVVPMKQKGTKQAELSNYSAL

A BLASTX search was performed against public protein databases. The disclosed NOV1a protein (SEQ ID NO:2) has good identity with calpactin-like proteins. For example, the full amino acid sequence of the protein of the invention was found to have 66 of 93 amino acid residues (70%) identical to, and 75 of 93 residues (80%) positive with, the 96 amino acid residue Calpactin I Light Chain protein from Homo sapiens (ptnr: SWISSPROT-ACC:P08206; E=5.4e⁻³¹). Public amino acid databases include the GenBank databases, SwissProt, PDB and PIR.

NOV1b

A NOV1a nucleic acid ( GMAC068831_A) was subjected to the exon linking process to confirm the sequence. PCR primers were designed by starting at the most upstream sequence available, for the forward primer, and at the most downstream sequence available for the reverse primer. In each case, the sequence was examined, walking inward from the respective termini toward the coding sequence, until a suitable sequence that is either unique or highly selective was encountered, or, in the case of the reverse primer, until the stop codon was reached. Such primers were designed based on in silico predictions for the full length cDNA, part (one or more exons) of the DNA or protein sequence of the target sequence, or by translated homology of the predicted exons to closely related human sequences sequences from other species. These primers were then employed in PCR amplification based on the following pool of human cDNAs: adrenal gland, bone marrow, brain-amygdala, brain-cerebellum, brain-hippocampus, brain-substantia nigra, brain-thalamus, brain-whole, fetal brain, fetal kidney, fetal liver, fetal lung, heart, kidney, lymphoma-Raji, mammary gland, pancreas, pituitary gland, placenta, prostate, salivary gland, skeletal muscle, small intestine, spinal cord, spleen, stomach, testis, thyroid, trachea, uterus. Usually the resulting amplicons were gel purified, cloned and sequenced to high redundancy. The resulting sequences from all clones were assembled with themselves, with other fragments in CuraGen Corporation's database and with public ESTs. Fragments and ESTs were included as components for an assembly when the extent of their identity with another component of the assembly was at least 95% over 50 bp. In addition, sequence traces were evaluated manually and edited for corrections if appropriate. These procedures provide the sequence reported below, which is designated Accession Number CG54593-02 (NOV1b) and CG54593-03 (NOV1c).

A NOV1b nucleic acid of 321 nucleotides (also referred to as CG54593-02) encoding a novel Calpactin I Light Chain-like protein is shown in Table 1C. An open reading frame was identified beginning with an ATG initiation codon at nucleotides 5-7 and ending with a TGA codon at nucleotides 314-316. A putative untranslated region upstream from the initiation codon and downstream from the termination codon is underlined in Table 1C, and the start and stop codons are in bold letters.

TABLE 1C
NOV1b nucleotide sequence (SEQ ID NO:3).

TCCAATGGAACACACCATGGAAACCGTGTTGTTTACGTTTCACAGATTTGCCGGGGATAAAGGCTACTTAAT
GAAGGAGGGCCTGAAAGTACTCATGGGAAAGGAGTTCCCTGGATTTTTGGAGAATCAAAAAGACCCTCTGGC
TGCAGACATAACAATGAAGGACATGGACCAGTGCCAAGACAGCACACTGAACTTCCAGAACTTGTTTTCACT
CACTGCGGGGCTCACCACTGTGGACAACAACTATTTTGTAGTACCTATGAGCAGAAGGGAACCAAGCAGGC
AGAACTAAGCAATTACTCAGCCCTGTGAGAGTA

In a search of public sequence databases, it was found, for example, that the NOV1b nucleic acid sequence disclosed in this invention has 268 of 322 bases (83%) identical to one region of a Homo Sapiens cellular ligand of annexin II (Calpactin I Light Chain; p11; p10 protein) mRNA, with an E-value of 4.6e⁻⁴⁴ (GENBANK-ID:HUMCLANNII|acc:M38591.1).

A disclosed encoded NOV1b protein has 103 amino acid residues, referred to as the NOV1b protein. The NOV1b protein was analyzed for signal peptide prediction and cellular localization. The SignalP and Psort results predict that NOV1b does not have a signal peptide and is likely to be localized to the microbody (peroxisome) with a certainty of 0.4633 and to the cytoplasm, with a certainty of 0.4500. Although PSORT suggests that NOV1b protein may be localized in the microbody (peroxisome), the NOV1b protein predicted here is similar to the Calpactin I Light Chain family, some members of which are secreted. Therefore it is likely that this NOV1b protein is localized to the same sub-cellular compartment. The disclosed NOV1b polypeptide sequence is presented in Table 1D using the one-letter amino acid code.

TABLE 1D
Encoded NOV1b protein sequence (SEQ ID NO:4).

MEHTMETVLFTFHRFAGDKGYLMKEGLKVLMGKEFPGFLENQKDPLAADITMKDMDQCQDSTLNFQNLFSLT
AGLTTVDNNYFVVPMKQKGTKQAELSNYSAL

The Calpactin-like protein (NOV1b) maps to chromosome 17.

A BLASTX search was performed against public protein databases. The disclosed NOV1b protein (SEQ ID NO:4) has good identity with calpactin-like proteins. For example, the full amino acid sequence of the protein of the invention was found to have 67 of 93 amino acid residues (72%) identical to, and 76 of 93 residues (81%) positive with, the 96 amino acid residue Calpactin I Light Chain protein from Homo sapiens (ptnr: SWISSPROT-ACC:P08206; E=2.3e⁻³²).

NOV1c

A NOV1c nucleic acid of 321 nucleotides (also referred to as CG54593-03) encoding a novel Calpactin I Light Chain-like protein is shown in Table 1E. An open reading frame was identified beginning at nucleotides 2-4 and ending at nucleotides 314-316. A putative untranslated region upstream from the initiation codon and downstream from the termination codon is underlined in Table 1E, and the start and stop codons are in bold letters.

TABLE 1E
NOV1C nucleotide sequence (SEQ ID NO:5).

TCCAATGGAACACACCATGGAAACCGTGTTGTTTACGTTTCACAGATTTGCCGGGGATAAAGGCTACTTAAT
GAAGGAGGGCCTGAAAGTACTCATGGGAAAGGAGTTCCCTGGATTTTTGGAGAATCAAAAAGACCCTCTGGC
TGCACACATAACAATGAAGGACATGCACCAGTGCCAAGACAGCACACTGAACTTCCAGAACTTGTTTTCACT
CACTGCGGGGCTCACCACTGTGGACAACAACTATTTTGTAGTACCTATGAAGCAGAAGGGAACGAAGCAGGC
AGAACTAAGCAATTACTCAGCCCTGTGAGAGTA

In a search of public sequence databases, it was found, for example, that the NOV 1c nucleic acid sequence disclosed in this invention has 268 of 322 bases (83%) identical to one region of a Homo Sapiens cellular ligand of annexin II (Calpactin I Light Chain; p11; p10 protein) mRNA, with an E-value of 4.6e⁻⁴⁴ (GENBANK-ID:HUMCLANNII|acc:M38591.1).

A disclosed encoded NOV1c protein has 104 amino acid residues, referred to as the NOV1c protein. The NOV1c protein was analyzed for signal peptide prediction and cellular localization. The SignalP and Psort results predict that NOV1c does not have a signal peptide and is likely to be localized to the microbody (peroxisome) with a certainty of 0.4332 and to the cytoplasm, with a certainty of 0.4500. Although PSORT suggests that NOV1c protein may be localized in the microbody (peroxisome), the NOV1c protein predicted here is similar to the Calpactin I Light Chain family, some members of which are secreted. Therefore it is likely that this NOV1c protein is localized to the same sub-cellular compartment. The disclosed NOV1c polypeptide sequence is presented in Table 1F using the one-letter amino acid code.

TABLE 1F
Encoded NOV1c protein sequence (SEQ ID NO:6).

MEHTMETVLFTFHRFAGDKGYLMKEGLKVLMGKEFPGFLENQKDPLAADITMKDMDQCQDSTLNFQNLFSLT
AGLTTVDNNYFVVPMKQKGTKQAELSNYSAL

The Calpactin-like protein (NOV1c) maps to chromosome 17.

A BLASTX search was performed against public protein databases. The disclosed NOV1b protein (SEQ ID NO:6) has good identity with calpactin-like proteins. For example, the full amino acid sequence of the protein of the invention was found to have 67 of 93 amino acid residues (72%) identical to, and 76 of 93 residues (81%) positive with, the 96 amino acid residue Calpactin I Light Chain protein from Homo sapiens (ptnr: SWISSPROT-ACC:P08206; E=2.3e⁻³²).

NOV1a, 1b and 1c are related to each other as shown in the alignment listed in Table 1G.

[image]

It was also found that NOV1a had homology to the amino acid sequences shown in the BLASTP data listed in Table 1H.

TABLE 1H
BLAST results for NOV1a

| Gene Index/ | | Length | Identity | Positives | |
|---|---|---|---|---|---|
| Identifier | Protein/Organism | (aa) | (%) | (%) | Expect |
| gi\|3212355\|pdb\|1A4P\|A | Chain A, P11 | 96 | 66/93 | 75/93 | 3e−29 |
| | (S100a10), Ligand | | (70%) | (79%) | |
| | Of Annexin Ii | | | | |
| gi\|4506761\|ref\|NP_0 | S100 calcium- | 97 | 66/93 | 75/93 | 5e−29 |
| 02957.1\| | binding protein | | (70%) | (79%) | |
| | A10; 42C; annexin | | | | |
| | II ligand, | | | | |
| | calpactin I, light | | | | |
| | polypeptide [Homo | | | | |
| | sapiens] | | | | |
| gi\|116488\|sp\|P04163 | CALPACTIN I LIGHT | 95 | 65/91 | 74/91 | 8e−29 |
| \|S110_PIG | CHAIN (P10 | | (71%) | (80%) | |
| | PROTEIN) (P11) | | | | |
| | (CELLULAR LIGAND | | | | |
| | OF ANNEXIN II) | | | | |
| gi\|116485\|sp\|P27003 | CALPACTIN I LIGHT | 97 | 61/93 | 73/93 | 4e−27 |
| \|S110_CHICK | CHAIN (P10 | | (65%) | (77%) | |
| | PROTEIN) (P11) | | | | |
| | (CELLULAR LIGAND | | | | |
| | OF ANNEXIN II) | | | | |
| gi\|6677833\|ref\|NP_0 | calcium binding | 97 | 62/93 | 72/93 | 6e−27 |
| 33138.1\| | protein A11 | | (66%) | (76%) | |
| | (calgizzarin) [Mus | | | | |
| | musculus] | | | | |

The homology of these and other sequences is shown graphically in the ClustalW analysis shown in Table 1I. In the ClustalW alignment of the NOV1a protein, as well as all other ClustalW analyses herein, the black outlined amino acid residues indicate regions of conserved sequence (i.e., regions that may be required to preserve structural or functional properties), whereas non-highlighted amino acid residues are less conserved and can potentially be mutated to a much broader extent without altering protein structure or function.

[image]

[image]

The homologies shown above are shared by NOV1b and 1c insofar as they are themselves homologous to NOV1a as shown in Table 1G.

The presence of identifiable domains in NOV1, as well as all other NOVX proteins, was determined by searches using software algorithms such as PROSITE, DOMAIN, Blocks, Pfam, ProDomain, and Prints, and then determining the Interpro number by crossing the domain match (or numbers) using the Interpro website (http:www.ebi.ac.uk/interpro). DOMAIN results for NOV1 as disclosed in Tables 1J, were collected from the Conserved Domain Database (CDD) with Reverse Position Specific BLAST analyses. This BLAST analysis software samples domains found in the Smart and Pfam collections. For Tables 1J and all successive DOMAIN sequence alignments, fully conserved single residues are indicated by black shading and “strong” semi-conserved residues are indicated by grey shading. The “strong” group of conserved amino acid residues may be any one of the following groups of amino acids: STA, NEQK, NHQK, NDEQ, QHRK, MILV, MILF, HY, FYW.

Table 1J lists the domain description from DOMAIN analysis results against NOV1a. This indicates that the NOV1 a sequence has properties similar to those of other proteins known to contain this domain.

[image]

BLAST results include sequences from the Patp database, which is a proprietary database that contains sequences published in patents and patent publications. Patp results include those listed in Table 1K.

TABLE 1K
Patp alignments of NOV1a

| | | | Smallest |
|---|---|---|---|
| | | | Sum |
| | Reading | High | Prob. |
| Sequences producing High-scoring Segment Pairs: | Frame | Score | P(N) |
| Pstp:AAB45540 Humen S100A10 protein, 97 aa | +1 | 346 | 1.1e−30 |
| Patp:AAY93605 Protein encoded by a gene [Homo sapiens], 97 aa | +1 | 346 | 1.1e−30 |
| Patp:AAB71655 Human colon associated protein [Homo sapiens], 97 aa | +1 | 346 | 1.1e−30 |

The response to elevation of cytoplasmic Ca(2+) levels following extra- or intracellular stimuli is mediated by proteins that are capable of binding divalent calcium ions. A particular class of these proteins is characterized by the so-called EF-hand, a helix-loop-helix motif involved in coordinating the Ca(2+) ion. Within the EF-hand superfamily, a distinct set of proteins is grouped in the so-called S-100 protein family. Protein p11 (calpactin I, light chain) is a member of the S-100 family. The p11 calpactin I light chain is an intracellular polypeptide of 97 amino acid residues that associates with the calpactin I heavy chain, p36, to form a calcium-binding complex (Saris et al., 1987). The p11 subunit is a protein kinase substrate and likely plays a role in the regulation of p36 phosphorylation/activity. p11 I has several unique features, as it has suffered crucial deletions and amino acid substitutions which are thought to render both Ca(2+)-binding sites inactive. In all tissues and cells studied, p11 is found in a heterotetrameric complex with another Ca(2+)-binding protein, annexin II (ANX2). Some of the biochemical properties of annexin II are modulated by p11-induced complex formation, which involves the binding of one p11 dimer to 2 annexin II monomers. Harder et al. (1992) isolated the gene encoding 11 kb p11 (CLP11) from a human genomic library. In separate studies, Kube et al. (1991) and Dooley et al. (1992) cloned and sequenced the cDNA for the full-length human p11 calpactin I light chain. Calpain, an intracellular protease that requires calcium for its catalytic activity, is associated with the S-100 protein family. Two isozymes (CANP1 and CANP2), with different calcium requirements, have been identified. Both are heterodimers composed of L (large, catalytic, 80 kD) and S (small, regulatory, 30 kD) subunits. The isozymes share an identical S subunit (114170); differences arise from the L subunits (L1 and L2).

S-100 protein family members have been implicated in various disorders and disease states, such as, tissue/organ inflammation, muscular dystrophy, neurodegenerative disorders, Duchenne muscular dystrophy (DMD), cardiovascular diseases and disorders, coagulation disorders and diseases and Mediterranean fever.

The antiinflammatory action of glucocorticoids has been attributed to the induction of a group of proteins, collectively called lipocortin, that inhibit phospholipase A2. These proteins are thought to control the biosynthesis of potent mediators of inflammation, prostaglandins and leukotrienes, by inhibiting release of their common precursor, arachidonic acid, a process that requires hydrolysis of phospholipids by phospholipase A2 (Wallner et al., 1986). Lipocortin I belongs to a structurally related family of annexins. They undergo Ca(2+)-dependent binding to phospholipids that are preferentially located on the cytosolic face of the plasma membrane (Huebner et al. 1987, 1988). Pepinsky et al. (1988) described the characteristics of 3 proteins they called lipocortin III, lipocortin V, and lipocortin VI. Shohat et al. (1989) advanced the hypothesis that familial Mediterranean fever patients are homozygous for a mutant allele for one of the lipocortin genes.

Efficient platelet adhesion and aggregation at sites of vascular injury requires the synergistic contribution of multiple adhesion receptors. The initial adhesion of platelets to subendothelial matrix proteins involves GPIb/V/IX and one or more platelet integrins, including integrin alpha IIb beta 3, alpha 2 beta 1, alpha 5 beta 1 and possibly alpha 6 beta 1. In contrast, platelet-platelet adhesion (platelet cohesion or aggregation) is mediated exclusively by GPIb/V/IX and integrin alpha IIb beta 3. Integrin alpha IIb beta 3 is a remarkable receptor that not only stabilizes platelet-vessel wall and platelet-platelet adhesion contacts, but also transduces signals necessary for a range of other functional responses. These signals are linked to cytoskeletal reorganization and platelet spreading, membrane vesiculation and fibrin clot formation, and tension development on a fibrin clot leading to clot retraction. This diverse functional role of integrin alpha IIb beta 3 is reflected by its ability to induce the activation of a broad range of signaling enzymes that are involved in membrane phospholipid metabolism, protein phosphorylation, calcium mobilization and activation of small GTPases. An important calcium-dependent signaling enzyme involved in integrin alpha IIb beta 3 outside-in signaling is the thiol protease, calpain. This enzyme proteolyses a number of key structural and signaling proteins involved in cytoskeletal remodeling and platelet activation. These proteolytic events appear to play a potentially important role in modulating the adhesive and signaling function of integrin alpha IIb beta 3 (Schoenwaelder et al., 2000).

The function of calpains in muscle has received increased interest because of the discoveries that the activation and concentration of the ubiquitous calpains increase in the mouse model of Duchenne muscular dystrophy (DMD), but null mutations of muscle specific calpain causes limb girdle muscular dystrophy 2A (LGMD2A). These findings indicate that modulation of calpain activity contributes to muscular dystrophies by disrupting normal regulatory mechanisms influenced by calpains, rather than through a general, nonspecific increase in proteolysis. Thus, modulation of calpain activity or expression through pharmacological or molecular genetic approaches may provide therapies for some muscular dystrophies (Tidball and Spencer, 2000).

Integrin-induced adhesion of cells activates intracellular signaling pathways that lead to cytoskeletal reorganizations and altered cell behavior. One of the signaling molecules that is activated as a consequence of integrin-induced signaling is calpain. Studies on platelets have demonstrated that the major substrates for calpain are proteins present in the complexes of integrin, cytoskeletal proteins, and signaling molecules that form as a consequence of integrin engagement. It has been shown that calpain is also active in cultured adherent cells and that the calpain-induced cleavage of proteins in these cells is required for integrin-induced changes in cell morphology and spreading. Investigation of the mechanisms involved has revealed that calpain induces integrin-induced formation of focal adhesions and actin filament reorganizations and that it does so by inducing the activation of both Rac1 and RhoA (Fox, 1999).

Neurons are an unusual type of cell in that they send processes (axons and dendrites) over great distances. This elaborate morphology, together with their excitability, places neurons at risk for multiple insults. Recent studies have demonstrated that apoptotic and excitotoxic mechanisms not only contribute to neuronal death, but also to synaptic dysfunction and a breakdown in neural circuitry (Mattson and Duan, 1999). Proteases of the caspase and calpain families have been implicated in neurodegenerative processes, as their activation can be triggered by calcium influx and oxidative stress. The substrates cleaved by caspases include cytoskeletal and associated proteins, kinases, members of the Bcl-2 family of apoptosis-related proteins, presenilins and amyloid precursor protein, and DNA-modulating enzymes. Calpain substrates include cytoskeletal and associated proteins, kinases and phosphatases, membrane receptors and transporters, and steroid receptors. Many of the substrates of caspases and calpains are localized in pre- and/or postsynaptic compartments of neurons. Emerging data suggest that, in addition to their roles in neurodegenerative processes, caspases and calpains play important roles in modulating synaptic plasticity. Emerging data suggest key roles for these proteases in the regulation of synaptic plasticity (Mattson and Duan, 1999).

Based on primary and secondary structural similiarity of NOV1 polypeptides to the “calpactin or S-100 family” of proteins, the NOV1 nucleic acids and proteins are useful in potential therapeutic applications implicated in (but not limited to) various pathologies and disorders as indicated below. For example, a cDNA encoding the calpactin-like protein may be useful in gene therapy, and the calpactin-like protein may be useful when administered to a subject in need thereof. The novel nucleic acid encoding NOV1 protein, or fragments thereof, may further be useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods.

The NOV1 nucleic acids and proteins of the invention are useful in potential therapeutic applications implicated in various diseases and disorders described below and/or other pathologies and disorders. The potential therapeutic applications for this invention include, but are not limited to; protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV1 nucleic acids and proteins of the invention are useful in potential therapeutic applications implicated in various tissue/organ inflammation, muscular dystrophy, neurodegenerative disorders, Duchenne muscular dystrophy (DMD), cardiovascular diseases and disorders, coagulation disorders and diseases, Mediterranean fever, other diseases involving prostaglandins and leukotrienes and also in cases of various cancers including but not limited to breast, lung, colorectal cancers and/or other pathologies and disorders. For example, a cDNA encoding the calpactin-like protein may be useful in gene therapy, and the calpactin-like protein may be useful when administered to a subject in need thereof. By way of nonlimiting example, the compositions of the present invention will have efficacy for treatment of patients suffering from various tissue/organ inflammation, Mediterranean fever, other diseases involving prostaglandins and leukotrienes and also in cases of various cancers including but not limited to breast, lung, colorectal cancers. The NOV1 nucleic acid, or fragments thereof, may further be useful in diagnostic applications, wherein the presence or amount of the nucleic acid or the protein are to be assessed.

NOV1 nucleic acids and polypeptides are further useful in the generation of antibodies that bind immuno-specifically to the novel NOV1 substances for use in therapeutic or diagnostic methods. These antibodies may be generated according to methods known in the art, using prediction from hydrophobicity charts, as described in the “Anti-NOVX Antibodies” section below. The disclosed NOV1 proteins have multiple hydrophilic regions, each of which can be used as an immunogen. In one embodiment, a contemplated NOV1 epitope is from about amino acids 8 to 14. In another embodiment, a NOV1 epitope is from about amino acids 18 to 30. In additional embodiments, NOV1 epitopes are from amino acids 35 to 65 and from amino acids 80 to 95. These novel proteins can be used in assay systems for functional analysis of various human disorders, which will help in understanding of pathology of the disease and development of new drug targets for various disorders.

NOV2

A NOV2 nucleic acid of 412 nucleotides (also referred to as GM_AC069022_A) encoding a novel Spermadhesin-like protein is shown in Table 2A. An open reading frame was identified beginning with an ATG initiation codon at nucleotides 5-7 and ending with a TAA codon at nucleotides 404-406. A putative untranslated region upstream from the initiation codon and downstream from the termination codon is underlined in Table 2A, and the start and stop codons are in bold letters.

TABLE 2A
NOV2 Nucleotide Sequence (SEQ ID NO:7)

TGGAATGACACAAGCCAATCCCAGACCTAGAGCATCCCTGCAGTGCCTGGGCCCCGTTTCTCCTCTTGTAG
CACCCAGTGACTGTGGGGGCCACTACACAGATGAATATGGCAGGATCTTCAACTACGCTGGGCCGAAAACT
GAATGCGTCTGGATCATCGAGTTGAACCCCGGGGAGATAGTCACGGTGGCCATTCCAGACCTCAAGTTCGC
ATGTGGCAAAGAATACGTGGAAGTGCTGGATGGACCTCCAGGGTCTGAGTCCTTGGACAGGATTTGTAAAG
CCTTCAGTACATTCTATTACTCTTCTTCCAACATCATCACCATCAAGTACTCCAGAGAACCCAGTCATCCA
CCCACCTTCTTTGAAATATATTACTTTGTTGACGCTTGGTCAACACATTAATAGGAA

A NOV2 nucleic acid was identified on chromosome 10 by TblastN using CuraGen Corporation's sequence file for Spermadhesin or homolog as run against the Genomic Daily Files made available by GenBank or from files downloaded from the individual sequencing centers. The nucleic acid sequence was predicted by splicing together regions 161044-160837 and 158878-158675 from the genomic file Genbank accession number: AC069022 by homology to a known Spermadhesin or homolog. Exons were predicted by homology and the intron/exon boundaries were determined using standard genetic rules. Exons were further selected and refined by means of similarity determination using multiple BLAST (for example, tBlastN, BlastX, and BlastN) searches, and, in some instances, GeneScan and Grail. Expressed sequences from both public and proprietary databases were also added when available to further define and complete the gene sequence. The DNA sequence was then manually corrected for apparent inconsistencies thereby obtaining the sequences encoding the full-length protein.

In a search of sequence databases, it was found, for example, that the nucleic acid sequence has 219 of 326 bases (67%) identical to a Sus scrofa (pig) Spermadhesin mRNA (GENBANK-ID: SSPSPI|acc:U02626)(E=2.7 e−20).

The disclosed NOV2 polypeptide (SEQ ID NO:8) encoded by SEQ ID NO:7 is 133 amino acid residues and is presented using the one-letter code in Table 2B. The NOV2 protein was analyzed for signal peptide prediction and cellular localization. SignalP, Psort and Hydropathy results predict that NOV2 does not appear to contain a predicted signal peptide and that NOV2 is likely to be localized in the cytoplasm with a certainty of 0.4500. NOV2 has a molecular weight of 14849.6 Daltons.

TABLE 2B
Encoded NOV2 Protein Sequence (SEQ ID NO:8).

MTQANPRPRASLQCLGPVSPLVAPSDCGGHYTDEYGRIFNYAGPKTECVWIIELNPGEIVTVAIPDLKFACG
KEYVEVLDGPPGSESLDRICKAFSTFYYSSSNIITIKYSREPSHPPTFFEIYYFVDAWSTH

The full amino acid sequence of the protein of the invention was found to have 53 of 106 amino acid residues (50%) identical to, and 66 of 106 residues (62%) positive with, the 111 amino acid residue Spermadhesin AQN-1 protein from Sus scrofa (ptnr: SWISSPROT-ACC:P26322). The global sequence homology (as defined by GAP global sequence alignment with the full length sequence of this protein) is 54% amino acid similarity and 48% amino acid identity. In addition, NOV2 contains (as defined by Interpro) a CUB protein domain at amino acid positions 27 to 125.

It was also found that NOV2 had homology to the amino acid sequences shown in the BLASTP data listed in Table 2C.

TABLE 2C
BLAST results for NOV2

| Gene Index/ | Protein/ | Length | Identity | Positives | |
|---|---|---|---|---|---|
| Identifier | Organism | (aa) | (%) | (%) | Expect |
| gi\|108346\|pir\| \|S239 | spermadhesin | 109 | 51/103 | 69/103 | 7e−23 |
| 42 | PSP-I [Sus | | (49%) | (66%) | |
| | scrofa] | | | | |
| gi\|108212\|pir\| \|S212 | spermadhesin | 111 | 54/106 | 66/106 | 8e−23 |
| 11 | AQN-1 | | (50%) | (61%) | |
| | protein [Sus | | | | |
| | scrofa] | | | | |
| gi\|1346879\|sp\|P3549 | MAJOR | 133 | 50/103 | 69/103 | 1e−22 |
| 5\|PSP1_PIG | SEMINAL | | (48%) | (66%) | |
| | PLASMA | | | | |
| | GLYCOPROTEIN | | | | |
| | PSP-I | | | | |
| | PRECURSOR | | | | |
| | (SP3) | | | | |
| gi\|114082\|sp\|P26322 | CARBOHYDRATE- | 111 | 53/106 | 66/106 | 2e−22 |
| \|AQN1_PIG | BINDING | | (50%) | (62%) | |
| | PROTEIN AQN-1 | | | | |
| | (ZONA | | | | |
| | PELLUCIDA- | | | | |
| | BINDING | | | | |
| | PROTEIN AQN- | | | | |
| | 1) | | | | |
| | (SPERMADHESIN | | | | |
| | AQN- 1) | | | | |
| gi\|543110\|pir\| \|S394 | spermadhesin | 109 | 50/103 | 69/103 | 1e−22 |
| 33 | PSP-I [Sus | | (48%) | (66%) | |
| | scrofa] | | | | |

The homology of these sequences is shown graphically in the ClustalW analysis shown in Table 2D.

[image]

[image]

Table 2E lists the domain description from DOMAIN analysis results against NOV2. This indicates that the NOV2 sequence has properties similar to those of other proteins known to contain this domain.

[image]

Spermadhesins are a novel family of secretory proteins expressed in the male genital tract of pig, horse and bull (Topfer-Petersen, 1998). They are major products of the seminal plasma and have been found to be peripherally associated to the sperm surface. The structure and function of spermadhesins have been thoroughly investigated in the pig, which exhibits the greatest diversity of members: AWN, AQN-1, AQN-2, PSP-I and PSP-II and its glycosylated isoforms. They are multifunctional proteins showing a range of ligand-binding abilities, e.g. carbohydrates, sulfated glycosaminoglycans, phospholipids and protease inhibitors, suggesting that they may be involved in different steps of fertilization. Isolated porcine spermadhesins bind the zona pellucida glycoproteins in a cation-dependent manner with a Kd in a low micromolar range, and AWN, AQN-1 and AQN-3 display similar binding affinity for glycoproteins containing Gal beta(1-3)-GalNAc and Gal beta(1-4)-GlcNAc sequences in O-linked and N-linked oligosaccharides, respectively.

Spermadhesins belong to the superfamily of proteins with a CUB domain. Several disorders which may be linked to CUB domain containing proteins. Megaloblastic anemia and neurologic disturbances are common symptoms of deficiency of the coenzyme vitamin B12 (cyanocobalamin). The cellular uptake of the vitamin and its modified forms depends on the binding to the carrier proteins, intrinsic factor (IF) produced in the stomach, and transcobalamin, present in the circulation and various tissue fluids. Hereditary forms of cobalamin deficiency are known to relate to qualitatively abnormal IF, to decreased synthesis of transcobalamin, and to a defect of the intestinal epithelium leading to decreased uptake of IF-cobalamin and failure to absorb cobalamin (Imerslund-Grasbeck disease (IGS), or megaloblastic anemia-1 (MGA1). Imerslund-Grasbeck disease has been shown by linkage studies to be caused by mutation in a region designated MGA1 (megaloblastic anemia-1), located on 10p between markers D10S548 and D10S466. The defect has been thought to be related to abnormal epithelial translocation of cobalamin, perhaps due to decreased receptor function/expression.

Studies in rodents showed that uptake of cobalamin in complex with IF is facilitated by an intestinal 460-kD protein (Birn et al., 1997; Seetharam et al., 1997), designated cubilin (Moestrup et al., 1998). Cubilin is suggested to traffic by means of megalin (LRP2), a 600-kD endocytic receptor expressed in the same tissues and mediating uptake of a number of ligands, including transcobalamin-cobalamin complexes. Like megalin, cubilin has a significantly higher expression in the renal proximal tubules compared with the intestine, and, because IF is only present in minute amounts in nongastrointestinal tissues, cubilin might also have multiligand properties. Cubilin binds receptor-associated protein (RAP), a 40-kD endoplasmic reticulum protein also binding with high affinity to the multiligand giant receptors (e.g., megalin) belonging to the low density lipoprotein receptor protein family. RAP may function as a chaperone during folding of the receptors. Moestrup et al. (1998) determined the primary structure of rat cubilin and showed that almost the entire sequence is accounted for by a cluster of 8 epidermal growth factor (EGF) repeats, followed by a large cluster of 27 CUB domains which led to the designation of the receptor.

By surface plasmon resistance analysis of ligand-affinity-purified human cubilin, Kozyraki et al. (1998) demonstrated a high affinity calcium- and cobalamin-dependent binding of IF-cobalamin. Complete cDNA cloning of the human receptor showed a 3,597-amino acid peripheral membrane protein with 69% identity to rat cubilin. Amino-terminal sequencing of the receptor indicated that the cDNA sequence encodes a precursor protein undergoing proteolytic processing due to cleavage at a recognition site (arg-7/glu-8/lys-9/arg-10) for the trans-Golgi proteinase furin. Using fluorescence in situ hybridization, radiation hybrid mapping, and screening of YAC clones, Kozyraki et al. (1998) mapped the human cubilin gene between markers D10S 1661 and WI-5445 on the short arm of chromosome 10. This was within the 6-cM region harboring the gene responsible for megaloblastic anemia-1 (MGA1). All of this was considered circumstantial evidence that an impaired synthesis, processing, or ligand binding of cubilin is the molecular basis of Imerslund-Grasbeck disease.

Megaloblastic anemia-1 is a rare, autosomal recessive disorder characterized by juvenile megaloblastic anemia, as well as neurologic symptoms that may be the only manifestations. At the cellular level, MGA1 is characterized by selective intestinal B12 malabsorption. MGA1 occurs worldwide, but its prevalence is higher in several Middle Eastern countries and in Norway, and highest in Finland (0.8 in 100,000). Aminoff et al. (1995) mapped the MGA1 locus by linkage analysis in Finnish and Norwegian families to a 6-cM region on 10p12.1. As the receptor for intrinsic factor-B12 complex (IF-B12), the CUBN gene is a logical candidate for the site of the mutation and is also a positional candidate because it maps to the same region. Aminoff et al. (1999) refined the MGA1 region by linkage disequilibrium (LD) mapping, fine-mapped the CUBN gene in 17 Finnish MGA1 families, and identified 2 independent disease-specific CUBN mutations.

Although cubilin is the intestinal receptor for the endocytosis of intrinsic factor-vitamin B12, several lines of evidence, including a high expression in kidney and yolk sac, indicated that it may have additional functions. Using cubilin affinity chromatography, Kozyraki et al. (1999) isolated apolipoprotein A-I (APOA1), the main protein of high density lipoprotein (HDL). They demonstrated a high-affinity binding of APOA1 and HDL to cubilin, and cubilin-expressing yolk sac cells showed efficient endocytosis of iodine-labeled HDL that could be inhibited by IgG antibodies against APOA1 and cubilin. The physiologic relevance of the cubilin-APOA1 interaction was further emphasized by urinary APOA 1 loss in some known cases of functional cubilin deficiency (Imerslund-Grasbeck syndrome). Therefore, cubilin is a receptor in epithelial APOA1/HDL metabolism.

Based upon sequence similarity of the NOV2 polypeptide to the “Spermadhesin family” of proteins, the NOV2 nucleic acids and proteins are useful in potential therapeutic applications implicated in (but not limited to) various pathologies and disorders such as infertility and other reproductive disorders and also birth control and/or other pathologies and disorders. For example, a cDNA encoding the Spermadhesin-like protein may be useful in gene therapy, and the Spermadhesin-like protein may be useful when administered to a subject in need thereof. By way of nonlimiting example, the compositions of the present invention will have efficacy for treatment of patients suffering from infertility and other reproductive disorders and for development of a birth control composition. The NOV2 nucleic acid, or fragments thereof, may further be useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods.

The NOV2 nucleic acid, or fragments thereof, may further be useful in diagnostic applications, wherein the presence or amount of the nucleic acid or the protein are to be assessed. NOV2 nucleic acids and polypeptides are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. The disclosed NOV2 proteins have multiple hydrophilic regions, each of which can be used as an immunogen. In one embodiment, a contemplated NOV2 epitope is from about amino acids 8 to 12. In another embodiment, a NOV2 epitope is from about amino acids 25 to 50. In additional embodiments, NOV2 epitopes are from amino acids 70 to 90 and from amino acids 100 to 125. These novel proteins can be used in assay systems for functional analysis of various human disorders, which are useful in understanding of pathology of the disease and development of new drug targets for various disorders.

NOV3

A NOV3 nucleic acid of 2220 nucleotides (also referred to as GM_AC036220_A) encoding a novel disintegrin-like protein is shown in Table 3A. An open reading frame was identified beginning with an ATG initiation codon at nucleotides 4-6 and ending with a TAA codon at nucleotides 2215-2117. A putative untranslated region upstream from the initiation codon and downstream from the termination codon is underlined in Table 3A, and the start and stop codons are in bold letters.

TABLE 3A
NOV3 Nucleotide Sequence (SEQ ID NO:9)

GACATGAGGCAGGCAGAGGCGCGGGTCACCCTTAGGGCCCCCCTCTTGCTGCTGGGGCTCTGGGTGCTCC
TGACTCCAGTCCGGTGTTCTCAAGGCCATCCCTCGTGGCACTACGCATCCTCCAAGGTGGTGATTCCCAG
GAAGGAGACGCACCACGGCAAAGACCTTCAGTTTCTGGGCTGGCTGTCCTACAGCCTGCATTTTGGGGGT
CAAAGACACATCATTCACATGCGGAGGAAACACCTTCTTTGGCCCAGACATCTGCTGGTGACAACTCAGG
ATGACCAAGGAGCCTTGCAGATGGATGACCCCTACATCCCTCCAGACTGCTACTATCTCAGCTACCTGGA
GGAGGTTCCTCTGTCCATGGTCACCGTGGACATGTGCTGTGGGGGCCTCAGAGGCATCATGAAGCTGGAC
GACCTTGCCTATGAAATCAAACCCCTCCAGGATTCCCGCAGGCTTGAACATGTTTCTCAGATAGTGGCCG
AGCCCAACGCAATGGGGCCCACATTTAGAGATGGTGACAATGAGGAGACAAACCCCCTGTTCTCTGAAGC
AAATGACAGCATGAATCCCAGGATATCTAATTGGCTGTATAGTTCTCATAGAGGCAATATAAAAGGCCAC
GTTCAATGTTCCAATTCATATTGTCGTGTAGATGACAATATTACAACTTGTTCCAAGGAGGTGGTCCAGA
TGTTCAGTCTCAGTGACAGCATTGTTCAAAATATTGATCTGCGGTACTATATTTATCTTTTGACCATATA
TAATAATTGTGACCCAGCCCCTGTGAATGACTATCGAGTTCAGAGTGCAATGTTTACCTATTTTAGAACA
ACCTTTTTTGATACTTTTCGTGTTCATTCACCCACACTACTTATTAAAGACGCACCACATGAATGTAACT
ATGAACCACAAAGGTATAGCTTCTGTACACATTTAGGCCTATTACACATTGGTACTCTAGGCAGACATTA
TTTATTAGTAGCCGTCATAACAACCCAGACACTGATGAGAAGTACTGTGAGAAGTACTGGTGATGATAAC
TACTGCACATGTCAGAAAAGGGCCTTCTGCATTATGCAGCAATATCCTGGGATGACAGATGCGTTCAGTA
ACTGTTCTTATGGACATGCACAAAATTGTTTTGTACATTCAGCCCGGTGTGTTTTCAAAACACTTGCTCC
TGTGTATAATGAAACCTGTCTCTTTTACCACTGTCAATCTTTTCTGTTGCTTTCTGAATCTACAAAATAC
AATGATGTGTGTTCAATGCCCTCGCATCACCCATGTTATTCTCGGTCACTCTGTGGGGGAAGCTGTCGAA
GGACACCAGGAGCAATCTGTCATATAGGAGAGTGCTGTACAAACTGCAGCTACTCCCCACCAGGGACTCT
CTGCAGACCTATCCAAAATATATGTGACCTTCCAGAGTACTGTCACGGGACCACCGTGACATGCCCCGCA
AACTTTTATATGCAAGATGGAACCCCGTGCACTGAAGAAGGCTACTGCTATCATGGGAACTGCACTGACC
GCAATGTGCTCTGCAAGGTAATCTTTGGTGTCAGTGCTGAGGAGGCTCCTGAGGTCTGCTATGACATAAA
TCTTGAAAGTTACCGATTTGGACATTGTACTCGACGACAAACAGCTCTCAACAACCAGGCTTGTGCAGGA
ATAGATAAGTTTTGTGGAAGACTGCAGTGTACCAGTGTGACCCATCTTCCCCGGCTGCAGGAACATGTTT
CATTCCATCACTCAGTGACAGGAGGATTTCAGTGTTTTGGACTGGATGACCACCGTGCAACAGACACAAC
TGATGTTGGGTGTGTGATAGATGGCACTCCTTGTGTTCATGGAAACTTCTGTAATAACACCAGGTGCAAT
GCGACTATCACTTCACTGGGCTATGACTGTCGCCCTGAGAAGTGCAGTCATAGAGGGGTGTGCAACAACA
GAAGGAACTGCCATTGCCATATAGGCTGGGATCCTCCACTGTGCCTAAGAAGAGGTGCTGGTGGGAGTGT
CGACAGCGGGCCACCTCCAAAAATAACACGTTCGGTCAAACAAAGCCAACAATCAGTGATGTATCTGAGA
GTGGTCTTTGGTCGTATTTACACCTTCATAATTGCACTGCTCTTTGGGATGGCCACAAATGTGAGAACTA
TCAGGACCACCACTGTTAAGGGATGGACAGTTACTAACCCTGAATAAGAC

A NOV3 nucleic acid was identified on chromosome 4 by TblastN using CuraGen Corporation's sequence file for disintegrin or homolog as run against the Genomic Daily Files made available by GenBank or from files downloaded from the individual sequencing centers. The nucleic acid sequence was predicted from the genomic file Genbank accession number:AC036220 by homology to a known disintegrin or homolog. Exons were predicted by homology and the intron/exon boundaries were determined using standard genetic rules. Exons were further selected and refined by means of similarity determination using multiple BLAST (for example, tBlastN, BlastX, and BlastN) searches, and, in some instances, GeneScan and Grail. Expressed sequences from both public and proprietary databases were also added when available to further define and complete the gene sequence. The DNA sequence was then manually corrected for apparent inconsistencies thereby obtaining the sequences encoding the full-length protein. Thus, regions 49682-48476, 49991-50101, 17895-17291, 59253-60129 were spliced from AC036220, and then the sequence was edited by removing a G from the end of GAGAAGTACTGG, removing N from the end of AGCTACTCCN and changing a T at the end of AAATGTGT to an A to remove an in-frame stop codon.

A search of sequence databases found that the NOV3 nucleic acid sequence has 1113 of 1206 bases (92%) identical to a Macaca fascicularis metalloprotease-like, disintegrin-like mRNA (GENBANK-ID: MFTMDCIVA|acc:X87205) and in a second segment has 872 of 996 bases (87%) identical to this mRNA. The nucleic acid sequence has 528 of 881 bases (59%) identical, and in a second segment has 376 of 581 bases (64%) identical, to a Homo sapiens metalloprotease-disintegrin (ADAM20) mRNA (GENBANK-ID: AF158643|acc:AF158643)(E=1.3 e−48).

The disclosed NOV3 polypeptide (SEQ ID NO:10) encoded by SEQ ID NO:9 is 737 amino acid residues and is presented using the one-letter code in Table 3B. The NOV3 protein was analyzed for signal peptide prediction and cellular localization. SignalP, Psort and Hydropathy results predict that NOV3 has a signal peptide with most likely cleavage site pos. 31 and 32, at: SQG-HP, and that NOV3 is likely to be localized in the plasma membrane with a certainty of 0.4600. NOV3 has a molecular weight of 83311.0 Daltons.

TABLE 3B
Encoded NOV3 Protein Sequence (SEQ ID NO:10).

MRQAEARVTLRAPLLLLGLWVLLTPVRCSQGHPSWHYASSKVVIPRKETHHGKDLQFLGWLSYSLHFGGQR
HIIHMRRKHLLWPRHLLVTTQDDQGALQMDDPYIPPDCYYLSYLEEVPLSMVTVDMCCGGLRGIMKLDDLA
YEIKPLQDSRRLEHVSQIVAEPNAMGPTFRDGDNEETNPLFSEANDSMNPRISNWLYSSHRGNIKGHVQCS
NSYCRVDDNITTCSKEVVQMFSLSDSIVQNIDLRYYIYLLTIYNNCDPAPVNDYRVQSAMFTYFRTTFFDT
FRVHSPTLLIKEAPHECNYEPQRYSFCTHLGLLHIGTLGRHYLLVAVITTQTLMRSTVRSTGDDNYCTCQK
RAFCIMQQYPGMTDAFSNCSYGHAQNCFVHSARCVFKTLAPVYNETCLFYHCQSFLLLSESTKYNDVCSMP
SHHPCYSRSLCGGSCRRTPGAICHIGECCTNCSYSPPGTLCRPIQNICDLPEYCHGTTVTCPANFYMQDGT
PCTEEGYCYHGNCTDRNVLCKVIFGVSAEEAPEVCYDINLESYRFGHCTRRQTALNNQACAGIDKFCGRLQ
CTSVTHLPRLQEHVSFHHSVTGGFQCFGLDDHRATDTTDVGCVIDGTPCVHGNFCNNTRCNATITSLGYDC
RPEKCSHRGVCNNRRNCHCHIGWDPPLCLRRGAGGSVDSGPPPKITRSVKQSQQSVMYLRVVFGRIYTFII
ALLFGMATNVRTIRTTTVKGWTVTNPE

A BLASTX search was performed against public protein databases (GenBank and/or GenSeq). The full amino acid sequence of the protein of the invention was found to have 591 of 737 amino acid residues (80%) identical to, and 640 of 737 residues (80%) positive with, the 732 amino acid residue Testicular Metalloprotease-like, Disintegrin-like, Cysteine-rich Protein IVA protein from Macaca fascicularis (ptnr: SPTREMBL-ACC:Q28484). The global sequence homology (as defined by FASTA alignment with the full length sequence of this protein) is 83% amino acid homology and 81% amino acid identity. In addition, NOV3 contains the following protein domains (as defined by Interpro). The NOV3 protein sequence has a reprolysin (M12B) family zinc metalloprotease domain at amino acid positions 75 to 190, a metalloprotease (ADAM type)/reprolysin (M12B) family domain at amino acid positions 213-394, a disintegrin domain at amino acid positions 440-493, a metallothionein domain at amino acid positions 603-660, and a EGF-like domain at amino acid positions 639-667.

It was also found that NOV3 had homology to the amino acid sequences shown in the BLASTP data listed in Table 3C.

TABLE 3C
BLAST results for NOV 3

| Gene Index/ | Protein/ | Length | Identity | Positives | |
|---|---|---|---|---|---|
| Identifier | Organism | (aa) | (%) | (%) | Expect |
| gi\|2134533\|pir\|\|I | testicular | 732 | 546/713 | 595/713 | 0.0 |
| 52361 | Metalloprotease | | (76%) | (82%) | |
| | -like, | | | | |
| | Disintegrin- | | | | |
| | like, Cysteine- | | | | |
| | rich protein | | | | |
| | IVa [Macace | | | | |
| | fascicularis] | | | | |
| gi\|7512247\|pir\|\|I | testicular | 713 | 550/713 | 601/713 | 0.0 |
| 65253 | Metalloprotease | | (77%) | (84%) | |
| | -like, | | | | |
| | Disintegrin- | | | | |
| | like, Cysteine- | | | | |
| | rich protein | | | | |
| | IVb [Macace | | | | |
| | fascicularis] | | | | |
| gi\|2228590\|gb\|AAC | cellular | 731 | 380/715 | 480/715 | 0.0 |
| 09475.1\| | disintegrin | | (53%) | (66%) | |
| (U82750) | ADAM 6d; | | | | |
| | tMDCIVd | | | | |
| | [Oryctolagus | | | | |
| | cuniculus] | | | | |
| gi\|2228592\|gb\|AAC | cellular | 730 | 376/715 | 483/715 | 0.0 |
| 09476.1\| | disintegrin | | (52%) | (66%) | |
| (U82751) | ADAM 6e; | | | | |
| | tMDCIVe | | | | |
| | [Oryctolagus | | | | |
| | cuniculus] | | | | |
| gi\|1657402\|emb\|CA | tMDC IV [Rattus | 751 | 352/704 | 453/704 | 0.0 |
| A70328.1\| | norvegicus] | | (50%) | (64%) | |
| (Y09111) | | | | | |

The homology of these sequences is shown graphically in the ClustalW analysis shown in Table 3D.

[image]

[image]

Tables 3E -3H list the domain description from DOMAIN analysis results against NOV3. This indicates that the NOV3 sequence has properties similar to those of other proteins known to contain this domain.

[image]

[image]

[image]

Fertilin alpha and beta, previously known as PH-30 alpha and beta, are two subunits of a guinea pig sperm integral membrane protein implicated in sperm-egg binding and fusion. These proteins are derived from sequence-similar precursors which contain a metalloprotease-like and a disintegrin-like domain and which are related to a family of metalloprotease and disintegrin domain-containing snake venom proteins. Wolfsberg et al (1995) reported the cloning, sequencing, and characterization of mouse fertilin alpha and beta as well as five additional sequence-similar cDNAs from guinea pig and mouse testis. This gene family was named ADAM, for proteins containing A disintegrin And Metalloprotease domain. In situ hybridization demonstrated that, in testis, RNA encoding these ADAMs is expressed only in spermatogenic cells and that this expression is developmentally regulated. Some ADAMs (e.g., fertilin alpha) have the consensus active-site sequence for a zinc-dependent metalloprotease in their metalloprotease-like domain. All have a disintegrin-like domain, which could bind integrins or other receptors. Some have sequences which may be active in membrane fusion. All encode potential membrane-spanning domains.

Members of the ADAM protein family are type I integral membrane proteins containing a signal peptide followed by proprotein, zinc metalloprotease, disintegrin, transmembrane, and cytoplasmic regions. In mammals, ADAM1 and ADAM2, also known as fertilin-alpha and -beta, respectively, are expressed as heterodimers on the posterior head of spermatocytes and play a role in oocyte adhesion and fusion. The human ADAM1 homolog, however, is nonfunctional. By RT-PCR of B-cell RNA with degenerate oligonucleotide primers based on the conserved zinc-binding and disintegrin domains of ADAM proteins, Hooft van Huijsduijnen (1998) isolated a partial cDNA encoding ADAM20. Sequence analysis of the cDNAs indicated that the 5-prime untranslated region of the ADAM20 mRNA is alternatively spliced. The predicted 726-amino acid ADAM20 protein contains a consensus zinc-binding site of metalloproteases, and both ADAM20 and ADAM21 have putative cell-fusion peptides required for sperm-egg fusion. Overall, the 2 proteins are 50% identical and share sequence similarity with ADAM9 and the fertilins. By analysis of a radiation hybrid panel, Hooft van Huijsduijnen (1998) mapped both the ADAM20 and the ADAM21 genes to 14q24.1. The exclusive expression of ADAM20 and ADAM21 in human testis and their sequence similarity with the fertilins suggested that they too are expressed on sperm cells and are involved in sperm maturation and/or fertilization. Hooft van Huijsduijnen (1998) proposed that ADAM20 and/or ADAM21 is the functional equivalent of sperm fertilin-alpha, since that gene is nonfunctional in human.

Members of the metalloproteinase-like, disintegrin-like, and cysteine-rich (MDC) protein family have been implicated in cell-cell or cell-extracellular matrix interactions. Rearrangements in both tumors involved multiple exons and disrupted the coding region of the gene. A number of sequence-related, cysteine-rich proteins containing metalloprotease-like and disintegrin-like domains (the MDC protein family), and have been shown to play a role in egg recognition during fertilization and play a more general role in integrin-mediated cell-cell recognition, adhesion or signalling.

Based upon sequence similarity of the NOV3 polypeptide to the “disintegrin family”, NOV3 nucleic acids and proteins are useful in potential therapeutic applications implicated in (but not limited to) various pathologies and disorders as indicated below. The potential therapeutic applications for NOV3 include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV3 nucleic acids and proteins of the invention are useful in potential therapeutic applications implicated in reproductive disorders, early developmental disorders, various cancers including but not limited to breast and ovarian cancers, and/or other pathologies and disorders. For example, a cDNA encoding the NOV3 protein may be useful in gene therapy, and the disintegrin-like protein may be useful when administered to a subject in need thereof. By way of nonlimiting example, the compositions of the present invention will have efficacy for treatment of patients suffering from reproductive disorders, early developmental disorders, various cancers including but not limited to breast and ovarian cancers.

The NOV3 novel nucleic acids, or fragments thereof, may further be useful in diagnostic applications, wherein the presence or amount of the nucleic acid or the protein are to be assessed. NOV3 nucleic acids and polypeptides are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. The disclosed NOV3 protein has multiple hydrophilic regions, each of which can be used as an immunogen. These NOV3 proteins can be used in assay systems for functional analysis of various human disorders, which will help in understanding of pathology of the disease and development of new drug targets for various disorders.

NOV4

A NOV4 nucleic acid of 1358 nucleotides (also referred to as GMAC023790_A) encoding a novel 5-hydroxytryptamine-7 receptor-like protein is shown in Table 4A. An open reading frame was identified beginning with an ATG initiation codon at nucleotides 5-7 and ending with a TGA codon at nucleotides 1349-1351. A putative untranslated region upstream from the initiation codon and downstream from the termination codon is underlined in Table 4A, and the start and stop codons are in bold letters.

TABLE 4A
NOV4 Nucleotide Sequence (SEQ ID NO:11).

AGTGATGGATGTTAACAGCAGCGGCCACCCGGACCTCTACGGGCGCCTCTGCTCTTTCCTCCTGCCGGAGG
TGGGGGGCAGGCTGCCCGACCTGAGCCCCGACGGTGGCGCCGAACCGGTCGCGGTCTCCGGGACGCCGCAT
CTGCTGAGCGAGGTGACGGCCAGCCCGGCGCCCACCTGGGACGCAACCCCGGGCAATGCCTCCGGCCGCGG
GGAGCAAATCAATCAAGAAAGGGCCGAGAAAGTTGTGATCGGCTCTGTCCTGACGCTCATCTCTCTGTCTG
CGATCGCGGGCAACTGCCTGGTGGTAATCTCTGTGTGCTTCGTCAAGAAGCTCCGCCAGCCCTCCAACTAC
CTCATCGTGTCCATGGCGCTGGCCAACCTCTCGGTGGCCATGGCGGTCATGCCCTTCATCAGTGTCACCGA
CCTCATCGGGGGCAAGTGGATCTTTGGACACTTTTTCTGTAACGTCTTCTCCGTGAATGTCATGTGCTGCA
CGGCCTGGATCTTGACCTTGTACGTGATCAGCATCGACAGGGACCTTGGGATCATGAAGCCTCTCACGTAC
CCTATGAGGCAGAAGGGGAAATGCATGACGAAGATGATTCTTTCTGTCTGCCTTCTTTCCGCCTTTGTCAC
TTTACCTACCATTTTTGGTCGGGCTCAGAATGTAAACGATGATAAGGTGTGCTTGGTCAATCAAGACTTTG
GCTACACGATTTACTCCCCGCATTTGGCAGCATTTATCCCCATGTGCGTCATGCTTTTCATGTACTATCAG
ATTTACAAGGCCGCCAGGAAAAGCGCGGCCAAACACAGGTTACCTGGCTTCCCTCGAGTGGAGCCAGACAG
CGTAGTCACCCTGAATGGCACAGTGAAGTTCCAGGAGGTGGAAGAGTGTGCAAACCTTTCGAGACTCCTCA
AGCATGAAAGGAAAAATATCTCCATCTTTAAGCGGAAACAGAAAGCAGCGACTACCTTGGGGATCATCGTC
TGGGCCTCCACCATGTGCTGGCCGCCCTTTTTCCTCCTGACAGCCAGACCCTTCTGTCTATGGCACTGCCC
TTCTGTCTATGGCACTGCCTGCAGCTGCATCCCACTGTGGGTGGAGAGGATATTTCCATGGCTGGGCTATG
CAAACTCTCTCATTAACCCTTTTATTTATGCCTTCTTCAACTGGGACCTGAGGACCACCTATTGCAGCCGG
CTCCAGTGCCAGTACCAGAATATCAACCAGACACTCTCAGCTGCAGGCATGCATGAAGCCCTGAAGCTTGC
TGAGAGGCCAGAGAGACCTGAGTTTGTCCTACAAAACTCTGACTACTGTAGAAAAAAAAGTCATGATTCAT
GACTGAAAG

A NOV4 nucleic acid was identified by TblastN using CuraGen Corporation's sequence file for 5-hydroxytryptamine-7 receptor or homolog as run against the Genomic Daily Files made available by GenBank or from files downloaded from the individual sequencing centers. The nucleic acid sequence was predicted from the genomic file Genbank ACCNO: AC023790_A by homology to a known 5-hydroxytryptamine-7 receptor or homolog. Exons were predicted by homology and the intron/exon boundaries were determined using standard genetic rules. Exons were further selected and refined by means of similarity determination using multiple BLAST (for example, tBlastN, BlastX, and BlastN) searches, and, in some instances, GenScan and Grail. Expressed sequences from both public and proprietary databases were also added when available to further define and complete the gene sequence. The DNA sequence was then manually corrected for apparent inconsistencies thereby obtaining the sequences encoding the full-length protein.

In a search of sequence databases, it was found, for example, that the nucleic acid sequence has, in one fragment, 1023 of 1080 bases (94%) identical, and in a second fragment, has 311 of 328 bases (94%) identical, to a Homo sapiens, serotonin-7 receptor pseudogene RNA (GENBANK-ID: HSU86813)(E=8.6 e−269). This 94% similarity of the gene of the present invention to a public EST sequence strongly suggests that the current invention represents an expressed gene.

The NOV4 polypeptide (SEQ ID NO: 12) encoded by SEQ ID NO: 11 is 448 amino acid residues and is presented using the one-letter amino acid code in Table 4B. The NOV4 protein was analyzed for cellular localization. Psort analysis predicts the protein of the invention to be localized in the plasma membrane with a certainty of 0.6000. NOV4 has a molecular weight of 49974.9 Daltons.

TABLE 4B
NOV4 Protein Sequence (SEQ ID NO:12)

MDVNSSGHPDLYGRLCSFLLPEVGGRLPDLSPDGGAEPVAVSGTPHLLSEVTASPAPTWDATPGNASGRGEQIN
QERAEKVVIGSVLTLISLSAIAGNCLVVISVCFVKKLRQPSNYLIVSMALANLSVAMAVMPFISVTDLIGGKWI
FGHFFCNVFSVNVMCCTAWILTLYVISIDRDLGIMKPLTYPMRQKGKCMTKMILSVCLLSAFVTLPTIFGRAQN
VNDDKVCLVNQDFGYTIYSPHLAAFIPMCVMLFMYYQIYKAARKSAAKHRLPGFPRVEPDSVVTLNGTVKFQEV
EECANLSRLLKHERKNISIFKRKQKAATTLGIIVWASTMCWPPFFLLTARPFCLWHCPSVYGTACSCIPLWVER
IFPWLGYANSLINPFIYAFFNWDLRTTYCSRLQCQYQNINQTLSAAGMHEALKLAERPERPEFVLQNSDYCRKK
SHDS

The full amino acid sequence of the protein of the invention was found to have 369 of 449 amino acid residues (82%) identical to, and 397 of 449 residues (88%) positive with, the 445 amino acid residue 5-hydroxytryptamine-7-receptor protein from Homo Sapiens (ptnr:SPTREMBL-ACC:_P34969; E=4.5 e−192).

It was also found that NOV4 had homology to the amino acid sequences shown in the BLASTP data listed in Table 4C.

TABLE 4C
BLAST results for NOV 4

| Gene Index/ | | Length | Identity | Positives | |
|---|---|---|---|---|---|
| Identifier | Protein/ Organism | (aa) | (%) | (%) | Expect |
| gi\|4504547\|ref\|NP_0 | 5- | 445 | 368/451 | 396/451 | 0.0 |
| 00863.1\| | hydroxytryptamine | | (818) | (87%) | |
| | receptor 7, | | | | |
| | isoform a; | | | | |
| | serotonin 5-HT-7 | | | | |
| | receptor [Homo | | | | |
| | sapiens] | | | | |
| gi\|10880129\|ref\|NP_{—} | 5- | 479 | 357/439 | 385/439 | 0.0 |
| 062873.1\| | hydroxytryptamine7 | | (81%) | (87%) | |
| | receptor isoform | | | | |
| | d; serotonin 5-HT- | | | | |
| | 7 receptor [Homo | | | | |
| | sapiens] | | | | |
| gi\|10880131\|ref\|NP_{—} | 5- | 432 | 357/438 | 384/438 | 0.0 |
| 062874.1\| | hydroxytryptamine | | (81%) | (87%) | |
| | receptor 7, | | | | |
| | isoform b; | | | | |
| | serotonin 5-HT-7 | | | | |
| | receptor [Homo | | | | |
| | sapiens] | | | | |
| gi\|461440\|sp\|P32305 | 5- | 448 | 357/454 | 389/454 | 0.0 |
| \|5E7_RAT | HYDROXYTRYPTAMINE | | (78%) | (85%) | |
| | 7 RECEPTOR (5-HT- | | | | |
| | 7) (5-HT-X) | | | | |
| | (SEROTONIN | | | | |
| | RECEPTOR) (5HT7) | | | | |
| | (GPRFO) | | | | |
| gi\|543452\|pir\|\|S406 | serotonin receptor | 448 | 353/454 | 384/454 | 0.0 |
| 87 | 7 [Rattus | | (77%) | (83%) | |
| | norvegicus] | | | | |

The homology of these sequences is shown graphically in the ClustalW analysis shown in Table 4D.

[image]

[image]

Table 4E lists the domain description from DOMAIN analysis results against NOV4. This indicates that the NOV4 sequence has properties similar to those of other proteins known to contain this domain.

[image]

[image]

Other BLAST results include sequences from the Patp database, which is a proprietary database that contains sequences published in patents and patent publications. Patp results include those listed in Table 4F.

TABLE 4F
Patp alignments of NOV4

| | | | Smallest |
|---|---|---|---|
| | | | Sum |
| | Reading | High | Prob. |
| Sequences producing High-scoring Segment Pairs: | Frame | Score | P(N) |
| Patp:AAR54782 Human brain serotonin receptor [Homo sap], 445 aa | +2 | 1869 | 4.4e−192 |
| Patp:AAR57200 Rat 5HT6 receptor [Rattus rat], 448 aa | +2 | 1797 | 1.9e−184 |
| patp:AAR58689 Rat REC20 serotonin receptor [Rattus rat], 435 aa | +2 | 1742 | 1.2e−187 |
| patp: AAW56756 Serotonin 5HT7 receptor allelic. [Homo sap], 345 aa | +2 | 1423 | 7.9e−145 |

The neurotransmitter serotonin (5-hydroxytryptamine; 5-HT) exerts a wide variety of physiologic functions through a multiplicity of receptors and may be involved in human neuropsychiatric disorders such as anxiety, depression, or migraine. These receptors consist of 4 main groups, 5-HT-1, 5-HT-2, 5-HT-3, and 5-HT-4, 5-HT7 subdivided into several distinct subtypes on the basis of their pharmacologic characteristics, coupling to intracellular second messengers, and distribution within the nervous system (Zifa and Fillion, 1992). The serotonergic receptors belong to the multi5-Hydroxytryptamine Receptor family of receptors coupled to guanine nucleotide-binding proteins.

The 5-HT7 receptor, predominantly localized in the hypothalamus, hippocampus, and frontal cortex, stimulates cyclic AMP formation and is thought to be involved in the regulation of sleep-wake cycles and predisposition to behavioral problems (Pesonen et al., 1998). Sleight et al. (1995) demonstrated that in rat hypothalamus, 5-HT7 receptors are down regulated in response to treatment with the antidepressant, fluoxetine. The glucocorticoid, dexamethasone has also been shown to decrease the expression of the 5-HT7 receptor gene in rat frontocortical astrocytes (Shimizu et al., 1997). These results indicate that the 5-HT7 receptor may be a potent neuromodulator suggesting therapeutic implications for corticosteroid therapy, as well as sleep, and behavioral disorders.

Based on primary and secondary structural similiarity of NOV4 polypeptides to the “serotonin receptor family”, the NOV4 nucleic acids and proteins identified are useful in potential therapeutic applications implicated in (but not limited to) various pathologies and disorders as indicated below. The potential therapeutic applications for NOV4 include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue re generation in vivo and in vitro of all tissues and cell types composing (but not limited to) those defined here.

The NOV4 nucleic acids and proteins of the invention are useful in potential therapeutic applications implicated in seizures, Alzheimer's disease, sleep disorders, appetite disorders, thermoregulation, pain perception, hormone secretion, and sexual behavior, mental depression, migraine, epilepsy, obsessive-compulsive behavior (schizophrenia), and affective disorder and/or other pathologies and disorders. By way of nonlimiting example, the compositions of the present invention will have efficacy for treatment of patients suffering from seizures, Alzheimer's disease, sleep disorders, appetite disorders, thermoregulation, pain perception, hormone secretion, and sexual behavior, mental depression, migraine, epilepsy, obsessive-compulsive behavior (schizophrenia), and affective disorder. The NOV4 nucleic acid, or fragments thereof for example, a cDNA encoding the gene-like protein may be useful in gene therapy, and the 5-hydroxytryptamine-7 receptor-like protein may be useful when administered to a subject in need thereof, may further be useful in diagnostic applications, wherein the presence or amount of the nucleic acid or the protein are to be assessed.

The NOV4 nucleic acids, or fragments thereof, may further be useful in diagnostic applications, wherein the presence or amount of the nucleic acid or the protein are to be assessed. NOV4 nucleic acids and polypeptides are further useful in the generation of antibodies that bind immunospecifically to the novel substances of the invention for use in therapeutic or diagnostic methods and other diseases, disorders and conditions of the like. NOV4 nucleic acids and polypeptides are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. For example, the disclosed NOV4 proteins have multiple hydrophilic regions, each of which can be used as an immunogen. In one embodiment, a contemplated NOV4 epitope is from about amino acids 50 to 80. In another embodiment, a NOV4 epitope is from about amino acids 175 to 200. In additional embodiments, NOV4 epitopes are from about amino acids 220 to 240, 250-275, 285-375 and from about amino acids 390 to 440. These novel proteins can be used in assay systems for functional analysis of various human disorders, which will help in understanding of pathology of the disease and development of new drug targets for various disorders.

NOV5

A NOV5 nucleic acid of 1197 nucleotides (also referred to as SC105318106_A) encoding a novel insulin growth factor binding protein-like protein is shown in Table 5A. An open reading frame was identified beginning with an ATG initiation codon at nucleotides 24-26 and ending with a TGA codon at nucleotides 870-872. A putative untranslated region upstream from the initiation codon and downstream from the termination codon is underlined in Table 5A, and the start and stop codons are in bold letters.

TABLE 5A
NOV5 Nucleotide Sequence (SEQ ID NO:13)

CCGCCGCTGTCCCGGAGCAAGCCATGCCGCGCTTGTCTCTGCTCTTGCCGCTGCTGCTTCTGC
TGCTGCTGCCGCTGCTGCCGCCGCTGTCCCCGAGCCTTGGGATCCGCGACGTGGGCGGTCGGC
GCCCCAAGTGTGGTCCGTGCCGGCCAGAGGGCTGCCCGGCGCCTGCGCCCTGCCCGGCGCCCG
GGATCTCGGCGCTCGACGAGTGCGGCTGCTGCGCCCGCTGCCTGGGAGCCGAGGGCGCGAGCT
GCGGGGGTCCGTGCCCGGGCGGGCGCTGTGGCCCCGGCCTGGTATGCGCGAGCCAGGCCGCTG
GGGCAGCGCCCGAGGGCACCGGGCTCTGCGTGTGCGCGCAGCGCGGCACCGTCTGCGGCTCCG
ACGGTCGCTCGTACCCCAGCGTCTGCGCGCTGCGCCTGCGCGCTCGGCACACGCCCCGCGCGC
ACCCCGGTCACCTGCACAAGGCGCGCGACGGCCCTTGCGAGTTCGCTCCTGTGGTCGTCGTTC
CTCCCCGAAGTGTTCACAACGTCACCGGGGCGCAGGTGGGCCTGTCCTGTGAAGTGAGGGCTG
TGCCTACCCCAGTCATCACGTGGAGAAAGGTAGTCACGAAGTCCCCTGAGGGCACCCAAGCAC
TGGAGGAGCTGCCTGGGGACCATGTCAATATAGCTGTCCAAGTGCGAGGGGGCCCTTCTGACC
ATGAGGCCACGGCCTGGATTTTGGTGAGTATCAACCCCCTGCGAAAGGAGGATGAGGGTGTGT
ACCAGTGCCATGCAGCCAACATGGTGGGAGAGGCTGAGTCCCACAGCACAGTGACGGTTCTAG
ATCTGAGTAAATACAGGAGCTTCCACTTCCCAGCTCCCGATGACCGCATGTGATGGAGAAATG
GTGTTAGAAACATTGATCATGGGATGATGGAAAAGTCAAATAACGGATCTTTGTGCTTCATGA
AGAGTTGGAAAACCTGTGTGTGTATATNACCCCTTTTGTGTGTTTTTAAAAATTATATGCAAA
CTAGATTTGTATGCAGATGTAGTTTTTAGCAGGGCAAACAGTGAGAAACGGATTTGCATGTGG
CTTTTTTATACTTTTGAAATGAATTGTTCCATGAGAAGTCTNTTTGTAATTACTCTCTTCCAG
GGAGATCACAGAATGGCATGTNTGCAATTTCGAAAGGGCTCGTGTCAGCTGTGACTCTGTACA

A NOV5 nucleic acid was identified on chromosome 9 by TblastN using CuraGen Corporation's sequence file for insulin growth factor binding protein or homolog as run against the Genomic Daily Files made available by GenBank or from files downloaded from the individual sequencing centers. The nucleic acid sequence was predicted from the genomic file Sequencing Center accession number:1 13024 by homology to a known insulin growth factor binding protein or homolog. Exons were predicted by homology and the intron/exon boundaries were determined using standard genetic rules. Exons were further selected and refined by means of similarity determination using multiple BLAST (for example, tBlastN, BlastX, and BlastN) searches, and, in some instances, GeneScan and Grail. Expressed sequences from both public and proprietary databases were also added when available to further define and complete the gene sequence. The DNA sequence was then manually corrected for apparent inconsistencies thereby obtaining the sequences encoding the full-length protein.

In a search of sequence databases, it was found, for example, that the NOV5 nucleic acid sequence has a first portion in which 824 of 1155 bases (71%) are identical (E=1.0 e−97) and a second portion in which 302 of 487 bases (62%) are identical (E=2.4 e−05), to a Mus musculus insulin growth factor binding protein mRNA (GENBANK-ID: AB006141). In a second similarity it was found that 281 of 440 bases (63%) are identical (E=5.9 e−14) to a Homo sapiens insulin-like growth factor binding protein (IGFBP) gene, exon 1 (GENBANK-ID:HUMIGFBPA1|acc:M69237).

The disclosed NOV5 polypeptide (SEQ ID NO:14) encoded by SEQ ID NO:13 is 282 amino acid residues and is presented using the one-letter code in Table 5B. The NOV5 protein was analyzed for signal peptide prediction and cellular localization. SignalP, Psort and Hydropathy results predict that NOV5 has a signal peptide with most likely cleavage site pos. 27 and 28, at: SLG-IR, and that NOV5 is likely to be localized in the endoplasmic reticulum (membrane) with a certainty of 0.8200, even though it is generally understood that known IGFBPs occur in extracellular spaces. It may be that the NOV5 protein resides in a cellular membrane, and modulates availability of IGF within a cell rather than outside the cell. NOV5 has a molecular weight of 29360.5 Daltons.

TABLE 5B
Encoded NOV5 Protein Sequence (SEQ ID NO:14)

MPRLSLLLPLLLLLLLPLLPPLSPSLGIRDVGGRRPKCGPCRPEGCPAPAPCPAPGISALDECGCCARCLGA
EGASCGGPCPGGRCGPGLVCASQAAGAAPEGTGLCVCAQRGTVCGSDGRSYPSVCALRLRARHTPRAHPGHL
HKARDGPCEFAPVVVVPPRSVHNVTGAQVGLSCEVRAVPTPVITWRKVVTKSPEGTQALEELPGDHVNIAVQ
VRGGPSDHEATAWILVSINPLRKEDEGVYQCHAANMVGEAESHSTVTVLDLSKYRSFHFPAPDDRM

The full amino acid sequence of the protein of the invention was found to have 210 of 273 amino acid residues (76%) identical to, and 233 of 273 residues (85%) positive with, the 270 amino acid residue IGBFP-like protein from Mus musculus (ptnr:SPTREMBL-ACC:BAA21725) (E=8.9 e−116). The global sequence homology (as defined by FASTA alignment with the full length sequence of this protein) is 82% amino acid homology and 77% amino acid identity. In addition, NOV5 contains (as defined by Interpro) a Kazal-type serine protease inhibitor protein domain at amino acid positions 92 to 152 and Immunoglobulin protein domain #2 at amino acid positions 170 to 249.

It was also found that NOV5 had homology to the amino acid sequences shown in the BLASTP data listed in Table 5C.

TABLE 5C
BLAST results for NOV 5

| Gene Index/ | | Length | Identity | Positives | |
|---|---|---|---|---|---|
| Identifier | Protein/ Organism | (aa) | (%) | (%) | Expect |
| gi\|9055246\|ref\|NP_0 | IGFBP-like | 270 | 172/254 | 188/254 | 1e-82 |
| 61211.1\| | protein [Mus | | (87%) | (73%) | |
| | musculus] | | | | |
| gi\|2135211\|pir\|\|I52 | Gene mac25 | 277 | 88/221 | 110/221 | 5e-30 |
| 825 | protein [Homo | | (39%) | (48%) | |
| | sapiens] | | | | |
| gi\|4504619\|ref\|NP_0 | insulin-like | 282 | 88/221 | 110/221 | 7e-30 |
| 01544.1\| | growth factor | | (39%) | (48%) | |
| | binding protein 7 | | | | |
| | [Homo sapiens] | | | | |
| gi\|6679869\|ref\|NP_0 | insulin-like | 281 | 84/216 | 107/216 | 3e-28 |
| 32074.1\| | growth factor | | (38%) | (48%) | |
| | binding protein 7 | | | | |
| | [Mus musculus] | | | | |
| gi\|1096891\|prf\|\|211 | mac25 protein | 277 | 88/221 | 110/221 | 4e-30 |
| 2365A | [Homo sapiens] | | (39%), | (48%), | |

The homology of these sequences is shown graphically in the ClustalW analysis shown in Table 5D.

[image]

Tables 5E and 5F lists the domain description from DOMAIN analysis results against NOV5. This indicates that the NOV5 sequence has properties similar to those of other proteins known to contain this domain.

[image]

Insulin-like growth factor binding proteins (IGFBPs) are soluble, extracellular proteins that bind IGF with high specificity and high affinity. Their principal functions are to regulate IGF availability in body fluids and tissues and to modulate IGF binding to its receptors. Six structurally distinct insulin-like growth factor binding proteins have been isolated and their cDNAs cloned: IGFBP1, IGFBP2, IGFBP3, IGFBP4, IGFBP5, and IGFBP6. The proteins display strong sequence homologies, suggesting that they are encoded by a closely related family of genes. The IGFBPs contain 3 structurally distinct domains each comprising approximately one-third of the molecule. The N-terminal domain 1 and the C-terminal domain 3 of the 6 human IGFBPs show moderate to high levels of sequence identity including 12 and 6 invariant cysteine residues in domains 1 and 3, respectively (IGFBP6 contains 10 cysteine residues in domain 1), and are thought to be the IGF binding domains. Domain 2 is defined primarily by a lack of sequence identity among the 6 IGFBPs and by a lack of cysteine residues, though it does contain 2 cysteines in IGFBP4. Domain 3 is homologous to the thyroglobulin type I repeat unit. Kiefer et al. (1992) characterized recombinant human insulin-like growth factor binding proteins 4, 5, and 6 by their expression in yeast as fusion proteins with ubiquitin. Oh et al. (1996) reported that the predicted 277-amino acid protein contains a 26-amino acid signal sequence and the IGFBP motif (GCGCCXXC) at the N terminus. The IGFBP motif is located in a region containing a cluster of 12 conserved cysteines, of which 11 are found in IGFBP7.

Based on primary and secondary structural similiarity of NOV5 polypeptides to the “insulin growth factor binding protein family”, the NOV5 nucleic acids and proteins are useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV5 nucleic acids and proteins of the invention are useful in potential therapeutic applications implicated in vertex balding (hair loss), cancers including but not limited to meningiomas, breast carcinomas, colorectal tumors, lung cancer, muscle fibre atrophy (motor neuron disease), Infantile neuronal ceroid lipofuscinosis (INCL) and/or other pathologies and disorders. For example, a cDNA encoding the insulin growth factor binding protein-like protein may be useful in gene therapy, and the insulin growth factor binding protein-like protein may be useful when administered to a subject in need thereof. By way of nonlimiting example, the compositions of NOV5 will have efficacy for treatment of patients suffering from vertex balding (Hair loss), cancers including but not limited to meningiomas, breast carcinomas, colorectal tumors, lung cancer, muscle fibre atrophy(motor neuron disease), Infantile neuronal ceroid lipofuscinosis (INCL).

The NOV5 nucleic acid, or fragments thereof, may further be useful in diagnostic applications, wherein the presence or amount of the nucleic acid or the protein are to be assessed. NOV5 nucleic acids and polypeptides are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. For example the disclosed NOV5 protein has multiple hydrophilic regions, each of which can be used as an immunogen. In one embodiment, a contemplated NOV5 epitope is from about amino acids 25 to 50. In another embodiment, a NOV5 epitope is from about amino acids 120 to 150. In an additional embodiment, a NOV5 epitope is from about amino acids 180 to 270. This novel protein also has value in development of powerful assay system for functional analysis of various human disorders, which will help in understanding of pathology of the disease and development of new drug targets for various disorders.

NOV6

A NOV6 nucleic acid of 1278 nucleotides (also referred to as 117478122-A) encoding a novel cell cycle P38-2G4 -like protein is shown in Table 6A. An open reading frame was identified beginning with an ATG initiation codon at nucleotides 37-39 and ending with a TGA codon at nucleotides 1225-1227. A putative untranslated region upstream from the initiation codon and downstream from the termination codon is underlined in Table 6A, and the start and stop codons are in bold letters.

TABLE 6A
NOV6 Nucleotide Sequence (SEQ ID NO:15)

CAGGAGAAACGAGGCTGCAGTGGTGGTAGTAGGAAGATGTTGGGCAAGGTCCAGCAGCAGGAGCAAACTA
TCGCCAAGGACCTGGTCGTGACCAAGTATAAGATGTGTGGGGGGGACCACATCGCCAACCGGGTACTTCG
GTCGTTGGTGGAAGCATCTAGCTCAGGTGTGTCGGTACTGAGCCTGTGCGAGAAAGGAGATGCCATGATT
ATGGAAGAAACAGGGAAAATCTTCAAGAAAGAAAAGGAAATGAAGAAACGTATTGCTTTTCCCACCAGCA
TTTCAGTAAATAACTGTGGATGTCACTTCTCCCCTTTGAAGAGAGGCCAGGATTATATTCTCAAGGAAGG
TGACTTGGTAAAAATTGACCTTGGGGTCCATGTGGATGGCTTCATCGCTAATGTAACTCATACTTTTGTG
GTTCATGTAGCTCAGGGGACCCAAGTAACAGGGAGGAAAGGAGATGTTATTAAGGCAGCTCAACTTTGTG
TTGAAGCTGCCTTATGCCTGGTCAAACCTGGAAATCAGAACATACAAGTGAGAGAAGCCTGGAGCAAAGT
TGCCCTCTCATTTAACTGCATGCCAATAGAAGGTATGCTGTCACACCAGTTGAAGCAGCATGTCATCGAT
GGTGAAAAAAACATTATCCAGAATCCTACAGACCAGCAGAAGAAGGACCATGAAAAAGCTGAATTTGAGG
TACATGAAGTATATGCTGCGGATGTTCTCGTCAGCTCAGGAGAGGGCAAGGCCAAGGATGCAGGACAGAG
AACCACTATTTACAAACGAGACTCCTCTAAACAGTATGGACTGAAGAGGAAAACTTCACGTGCCTTCTTC
AGTGAGGTGGAAAGGCATTTTGATGCCATGCCGTTTACTTTAAGAGCATTTGAAGATGAGAAGAAGGCTC
CGATGGATGTGGTGGAGTGCACCAAACATAGACTGCTGCAACCGTTTAATGTTCTCTATGAGAAGGAGGG
CGAATTTGTTGCCCAGTTTAAATTTACAATTCTGCTCATGCCCAATGGCCCCATGCACAAAACCAGTGGT
CCCTTCAAGCCTGACCTCTACAGGTCTGAGATGGAGGTCCAGGATGCAGAGCTAAAGGCCCTCCTCCAGA
GTTCTGCAAGTCGAGAAACCCAGAAAAAGAAAAGAAAAGAAGCCTCCAAGACTGCGGAGAATGCCACCAG
TGGGGAAACATTAGAAGAAAGTGAAGCTGGGGACTGAGGTGGGTCCCATCTCCCCAGCTTGCTACTTCTG
CTCCATCCCCTTCCCACC

A NOV6 nucleic acid was identified on chromosome 20 by TblastN using CuraGen Corporation's sequence file for cell cycle P38-2G4 or homolog as run against the Genomic Daily Files made available by GenBank or from files downloaded from the individual sequencing centers. The nucleic acid sequence was predicted from the genomic file gcsh:sggc_draft_ba102j14_{—}20000615 by homology to a known cell cycle P38-2G4. Exons were predicted by homology and the intron/exon boundaries were determined using standard genetic rules. Exons were further selected and refined by means of similarity determination using one or more procedures from among multiple BLAST searches (for example, tBlastN, BlastX, and BlastN), and GenScan and Grail. Expressed sequences from both public and proprietary databases were also added when available to further define and complete the Cell Cycle P38-2G4-like sequence. The DNA sequence was then manually corrected for apparent inconsistencies thereby obtaining the sequences encoding the full-length protein.

In a search of sequence databases, it was found, for example, that the nucleic acid sequence has 1130 of 1191 bases (94%) identical to a cell cycle P38-2G4 mRNA (GENBANK-ID: HSU59435|acc:U59435)(E=6.7 e−236).

The NOV6 polypeptide (SEQ ID NO:16) encoded by SEQ ID NO:15 is 396 amino acid residues and is presented using the one-letter amino acid code in Table 6B. The NOV6 protein was analyzed for cellular localization. Psort analysis predicts the protein of the invention to be localized in the nucleus with a certainty of 0.6000. NOV6 has a molecular weight of 44198.2 Daltons.

TABLE 6B
Encoded NOV6 protein sequence (SEQ ID NO:16).

MLGKVQQQEQTIAKDLVVTKYKMCGGDHIANRVLRSLVEASSSGVSVLSLCEKGDAMIMEETGKIFKKEKEMKKRI
AFPTSISVNNCGCHFSPLKRGQDYILKEGDLVKIDLGVHVDGFIANVTHTFVVDVAQGTQVTGRKGDVIKAAQLCV
EAALCLVKPGNQNIQVREAWSKVALSFNCMPIEGMLSHQLKQHVIDGEKNIIQNPTDQQKKDHEKAEFEVHEVYAA
DVLVSSGEGKAKDAGQRTTIYKRDSSKQYGLKRKTSRAFFSEVERHFDAMPFTLRAFEDEKKAPMDVVECTKHRLL
QPFNVLYEKEGEFVAQFKFTILLMPNGPMQKTSGPFKPDLYRSEMEVQDAELKALLQSSASRETQKKKRKEASKTA
ENATSGETLEESEAGD

The full amino acid sequence of the protein of the invention was found to have 355 of 395 amino acid residues (89%) identical to, and 367 of 395 residues (92%) positives with, the 394 amino acid cell cycle PROTEIN P38-2G4 HOMOLOG protein from Homo sapiens (ptnr:SPTREMBL-ACC:O43846). The global sequence homology (as defined by FASTA alignment with the full length sequence of this protein) is 91.4% amino acid homology and 90.4% amino acid identity. In addition, NOV6 contains (as defined by Interpro) a metallopeptidase family M24 protein domain at amino acid positions 11 to 270.

It was also found that NOV6 had homology to the amino acid sequences shown in the BLASTP data listed in Table 6C.

TABLE 6C
BLAST results for NOV 6

| Gene Index/ | | Length | Identity | Positives | |
|---|---|---|---|---|---|
| Identifier | Protein/ Organism | (aa) | (%) | (%) | Expect |
| gi\|13650237\|ref\|XP_{—} | proliferation- | 394 | 346/396 | 358/396 | 1e-171 |
| 012178.2\| | associated 2G4, | | (87%) | (90%) | |
| | 38kD [Homo | | | | |
| | sapiens] | | | | |
| gi\|5453842\|ref\|NP_0 | proliferation- | 394 | 345/396 | 357/396 | 1e-170 |
| 06182.1\| | associated 2G4, | | (87%) | (90%) | |
| | 38kD [Homo | | | | |
| | sapiens] | | | | |
| gi\|6755100\|ref\|NP_0 | proliferation- | 394 | 342/396 | 355/396 | 1e-170 |
| 35249.1\| | associated | | (86%) | (89%) | |
| | protein 1 [Mus | | | | |
| | musculus] | | | | |
| gi\|4099506\|gb\|AAD00 | erbH3 binding | 340 | 297/340 | 306/340 | 1e-153 |
| 646.1\| | protein EBP1 | | (87%) | (89%) | |
| (U87954) | [Homo sapiens] | | | | |
| gi\|1083448\|pir\|\|S54 | p38-2G4 [Mus | 340 | 293/340 | 303/340 | 1e-152 |
| 181 | musculus] | | (86%) | (88%) | |

The homology of these sequences is shown graphically in the ClustalW analysis shown in Table 6D.

[image]

[image]

Table 6E lists the domain description from DOMAIN analysis results against NOV6. This indicates that the NOV6 sequence has properties similar to those of other proteins known to contain this domain.

[image]

[image]

Other BLAST results include sequences from the Patp database, which is a proprietary database that contains sequences published in patents and patent publications. Patp results include those listed in Table 6F.

TABLE 6F
Patp alignments of NOVG

| | | | Smallest |
|---|---|---|---|
| | | | Sum |
| | Reading | High | Prob. |
| Sequences producing High-scoring Segment Pairs: | Frame | Score | P(N) |
| Patp:AAY23784 Human cell div regulator (HCDR)3 [Homo sap], 394 aa | +1 | 1792 | 6.3e−184 |
| Patp:AAW73973 Human HCDR-3 protein sequence [Homo sap], 394 aa | +1 | 1792 | 6.3e−184 |
| Patp:AAB23261 Human cell div regulator (HCDR)3 [Homo sap], 394 aa | +1 | 1792 | 6.3e−184 |

Members of the p38-2G4 family belong to a class of nuclear proteins which function in the cell cycle. A composite PA2G4 cDNA of 1697 nucleotides was isolated encoding a protein of 394 amino acids. The deduced amino acid sequence of the PA2G4, a human cell cycle protein, shows very strong homology to the mouse protein p38-2G4 (Lamartine et al., 1997).

Ebp1 is the human homologue of the cell cycle-regulated mouse protein p38-2G4. The interaction of Ebp1 with ErbB-3 was examined in vitro and in vivo. The first 15 amino acids of the juxtamembrane domain of ErbB-3 were essential for Ebp1 binding in vitro. Treatment of AU565 cells with the ErbB-3 ligand heregulin resulted in dissociation of Ebp1 from ErbB-3. Ebp1 translocated from the cytoplasm into the nucleus following heregulin stimulation. These findings suggest that Ebp1 may be a downstream member of an ErbB-3-regulated signal transduction pathway (Yoo et al., 2000). The involvement of the novel the p38-2G4 family, members of the class of nuclear proteins, in the cell cycle, make them possible targets for various cancer therapeutic approaches.

Based on primary and secondary structural similiarity of NOV6 polypeptides to the “cell cycle P38-2G4 family”, the NOV6 nucleic acids and proteins are useful as a protein therapeutic, a small molecule drug target, an antibody target (therapeutic, diagnostic, drug targeting/cytotoxic antibody), diagnostic and/or prognostic marker, gene therapy (Cell Cycle P38-2G4 delivery/Cell Cycle P38-2G4 ablation), research tools, tissue regeneration in vivo and in vitro of all tissues and cell types including those in which the genomic sequence has been identified.

The NOV6 nucleic acids and proteins of the invention are useful in potential therapeutic applications implicated in name of diseases or disorders related to aberrant expression, aberrant function or aberrant physiologic interactions of the cell cycle P38-2G4-like nucleic acid or protein. For example, a cDNA encoding the cell cycle P38-2G4 -like protein may be useful in gene therapy, and the cell cycle P38-2G4 -like protein may be useful when administered to a subject in need thereof. By way of nonlimiting example, the compositions of the present invention will have efficacy for treatment of patients suffering from cell cycle affected diseases/disorders. The NOV6 nucleic acid, or fragments thereof, may further be useful in diagnostic applications, wherein the presence or amount of the nucleic acid or the protein are to be assessed. Moreover, the NOV6 polypeptides can be used as immunogens to produce antibodies specific for the invention, and as vaccines. They can also be used to screen for potential agonist and antagonist compounds. NOV6 nucleic acids and polypeptides are further useful in the generation of antibodies that bind immuno-specifically to the novel NOV6 substances for use in therapeutic or diagnostic methods. These antibodies may be generated according to methods known in the art, using prediction from hydrophobicity charts, as described in the “Anti-NOVX Antibodies” section below. For example the disclosed NOV6 protein has multiple hydrophilic regions, each of which can be used as an immunogen. In one embodiment, a contemplated NOV6 epitope is from about amino acids 10 to 20. In another embodiment, a NOV6 epitope is from about amino acids 60 to 80. In additional embodiments, NOV6 epitopes are from about amino acids 85 to 110, 125-140, 150-170, 180-220, 230-320 and from about amino acids 325to 375. This novel protein also has value in development of powerful assay system for functional analysis of various human disorders, which will help in understanding of pathology of the disease and development of new drug targets for various disorders.

NOV7

NOV7 includes three novel microsomal signal peptidase 18KDa-like proteins disclosed below. The disclosed proteins have been named NOV7a, NOV7b and NOV7c.

NOV7a

A NOV7a nucleic acid of 602 nucleotides (also referred to as SC117873416-A) encoding a novel microsomal signal peptidase 18KDa-like protein is shown in Table 7A. An open reading frame was identified beginning with a CTG initiation codon at nucleotides 29-31 and ending with a TAA codon at nucleotides 560-562. A putative untranslated region upstream from the initiation codon and downstream from the termination codon is underlined in Table 7A, and the start and stop codons are in bold letters.

TABLE 7A
NOV7a Nucleotide Sequence (SEQ ID NO:17)

CGGTGTCCTGCTCGCCTTCCCCTCCTTTCTGTCTCTTGTCTTTTTGGATGAAGTGCAGTGGATGAACAAGT
GGCGGCTCTATTATCAAGTCCTAAATTTTGGAATGATTGTCTCATCAGCACTCATGATCTGGAAGGGGTTA
ATGGTAATAACTGGAAGTGAAAGTCCAATTGTGTTGCTCAGTGGCAGCATGGAACCTGCATTTCATAGAGG
ATATCTTCTCTTTCTAACAAATCGAGTTGAAGATCCCATACGAGTGGGAGAAATCGCTGTTCTGAGGATAG
AAAGAAAAAAGATTCCTATAGTTCACCGAGTTCTGAAGATTCATGAAGAGCAAAATGGGCCTCTCAAGTTT
TTGACCCAAGGAGATAATAATGCGGTTGATGACAGAGGCCTCTATAAACCAGATCAACATTGGCTAGAGAA
AAAAGATGTCCTGGGGAGAGCCACGGGATTTGTTCCTTATATTGGAATTGGGACGAGCCTCATGAATGACT
ATCCTAAACATAAGTATGAAGTGCTCTTTTTGCTGGGTTTATTTGTGCTGGTCCATCGTGAGTAAGAAGTC
TGCCTTGCTGTTCCTGGGAAGATGCCACATTTTT

A NOV7a nucleic acid was identified on chromosome 8 by TblastN using CuraGen Corporation's sequence file for microsomal signal peptidase 18KDa-like protein as run against the Genomic Daily Files made available by GenBank or from files downloaded from the individual sequencing centers. The nucleic acid sequence was predicted from the genomic file AF215846 by homology to a known microsomal signal peptidase 18 KDa subunit. Exons were predicted by homology and the intron/exon boundaries were determined using standard genetic rules. Exons were further selected and refined by means of similarity determination using procedures chosen from among multiple BLAST searches (for example, tBlastN, BlastX, and BlastN), GenScan and Grail. Expressed sequences from both public and proprietary databases were also added when available to further define and complete the microsomal signal peptidase 18KDa-like sequence. The DNA sequence was then manually corrected for apparent inconsistencies thereby obtaining the sequences encoding the full-length protein.

In a search of sequence databases, it was found, for example, that the nucleic acid sequence has 494 of 537 bases (91%) identical to a microsomal signal peptidase 18KDa mRNA (GENBANK-ID: AF061737|acc:AF061737)(E=7.1 e−97).

The NOV7a protein encoded by SEQ ID NO: 17 has 177 amino acid residues, and is presented using the one-letter code in Table 7B (SEQ ID NO: 18). The NOV7a protein was analyzed for cellular localization. Psort analysis predicts the protein of the invention to be localized in the microbody (peroxisome) with a certainty of 0.6351. NOV7a has a molecular weight of 20449.8 Daltons.

TABLE 7B
Encoded NOV7a protein sequence (SEQ ID NO:18).

LSLVFLDEVQWMNKWRLYYQVLNFGMIVSSALMIWKGLMVITGSESPIVLLSGSMEPAFHRGYLLFLTNRV
EDPIRVGEIAVLRIERKKIPIVHRVLKIHEEQNGPLKFLTQGDNNAVDDRGLYKPDQHWLEKKDVLGRATG
FVPYIGIGTSLMNDYPKHKYEVLFLLGLFVLVHRE

The full amino acid sequence of the protein of the invention was found to have 150 of 179 amino acid residues (84%) identical to, and 160 of 178 residues (89%) positive with, the 178 amino acid residue microsomal signal peptidase 18KDa SUBUNIT protein from Canis familiaris (Dog) (ptnr:SPTREMBL-ACC: ACC:P21378)(E=4.5 e−73). The global sequence homology (as defined by FASTA alignment with the full length sequence of this protein) is 87.6% amino acid homology and 85.9% amino acid identity. In addition, the NOV7b protein was found to have 148 of 178 residues (83%) identical to, and 159 of 178 residues (89%) positive with, the human microsomal signal peptidase 18 KDA subunit (EC 3.4.-.-) (SPC 18) having 179 residues (E=8.5 e−72). Furthermore, NOV 7a contains (as defined by Interpro) a signal peptidase I protein domain at amino acid positions 12 to 169.

NOV7b

A NOV7b nucleic acid of 568 nucleotides (also referred to as CG57520-01) encoding a novel Microsomal Signal Peptidase-like protein is shown in Table 7C. An open reading frame was identified beginning at nucleotides 20-22 and ending at nucleotides 554-556. A putative untranslated region upstream from the initiation codon and downstream from the termination codon is underlined in Table 7C, and the start and stop codons are in bold letters.

TABLE 7C
NOV7b Nucleotide Sequence (SEQ ID NO:19)

ATTCCTCGCCGTCCCCGCCATGCTGTCTCTAGACTTTTTGGACGATGTGCGGCGGATGAACAA
GTGGCGGCTCTATTATCAAGTCCTAAATTTTGGAATGATTGTCTCATCAGCACTCATGATCTG
GAAGGGGTTAATGGTAATAACTGGAAGTGAAAGTCCAATTGTGTTGCTCAGTGGCAGCATGGA
ACCTGCATTTCATAGAGGATATCTTCTCTTTCTAACAAATCGAGTTGAAGATCCCATACGAGT
GGGAGAAATTGCTGTTCTAAGGATAGAAGGAAGAAAGATTCCTATAGTTCACCGAGTCTTGAA
GATTCATGAAAAGCAAAATGGGCATATCAAGTTTTTGACCAAAGGAGATAATAATGCGGTTGA
TGACAGAGGCCTCTATAAACAAGATCAACATTGGCTAGAGAAAAAAGATGTCGTGGGGAGAGC
CAGGGGATTTGTTCCTTATATTGGAATTGGGACGAGCCTCATGAATGACTATCCTAAACATAA
GTATGAAGTGCTCTTTTTGCTGGGTTTATTTGTGCTGGTCCATCGTGAGTAAGAAGTCTGCCTT

In the present invention, the target sequence identified previously, NOV7a, was subjected to the exon linking process to confirm the sequence. PCR primers were designed by starting at the most upstream sequence available, for the forward primer, and at the most downstream sequence available for the reverse primer. In each case, the sequence was examined, walking inward from the respective termini toward the coding sequence, until a suitable sequence that is either unique or highly selective was encountered, or, in the case of the reverse primer, until the stop codon was reached. Such primers were designed based on in silico predictions for the full length cDNA, part (one or more exons) of the DNA or protein sequence of the target sequence, or by translated homology of the predicted exons to closely related human sequences sequences from other species. These primers were then employed in PCR amplification based on the following pool of human cDNAs: adrenal gland, bone marrow, brain-amygdala, brain-cerebellum, brain-hippocampus, brain-substantia nigra, brain-thalamus, brain-whole, fetal brain, fetal kidney, fetal liver, fetal lung, heart, kidney, lymphoma-Raji, mammary gland, pancreas, pituitary gland, placenta, prostate, salivary gland, skeletal muscle, small intestine, spinal cord, spleen, stomach, testis, thyroid, trachea, uterus. Usually the resulting amplicons were gel purified, cloned and sequenced to high redundancy. The resulting sequences from all clones were assembled with themselves, with other fragments in CuraGen Corporation's database and with public ESTs. Fragments and ESTs were included as components for an assembly when the extent of their identity with another component of the assembly was at least 95% over 50 bp. In addition, sequence traces were evaluated manually and edited for corrections if appropriate. These procedures provide the sequence reported below, which is designated Accession Number CG57520-01 (update of SC117873416_A) to which sequence for 12 more aminoacids were added based on the similarities shared by this peptidase family.

In a search of sequence databases, it was found, for example, that the nucleic acid sequence of this invention has 542 of 569 bases (95%) identical to a gb:GENBANK-ID:AF061737|acc:AF061737.1 mRNA from Homo sapiens (Homo sapiens microsomal signal peptidase mRNA, complete cds) (E=8.8 e−111).

The NOV7b protein encoded by SEQ ID NO:19 has 178 amino acid residues, and is presented using the one-letter code in Table 7D (SEQ ID NO:20). SignalP, Psort and Hydropathy results predict that NOV7b has no signal peptide and that NOV7b is likely to be localized in the cytoplasm with a certainty of 0.5726.

TABLE 7D
Encoded NOV7b protein sequence (SEQ ID NO:20).

MLSLDFLDDVRRMNKWRLYYQVLNFGMIVSSALMIWKGLMVITGSESPIV
LLSGSMEPAFHRGYLLFLTNRVEDPIRVGEIAVLRIEGRKIPIVHRVLKI
HEKQNGHIKFLTKGDNNAVDDRGLYKQDQHWLEKKDVVGRARGFVPYIGI
GTSLMNDYPKHKYEVLFLLGLFVLVHRE

The full amino acid sequence of the protein of NOV7b was found to have 166 of 179 amino acid residues (92%) identical to, and 169 of 179 amino acid residues (94%) similar to, the 179 amino acid residue ptnr:SWISSNEW-ACC:P21378 protein from Bos taurus (Bovine), and (MICROSOMAL SIGNAL PEPTIDASE 18 KDA SUBUNIT (EC 3.4.-.-) (SPC 18) (ENDOPEPTIDASE SP18)) (E=1.2 e−83).

NOV7c

A NOV7c nucleic acid of 657 nucleotides (also referred to as CG57520-02) encoding a novel Microsomal Signal Peptidase-like protein is shown in Table 7E. An open reading frame was identified beginning at nucleotides 78-80and ending at nucleotides 615-617. A putative untranslated region upstream from the initiation codon and downstream from the termination codon is underlined in Table 7E, and the start and stop codons are in bold letters.

TABLE 7E
NOV7c Nueleotide Sequence (SEQ ID NO:21)

CCGACCCGAGCTCCAGTAGTCCGCCCGCTGGTCATCGCGCCCTTTCCCCTGCCGGTGTCCTGC
TCGCCGTCCCCGCCATGCTGTCTCTAGACTTTTTGGACGATGTGCGGCGGATGAACAAGTGGC
GGCTCTATTATCAAGTCCTAAATTTTGGAATGATTGTCTCATCAGCACTAATGATCTGGAAGG
GGTTAATGGTAATAACTGGAAGTGAAAGTCCGATTGTAGTGGTGCTCAGTGGCAGCATGGAAC
CTGCATTTCATAGAGGATATCTTCTCTTTCTAACAAATCGAGTTGAAGATCCCATACGAGTGG
GAGAAATTGCTGTTCTAAGGATAGAAGGAAGAAAGATTCCTATAGTTCACCGAGTCTTGAAGA
TTCATGAAAAGCAAAATGGGCATATCAAGTTTTTGACCAAAGGAGATAATAATGCGGTTGATG
ACCGAGGCCTCTATAAACAAGATCAACATTGGCTAGAGAAAAAAGATGTTGTGGGGAGAGCCA
GGGGATTTGTTCCTTATATTGGAATTGGGACGAGCCTCATGAATGACTATCCTAAACATAAGT
ATGAAGTGCTCTTTTTGCTGGGTTTATTTGTGCTGGTTCATCGTGAGTAAGAAGTCTGCCTTG
CTGTTCCTGGGAAGATGCCACATTTTT

In the present invention, the target sequence identified previously, NOV7a, was subjected to the exon linking process to confirm the sequence. PCR primers were designed by starting at the most upstream sequence available, for the forward primer, and at the most downstream sequence available for the reverse primer. In each case, the sequence was examined, walking inward from the respective termini toward the coding sequence, until a suitable sequence that is either unique or highly selective was encountered, or, in the case of the reverse primer, until the stop codon was reached. Such primers were designed based on in silico predictions for the full length cDNA, part (one or more exons) of the DNA or protein sequence of the target sequence, or by translated homology of the predicted exons to closely related human sequences sequences from other species. These primers were then employed in PCR amplification based on the following pool of human cDNAs: adrenal gland, bone marrow, brain-amygdala, brain-cerebellum, brain-hippocampus, brain-substantia nigra, brain-thalamus, brain-whole, fetal brain, fetal kidney, fetal liver, fetal lung, heart, kidney, lymphoma-Raji, mammary gland, pancreas, pituitary gland, placenta, prostate, salivary gland, skeletal muscle, small intestine, spinal cord, spleen, stomach, testis, thyroid, trachea, uterus. Usually the resulting amplicons were gel purified, cloned and sequenced to high redundancy. The resulting sequences from all clones were assembled with themselves, with other fragments in CuraGen Corporation's database and with public ESTs. Fragments and ESTs were included as components for an assembly when the extent of their identity with another component of the assembly was at least 95% over 50 bp. In addition, sequence traces were evaluated manually and edited for corrections if appropriate. These procedures provide the sequence reported below, which is designated Accession Number CG57520-02 (update of SC117873416_A).

In a search of sequence databases, it was found, for example, that the nucleic acid sequence of this invention has 637 of 657 bases (96%) identical to a gb:GENBANK-ID:AF061737|acc:AF061737.1 mRNA from Homo sapiens (Homo sapiens microsomal signal peptidase mRNA, complete cds) (E=2.8 e−134).

The NOV7c protein encoded by SEQ ID NO:21 has 179 amino acid residues, and is presented using the one-letter code in Table 7F (SEQ ID NO:22). SignalP, Psort and Hydropathy results predict that NOV7c has no signal peptide and that NOV7b is likely to be localized in the microbody (peroxisome) with a certainty of 0.5624.

TABLE 7F
Encoded NOV7c protein sequence (SEQ ID NO:22).

MLSLDFLDDVRRMNKWRLYYQVLNFGMIVSSALMIWKGLMVITGSESPIVVVLSGSMEPA
FHRGYLLFLTNRVEDPIRVGEIAVLRIEGRKIPIVHRVLKIHEKQNGHIKFLTKGDNNAV
DDRGLYKQDQHWLEKKDVVGRARGFVPYIGIGTSLMNDYPKHKYEVLFLLGLFVLVHRE

The microsomal signal peptidase-like gene disclosed in this invention (NOV7c) is expressed in at least the following tissues: adrenal gland, bone marrow, brain-amygdala, brain-cerebellum, brain-hippocampus, brain-substantia nigra, brain-thalamus, brain-whole, fetal brain, fetal kidney, fetal liver, fetal lung, heart, kidney, lymphoma - Raji, mammary gland, pancreas, pituitary gland, placenta, prostate, salivary gland, skeletal muscle, small intestine, spinal cord, spleen, stomach, testis, thyroid, trachea, uterus, Vein, Whole Organism. Expression information was derived from the tissue sources of the sequences that were included in the derivation of the sequence of NOV7C (CG57520-02). Accordingly, NOV7c nucleic acids, polypeptides or antibodies can be used as a marker to identify these tissues. NOV7c maps to chromosome 15.

The full amino acid sequence of the protein of NOV7c was found to have 166 of 179 amino acid residues (92%) identical to, and 169 of 179 amino acid residues (94%) similar to, the 179 amino acid residue ptnr:SWISSNEW-ACC:O75957 protein from Homo sapiens (Human) (MICROSOMAL SIGNAL PEPTIDASE 18 KDA SUBUNIT (EC3.4.-.-) (SPC18) (ENDOPEPTIDASE SP18))(E=4.2 e−84).

Possible SNPs found for NOV7c are listed in Table 7G.

TABLE 7G
SNPs

| Consensus | | Base | |
|---|---|---|---|
| Position | Depth | Change | PAF |
| 78 | 42 | A>G | 0.310 |
| 87 | 42 | A>C | 0.476 |
| 135 | 43 | G>A | 0.465 |
| 265 | 44 | G>A | 0.500 |
| 382 | 43 | G>A | 0.488 |
| 542 | 38 | T>C | 0.395 |

[image]

It was also found that NOV7a had homology to the amino acid sequences shown in the BLASTP data listed in Table 7I.

TABLE 7I
BLAST results for NOV 7a

| Gene Index/ | | Length | Identity | Positives | |
|---|---|---|---|---|---|
| Identifier | Protein/ Organism | (aa) | (%) | (%) | Expect |
| gi\|76576O9\|ref\|NP_05 | signal peptidase | 179 | 141/178 | 151/178 | 2e-70 |
| 5115.1\| | complex (18kD) | | (79%) | (84%) | |
| | [Homo sapiens] | | | | |
| gi\|9910550\|ref\|NP_06 | signal peptidase | 179 | 140/178 | 151/178 | 6e-70 |
| 4335.1\| | complex (18kD); | | (78%) | (84%) | |
| | sid2895p [Mus | | | | |
| | musculus] | | | | |
| gi\|6647849\|sp\|075957 | MICROSOMAL SIGNAL | 179 | 139/178 | 150/178 | 4e-69 |
| \|SPC4_HUMAN | PEPTIDASE 18 EDA | | (78%) | (84%) | |
| | SUBUNIT (SPC18) | | | | |
| | (ENDOPEPTIDASE | | | | |
| | SP18) | | | | |
| gi\|13829016\|ref\|NP_1 | signal peptidase | 179 | 138/178 | 150/178 | 4e-69 |
| 13911.1\| | complex (18kD) | | (77%) | (83%) | |
| | [Rattus | | | | |
| | norvegicus] | | | | |
| gi\|3641344\|gb\|AAC363 | signal peptidase | 167 | 132/167 | 141/167 | 6e-66 |
| 54.1\| | complex 18 kDa | | (79%) | (84%) | |
| (AF090315) | subunit [Homo | | | | |
| | sapiens] | | | | |

The homology of these sequences is shown graphically in the ClustalW analysis shown in Table 7J.

[image]

The homologies shown above are shared by NOV7b and 7c insofar as they are themselves homologous to NOV7a as shown in Table 7H.

Table 7K lists the domain description from DOMAIN analysis results against NOV7. This indicates that the NOV7 sequence has properties similar to those of other proteins known to contain this domain.

[image]

Other BLAST results include sequences from the Patp database, which is a proprietary database that contains sequences published in patents and patent publications. Patp results include those listed in Table 7L.

TABLE 7L
Patp alignments of NOV7a

| | | | Smallest |
|---|---|---|---|
| | | | Sum |
| | Reading | High | Prob. |
| Sequences producing High-scoring Segment Pairs: | Frame | Score | P(N) |
| Patp:AAY59726 Human normal ovarian tissue.. [Homo sap], 248 aa | +2 | 718 | 1.2e−75 |
| Patp:AAW64536 Human stomach cancer clone.. [Homo sap], 179 aa | +2 | 764 | 5 4e−75 |
| Patp:AAY49909 Human microsomal signal pept.. [Homo sap], 179 aa | +2 | 764 | 5 4e−75 |

The cleavage of signal sequences of secretory and membrane proteins by the signal peptidase complex occurs in the lumen of the endoplasmic reticulum. Mammalian signal peptidase consists of five subunits. Four have been cloned, SPC 18, SPC21, SPC22/23, and SPC25 (Greenburg et al, 1994; Kalies et al, 1996).

Canine microsomal signal peptidase activity was previously isolated as a complex of five subunits (25, 22/23, 21, 18, and 12 kDa). Two of the signal peptidase complex (SPC) subunits (23/23 and 21 kDa) have been cloned and sequenced. One of these, the 21-kDa subunit, was observed to be a mammalian homolog of SEC11 protein (Sec11p) (Greenburg et al, 1989) a gene product essential for signal peptide processing and cell growth in yeast (Bohni et al, 1988). Both the 18- and 21-kDa proteins are found in a complex with the 22/23 kDa SPC subunit, the only SPC subunit containing N-linked oligosaccharide. The existence of homologous subunits is common to a number of known protein complexes and provides further evidence that the association between SPC proteins observed in vitro may be physiologically relevant to the mechanism of signal peptide processing and perhaps protein translocation.

Because signal peptidases play an essential role in protein modification, their activity is essential to cell viability, thus NOV7 nucleic acids or polypeptides can be used to target the signal peptidases using small molecules in order to physically compromise certain cell types, specifically in the treatment of cancer.

Based on primary and secondary structural similiarity of NOV7 polypeptides to the “Peptidase S26 protein family, NOV7 nucleic acids and proteins are useful in potential diagnostic and therapeutic applications and as a research tool. These include serving as a specific or selective nucleic acid or protein diagnostic and/or prognostic marker, wherein the presence or amount of the nucleic acid or the protein are to be assessed. These also include potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), (v) an agent promoting tissue regeneration in vitro and in vivo, and (vi) a biological defense weapon.

The NOV7 nucleic acids and proteins of the invention have applications in the diagnosis and/or treatment of various diseases and disorders. For example, the compositions of NOV7 will have efficacy for the treatment of patients suffering from: smooth muscle disorder; immunological disorder; Addison's disease; bronchitis; dermatomyositis; polymyositis; Crohn's disease; diabetes mellitus; lupus erythematosus; multiple sclerosis; ulcerative colitis; anaemia; osteoarthritis; rheumatoid arthritis; gout; hypertension; myocardial infarction; cardiovascular shock; angina; asthma; migraine; adenocarcinoma; leukemia; lymphoma; melanoma; myeloma; sarcoma as well as other diseases, disorders and conditions.

The NOV7 nucleic acid, or fragments thereof, may further be useful in diagnostic applications, wherein the presence or amount of the nucleic acid or the protein are to be assessed. NOV7 nucleic acids and polypeptides are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. For example the disclosed NOV7 proteins have multiple hydrophilic regions, each of which can be used as an immunogen. In one embodiment, a contemplated NOV7 epitope is from about amino acids 10 to 22. In another embodiment, a NOV7 epitope is from about amino acids 60 to 75. In additional embodiments, NOV7 epitopes are from amino acids 90 to 140 and from amino acids 150 to 165. This novel protein also has value in development of powerful assay system for functional analysis of various human disorders, which will help in understanding of pathology of the disease and development of new drug targets for various disorders.

NOV8

A NOV8 nucleic acid of 2086 nucleotides (also referred to as GMAC006928_{—}1) encoding a novel stromal interaction molecule-like protein is shown in Table 8A. An open reading frame was identified beginning with a ATG initiation codon at nucleotides 41-43 and ending with a TGA codon at nucleotides 2078-2080. A putative untranslated region upstream from the initiation codon and downstream from the termination codon is underlined in Table 8A, and the start and stop codons are in bold letters.

TABLE 8A
NOV8 Nucleotide Sequence (SEQ ID NO:23)

TTGGGTATATAAGGAGCATACCTCAACACAATAAAGGCCAATGAGTTTTTTGGCAGTGAGGACAGGGCAATT
CTCAATGCAGTTCATCTTGAATAGGAAGAAACTTAAGAGTAAAAAGTCTACTTTGAATGCACTAAATTCAGT
GGAAAACACCCAGGGCCTGAACCTTCAGCTGACATCCTGTTACTTGGTTAAATGGATACAGCAGCCTCACGC
CCATGTCCAGGTTTTATGGATAGCAGTGCACGAACCTTCATTTATGATCTCCCAGTTGAAAATCAGTGACCG
GAGTCACAGACAAAAACTTCAGCTCAAGGCATTGGATGTGGTTTTGTTTGGACCTCTAACACGCCCACCTCA
TAACTGGATGAAAGATTTTATCCTCACAGTTTCTATAGTAATTGGTGTTGGAGGCTGCTGGTTTGCTTATAC
GCAGAATAAGACATCAAAAGAACATGTTGCAAAAATGATGAAAGATTTAGAGAGCTTACAAACTGCAGAGCA
AAGTCTAATGGACTTACAGGAGAGGCTTGAAAAGGCACAGGAAGAAAACAGAAATGTTGCTGTAGAAAAGCA
AAATTTAGAGCGCAAAATGATGCATGAAATCAATTATGCAAAGGAGGAGGCTTGTCGGCTCAGAGAGCTAAG
GGAGGGAGCTGAATGTGAATTGAGTAGACGTCAGTATGCAGAACAGGAATTGGAACAGGTTCGCATGGCTCT
GAAAAAGGCCGAAAAAGAATTTCAACTGAGAAGCAGTTGGTCTGTTCCAGATGCACTTCACAAATCGCTTCA
GTTAACACATGAAGTAGAAGTGCAATACTACAATATTAAAAGACAAAACGCTGAAATGCAGCTAGCTATTGC
TAAAGATGAGGCAGAAAAAATTAAAAAGAAGAGAAGCACAGTCTTTGGGACTCTGCACGTTGCACACAGCTC
CTCCCTAGATGAGGTAGACCACAAAATTCTGCAAGCAAAGAAAGCTCTCTCTGAGTTGACAACTTGTTTACG
AGAACGACTTTTTCGCTCGCAACAAATTGAGAAGATCTGTGGCTTTCAGATAGCCCATAACTCAGGACTCCC
CAGCCTGACCTCTTCCCTTTATTCTGATCACAGCTGGGTGCTGATGCCCAGAGTCTCCATTCCACCCTATCC
AATTGCTGCACGAGTTGATGACTTAGATGAAGACACACCCCCAATAGTGTCACAATTTCCCGGGACCATGGC
TAAACCTCCTGGATCATTAGCCAGAAGCAGCAGCCTGTGCCGTTCACGCCGCAGCATTGTGCCGTCCTCGCC
TCAGCCTCAGCGAGCTCAGCTTGCTCCACACGCCCCCCACCCGTCACACCCTCGGCACCCTCACCACCCGCA
ACACACACCACACTCCTTGCCTTCCCCTGATCCAGATATCCTCTCAGTGTCAAGTTGCCCTGCGCTTTATCG
AAATGAACACGAGGAAGAGGCCATTTACTTCTCTGCTGAAAAGCAATCCAACACAAGGGAGTGTGCAGTTGG
AGACAGCCACGGACCACATGTACACGGCCTGGTACGCTTTGACAAGGACTTTGGATCTTACTCTGAGTATGA
GAGAAAGCACTGGGAAGTTTCAATGCCAGACACAGCTTCAGAATGTGACTCCTTAAATTCTTCCATTGGAAG
GAAACAGTCTCCTCCTTTAAGCCTCGACATATACCAAACATTATCTCCGCCAAAGATATCAAGAGATGAGGT
GTCCCTAGAGGATTCCTCCCGAGGGGATTCGCCTGTAACTGTGGATGTGTCTTGGGGTTCTCCCGACTGTGT
ACGTCTGACAGAAACTAAGAGTAWGATCTTCAGTCCTGCAAGCAAAGTGTACAATGGCATTTTGGAGAAATC
CTGTAGCATGAACCAGCTTTCCAGTGGCATCCCGGTGCCTAAACCTCGCCACACATCATGTTCCTCAGCTGG
CAACGACAGTAAACCAGTTCAGGAAGCCCCAAGTGTTGCCAGAATAAGCAGCATCCCACATGACCTTTGTCA
TAATGGAGAGAAAAGCAAAAAGCCATCAAAAATCAAAAGCCTTTTTAAGAAGAAATCTAAGTGAAATAAA

A NOV8 nucleic acid GMAC006928_{—}1 was identified on chromosome 4 by TblastN using CuraGen Corporation's sequence file for (SC17716722), run against the Genomic Daily Files made available by GenBank. Genomic clone AC006928 was analyzed by Genscan and Grail to identify exons and putative coding sequences. These clones were also analyzed by TblastN, BlastX and other programs to identify genomic regions translating to proteins with similarity to the original protein or protein family of interest. Genscan indicated a putative gene having homology to Stromal interaction molecule. The results of these analyses were integrated and manually corrected for apparent inconsistencies that may have arisen, for example, from miscalled bases in the original fragments used. The sequences obtained encode the full-length proteins disclosed herein. When necessary, the process to identify and analyze cDNAs, ESTs and genomic clones was reiterated to derive the full length sequence. This invention describes the resulting full-length DNA sequence, any alternative splice forms identified, and the full-length protein sequence which they encode. The nucleic acid and protein sequence are referred to here as GMAC006928_{—}1 (NOV8).

In a search of sequence databases, it was found, for example, that the nucleic acid sequence of this invention has 669 of 990 bases (67%) identical to a human GOK1 mRNA (GENBANK-ID: HSU52426)(E=3.2 e−76).

A disclosed NOV8 protein encoded by SEQ ID NO:23 has 679 amino acid residues, and is presented using the one-letter code in Table 8B (SEQ ID NO:24). The SignalP, Psort and/or Hydropathy profile for NOV8 indicate that this sequence does not contain a signal peptide is likely to be localized at mitochondrial inner membrane and plasma membrane with a certainty of 0.7690 and 0.6500, respectively. Although SignalP, Psort and/or hydropathy suggest that the protein may be localized to mitochondrial membrane, the protein predicted here is similar to the “Stromal interaction molecule Family”, some members of which have membrane localization presented at the plasma membrane. Therefore it is likely that this novel stromal interaction molecule is available at the appropriate sub-cellular localization and hence accessible for the therapeutic uses described in this application. NOV8 has a molecular weight of 76691.4 Daltons.

TABLE 8B
Encoded NOV8 protein sequence (SEQ ID NO:24).

MSFLAVRTGQFSMQFILNRKKLKSKKSTLNALNSVENTQGLNLQLTSCYLVKWIQQPQAHVQVLWIAVHEPS
FMISQLKISDRSHRQKLQLKALDVVLFGPLTRPPHNWMKDFILTVSIVIGVGGCWFAYTQNKTSKEHVAKMM
KDLESLQTAEQSLMDLQERLEKAQEENRNVAVEKQNLERKpMMDEINYAKEEACRLRELREGAECELSRRQYA
EQELEQVRMALKKAEKEFELRSSWSVPDALQKWLQLTHEVEVQYYNIKRQNAEMQLAIAKDEAEKIKKKRST
VFGTLHVAHSSSLDEVDHKILEAKKALSELTTCLRERLFRWQQTEKICGFQIAHNSGLPSLTSSLYSDHSWV
VMPRVSIPPYPIAGGVDDLDEDTPPIVSQFPGTMAKPPGSLARSSSLCRSRRSIVPSSPQPQRAQLAPHAPH
PSHPRHPHHPQHTPHSLPSPDPDTISVSSCPALYRNEEEEEAIYFSAEKQWNTRECAVGDSQGPHVHGLVRF
DKDFGSYSEYERKHWEVSMPDTASECDSLNSSIGRKQSPPLSLEIYQTLSPRKISRDEVSLEDSSRGDSPVT
VDVSWGSPDCVGLTETKSMIFSPASKVYNGILEKSCSMNQLSSGIPVPKPRHTSCSSAGNDSKPVQEAPSVA
RISSIPHDLCHNGEKSKKPSKIKSLFKKKSK

The stromal interaction molecule-like protein disclosed in this invention (NOV8) was found to be expressed in at least the following tissues: (derived from EST data) aorta, brain, breast, cns, colon, ear, esophagus, foreskin, germ cell, heart, kidney, lung, lymph, muscle, ovary, pancreas, parathyroid, placenta pooled, prostate, spleen, stomach, testis, thymus, tonsil, uterus, whole embryo and genitourinary tract. Accordingly, NOV8 nucleic acids, polypeptides or antibodies can be used as a marker to identify these tissues. NOV8 maps to the Unigene entry Hs.5683 which maps to chromosome 4p15.3 between markers D4S1551-D4S391.

The full amino acid sequence of the protein of the invention was found to have 228 of 328 (69%) residues identical, and 249 of 328 residues (75%) similar to, the 685 amino acid residue GOK protein from human (ptnr:SPTREMBL-ACC: Q13586)(E=1.2 e−169).

It was also found that NOV8 had homology to the amino acid sequences shown in the BLASTP data listed in Table 8C.

TABLE 8C
BLAST results for NOV8

| Gene Index/ | Protein/ | Length | Identity | Positives | |
|---|---|---|---|---|---|
| Identifier | Organism | (aa) | (%) | (%) | Expect |
| gi\|7959225\|dbj\|BAA96 | KIAA1482 | 818 | 500/597 | 501/597 | 0.0 |
| 006.1\| | protein | | (83%) | (83%) | |
| (AB040915) | [Homo sapiens] | | | | |
| gi\|13276657\|emb\|CAB6 | hypothetical | 698 | 500/597 | 501/597 | 0.0 |
| 6512.1\| | protein [Homo | | (83%) | (83%) | |
| (AL136577) | sapiens] | | | | |
| gi\|10435275\|dbj\|BAB1 | unnamed protein | 310 | 240/298 | 240/298 | 1e−130 |
| 4545.1\| | product [Homo | | (80%) | (80%) | |
| (AK023369) | sapiens] | | | | |
| gi\|4507269\|ref\|NP_00 | stromal | 685 | 207/327 | 245/327 | 1e−105 |
| 3147.1\| | interaction | | (63%) | (74%) | |
| | molecule 1 | | | | |
| | [Homo sapiens] | | | | |
| gi\|6678161\|ref\|NP_03 | stromal | 685 | 206/327 | 245/327 | 1e−104 |
| 3313.1\| | interaction | | (62%) | (73%) | |
| | molecule 1 | | | | |
| | [Mus musculus] | | | | |

The homology of these sequences is shown graphically in the ClustalW analysis shown in Table 8D.

[image]

[image]

[image]

The NOV8 protein contains an RGD domain that is responsible for cell adhesion and is homologous to stromal interaction molecule I that is suggested to play a role in pediatric cancers.

Based on primary and secondary structural similiarity of NOV8 polypeptides to the “Stromal interaction molecule family”, NOV8 nucleic acids and proteins are useful in potential therapeutic applications implicated, for example but not limited to, in various pathologies/disorders as described below and/or other pathologies/disorders. Potential therapeutic uses for the invention(s) are, for example but not limited to, the following: (i) Protein therapeutic, (ii) small molecule drug target, (iii) antibody target (therapeutic, diagnostic, drug targeting/cytotoxic antibody), (iv) diagnostic and/or prognostic marker, (v) gene therapy (gene delivery/gene ablation), (vi) research tools, and (vii) tissue regeneration in vitro and in vivo (regeneration for all these tissues and cell types composing these tissues and cell types derived from these tissues

The NOV8 nucleic acids and proteins of the invention are useful in potential therapeutic applications implicated in various diseases and disorders described below and/or other pathologies and disorders. For example, but not limited to, a cDNA encoding the Stromal interaction molecule-like protein may be useful in gene therapy, and the Stromal interaction molecule-like protein may be useful when administered to a subject in need thereof. By way of nonlimiting example, the compositions of NOV8 will have efficacy for treatment of patients suffering from cancer, trauma, tissue regeneration (in vitro and in vivo), viral/bacterial/parasitic infections, immunological disease, respiratory disease, gastro-intestinal diseases, reproductive health, neurological and neurodegenerative diseases, endocrine diseases, allergy and inflammation, nephrological disorders, cardiovascular diseases, muscle, bone disorders, hematopoietic disorders, urinary system disorders and developmental disorders.

NOV8 nucleic acid, or fragments thereof, may further be useful in diagnostic applications, wherein the presence or amount of the nucleic acid or the protein are to be assessed. NOV8 nucleic acids and polypeptides are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. For example the disclosed NOV8 proteins have multiple hydrophilic regions, each of which can be used as an immunogen. In one embodiment, a contemplated NOV8 epitope is from about amino acids 10 to 50. In another embodiment, a NOV8 epitope is from about amino acids 70 to 80. In additional embodiments, NOV8 epitopes are from amino acids 120 to 350, from about amino acids 375 to 575 and from amino acids 610 to 675. This novel protein also has value in development of powerful assay system for functional analysis of various human disorders, which will help in understanding of pathology of the disease and development of new drug targets for various disorders.

NOVX Nucleic Acids and Polypeptides

One aspect of the invention pertains to isolated nucleic acid molecules that encode NOVX polypeptides or biologically active portions thereof. Also included in the invention are nucleic acid fragments sufficient for use as hybridization probes to identify NOVX-encoding nucleic acids (e.g., NOVX mRNAs) and fragments for use as PCR primers for the amplification and/or mutation of NOVX nucleic acid molecules. As used herein, the term “nucleic acid molecule” is intended to include DNA molecules (e.g., cDNA or genomic DNA), RNA molecules (e.g., mRNA), analogs of the DNA or RNA generated using nucleotide analogs, and derivatives, fragments and homologs thereof. The nucleic acid molecule may be single-stranded or double-stranded, but preferably is comprised double-stranded DNA.

An NOVX nucleic acid can encode a mature NOVX polypeptide. As used herein, a “mature” form of a polypeptide or protein disclosed in the present invention is the product of a naturally occurring polypeptide or precursor form or proprotein. The naturally occurring polypeptide, precursor or proprotein includes, by way of nonlimiting example, the full-length gene product, encoded by the corresponding gene. Alternatively, it may be defined as the polypeptide, precursor or proprotein encoded by an ORF described herein. The product “mature” form arises, again by way of nonlimiting example, as a result of one or more naturally occurring processing steps as they may take place within the cell, or host cell, in which the gene product arises. Examples of such processing steps leading to a “mature” form of a polypeptide or protein include the cleavage of the N-terminal methionine residue encoded by the initiation codon of an ORF, or the proteolytic cleavage of a signal peptide or leader sequence. Thus a mature form arising from a precursor polypeptide or protein that has residues 1 to N, where residue 1 is the N-terminal methionine, would have residues 2 through N remaining after removal of the N-terminal methionine. Alternatively, a mature form arising from a precursor polypeptide or protein having residues 1 to N, in which an N-terminal signal sequence from residue 1 to residue M is cleaved, would have the residues from residue M+1 to residue N remaining. Further as used herein, a “mature” form of a polypeptide or protein may arise from a step of post-translational modification other than a proteolytic cleavage event. Such additional processes include, by way of non-limiting example, glycosylation, myristoylation or phosphorylation. In general, a mature polypeptide or protein may result from the operation of only one of these processes, or a combination of any of them.

The term “probes”, as utilized herein, refers to nucleic acid sequences of variable length, preferably between at least about 10 nucleotides (nt), 100 nt, or as many as approximately, e.g., 6,000 nt, depending upon the specific use. Probes are used in the detection of identical, similar, or complementary nucleic acid sequences. Longer length probes are generally obtained from a natural or recombinant source, are highly specific, and much slower to hybridize than shorterlength oligomer probes. Probes may be single- or double-stranded and designed to have specificity in PCR, membrane-based hybridization technologies, or ELISA-like technologies.

The term “isolated” nucleic acid molecule, as utilized herein, is one, which is separated from other nucleic acid molecules which are present in the natural source of the nucleic acid. Preferably, an “isolated” nucleic acid is free of sequences which naturally flank the nucleic acid (i.e., sequences located at the 5′- and 3′-termini of the nucleic acid) in the genomic DNA of the organism from which the nucleic acid is derived. For example, in various embodiments, the isolated NOVX nucleic acid molecules can contain less than about 5 kb, 4 kb, 3 kb, 2 kb, 1 kb, 0.5 kb or 0.1 kb of nucleotide sequences which naturally flank the nucleic acid molecule in genomic DNA of the cell/tissue from which the nucleic acid is derived (e.g., brain, heart, liver, spleen, etc.). Moreover, an “isolated” nucleic acid molecule, such as a cDNA molecule, can be substantially free of other cellular material or culture medium when produced by recombinant techniques, or of chemical precursors or other chemicals when chemically synthesized.

A nucleic acid molecule of the invention, e.g., a nucleic acid molecule having the nucleotide sequence SEQ ID NOS: 1, 3, 5, 7, 9, 11, 13, 15, 17, 19, 21, or 23, or a complement of this aforementioned nucleotide sequence, can be isolated using standard molecular biology techniques and the sequence information provided herein. Using all or a portion of the nucleic acid sequence of SEQ ID NOS 1, 3, 5, 7, 9, 11, 13, 15, 17, 19, 21, or 23 as a hybridization probe, NOVX molecules can be isolated using standard hybridization and cloning techniques (e.g., as described in Sambrook, et al., (eds.), MOLECULAR CLONING: A LABORATORY MANUAL ₂nd Ed., Cold Spring Harbor Laboratory Press, Cold Spring Harbor, N.Y., 1989; and Ausubel, et al., (eds.), CURRENT PROTOCOLS IN MOLECULAR BIOLOGY, John Wiley & Sons, New York, N.Y., 1993.)

A nucleic acid of the invention can be amplified using cDNA, mRNA or alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to NOVX nucleotide sequences can be prepared by standard synthetic techniques, e.g., using an automated DNA synthesizer.

As used herein, the term “oligonucleotide” refers to a series of linked nucleotide residues, which oligonucleotide has a sufficient number of nucleotide bases to be used in a PCR reaction. A short oligonucleotide sequence may be based on, or designed from, a genomic or cDNA sequence and is used to amplify, confirm, or reveal the presence of an identical, similar or complementary DNA or RNA in a particular cell or tissue. Oligonucleotides comprise portions of a nucleic acid sequence having about 10 nt, 50 nt, or 100 nt in length, preferably about 15 nt to 30 nt in length. In one embodiment of the invention, an oligonucleotide comprising a nucleic acid molecule less than 100 nt in length would further comprise at least 6 contiguous nucleotides SEQ ID NOS: 1, 3, 5, 7, 9, 11, 13, 15, 17, 19, 21, and 23, or a complement thereof. Oligonucleotides may be chemically synthesized and may also be used as probes.

In another embodiment, an isolated nucleic acid molecule of the invention comprises a nucleic acid molecule that is a complement of the nucleotide sequence shown in SEQ ID NOS: 1, 3, 5, 7, 9, 11, 13, 15, 17, 19, 21, or 23, or a portion of this nucleotide sequence (e.g., a fragment that can be used as a probe or primer or a fragment encoding a biologically-active portion of an NOVX polypeptide). A nucleic acid molecule that is complementary to the nucleotide sequence shown SEQ ID NOS:1, 3, 5, 7, 9, 11, 13, 15, 17, 19, 21, or 23 is one that is sufficiently complementary to the nucleotide sequence shown SEQ ID NOS: 1, 3, 5, 7, 9, 11, 13, 15, 17, 19, 21, or 23 that it can hydrogen bond with little or no mismatches to the nucleotide sequence shown SEQ ID NOS: 1, 3, 5, 7, 9, 11, 13, 15, 17, 19, 21, or 23, thereby forming a stable duplex.

As used herein, the term “complementary” refers to Watson-Crick or Hoogsteen base pairing between nucleotides units of a nucleic acid molecule, and the term “binding” means the physical or chemical interaction between two polypeptides or compounds or associated polypeptides or compounds or combinations thereof. Binding includes ionic, non-ionic, van der Waals, hydrophobic interactions, and the like. A physical interaction can be either direct or indirect. Indirect interactions may be through or due to the effects of another polypeptide or compound. Direct binding refers to interactions that do not take place through, or due to, the effect of another polypeptide or compound, but instead are without other substantial chemical intermediates.

Fragments provided herein are defined as sequences of at least 6 (contiguous) nucleic acids or at least 4 (contiguous) amino acids, a length sufficient to allow for specific hybridization in the case of nucleic acids or for specific recognition of an epitope in the case of amino acids, respectively, and are at most some portion less than a full length sequence. Fragments may be derived from any contiguous portion of a nucleic acid or amino acid sequence of choice. Derivatives are nucleic acid sequences or amino acid sequences formed from the native compounds either directly or by modification or partial substitution. Analogs are nucleic acid sequences or amino acid sequences that have a structure similar to, but not identical to, the native compound but differs from it in respect to certain components or side chains. Analogs may be synthetic or from a different evolutionary origin and may have a similar or opposite metabolic activity compared to wild type. Homologs are nucleic acid sequences or amino acid sequences of a particular gene that are derived from different species.

Derivatives and analogs may be full length or other than full length, if the derivative or analog contains a modified nucleic acid or amino acid, as described below. Derivatives or analogs of the nucleic acids or proteins of the invention include, but are not limited to, molecules comprising regions that are substantially homologous to the nucleic acids or proteins of the invention, in various embodiments, by at least about 70%, 80%, or 95% identity (with a preferred identity of 80-95%) over a nucleic acid or amino acid sequence of identical size or when compared to an aligned sequence in which the alignment is done by a computer homology program known in the art, or whose encoding nucleic acid is capable of hybridizing to the complement of a sequence encoding the aforementioned proteins under stringent, moderately stringent, or low stringent conditions. See e.g. Ausubel, et al., CURRENT PROTOCOLS IN MOLECULAR BIOLOGY, John Wiley & Sons, New York, N.Y., 1993, and below.

A “homologous nucleic acid sequence” or “homologous amino acid sequence,” or variations thereof, refer to sequences characterized by a homology at the nucleotide level or amino acid level as discussed above. Homologous nucleotide sequences encode those sequences coding for isoforms of NOVX polypeptides. Isoforms can be expressed in different tissues of the same organism as a result of, for example, alternative splicing of RNA. Alternatively, isoforms can be encoded by different genes. In the invention, homologous nucleotide sequences include nucleotide sequences encoding for an NOVX polypeptide of species other than humans, including, but not limited to: vertebrates, and thus can include, e.g., frog, mouse, rat, rabbit, dog, cat cow, horse, and other organisms. Homologous nucleotide sequences also include, but are not limited to, naturally occurring allelic variations and mutations of the nucleotide sequences set forth herein. A homologous nucleotide sequence does not, however, include the exact nucleotide sequence encoding human NOVX protein. Homologous nucleic acid sequences include those nucleic acid sequences that encode conservative amino acid substitutions (see below) in SEQ ID NOS: 1, 3, 5, 7, 9, 11, 13, 15, 17, 19, 21, or 23, as well as a polypeptide possessing NOVX biological activity. Various biological activities of the NOVX proteins are described below.

An NOVX polypeptide is encoded by the open reading frame (“ORF”) of an NOVX nucleic acid. An ORF corresponds to a nucleotide sequence that could potentially be translated into a polypeptide. A stretch of nucleic acids comprising an ORF is uninterrupted by a stop codon. An ORF that represents the coding sequence for a full protein begins with an ATG “start” codon and terminates with one of the three “stop” codons, namely, TAA, TAG, or TGA. For the purposes of this invention, an ORF may be any part of a coding sequence, with or without a start codon, a stop codon, or both. For an ORF to be considered as a good candidate for coding for a bonafide cellular protein, a minimum size requirement is often set, e.g., a stretch of DNA that would encode a protein of 50 amino acids or more.

The nucleotide sequences determined from the cloning of the human NOVX genes allows for the generation of probes and primers designed for use in identifying and/or cloning NOVX homologues in other cell types, e.g. from other tissues, as well as NOVX homologues from other vertebrates. The probe/primer typically comprises substantially purified oligonucleotide. The oligonucleotide typically comprises a region of nucleotide sequence that hybridizes under stringent conditions to at least about 12, 25, 50, 100, 150, 200, 250, 300, 350 or 400 consecutive sense strand nucleotide sequence SEQ ID NOS:1, 3, 5, 7, 9, 11, 13, 15, 17, 19, 21, or 23; or an anti-sense strand nucleotide sequence of SEQ ID NOS:1, 3, 5, 7, 9, 11, 13, 15, 17, 19, 21, or 23 ; or of a naturally occurring mutant of SEQ ID NOS: 1, 3, 5, 7, 9, 11, 13, 15, 17, 19, 21, or 23.

Probes based on the human NOVX nucleotide sequences can be used to detect transcripts or genomic sequences encoding the same or homologous proteins. In various embodiments, the probe further comprises a label group attached thereto, e.g. the label group can be a radioisotope, a fluorescent compound, an enzyme, or an enzyme co-factor. Such probes can be used as a part of a diagnostic test kit for identifying cells or tissues which mis-express an NOVX protein, such as by measuring a level of an NOVX-encoding nucleic acid in a sample of cells from a subject e.g., detecting NOVX mRNA levels or determining whether a genomic NOVX gene has been mutated or deleted.

“A polypeptide having a biologically-active portion of an NOVX polypeptide” refers to polypeptides exhibiting activity similar, but not necessarily identical to, an activity of a polypeptide of the invention, including mature forms, as measured in a particular biological assay, with or without dose dependency. A nucleic acid fragment encoding a “biologically-active portion of NOVX” can be prepared by isolating a portion SEQ ID NOS:1, 3, 5, 7, 9, 11, 13, 15, 17, 19, 21, or 23, that encodes a polypeptide having an NOVX biological activity (the biological activities of the NOVX proteins are described below), expressing the encoded portion of NOVX protein (e.g., by recombinant expression in vitro) and assessing the activity of the encoded portion of NOVX.

NOVX Nucleic Acid and Polypeptide Variants

The invention further encompasses nucleic acid molecules that differ from the nucleotide sequences shown in SEQ ID NOS: 1, 3, 5, 7, 9, 11, 13, 15, 17, 19, 21, or 23 due to degeneracy of the genetic code and thus encode the same NOVX proteins as that encoded by the nucleotide sequences shown in SEQ ID NOS: 1, 3, 5, 7, 9, 11, 13, 15, 17, 19, 21, or 23. In another embodiment, an isolated nucleic acid molecule of the invention has a nucleotide sequence encoding a protein having an amino acid sequence shown in SEQ ID NOS:2, 4, 6, 8, 10, 12, 14, 16, 18, 20, 22,or24.

In addition to the human NOVX nucleotide sequences shown in SEQ ID NOS: 1, 3, 5, 7, 9, 11, 13, 15, 17, 19, 21, or 23, it will be appreciated by those skilled in the art that DNA sequence polymorphisms that lead to changes in the amino acid sequences of the NOVX polypeptides may exist within a population (e.g., the human population). Such genetic polymorphism in the NOVX genes may exist among individuals within a population due to natural allelic variation. As used herein, the terms “gene” and “recombinant gene” refer to nucleic acid molecules comprising an open reading frame (ORF) encoding an NOVX protein, preferably a vertebrate NOVX protein. Such natural allelic variations can typically result in 1-5% variance in the nucleotide sequence of the NOVX genes. Any and all such nucleotide variations and resulting amino acid polymorphisms in the NOVX polypeptides, which are the result of natural allelic variation and that do not alter the functional activity of the NOVX polypeptides, are intended to be within the scope of the invention.

Moreover, nucleic acid molecules encoding NOVX proteins from other species, and thus that have a nucleotide sequence that differs from the human SEQ ID NOS: 1, 3, 5, 7, 9, 11, 13, 15, 17, 19, 21, or 23 are intended to be within the scope of the invention. Nucleic acid molecules corresponding to natural allelic variants and homologues of the NOVX cDNAs of the invention can be isolated based on their homology to the human NOVX nucleic acids disclosed herein using the human cDNAs, or a portion thereof, as a hybridization probe according to standard hybridization techniques under stringent hybridization conditions.

Accordingly, in another embodiment, an isolated nucleic acid molecule of the invention is at least 6 nucleotides in length and hybridizes under stringent conditions to the nucleic acid molecule comprising the nucleotide sequence of SEQ ID NOS: 1, 3, 5, 7, 9, 11, 13, 15, 17, 19, 21, or 23. In another embodiment, the nucleic acid is at least 10, 25, 50, 100, 250, 500, 750, 1000, 1500, or 2000 or more nucleotides in length. In yet another embodiment, an isolated nucleic acid molecule of the invention hybridizes to the coding region. As used herein, the term “hybridizes under stringent conditions” is intended to describe conditions for hybridization and washing under which nucleotide sequences at least 60% homologous to each other typically remain hybridized to each other.

Homologs (i.e., nucleic acids encoding NOVX proteins derived from species other than human) or other related sequences (e.g., paralogs) can be obtained by low, moderate or high stringency hybridization with all or a portion of the particular human sequence as a probe using methods well known in the art for nucleic acid hybridization and cloning.

As used herein, the phrase “stringent hybridization conditions” refers to conditions under which a probe, primer or oligonucleotide will hybridize to its target sequence, but to no other sequences. Stringent conditions are sequence-dependent and will be different in different circumstances. Longer sequences hybridize specifically at higher temperatures than shorter sequences. Generally, stringent conditions are selected to be about 5° C. lower than the thermal melting point (Tm) for the specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present at excess, at Tm, 50% of the probes are occupied at equilibrium. Typically, stringent conditions will be those in which the salt concentration is less than about 1.0 M sodium ion, typically about 0.01 to 1.0 M sodium ion (or other salts) at pH 7.0 to 8.3 and the temperature is at least about 30° C. for short probes, primers or oligonucleotides (e.g., 10 nt to 50 nt) and at least about 60° C. for longer probes, primers and oligonucleotides. Stringent conditions may also be achieved with the addition of destabilizing agents, such as formamide.

Stringent conditions are known to those skilled in the art and can be found in Ausubel, et al., (eds.), CURRENT PROTOCOLS IN MOLECULAR BIOLOGY, John Wiley & Sons, N.Y. (1989), 6.3.1-6.3.6. Preferably, the conditions are such that sequences at least about 65%, 70%, 75%, 85%, 90%, 95%, 98%, or 99% homologous to each other typically remain hybridized to each other. A non-limiting example of stringent hybridization conditions are hybridization in a high salt buffer comprising 6×SSC, 50 mM Tris-HCl (pH 7.5), 1 mM EDTA, 0.02% PVP, 0.02% Ficoll, 0.02% BSA, and 500 mg/ml denatured salmon sperm DNA at 65° C., followed by one or more washes in 0.2×SSC, 0.01% BSA at 50° C. An isolated nucleic acid molecule of the invention that hybridizes under stringent conditions to the sequences SEQ ID NOS: 1, 3, 5, 7, 9, 11, 13, 15, 17, 19, 21, or 23, corresponds to a naturally-occurring nucleic acid molecule. As used herein, a “naturally-occurring” nucleic acid molecule refers to an RNA or DNA molecule having a nucleotide sequence that occurs in nature (e.g., encodes a natural protein).

In a second embodiment, a nucleic acid sequence that is hybridizable to the nucleic acid molecule comprising the nucleotide sequence of SEQ ID NOS: 1, 3, 5, 7, 9, 11, 13, 15, 17, 19, 21, or 23, or fragments, analogs or derivatives thereof, under conditions of moderate stringency is provided. A non-limiting example of moderate stringency hybridization conditions are hybridization in 6×SSC, 5X Denhardt's solution, 0.5% SDS and 100 mg/ml denatured salmon sperm DNA at 55° C., followed by one or more washes in 1×SSC, 0.1% SDS at 37° C. Other conditions of moderate stringency that may be used are well-known within the art. See, e.g., Ausubel, et al. (eds.), 1993, CURRENT PROTOCOLS IN MOLECULAR BIOLOGY, John Wiley & Sons, NY, and Kriegler, 1990; GENE TRANSFER AND EXPRESSION, A LABORATORY MANUAL, Stockton Press, NY.

In a third embodiment, a nucleic acid that is hybridizable to the nucleic acid molecule comprising the nucleotide sequences SEQ ID NOS: 1, 3, 5, 7, 9, 11, 13, 15, 17, 19, 21, or 23, or fragments, analogs or derivatives thereof, under conditions of low stringency, is provided. A non-limiting example of low stringency hybridization conditions are hybridization in 35% formamide, 5×SSC, 50 mM Tris-HCl (pH 7.5), 5 mM EDTA, 0.02% PVP, 0.02% Ficoll, 0.2% BSA, 100 mg/ml denatured salmon sperm DNA, 10% (wt/vol) dextran sulfate at 40° C., followed by one or more washes in 2×SSC, 25 mM Tris-HCI (pH 7.4), 5 mM EDTA, and 0.1% SDS at 50° C. Other conditions of low stringency that may be used are well known in the art (e.g., as employed for cross-species hybridizations). See, e.g., Ausubel, et al. (eds.), 1993, CURRENT PROTOCOLS IN MOLECULAR BIOLOGY, John Wiley & Sons, NY, and Kriegler, 1990, GENE TRANSFER AND EXPRESSION, A LABORATORY MANUAL, Stockton Press, NY; Shilo and Weinberg, 1981. Proc Natl Acad Sci USA 78: 6789-6792.

Conservative Mutations

In addition to naturally-occurring allelic variants of NOVX sequences that may exist in the population, the skilled artisan will further appreciate that changes can be introduced by mutation into the nucleotide sequences SEQ ID NOS: 1, 3, 5, 7, 9, 11, 13, 15, 17, 19, 21, or 23, thereby leading to changes in the amino acid sequences of the encoded NOVX proteins, without altering the functional ability of said NOVX proteins. For example, nucleotide substitutions leading to amino acid substitutions at “non-essential” amino acid residues can be made in the sequence SEQ ID NOS:2, 4, 6, 8, 10, 12, 14, 16, 18, 20, 22, or 24. A “non-essential” amino acid residue is a residue that can be altered from the wild-type sequences of the NOVX proteins without altering their biological activity, whereas an “essential” amino acid residue is required for such biological activity. For example, amino acid residues that are conserved among the NOVX proteins of the invention are predicted to be particularly non-amenable to alteration. Amino acids for which conservative substitutions can be made are well-known within the art.

Another aspect of the invention pertains to nucleic acid molecules encoding NOVX proteins that contain changes in amino acid residues that are not essential for activity. Such NOVX proteins differ in amino acid sequence from SEQ ID NOS: 1, 3, 5, 7, 9, 11, 13, 15, 17, 19, 21, or 23 yet retain biological activity. In one embodiment, the isolated nucleic acid molecule comprises a nucleotide sequence encoding a protein, wherein the protein comprises an amino acid sequence at least about 45% homologous to the amino acid sequences SEQ ID NOS:2, 4, 6, 8, 10, 12, 14, 16, 18, 20, 22, and 24. Preferably, the protein encoded by the nucleic acid molecule is at least about 60% homologous to SEQ ID NOS: 2, 4, 6, 8, 10, 12, 14, 16, 18, 20, 22, and 24; more preferably at least about 70% homologous SEQ ID NOS:2, 4, 6, 8, 10, 12, 14, 16, 18, 20, 22, or 24; still more preferably at least about 80% homologous to SEQ ID NOS:2, 4, 6, 8, 10, 12, 14, 16, 18, 20, 22, or 24; even more preferably at least about 90% homologous to SEQ ID NOS:2, 4, 6, 8, 10, 12, 14, 16, 18, 20, 22, or 24; and most preferably at least about 95% homologous to SEQ ID NOS:2, 4, 6, 8, 10, 12, 14, 16, 18, 20, 22, or 24.

An isolated nucleic acid molecule encoding an NOVX protein homologous to the protein of SEQ ID NOS:2, 4, 6, 8, 10, 12, 14, 16, 18, 20, 22, or 24 can be created by introducing one or more nucleotide substitutions, additions or deletions into the nucleotide sequence of SEQ ID NOS: 1, 3, 5, 7, 9, 11, 13, 15, 17, 19, 21, or 23, such that one or more amino acid substitutions, additions or deletions are introduced into the encoded protein.

Mutations can be introduced into SEQ ID NOS: 1, 3, 5, 7, 9, 11, 13, 15, 17, 19, 21, or 23 by standard techniques, such as site-directed mutagenesis and PCR-mediated mutagenesis. Preferably, conservative amino acid substitutions are made at one or more predicted, non-essential amino acid residues. A “conservative amino acid substitution” is one in which the amino acid residue is replaced with an amino acid residue having a similar side chain. Families of amino acid residues having similar side chains have been defined within the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted non-essential amino acid residue in the NOVX protein is replaced with another amino acid residue from the same side chain family. Alternatively, in another embodiment, mutations can be introduced randomly along all or part of an NOVX coding sequence, such as by saturation mutagenesis, and the resultant mutants can be screened for NOVX biological activity to identify mutants that retain activity. Following mutagenesis SEQ ID NOS: 1, 3, 5, 7, 9, 11, 13, 15, 17, 19, 21, or 23, the encoded protein can be expressed by any recombinant technology known in the art and the activity of the protein can be determined.

The relatedness of amino acid families may also be determined based on side chain interactions. Substituted amino acids may be fully conserved “strong” residues or fully conserved “weak” residues. The “strong” group of conserved amino acid residues may be any one of the following groups: STA, NEQK, NHQK, NDEQ, QHRK, MILV, MILF, HY, FYW, wherein the single letter amino acid codes are grouped by those amino acids that may be substituted for each other. Likewise, the “weak” group of conserved residues may be any one of the following: CSA, ATV, SAG, STNK, STPA, SGND, SNDEQK, NDEQHK, NEQHRK, VLIM, HFY, wherein the letters within each group represent the single letter amino acid code.

In one embodiment, a mutant NOVX protein can be assayed for (i) the ability to form protein:protein interactions with other NOVX proteins, other cell-surface proteins, or biologically-active portions thereof, (ii) complex formation between a mutant NOVX protein and an NOVX ligand; or (iii) the ability of a mutant NOVX protein to bind to an intracellular target protein or biologically-active portion thereof; (e.g. avidin proteins).

In yet another embodiment, a mutant NOVX protein can be assayed for the ability to regulate a specific biological function (e.g., regulation of insulin release).

Antisense Nucleic Acids

Another aspect of the invention pertains to isolated antisense nucleic acid molecules that are hybridizable to or complementary to the nucleic acid molecule comprising the nucleotide sequence of SEQ ID NOS: 1, 3, 5, 7, 9, 11, 13, 15, 17, 19, 21, or 23, or fragments, analogs or derivatives thereof. An “antisense” nucleic acid comprises a nucleotide sequence that is complementary to a “sense” nucleic acid encoding a protein (e.g., complementary to the coding strand of a double-stranded cDNA molecule or complementary to an mRNA sequence). In specific aspects, antisense nucleic acid molecules are provided that comprise a sequence complementary to at least about 10, 25, 50, 100, 250 or 500 nucleotides or an entire NOVX coding strand, or to only a portion thereof. Nucleic acid molecules encoding fragments, homologs, derivatives and analogs of an NOVX protein of SEQ ID NOS:2, 4, 6, 8, 10, 12, 14, 16, 18, 20, 22, or 24, or antisense nucleic acids complementary to an NOVX nucleic acid sequence of SEQ ID NOS: 1, 3, 5, 7, 9, 11, 13, 15, 17, 19, 21, or 23, are additionally provided.

In one embodiment, an antisense nucleic acid molecule is antisense to a “coding region” of the coding strand of a nucleotide sequence encoding an NOVX protein. The term “coding region” refers to the region of the nucleotide sequence comprising codons which are translated into amino acid residues. In another embodiment, the antisense nucleic acid molecule is antisense to a “noncoding region” of the coding strand of a nucleotide sequence encoding the NOVX protein. The term “noncoding region” refers to 5′ and 3′ sequences which flank the coding region that are not translated into amino acids (i.e., also referred to as 5′ and 3′ untranslated regions).

Given the coding strand sequences encoding the NOVX protein disclosed herein, antisense nucleic acids of the invention can be designed according to the rules of Watson and Crick or Hoogsteen base pairing. The antisense nucleic acid molecule can be complementary to the entire coding region of NOVX mRNA, but more preferably is an oligonucleotide that is antisense to only a portion of the coding or noncoding region of NOVX mRNA. For example, the antisense oligonucleotide can be complementary to the region surrounding the translation start site of NOVX mRNA. An antisense oligonucleotide can be, for example, about 5, 10, 15, 20, 25, 30, 35, 40, 45 or 50 nucleotides in length. An antisense nucleic acid of the invention can be constructed using chemical synthesis or enzymatic ligation reactions using procedures known in the art. For example, an antisense nucleic acid (e.g., an antisense oligonucleotide) can be chemically synthesized using naturally-occurring nucleotides or variously modified nucleotides designed to increase the biological stability of the molecules or to increase the physical stability of the duplex formed between the antisense and sense nucleic acids (e.g., phosphorothioate derivatives and acridine substituted nucleotides can be used).

Examples of modified nucleotides that can be used to generate the antisense nucleic acid include: 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methyl guanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding an NOVX protein to thereby inhibit expression of the protein (e.g., by inhibiting transcription and/or translation). The hybridization can be by conventional nucleotide complementarity to form a stable duplex, or, for example, in the case of an antisense nucleic acid molecule that binds to DNA duplexes, through specific interactions in the major groove of the double helix. An example of a route of administration of antisense nucleic acid molecules of the invention includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface (e.g., by linking the antisense nucleic acid molecules to peptides or antibodies that bind to cell surface receptors or antigens). The antisense nucleic acid molecules can also be delivered to cells using the vectors described herein. To achieve sufficient nucleic acid molecules, vector constructs in which the antisense nucleic acid molecule is placed under the control of a strong pol II or pol III promoter are preferred.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other. See, eg., Gaultier, et al., 1987. Nucl. Acids Res. 15: 6625-6641. The antisense nucleic acid molecule can also comprise a 2′-o-methylribonucleotide (See, e.g., Inoue, et al. 1987. Nucl. Acids Res. 15: 6131-6148) or a chimeric RNA-DNA analogue (See, e.g., Inoue, et al., 1987. FEBS Lett. 215: 327-330.

Ribozymes and PNA Moieties

Nucleic acid modifications include, by way of non-limiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject.

In one embodiment, an antisense nucleic acid of the invention is a ribozyme. Ribozymes are catalytic RNA molecules with ribonuclease activity that are capable of cleaving a single-stranded nucleic acid, such as an mRNA, to which they have a complementary region. Thus, ribozymes (e.g., hammerhead ribozymes as described in Haselhoff and Gerlach 1988. Nature 334: 585-591) can be used to catalytically cleave NOVX mRNA transcripts to thereby inhibit translation of NOVX mRNA. A ribozyme having specificity for an NOVX-encoding nucleic acid can be designed based upon the nucleotide sequence of an NOVX cDNA disclosed herein (i.e., SEQ ID NOS: 1, 3, 5, 7, 9, 11, 13, 15, 17, 19, 21, or 23). For example, a derivative of a Tetrahymena L-19 IVS RNA can be constructed in which the nucleotide sequence of the active site is complementary to the nucleotide sequence to be cleaved in an NOVX-encoding mRNA. See, e.g., U.S. Pat. No. 4,987,071 to Cech, et al. and U.S. Pat. No. 5,116,742 to Cech, et al. NOVX mRNA can also be used to select a catalytic RNA having a specific ribonuclease activity from a pool of RNA molecules. See, e.g., Bartel et al., (1993) Science 261:1411-1418.

Alternatively, NOVX gene expression can be inhibited by targeting nucleotide sequences complementary to the regulatory region of the NOVX nucleic acid (e.g., the NOVX promoter and/or enhancers) to form triple helical structures that prevent transcription of the NOVX gene in target cells. See, e.g., Helene, 1991. Anticancer Drug Des. 6: 569-84; Helene, et al. 1992. Ann. N.Y. Acad. Sci. 660: 27-36; Maher, 1992. Bioassays 14: 807-15.

In various embodiments, the NOVX nucleic acids can be modified at the base moiety, sugar moiety or phosphate backbone to improve, e.g., the stability, hybridization, or solubility of the molecule. For example, the deoxyribose phosphate backbone of the nucleic acids can be modified to generate peptide nucleic acids. See, e.g., Hyrup, et al., 1996. Bioorg Med Chem 4: 5-23. As used herein, the terms “peptide nucleic acids” or “PNAs” refer to nucleic acid mimics (e.g., DNA mimics) in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup, et al., 1996.supra; Perry-O'Keefe, et al., 1996. Proc. Natl. Acad. Sci. USA 93: 14670-14675.

PNAs of NOVX can be used in therapeutic and diagnostic applications. For example, PNAs can be used as antisense or antigene agents for sequence-specific modulation of gene expression by, e.g., inducing transcription or translation arrest or inhibiting replication. PNAs of NOVX can also be used, for example, in the analysis of single base pair mutations in a gene (e.g., PNA directed PCR clamping; as artificial restriction enzymes when used in combination with other enzymes, e.g., S₁ nucleases (See, Hyrup, et al., 1996.supra); or as probes or primers for DNA sequence and hybridization (See, Hyrup, et al., 1996,supra; Perry-O'Keefe, et al, 1996.supra).

In another embodiment, PNAs of NOVX can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras of NOVX can be generated that may combine the advantageous properties of PNA and DNA. Such chimeras allow DNA recognition enzymes (e.g., RNase H and DNA polymerases) to interact with the DNA portion while the PNA portion would provide high binding affinity and specificity. PNA-DNA chimeras can be linked using linkers of appropriate lengths selected in terms of base stacking, number of bonds between the nucleobases, and orientation (see, Hyrup, et al., 1996.supra). The synthesis of PNA-DNA chimeras can be performed as described in Hyrup, et al., 1996. supra and Finn, et al., 1996. Nucl Acids Res 24: 3357-3363. For example, a DNA chain can be synthesized on a solid support using standard phosphoramidite coupling chemistry, and modified nucleoside analogs, e.g., 5′-(4-methoxytrityl)amino-5′-deoxy-thymidine phosphoramidite, can be used between the PNA and the 5′ end of DNA. See, e.g., Mag, et al., 1989. Nucl Acid Res 17: 5973-5988. PNA monomers are then coupled in a stepwise manner to produce a chimeric molecule with a 5′ PNA segment and a 3′ DNA segment. See, e.g., Finn, et al., 1996. supra. Alternatively, chimeric molecules can be synthesized with a 5′ DNA segment and a 3′ PNA segment. See, e.g., Petersen, et al., 1975. Bioorg. Med. Chem. Lett. 5: 1119-11124.

In other embodiments, the oligonucleotide may include other appended groups such as peptides (e.g., for targeting host cell receptors in vivo), or agents facilitating transport across the cell membrane (see, e.g., Letsinger, et al., 1989. Proc. Natl. Acad. Sci. U.S.A. 86: 6553-6556; Lemaitre, et al., 1987. Proc. Natl. Acad. Sci. 84: 648-652; PCT Publication No. WO088/09810) or the blood-brain barrier (see, e.g., PCT Publication No. WO 89/10134). In addition, oligonucleotides can be modified with hybridization triggered cleavage agents (see, e.g., Krol, et al., 1988. BioTechniques 6:958-976) or intercalating agents (see, e.g., Zon, 1988. Pharm. Res. 5: 539-549). To this end, the oligonucleotide may be conjugated to another molecule, e.g., a peptide, a hybridization triggered cross-linking agent, a transport agent, a hybridization-triggered cleavage agent, and the like.

NOVX Polypeptides

A polypeptide according to the invention includes a polypeptide including the amino acid sequence of NOVX polypeptides whose sequences are provided in SEQ ID NOS:2, 4, 6, 8, 10, 12, 14, 16, 18, 20, 22, or 24. The invention also includes a mutant or variant protein any of whose residues may be changed from the corresponding residues shown in SEQ ID NOS:2, 4, 6, 8, 10, 12, 14, 16, 18, 20, 22, or 24 while still encoding a protein that maintains its NOVX activities and physiological functions, or a functional fragment thereof.

In general, an NOVX variant that preserves NOVX-like function includes any variant in which residues at a particular position in the sequence have been substituted by other amino acids, and further include the possibility of inserting an additional residue or residues between two residues of the parent protein as well as the possibility of deleting one or more residues from the parent sequence. Any amino acid substitution, insertion, or deletion is encompassed by the invention. In favorable circumstances, the substitution is a conservative substitution as defined above.

One aspect of the invention pertains to isolated NOVX proteins, and biologically-active portions thereof, or derivatives, fragments, analogs or homologs thereof. Also provided are polypeptide fragments suitable for use as immunogens to raise anti-NOVX antibodies. In one embodiment, native NOVX proteins can be isolated from cells or tissue sources by an appropriate purification scheme using standard protein purification techniques. In another embodiment, NOVX proteins are produced by recombinant DNA techniques. Alternative to recombinant expression, an NOVX protein or polypeptide can be synthesized chemically using standard peptide synthesis techniques.

An “isolated” or “purified” polypeptide or protein or biologically-active portion thereof is substantially free of cellular material or other contaminating proteins from the cell or tissue source from which the NOVX protein is derived, or substantially free from chemical precursors or other chemicals when chemically synthesized. The language “substantially free of cellular material” includes preparations of NOVX proteins in which the protein is separated from cellular components of the cells from which it is isolated or recombinantly-produced. In one embodiment, the language “substantially free of cellular material” includes preparations of NOVX proteins having less than about 30% (by dry weight) of non-NOVX proteins (also referred to herein as a “contaminating protein”), more preferably less than about 20% of non-NOVX proteins, still more preferably less than about 10% of non-NOVX proteins, and most preferably less than about 5% of non-NOVX proteins. When the NOVX protein or biologically-active portion thereof is recombinantly-produced, it is also preferably substantially free of culture medium, i.e., culture medium represents less than about 20%, more preferably less than about 10%, and most preferably less than about 5% of the volume of the NOVX protein preparation.

The language “substantially free of chemical precursors or other chemicals” includes preparations of NOVX proteins in which the protein is separated from chemical precursors or other chemicals that are involved in the synthesis of the protein. In one embodiment, the language “substantially free of chemical precursors or other chemicals” includes preparations of NOVX proteins having less than about 30% (by dry weight) of chemical precursors or non-NOVX chemicals, more preferably less than about 20% chemical precursors or non-NOVX chemicals, still more preferably less than about 10% chemical precursors or non-NOVX chemicals, and most preferably less than about 5% chemical precursors or non-NOVX chemicals.

Biologically-active portions of NOVX proteins include peptides comprising amino acid sequences sufficiently homologous to or derived from the amino acid sequences of the NOVX proteins (e.g., the amino acid sequence shown in SEQ ID NOS:2, 4, 6, 8, 10, 12, 14, 16, 18, 20, 22, or 24) that include fewer amino acids than the full-length NOVX proteins, and exhibit at least one activity of an NOVX protein. Typically, biologically-active portions comprise a domain or motif with at least one activity of the NOVX protein. A biologically-active portion of an NOVX protein can be a polypeptide which is, for example, 10, 25, 50, 100 or more amino acid residues in length.

Moreover, other biologically-active portions, in which other regions of the protein are deleted, can be prepared by recombinant techniques and evaluated for one or more of the functional activities of a native NOVX protein.

In an embodiment, the NOVX protein has an amino acid sequence shown SEQ ID NOS:2, 4, 6, 8, 10, 12, 14, 16, 18, 20, 22, or 24. In other embodiments, the NOVX protein is substantially homologous to SEQ ID NOS:2, 4, 6, 8, 10, 12, 14, 16, 18, 20, 22, or 24, and retains the functional activity of the protein of SEQ ID NOS:2, 4, 6, 8, 10, 12, 14, 16, 18, 20, 22, or 24, yet differs in amino acid sequence due to natural allelic variation or mutagenesis, as described in detail, below. Accordingly, in another embodiment, the NOVX protein is a protein that comprises an amino acid sequence at least about 45% homologous to the amino acid sequence SEQ ID NOS:2, 4, 6, 8, 10, 12, 14, 16, 18, 20, 22, or 24, and retains the functional activity of the NOVX proteins of SEQ ID NOS:2, 4, 6, 8, 10, 12, 14, 16, 18, 20, 22, or 24.

Determining Homology Between Two or More Sequences

To determine the percent homology of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are homologous at that position (i.e., as used herein amino acid or nucleic acid “homology” is equivalent to amino acid or nucleic acid “identity”).

The nucleic acid sequence homology may be determined as the degree of identity between two sequences. The homology may be determined using computer programs known in the art, such as GAP software provided in the GCG program package. See, Needleman and Wunsch, 1970. J Mol Biol 48: 443-453. Using GCG GAP software with the following settings for nucleic acid sequence comparison: GAP creation penalty of 5.0 and GAP extension penalty of 0.3, the coding region of the analogous nucleic acid sequences referred to above exhibits a degree of identity preferably of at least 70%, 75%, 80%, 85%, 90%, 95%, 98%, or 99%, with the CDS (encoding) part of the DNA sequence shown in SEQ ID NOS: 1, 3, 5, 7, 9, 11, 13, 15, 17, 19, 21,or23.

The term “sequence identity” refers to the degree to which two polynucleotide or polypeptide sequences are identical on a residue-by-residue basis over a particular region of comparison. The term “percentage of sequence identity” is calculated by comparing two optimally aligned sequences over that region of comparison, determining the number of positions at which the identical nucleic acid base (e.g., A, T, C, G, U, or I, in the case of nucleic acids) occurs in both sequences to yield the number of matched positions, dividing the number of matched positions by the total number of positions in the region of comparison (i.e., the window size), and multiplying the result by 100 to yield the percentage of sequence identity. The term “substantial identity” as used herein denotes a characteristic of a polynucleotide sequence, wherein the polynucleotide comprises a sequence that has at least 80 percent sequence identity, preferably at least 85 percent identity and often 90 to 95 percent sequence identity, more usually at least 99 percent sequence identity as compared to a reference sequence over a comparison region.

Chimeric and Fusion Proteins

The invention also provides NOVX chimeric or fusion proteins. As used herein, an NOVX “chimeric protein” or “fusion protein” comprises an NOVX polypeptide operatively-linked to a non-NOVX polypeptide. An “NOVX polypeptide” refers to a polypeptide having an amino acid sequence corresponding to an NOVX protein SEQ ID NOS:2, 4, 6, 8, 10, 12, 14, 16, 18, 20, 22, or 24), whereas a “non-NOVX polypeptide” refers to a polypeptide having an amino acid sequence corresponding to a protein that is not substantially homologous to the NOVX protein, e.g., a protein that is different from the NOVX protein and that is derived from the same or a different organism. Within an NOVX fusion protein the NOVX polypeptide can correspond to all or a portion of an NOVX protein. In one embodiment, an NOVX fusion protein comprises at least one biologically-active portion of an NOVX protein. In another embodiment, an NOVX fusion protein comprises at least two biologically-active portions of an NOVX protein. In yet another embodiment, an NOVX fusion protein comprises at least three biologically-active portions of an NOVX protein. Within the fusion protein, the term “operatively-linked” is intended to indicate that the NOVX polypeptide and the non-NOVX polypeptide are fused in-frame with one another. The non-NOVX polypeptide can be fused to the N-terminus or C-terminus of the NOVX polypeptide.

In one embodiment, the fusion protein is a GST-NOVX fusion protein in which the NOVX sequences are fused to the C-terminus of the GST (glutathione S-transferase) sequences. Such fusion proteins can facilitate the purification of recombinant NOVX polypeptides.

In another embodiment, the fusion protein is an NOVX protein containing a heterologous signal sequence at its N-terminus. In certain host cells (e.g., mammalian host cells), expression and/or secretion of NOVX can be increased through use of a heterologous signal sequence.

In yet another embodiment, the fusion protein is an NOVX-immunoglobulin fusion protein in which the NOVX sequences are fused to sequences derived from a member of the immunoglobulin protein family. The NOVX-immunoglobulin fusion proteins of the invention can be incorporated into pharmaceutical compositions and administered to a subject to inhibit an interaction between an NOVX ligand and an NOVX protein on the surface of a cell, to thereby suppress NOVX-mediated signal transduction in vivo. The NOVX-immunoglobulin fusion proteins can be used to affect the bioavailability of an NOVX cognate ligand. Inhibition of the NOVX ligand/NOVX interaction may be useful therapeutically for both the treatment of proliferative and differentiative disorders, as well as modulating (e.g. promoting or inhibiting) cell survival. Moreover, the NOVX-immunoglobulin fusion proteins of the invention can be used as immunogens to produce anti-NOVX antibodies in a subject, to purify NOVX ligands, and in screening assays to identify molecules that inhibit the interaction of NOVX with an NOVX ligand.

An NOVX chimeric or fusion protein of the invention can be produced by standard recombinant DNA techniques. For example, DNA fragments coding for the different polypeptide sequences are ligated together in-frame in accordance with conventional techniques, e.g., by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers that give rise to complementary overhangs between two consecutive gene fragments that can subsequently be annealed and reamplified to generate a chimeric gene sequence (see, e.g., Ausubel, et al. (eds.) CURRENT PROTOCOLS IN MOLECULAR BIOLOGY, John Wiley & Sons, 1992). Moreover, many expression vectors are commercially available that already encode a fusion moiety (e.g., a GST polypeptide). An NOVX-encoding nucleic acid can be cloned into such an expression vector such that the fusion moiety is linked in-frame to the NOVX protein.

NOVX Agonists and Antagonists

The invention also pertains to variants of the NOVX proteins that function as either NOVX agonists (i.e., mimetics) or as NOVX antagonists. Variants of the NOVX protein can be generated by mutagenesis (e.g., discrete point mutation or truncation of the NOVX protein). An agonist of the NOVX protein can retain substantially the same, or a subset of, the biological activities of the naturally occurring form of the NOVX protein. An antagonist of the NOVX protein can inhibit one or more of the activities of the naturally occurring form of the NOVX protein by, for example, competitively binding to a downstream or upstream member of a cellular signaling cascade which includes the NOVX protein. Thus, specific biological effects can be elicited by treatment with a variant of limited function. In one embodiment, treatment of a subject with a variant having a subset of the biological activities of the naturally occurring form of the protein has fewer side effects in a subject relative to treatment with the naturally occurring form of the NOVX proteins.

Variants of the NOVX proteins that function as either NOVX agonists (i.e., mimetics) or as NOVX antagonists can be identified by screening combinatorial libraries of mutants (e.g., truncation mutants) of the NOVX proteins for NOVX protein agonist or antagonist activity. In one embodiment, a variegated library of NOVX variants is generated by combinatorial mutagenesis at the nucleic acid level and is encoded by a variegated gene library. A variegated library of NOVX variants can be produced by, for example, enzymatically ligating a mixture of synthetic oligonucleotides into gene sequences such that a degenerate set of potential NOVX sequences is expressible as individual polypeptides, or alternatively, as a set of larger fusion proteins (e.g., for phage display) containing the set of NOVX sequences therein. There are a variety of methods which can be used to produce libraries of potential NOVX variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential NOVX sequences. Methods for synthesizing degenerate oligonucleotides are well-known within the art. See, e.g., Narang, 1983. Tetrahedron 39: 3; Itakura, et al., 1984. Annu. Rev. Biochem. 53: 323; Itakura, et al., 1984. Science 198: 1056; Ike, et al., 1983. Nucl. Acids Res. 11: 477.

Polypeptide Libraries

In addition, libraries of fragments of the NOVX protein coding sequences can be used to generate a variegated population of NOVX fragments for screening and subsequent selection of variants of an NOVX protein. In one embodiment, a library of coding sequence fragments can be generated by treating a double stranded PCR fragment of an NOVX coding sequence with a nuclease under conditions wherein nicking occurs only about once per molecule, denaturing the double stranded DNA, renaturing the DNA to form double-stranded DNA that can include sense/antisense pairs from different nicked products, removing single stranded portions from reformed duplexes by treatment with S₁ nuclease, and ligating the resulting fragment library into an expression vector. By this method, expression libraries can be derived which encodes N-terminal and internal fragments of various sizes of the NOVX proteins.

Various techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of NOVX proteins. The most widely used techniques, which are amenable to high throughput analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique that enhances the frequency of functional mutants in the libraries, can be used in combination with the screening assays to identify NOVX variants. See, e.g., Arkin and Yourvan, 1992. Proc. Natl. Acad. Sci. USA 89: 7811-7815; Delgrave, et al., 1993. Protein Engineering 6:327-331.

Anti-NOVX Antibodies

The invention encompasses antibodies and antibody fragments, such as F_{ab} or (F_{ab})₂, that bind immunospecifically to any of the NOVX polypeptides of said invention.

An isolated NOVX protein, or a portion or fragment thereof, can be used as an immunogen to generate antibodies that bind to NOVX polypeptides using standard techniques for polyclonal and monoclonal antibody preparation. The full-length NOVX proteins can be used or, alternatively, the invention provides antigenic peptide fragments of NOVX proteins for use as immunogens. The antigenic NOVX peptides comprises at least 4 amino acid residues of the amino acid sequence shown SEQ ID NOS:2, 4, 6, 8, 10, 12, 14, 16, 18, 20, 22, or 24 and encompasses an epitope of NOVX such that an antibody raised against the peptide forms a specific immune complex with NOVX. Preferably, the antigenic peptide comprises at least 6, 8, 10, 15, 20, or 30 amino acid residues. Longer antigenic peptides are sometimes preferable over shorter antigenic peptides, depending on use and according to methods well known to someone skilled in the art.

In certain embodiments of the invention, at least one epitope encompassed by the antigenic peptide is a region of NOVX that is located on the surface of the protein (e.g., a hydrophilic region). As a means for targeting antibody production, hydropathy plots showing regions of hydrophilicity and hydrophobicity may be generated by any method well known in the art, including, for example, the Kyte Doolittle or the Hopp Woods methods, either with or without Fourier transformation (see, e.g., Hopp and Woods, 1981. Proc. Nat. Acad. Sci. USA 78: 3824-3828; Kyte and Doolittle, 1982. J. Mol Biol. 157: 105-142, each incorporated herein by reference in their entirety).

As disclosed herein, NOVX protein sequences of SEQ ID NOS: 2, 4, 6, 8, 10, 12, 14, 16, 18, 20, 22, and 24, or derivatives, fragments, analogs or homologs thereof, may be utilized as immunogens in the generation of antibodies that immunospecifically-bind these protein components. The term “antibody” as used herein refers to immunoglobulin molecules and immunologically-active portions of immunoglobulin molecules, i.e., molecules that contain an antigen binding site that specifically-binds (immunoreacts with) an antigen, such as NOVX. Such antibodies include, but are not limited to, polyclonal, monoclonal, chimeric, single chain, F_{ab} and F(_{ab′})₂ fragments, and an F_{ab} expression library. In a specific embodiment, antibodies to human NOVX proteins are disclosed. Various procedures known within the art may be used for the production of polyclonal or monoclonal antibodies to an NOVX protein sequence of SEQ ID NOS: 2, 4, 6, 8, 10, 12, 14, 16, 18, 20, 22, and 24, or a derivative, fragment, analog or homolog thereof. Some of these proteins are discussed below.

For the production of polyclonal antibodies, various suitable host animals (e.g., rabbit, goat, mouse or other mammal) may be immunized by injection with the native protein, or a synthetic variant thereof, or a derivative of the foregoing. An appropriate immunogenic preparation can contain, for example, recombinantly-expressed NOVX protein or a chemicallysynthesized NOVX polypeptide. The preparation can further include an adjuvant. Various adjuvants used to increase the immunological response include, but are not limited to, Freund's (complete and incomplete), mineral gels (e.g., aluminum hydroxide), surface active substances (e.g., lysolecithin, pluronic polyols, polyanions, peptides, oil emulsions, dinitrophenol, etc.), human adjuvants such as Bacille Calmette-Guerin and Corynebacterium parvum, or similar immunostimulatory agents. If desired, the antibody molecules directed against NOVX can be isolated from the mammal (e.g., from the blood) and further purified by well known techniques, such as protein A chromatography to obtain the IgG fraction.

The term “monoclonal antibody” or “monoclonal antibody composition”, as used herein, refers to a population of antibody molecules that contain only one species of an antigen binding site capable of immunoreacting with a particular epitope of NOVX. A monoclonal antibody composition thus typically displays a single binding affinity for a particular NOVX protein with which it immunoreacts. For preparation of monoclonal antibodies directed towards a particular NOVX protein, or derivatives, fragments, analogs or homologs thereof, any technique that provides for the production of antibody molecules by continuous cell line culture may be utilized. Such techniques include, but are not limited to, the hybridoma technique (see, e.g., Kohler & Milstein, 1975. Nature 256: 495-497); the trioma technique; the human B-cell hybridoma technique (see, e.g., Kozbor, et al, 1983. Immunol. Today 4: 72) and the EBV hybridoma technique to produce human monoclonal antibodies (see, e.g., Cole, et al., 1985. In: MONOCLONAL ANTIBODIES AND CANCER THERAPY, Alan R. Liss, Inc., pp. 77-96). Human monoclonal antibodies may be utilized in the practice of the invention and may be produced by using human hybridomas (see, e.g., Cote, et al., 1983. Proc Natl Acad Sci USA 80: 2026-2030) or by transforming human B-cells with Epstein Barr Virus in vitro (see, e.g., Cole, et al., 1985. In: MONOCLONAL ANTIBODIES AND CANCER THERAPY, Alan R. Liss, Inc., pp. 77-96). Each of the above citations is incorporated herein by reference in their entirety.

According to the invention, techniques can be adapted for the production of single-chain antibodies specific to an NOVX protein (see, e.g., U.S. Pat. No. 4,946,778). In addition, methods can be adapted for the construction of F_{ab} expression libraries (see, e.g., Huse, et al., 1989. Science 246: 1275-1281) to allow rapid and effective identification of monoclonal F_{ab} fragments with the desired specificity for an NOVX protein or derivatives, fragments, analogs or homologs thereof. Non-human antibodies can be “humanized” by techniques well known in the art. See, e.g., U.S. Pat. No. 5,225,539. Antibody fragments that contain the idiotypes to an NOVX protein may be produced by techniques known in the art including, but not limited to: (i) an F(_{ab′})₂ fragment produced by pepsin digestion of an antibody molecule; (ii) an F_{ab} fragment generated by reducing the disulfide bridges of an F(_{ab′})₂ fragment; (iii) an F_{ab} fragment generated by the treatment of the antibody molecule with papain and a reducing agent; and (iv) Fᵥ fragments.

Additionally, recombinant anti-NOVX antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art, for example using methods described in International Application No. PCT/US86/02269; European Patent Application No. 184,187; European Patent Application No. 171,496; European Patent Application No. 173,494; PCT International Publication No. WO 86/01533; U.S. Pat. No. 4,816,567; U.S. Pat. No. 5,225,539; European Patent Application No. 125,023; Better, et al., 1988. Science 240: 1041-1043; Liu, et al., 1987. Proc. Natl. Acad. Sci. USA 84: 3439-3443; Liu, et al., 1987. J. Immunol. 139: 3521-3526; Sun, et al., 1987. Proc. Natl. Acad. Sci. USA 84: 214-218; Nishimura, et al., 1987. Cancer Res. 47: 999-1005; Wood, et al., 1985. Nature 314 :446-449; Shaw, et al., 1988. J. Natl. Cancer Inst. 80: 1553-1559); Morrison(1985) Science 229:1202-1207; Oi, et al. (1986) BioTechniques 4:214; Jones, et al., 1986. Nature 321: 552-525; Verhoeyan, et al., 1988. Science 239: 1534; and Beidler, et al., 1988. J. Immunol. 141: 4053-4060. Each of the above citations are incorporated herein by reference in their entirety.

In one embodiment, methods for the screening of antibodies that possess the desired specificity include, but are not limited to, enzyme-linked immunosorbent assay (ELISA) and other immunologically-mediated techniques known within the art. In a specific embodiment, selection of antibodies that are specific to a particular domain of an NOVX protein is facilitated by generation of hybridomas that bind to the fragment of an NOVX protein possessing such a domain. Thus, antibodies that are specific for a desired domain within an NOVX protein, or derivatives, fragments, analogs or homologs thereof, are also provided herein.

Anti-NOVX antibodies may be used in methods known within the art relating to the localization and/or quantitation of an NOVX protein (e.g., for use in measuring levels of the NOVX protein within appropriate physiological samples, for use in diagnostic methods, for use in imaging the protein, and the like). In a given embodiment, antibodies for NOVX proteins, or derivatives, fragments, analogs or homologs thereof, that contain the antibody derived binding domain, are utilized as pharmacologically-active compounds (hereinafter “Therapeutics”).

An anti-NOVX antibody (e.g., monoclonal antibody) can be used to isolate an NOVX polypeptide by standard techniques, such as affinity chromatography or immunoprecipitation. An anti-NOVX antibody can facilitate the purification of natural NOVX polypeptide from cells and of recombinantly-produced NOVX polypeptide expressed in host cells. Moreover, an anti-NOVX antibody can be used to detect NOVX protein (e.g., in a cellular lysate or cell supernatant) in order to evaluate the abundance and pattern of expression of the NOVX protein. Anti-NOVX antibodies can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

NOVX Recombinant Expression Vectors and Host Cells

Another aspect of the invention pertains to vectors, preferably expression vectors, containing a nucleic acid encoding an NOVX protein, or derivatives, fragments, analogs or homologs thereof. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, that is operatively-linked to the nucleic acid sequence to be expressed. Within a recombinant expression vector, “operably-linked” is intended to mean that the nucleotide sequence of interest is linked to the regulatory sequence(s) in a manner that allows for expression of the nucleotide sequence (e.g., in an in vitro transcription/translation system or in a host cell when the vector is introduced into the host cell).

The term “regulatory sequence” is intended to includes promoters, enhancers and other expression control elements (e.g., polyadenylation signals). Such regulatory sequences are described, for example, in Goeddel, GENE EXPRESSION TECHNOLOGY: METHODS IN ENZYMOLOGY 185, Academic Press, San Diego, Calif. (1990). Regulatory sequences include those that direct constitutive expression of a nucleotide sequence in many types of host cell and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g., NOVX proteins, mutant forms of NOVX proteins, fusion proteins, etc.).

The recombinant expression vectors of the invention can be designed for expression of NOVX proteins in prokaryotic or eukaryotic cells. For example, NOVX proteins can be expressed in bacterial cells such as Escherichia coli, insect cells (using baculovirus expression vectors) yeast cells or mammalian cells. Suitable host cells are discussed further in Goeddel, GENE EXPRESSION TECHNOLOGY: METHODS IN ENZYMOLOGY 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in Escherichia coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: (i) to increase expression of recombinant protein; (ii) to increase the solubility of the recombinant protein; and (iii) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith and Johnson, 1988. Gene 67: 31-40), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein.

Examples of suitable inducible non-fusion E. coli expression vectors include pTrc (Amrann et al., (1988) Gene 69:301-315) and pET 11d (Studier et al., GENE EXPRESSION TECHNOLOGY: METHODS IN ENZYMOLOGY 185, Academic Press, San Diego, Calif. (1990) 60-89).

One strategy to maximize recombinant protein expression in E. coli is to express the protein in a host bacteria with an impaired capacity to proteolytically cleave the recombinant protein. See, e.g., Gottesman, GENE EXPRESSION TECHNOLOGY: METHODS IN ENZYMOLOGY 185, Academic Press, San Diego, Calif. (1990) 119-128. Another strategy is to alter the nucleic acid sequence of the nucleic acid to be inserted into an expression vector so that the individual codons for each amino acid are those preferentially utilized in E. coli (see, e.g., Wada, et al., 1992. Nucl. Acids Res. 20: 2111-2118). Such alteration of nucleic acid sequences of the invention can be carried out by standard DNA synthesis techniques.

In another embodiment, the NOVX expression vector is a yeast expression vector. Examples of vectors for expression in yeast Saccharomyces cerivisae include pYepSec1 (Baldari, et al., 1987. EMBO J. 6: 229-234), pMFa (Kurjan and Herskowitz, 1982. Cell 30: 933-943), pJRY88 (Schultz et al., 1987. Gene 54:113-123), pYES2 (Invitrogen Corporation, San Diego, Calif.), and picZ (InVitrogen Corp, San Diego, Calif.).

Alternatively, NOVX can be expressed in insect cells using baculovirus expression vectors. Baculovirus vectors available for expression of proteins in cultured insect cells (e.g., SF9 cells) include the pAc series (Smith, et al., 1983. Mol. Cell. Biol. 3: 2156-2165) and the pVL series (Lucklow and Summers, 1989. Virology 170: 31-39).

In yet another embodiment, a nucleic acid of the invention is expressed in mammalian cells using a mammalian expression vector. Examples of mammalian expression vectors include pCDM8 (Seed, 1987. Nature 329: 840) and pMT2PC (Kaufman, et al., 1987. EMBO J. 6: 187-195). When used in mammalian cells, the expression vector's control functions are often provided by viral regulatory elements. For example, commonly used promoters are derived from polyoma, adenovirus 2, cytomegalovirus, and simian virus 40. For other suitable expression systems for both prokaryotic and eukaryotic cells see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al., 1987. Genes Dev. 1: 268-277), lymphoid-specific promoters (Calame and Eaton, 1988. Adv. Immunol. 43: 235-275), in particular promoters of T cell receptors (Winoto and Baltimore, 1989. EMBO J. 8: 729-733) and immunoglobulins (Banerji, et al., 1983. Cell 33: 729-740; Queen and Baltimore, 1983. Cell 33: 741-748), neuron-specific promoters (e.g., the neurofilament promoter; Byrne and Ruddle, 1989. Proc. Natl. Acad. Sci. USA 86: 5473-5477), pancreas-specific promoters (Edlund, et al., 1985. Science 230: 912-916), and mammary gland-specific promoters (e.g., milk whey promoter; U.S. Pat. No.4,873,316 and European Application Publication No.264,166). Developmentallyregulated promoters are also encompassed, e.g., the murine hox promoters (Kessel and Gruss, 1990. Science 249: 374-379) and the α-fetoprotein promoter (Campes and Tilghman, 1989. Genes Dev. 3: 537-546).

The invention further provides a recombinant expression vector comprising a DNA molecule of the invention cloned into the expression vector in an antisense orientation. That is, the DNA molecule is operatively-linked to a regulatory sequence in a manner that allows for expression (by transcription of the DNA molecule) of an RNA molecule that is antisense to NOVX mRNA. Regulatory sequences operatively linked to a nucleic acid cloned in the antisense orientation can be chosen that direct the continuous expression of the antisense RNA molecule in a variety of cell types, for instance viral promoters and/or enhancers, or regulatory sequences can be chosen that direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see, e.g., Weintraub, et al., “Antisense RNA as a molecular tool for genetic analysis,” Reviews-Trends in Genetics, Vol. 1(1) 1986.

Another aspect of the invention pertains to host cells into which a recombinant expression vector of the invention has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, NOVX protein can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (MOLECULAR CLONING: A LABORATORY MANUAL. 2nd ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Various selectable markers include those that confer resistance to drugs, such as G418, hygromycin and methotrexate. Nucleic acid encoding a selectable marker can be introduced into a host cell on the same vector as that encoding NOVX or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) NOVX protein. Accordingly, the invention further provides methods for producing NOVX protein using the host cells of the invention. In one embodiment, the method comprises culturing the host cell of invention (into which a recombinant expression vector encoding NOVX protein has been introduced) in a suitable medium such that NOVX protein is produced. In another embodiment, the method further comprises isolating NOVX protein from the medium or the host cell.

Transgenic NOVX Animals

The host cells of the invention can also be used to produce non-human transgenic animals. For example, in one embodiment, a host cell of the invention is a fertilized oocyte or an embryonic stem cell into which NOVX protein-coding sequences have been introduced. Such host cells can then be used to create non-human transgenic animals in which exogenous NOVX sequences have been introduced into their genome or homologous recombinant animals in which endogenous NOVX sequences have been altered. Such animals are useful for studying the function and/or activity of NOVX protein and for identifying and/or evaluating modulators of NOVX protein activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, etc. A transgene is exogenous DNA that is integrated into the genome of a cell from which a transgenic animal develops and that remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous NOVX gene has been altered by homologous recombination between the endogenous gene and an exogenous DNA molecule introduced into a cell of the animal, e.g., an embryonic cell of the animal, prior to development of the animal.

A transgenic animal of the invention can be created by introducing NOVX-encoding nucleic acid into the male pronuclei of a fertilized oocyte (e.g., by microinjection, retroviral infection) and allowing the oocyte to develop in a pseudopregnant female foster animal. The human NOVX cDNA sequences SEQ ID NOS: 1, 3, 5, 7, 9, 11, 13, 15, 17, 19, 21, or 23 can be introduced as a transgene into the genome of a non-human animal. Alternatively, a non-human homologue of the human NOVX gene, such as a mouse NOVX gene, can be isolated based on hybridization to the human NOVX cDNA (described further supra) and used as a transgene. Intronic sequences and polyadenylation signals can also be included in the transgene to increase the efficiency of expression of the transgene. A tissue-specific regulatory sequence(s) can be operably-linked to the NOVX transgene to direct expression of NOVX protein to particular cells. Methods for generating transgenic animals via embryo manipulation and microinjection, particularly animals such as mice, have become conventional in the art and are described, for example, in U.S. Pat. Nos. 4,736,866; 4,870,009; and 4,873,191; and Hogan, 1986. In: MANIPULATING THE MOUSE EMBRYO, Cold Spring Harbor Laboratory Press, Cold Spring Harbor, N.Y. Similar methods are used for production of other transgenic animals. A transgenic founder animal can be identified based upon the presence of the NOVX transgene in its genome and/or expression of NOVX mRNA in tissues or cells of the animals. A transgenic founder animal can then be used to breed additional animals carrying the transgene. Moreover, transgenic animals carrying a transgene-encoding NOVX protein can further be bred to other transgenic animals carrying other transgenes.

To create a homologous recombinant animal, a vector is prepared which contains at least a portion of an NOVX gene into which a deletion, addition or substitution has been introduced to thereby alter, e.g., functionally disrupt, the NOVX gene. The NOVX gene can be a human gene (e.g., the cDNA of SEQ ID NOS: 1, 3, 5, 7, 9, 11, 13, 15, 17, 19, 21, or 23), but more preferably, is a non-human homologue of a human NOVX gene. For example, a mouse homologue of human NOVX gene of SEQ ID NOS: 1, 3, 5, 7, 9, 11, 13, 15, 17,19, 21, or 23 can be used to construct a homologous recombination vector suitable for altering an endogenous NOVX gene in the mouse genome. In one embodiment, the vector is designed such that, upon homologous recombination, the endogenous NOVX gene is functionally disrupted (i.e., no longer encodes a functional protein; also referred to as a “knock out” vector).

Alternatively, the vector can be designed such that, upon homologous recombination, the endogenous NOVX gene is mutated or otherwise altered but still encodes functional protein (e.g., the upstream regulatory region can be altered to thereby alter the expression of the endogenous NOVX protein). In the homologous recombination vector, the altered portion of the NOVX gene is flanked at its 5′-and 3′-termini by additional nucleic acid of the NOVX gene to allow for homologous recombination to occur between the exogenous NOVX gene carried by the vector and an endogenous NOVX gene in an embryonic stem cell. The additional flanking NOVX nucleic acid is of sufficient length for successful homologous recombination with the endogenous gene. Typically, several kilobases of flanking DNA (both at the 5′-and 3′-termini) are included in the vector. See, e.g., Thomas, et al., 1987. Cell 51: 503 for a description of homologous recombination vectors. The vector is ten introduced into an embryonic stem cell line (e.g., by electroporation) and cells in which the introduced NOVX gene has homologously-recombined with the endogenous NOVX gene are selected. See, e.g., Li, et al., 1992. Cell 69: 915.

The selected cells are then injected into a blastocyst of an animal (e.g., a mouse) to form aggregation chimeras. See, e.g., Bradley, 1987. In: TERATOCARCINOMAS AND EMBRYONIC STEM CELLS: A PRACTICAL APPROACH, Robertson, ed. IRL, Oxford, pp. 113- 152. A chimeric embryo can then be implanted into a suitable pseudopregnant female foster animal and the embryo brought to term. Progeny harboring the homologously-recombined DNA in their germ cells can be used to breed animals in which all cells of the animal contain the homologously-recombined DNA by germline transmission of the transgene. Methods for constructing homologous recombination vectors and homologous recombinant animals are described further in Bradley, 1991. Curr. Opin. Biotechnol. 2: 823-829; PCT International Publication Nos.: WO 90/11354; WO 91/01140; WO 92/0968; and WO 93/04169.

In another embodiment, transgenic non-humans animals can be produced that contain selected systems that allow for regulated expression of the transgene. One example of such a system is the cre/loxP recombinase system of bacteriophage P1. For a description of the cre/loxP recombinase system, See, e.g., Lakso, et al., 1992. Proc. Natl. Acad. Sci. USA 89: 6232-6236. Another example of a recombinase system is the FLP recombinase system of Saccharomyces cerevisiae. See, O'Gorman, et al., 1991. Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut, et al., 1997. Nature 385: 810-813. In brief, a cell (e.g., a somatic cell) from the transgenic animal can be isolated and induced to exit the growth cycle and enter G₀ phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyte and then transferred to pseudopregnant female foster animal. The offspring borne of this female foster animal will be a clone of the animal from which the cell (e.g., the somatic cell) is isolated.

Pharmaceutical Compositions

The NOVX nucleic acid molecules, NOVX proteins, and anti-NOVX antibodies (also referred to herein as “active compounds”) of the invention, and derivatives, fragments, analogs and homologs thereof, can be incorporated into pharmaceutical compositions suitable for administration. Such compositions typically comprise the nucleic acid molecule, protein, or antibody and a pharmaceutically acceptable carrier. As used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, finger's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., an NOVX protein or anti-NOVX antibody) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No.4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The nucleic acid molecules of the invention can be inserted into vectors and used as gene therapy vectors. Gene therapy vectors can be delivered to a subject by, for example, intravenous injection, local administration (see, e.g., U.S. Pat. No. 5,328,470) or by stereotactic injection (see, e.g., Chen, et al., 1994. Proc. Natl. Acad. Sci. USA 91: 3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system.

The pharmaceutical compositions can be included in a container, pack, or dispenser together with instructions for administration.

Screening and Detection Methods

The isolated nucleic acid molecules of the invention can be used to express NOVX protein (e.g., via a recombinant expression vector in a host cell in gene therapy applications), to detect NOVX mRNA (e.g., in a biological sample) or a genetic lesion in an NOVX gene, and to modulate NOVX activity, as described further, below. In addition, the NOVX proteins can be used to screen drugs or compounds that modulate the NOVX protein activity or expression as well as to treat disorders characterized by insufficient or excessive production of NOVX protein or production of NOVX protein forms that have decreased or aberrant activity compared to NOVX wild-type protein (e g.; diabetes (regulates insulin release); obesity (binds and transport lipids); metabolic disturbances associated with obesity, the metabolic syndrome X as well as anorexia and wasting disorders associated with chronic diseases and various cancers, and infectious disease(possesses anti-microbial activity) and the various dyslipidemias. In addition, the anti-NOVX antibodies of the invention can be used to detect and isolate NOVX proteins and modulate NOVX activity. In yet a further aspect, the invention can be used in methods to influence appetite, absorption of nutrients and the disposition of metabolic substrates in both a positive and negative fashion.

The invention further pertains to novel agents identified by the screening assays described herein and uses thereof for treatments as described, supra.

Screening Assays

The invention provides a method (also referred to herein as a “screening assay”) for identifying modulators, i.e., candidate or test compounds or agents (e.g., peptides, peptidomimetics, small molecules or other drugs) that bind to NOVX proteins or have a stimulatory or inhibitory effect on, e.g., NOVX protein expression or NOVX protein activity. The invention also includes compounds identified in the screening assays described herein.

In one embodiment, the invention provides assays for screening candidate or test compounds which bind to or modulate the activity of the membrane-bound form of an NOVX protein or polypeptide or biologically-active portion thereof. The test compounds of the invention can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds. See, e.g., Lam, 1997. Anticancer Drug Design 12: 145.

A “small molecule” as used herein, is meant to refer to a composition that has a molecular weight of less than about 5 kD and most preferably less than about 4 kD. Small molecules can be, e.g., nucleic acids, peptides, polypeptides, peptidomimetics, carbohydrates, lipids or other organic or inorganic molecules. Libraries of chemical and/or biological mixtures, such as fungal, bacterial, or algal extracts, are known in the art and can be screened with any of the assays of the invention.

Examples of methods for the synthesis of molecular libraries can be found in the art, for example in: DeWitt, et al., 1993. Proc. Natl. Acad. Sci. U.S.A. 90: 6909; Erb, et al., 1994. Proc. Natl. Acad. Sci. U.S.A. 91: 11422; Zuckermann, et al., 1994. J. Med. Chem. 37: 2678; Cho, et al., 1993. Science 261: 1303; Carrell, et al., 1994. Angew. Chem. Int. Ed. Engl. 33: 2059; Carell, et al., 1994. Angew. Chem. Int. Ed. Engl. 33: 2061; and Gallop, et al., 1994. J. Med. Chem. 37: 1233.

Libraries of compounds may be presented in solution (e.g., Houghten, 1992. Biotechniques 13: 412-421), or on beads (Lam, 1991. Nature 354: 82-84), on chips (Fodor, 1993. Nature 364: 555-556), bacteria (Ladner, U.S. Pat. No.5,223,409), spores (Ladner, U.S. Pat. No. 5,233,409), plasmids (Cull, et al., 1992. Proc. Natl. Acad. Sci. USA 89: 1865-1869) or on phage (Scott and Smith, 1990. Science 249: 386-390; Devlin, 1990. Science 249: 404-406; Cwirla, et al., 1990. Proc. Natl. Acad. Sci. U.S.A. 87: 6378-6382; Felici, 1991. J. Mol. Biol. 222: 301-310; Ladner, U.S. Pat. No.5,233,409.).

In one embodiment, an assay is a cell-based assay in which a cell which expresses a membrane-bound form of NOVX protein, or a biologically-active portion thereof, on the cell surface is contacted with a test compound and the ability of the test compound to bind to an NOVX protein determined. The cell, for example, can of mammalian origin or a yeast cell. Determining the ability of the test compound to bind to the NOVX protein can be accomplished, for example, by coupling the test compound with a radioisotope or enzymatic label such that binding of the test compound to the NOVX protein or biologically-active portion thereof can be determined by detecting the labeled compound in a complex. For example, test compounds can be labeled with ¹²⁵I, ³⁵S, ¹⁴C, or ³H, either directly or indirectly, and the radioisotope detected by direct counting of radioemission or by scintillation counting. Alternatively, test compounds can be enzymatically-labeled with, for example, horseradish peroxidase, alkaline phosphatase, or luciferase, and the enzymatic label detected by determination of conversion of an appropriate substrate to product. In one embodiment, the assay comprises contacting a cell which expresses a membrane-bound form of NOVX protein, or a biologically-active portion thereof, on the cell surface with a known compound which binds NOVX to form an assay mixture, contacting the assay mixture with a test compound, and determining the ability of the test compound to interact with an NOVX protein, wherein determining the ability of the test compound to interact with an NOVX protein comprises determining the ability of the test compound to preferentially bind to NOVX protein or a biologically-active portion thereof as compared to the known compound.

In another embodiment, an assay is a cell-based assay comprising contacting a cell expressing a membrane-bound form of NOVX protein, or a biologically-active portion thereof, on the cell surface with a test compound and determining the ability of the test compound to modulate (e.g., stimulate or inhibit) the activity of the NOVX protein or biologically-active portion thereof. Determining the ability of the test compound to modulate the activity of NOVX or a biologically-active portion thereof can be accomplished, for example, by determining the ability of the NOVX protein to bind to or interact with an NOVX target molecule. As used herein, a “target molecule” is a molecule with which an NOVX protein binds or interacts in nature, for example, a molecule on the surface of a cell which expresses an NOVX interacting protein, a molecule on the surface of a second cell, a molecule in the extracellular milieu, a molecule associated with the internal surface of a cell membrane or a cytoplasmic molecule. An NOVX target molecule can be a non-NOVX molecule or an NOVX protein or polypeptide of the invention. In one embodiment, an NOVX target molecule is a component of a signal transduction pathway that facilitates transduction of an extracellular signal (e.g. a signal generated by binding of a compound to a membrane-bound NOVX molecule) through the cell membrane and into the cell. The target, for example, can be a second intercellular protein that has catalytic activity or a protein that facilitates the association of downstream signaling molecules with NOVX.

Determining the ability of the NOVX protein to bind to or interact with an NOVX target molecule can be accomplished by one of the methods described above for determining direct binding. In one embodiment, determining the ability of the NOVX protein to bind to or interact with an NOVX target molecule can be accomplished by determining the activity of the target molecule. For example, the activity of the target molecule can be determined by detecting induction of a cellular second messenger of the target (i.e. intracellular Ca²⁺, diacylglycerol, IP_{3,} etc.), detecting catalytic/enzymatic activity of the target an appropriate substrate, detecting the induction of a reporter gene (comprising an NOVX-responsive regulatory element operatively linked to a nucleic acid encoding a detectable marker, e.g., luciferase), or detecting a cellular response, for example, cell survival, cellular differentiation, or cell proliferation.

In yet another embodiment, an assay of the invention is a cell-free assay comprising contacting an NOVX protein or biologically-active portion thereof with a test compound and determining the ability of the test compound to bind to the NOVX protein or biologically-active portion thereof. Binding of the test compound to the NOVX protein can be determined either directly or indirectly as described above. In one such embodiment, the assay comprises contacting the NOVX protein or biologically-active portion thereof with a known compound which binds NOVX to form an assay mixture, contacting the assay mixture with a test compound, and determining the ability of the test compound to interact with an NOVX protein, wherein determining the ability of the test compound to interact with an NOVX protein comprises determining the ability of the test compound to preferentially bind to NOVX or biologically-active portion thereof as compared to the known compound.

In still another embodiment, an assay is a cell-free assay comprising contacting NOVX protein or biologically-active portion thereof with a test compound and determining the ability of the test compound to modulate (e.g. stimulate or inhibit) the activity of the NOVX protein or biologically-active portion thereof. Determining the ability of the test compound to modulate the activity of NOVX can be accomplished, for example, by determining the ability of the NOVX protein to bind to an NOVX target molecule by one of the methods described above for determining direct binding. In an alternative embodiment, determining the ability of the test compound to modulate the activity of NOVX protein can be accomplished by determining the ability of the NOVX protein further modulate an NOVX target molecule. For example, the catalytic/enzymatic activity of the target molecule on an appropriate substrate can be determined as described, supra.

In yet another embodiment, the cell-free assay comprises contacting the NOVX protein or biologically-active portion thereof with a known compound which binds NOVX protein to form an assay mixture, contacting the assay mixture with a test compound, and determining the ability of the test compound to interact with an NOVX protein, wherein determining the ability of the test compound to interact with an NOVX protein comprises determining the ability of the NOVX protein to preferentially bind to or modulate the activity of an NOVX target molecule.

The cell-free assays of the invention are amenable to use of both the soluble form or the membrane-bound form of NOVX protein. In the case of cell-free assays comprising the membrane-bound form of NOVX protein, it may be desirable to utilize a solubilizing agent such that the membrane-bound form of NOVX protein is maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-114 Thesit®, Isotridecypoly(ethylene glycol ether)ₙ, N-dodecyl—N,N-dimethyl-3-ammonio-1-propane sulfonate, 3-(3-cholamidopropyl) dimethylamminiol-1-propane sulfonate (CHAPS), or 3-(3-cholamidopropyl)dimethylamminiol-2-hydroxy-1-propane sulfonate (CHAPSO).

In more than one embodiment of the above assay methods of the invention, it may be desirable to immobilize either NOVX protein or its target molecule to facilitate separation of complexed from uncomplexed forms of one or both of the proteins, as well as to accommodate automation of the assay. Binding of a test compound to NOVX protein, or interaction of NOVX protein with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided that adds a domain that allows one or both of the proteins to be bound to a matrix. For example, GST-NOVX fusion proteins or GST-target fusion proteins can be adsorbed onto glutathione sepharose beads (Sigma Chemical, St. Louis, Mo.) or glutathione derivatized microtiter plates, that are then combined with the test compound or the test compound and either the non-adsorbed target protein or NOVX protein, and the mixture is incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described, supra. Alternatively, the complexes can be dissociated from the matrix, and the level of NOVX protein binding or activity determined using standard techniques.

Other techniques for immobilizing proteins on matrices can also be used in the screening assays of the invention. For example, either the NOVX protein or its target molecule can be immobilized utilizing conjugation of biotin and streptavidin. Biotinylated NOVX protein or target molecules can be prepared from biotin-NHS (N-hydroxy-succinimide) using techniques well-known within the art (e.g., biotinylation kit, Pierce Chemicals, Rockford, Ill.), and immobilized in the wells of streptavidin-coated 96 well plates (Pierce Chemical). Alternatively, antibodies reactive with NOVX protein or target molecules, but which do not interfere with binding of the NOVX protein to its target molecule, can be derivatized to the wells of the plate, and unbound target or NOVX protein trapped in the wells by antibody conjugation. Methods for detecting such complexes, in addition to those described above for the GST-immobilized complexes, include immunodetection of complexes using antibodies reactive with the NOVX protein or target molecule, as well as enzyme-linked assays that rely on detecting an enzymatic activity associated with the NOVX protein or target molecule.

In another embodiment, modulators of NOVX protein expression are identified in a method wherein a cell is contacted with a candidate compound and the expression of NOVX mRNA or protein in the cell is determined. The level of expression of NOVX mRNA or protein in the presence of the candidate compound is compared to the level of expression of NOVX mRNA or protein in the absence of the candidate compound. The candidate compound can then be identified as a modulator of NOVX mRNA or protein expression based upon this comparison. For example, when expression of NOVX mRNA or protein is greater (i.e., statistically significantly greater) in the presence of the candidate compound than in its absence, the candidate compound is identified as a stimulator of NOVX mRNA or protein expression. Alternatively, when expression of NOVX mRNA or protein is less (statistically significantly less) in the presence of the candidate compound than in its absence, the candidate compound is identified as an inhibitor of NOVX mRNA or protein expression. The level of NOVX mRNA or protein expression in the cells can be determined by methods described herein for detecting NOVX mRNA or protein.

In yet another aspect of the invention, the NOVX proteins can be used as “bait proteins” in a two-hybrid assay or three hybrid assay (see, e.g., U.S. Pat. No. 5,283,317; Zervos, et al., 1993. Cell 72: 223-232; Madura, et al., 1993. J. Biol Chem. 268: 12046-12054; Bartel, et al., 1993. Biotechniques 14: 920-924; Iwabuchi, et al., 1993. Oncogene 8: 1693-1696; and Brent WO 94/10300), to identify other proteins that bind to or interact with NOVX (“NOVX-binding proteins” or “NOVX-bp”) and modulate NOVX activity. Such NOVX-binding proteins are also likely to be involved in the propagation of signals by the NOVX proteins as, for example, upstream or downstream elements of the NOVX pathway.

The two-hybrid system is based on the modular nature of most transcription factors, which consist of separable DNA-binding and activation domains. Briefly, the assay utilizes two different DNA constructs. In one construct, the gene that codes for NOVX is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming an NOVX-dependent complex, the DNA-binding and activation domains of the transcription factor are brought into close proximity. This proximity allows transcription of a reporter gene (e.g., LacZ) that is operably linked to a transcriptional regulatory site responsive to the transcription factor. Expression of the reporter gene can be detected and cell colonies containing the functional transcription factor can be isolated and used to obtain the cloned gene that encodes the protein which interacts with NOVX.

The invention further pertains to novel agents identified by the aforementioned screening assays and uses thereof for treatments as described herein.

Detection Assays

Portions or fragments of the cDNA sequences identified herein (and the corresponding complete gene sequences) can be used in numerous ways as polynucleotide reagents. By way of example, and not of limitation, these sequences can be used to: (i) map their respective genes on a chromosome; and, thus, locate gene regions associated with genetic disease; (ii) identify an individual from a minute biological sample (tissue typing); and (iii) aid in forensic identification of a biological sample. Some of these applications are described in the subsections, below.

Chromosome Mapping

Once the sequence (or a portion of the sequence) of a gene has been isolated, this sequence can be used to map the location of the gene on a chromosome. This process is called chromosome mapping. Accordingly, portions or fragments of the NOVX sequences, SEQ ID NOS: 1, 3, 5, 7, 9, 11, 13, 15, 17, 19, 21, or 23, or fragments or derivatives thereof, can be used to map the location of the NOVX genes, respectively, on a chromosome. The mapping of the NOVX sequences to chromosomes is an important first step in correlating these sequences with genes associated with disease.

Briefly, NOVX genes can be mapped to chromosomes by preparing PCR primers (preferably 15-25 bp in length) from the NOVX sequences. Computer analysis of the NOVX, sequences can be used to rapidly select primers that do not span more than one exon in the genomic DNA, thus complicating the amplification process. These primers can then be used for PCR screening of somatic cell hybrids containing individual human chromosomes. Only those hybrids containing the human gene corresponding to the NOVX sequences will yield an amplified fragment. Somatic cell hybrids are prepared by fusing somatic cells from different mammals (e.g., human and mouse cells). As hybrids of human and mouse cells grow and divide, they gradually lose human chromosomes in random order, but retain the mouse chromosomes. By using media in which mouse cells cannot grow, because they lack a particular enzyme, but in which human cells can, the one human chromosome that contains the gene encoding the needed enzyme will be retained. By using various media, panels of hybrid cell lines can be established. Each cell line in a panel contains either a single human chromosome or a small number of human chromosomes, and a full set of mouse chromosomes, allowing easy mapping of individual genes to specific human chromosomes. See, e.g., D'Eustachio, et al., 1983. Science 220: 919-924. Somatic cell hybrids containing only fragments of human chromosomes can also be produced by using human chromosomes with translocations and deletions.

PCR mapping of somatic cell hybrids is a rapid procedure for assigning a particular sequence to a particular chromosome. Three or more sequences can be assigned per day using a single thermal cycler. Using the NOVX sequences to design oligonucleotide primers, sublocalization can be achieved with panels of fragments from specific chromosomes.

Fluorescence in situ hybridization (FISH) of a DNA sequence to a metaphase chromosomal spread can further be used to provide a precise chromosomal location in one step. Chromosome spreads can be made using cells whose division has been blocked in metaphase by a chemical like colcemid that disrupts the mitotic spindle. The chromosomes can be treated briefly with trypsin, and then stained with Giemsa. A pattern of light and dark bands develops on each chromosome, so that the chromosomes can be identified individually. The FISH technique can be used with a DNA sequence as short as 500 or 600 bases. However, clones larger than 1,000 bases have a higher likelihood of binding to a unique chromosomal location with sufficient signal intensity for simple detection. Preferably 1,000 bases, and more preferably 2,000 bases, will suffice to get good results at a reasonable amount of time. For a review of this technique, see, Verma, et al., HUMAN CHROMOSOMES: A MANUAL OF BASIC TECHNIQUES (Pergamon Press, New York 1988).

Reagents for chromosome mapping can be used individually to mark a single chromosome or a single site on that chromosome, or panels of reagents can be used for marking multiple sites and/or multiple chromosomes. Reagents corresponding to noncoding regions of the genes actually are preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data. Such data are found, e.g. in McKusick, MENDELIAN INHERITANCE IN MAN, available on-line through Johns Hopkins University Welch Medical Library). The relationship between genes and disease, mapped to the same chromosomal region, can then be identified through linkage analysis (co-inheritance of physically adjacent genes), described in, e.g., Egeland, et al., 1987. Nature, 325: 783-787.

Moreover, differences in the DNA sequences between individuals affected and unaffected with a disease associated with the NOVX gene, can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes, such as deletions or translocations that are visible from chromosome spreads or detectable using PCR based on that DNA sequence. Ultimately, complete sequencing of genes from several individuals can be performed to confirm the presence of a mutation and to distinguish mutations from polymorphisms.

Tissue Typing

The NOVX sequences of the invention can also be used to identify individuals from minute biological samples. In this technique, an individual's genomic DNA is digested with one or more restriction enzymes, and probed on a Southern blot to yield unique bands for identification. The sequences of the invention are useful as additional DNA markers for RFLP (“restriction fragment length polymorphisms,” described in U.S. Pat. No. 5,272,057).

Furthermore, the sequences of the invention can be used to provide an alternative technique that determines the actual base-by-base DNA sequence of selected portions of an individual's genome. Thus, the NOVX sequences described herein can be used to prepare two PCR primers from the 5′-and 3′-termini of the sequences. These primers can then be used to amplify an individual's DNA and subsequently sequence it.

Panels of corresponding DNA sequences from individuals, prepared in this manner, can provide unique individual identifications, as each individual will have a unique set of such DNA sequences due to allelic differences. The sequences of the invention can be used to obtain such identification sequences from individuals and from tissue. The NOVX sequences of the invention uniquely represent portions of the human genome. Allelic variation occurs to some degree in the coding regions of these sequences, and to a greater degree in the noncoding regions. It is estimated that allelic variation between individual humans occurs with a frequency of about once per each 500 bases. Much of the allelic variation is due to single nucleotide polymorphisms (SNPs), which include restriction fragment length polymorphisms (RFLPs).

Each of the sequences described herein can, to some degree, be used as a standard against which DNA from an individual can be compared for identification purposes. Because greater numbers of polymorphisms occur in the noncoding regions, fewer sequences are necessary to differentiate individuals. The noncoding sequences can comfortably provide positive individual identification with a panel of perhaps 10 to 1,000 primers that each yield a noncoding amplified sequence of 100 bases. If predicted coding sequences, such as those in SEQ ID NOS: 1, 3, 5, 7, 9, 11, 13, 15, 17, 19, 21, or 23 are used, a more appropriate number of primers for positive individual identification would be 500-2,000.

Predictive Medicine

The invention also pertains to the field of predictive medicine in which diagnostic assays, prognostic assays, pharmacogenomics, and monitoring clinical trials are used for prognostic (predictive) purposes to thereby treat an individual prophylactically. Accordingly, one aspect of the invention relates to diagnostic assays for determining NOVX protein and/or nucleic acid expression as well as NOVX activity, in the context of a biological sample (e.g., blood, serum, cells, tissue) to thereby determine whether an individual is afflicted with a disease or disorder, or is at risk of developing a disorder, associated with aberrant NOVX expression or activity. The disorders include metabolic disorders, diabetes, obesity, infectious disease, anorexia, cancer-associated cachexia, cancer, neurodegenerative disorders, Alzheimer's Disease, Parkinson's Disorder, immune disorders, and hematopoietic disorders, and the various dyslipidemias, metabolic disturbances associated with obesity, the metabolic syndrome X and wasting disorders associated with chronic diseases and various cancers. The invention also provides for prognostic (or predictive) assays for determining whether an individual is at risk of developing a disorder associated with NOVX protein, nucleic acid expression or activity. For example, mutations in an NOVX gene can be assayed in a biological sample. Such assays can be used for prognostic or predictive purpose to thereby prophylactically treat an individual prior to the onset of a disorder characterized by or associated with NOVX protein, nucleic acid expression, or biological activity.

Another aspect of the invention provides methods for determining NOVX protein, nucleic acid expression or activity in an individual to thereby select appropriate therapeutic or prophylactic agents for that individual (referred to herein as “pharmacogenomics”). Pharmacogenomics allows for the selection of agents (e.g., drugs) for therapeutic or prophylactic treatment of an individual based on the genotype of the individual (e.g., the genotype of the individual examined to determine the ability of the individual to respond to a particular agent.)

Yet another aspect of the invention pertains to monitoring the influence of agents (e.g., drugs, compounds) on the expression or activity of NOVX in clinical trials.

These and other agents are described in further detail in the following sections.

Diagnostic Assays

An exemplary method for detecting the presence or absence of NOVX in a biological sample involves obtaining a biological sample from a test subject and contacting the biological sample with a compound or an agent capable of detecting NOVX protein or nucleic acid (e.g., mRNA, genomic DNA) that encodes NOVX protein such that the presence of NOVX is detected in the biological sample. An agent for detecting NOVX mRNA or genomic DNA is a labeled nucleic acid probe capable of hybridizing to NOVX mRNA or genomic DNA. The nucleic acid probe can be, for example, a full-length NOVX nucleic acid, such as the nucleic acid of SEQ ID NOS: 1, 3, 5, 7, 9, 11, 13, 15, 17, 19, 21, or 23, or a portion thereof, such as an oligonucleotide of at least 15, 30, 50, 100, 250 or 500 nucleotides in length and sufficient to specifically hybridize under stringent conditions to NOVX mRNA or genomic DNA. Other suitable probes for use in the diagnostic assays of the invention are described herein.

An agent for detecting NOVX protein is an antibody capable of binding to NOVX protein, preferably an antibody with a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin. The term “biological sample” is intended to include tissues, cells and biological fluids isolated from a subject, as well as tissues, cells and fluids present within a subject. That is, the detection method of the invention can be used to detect NOVX mRNA, protein, or genomic DNA in a biological sample in vitro as well as in vivo. For example, in vitro techniques for detection of NOVX mRNA include Northern hybridizations and in situ hybridizations. In vitro techniques for detection of NOVX protein include enzyme linked immunosorbent assays (ELISAs), Western blots, immunoprecipitations, and immunofluorescence. In vitro techniques for detection of NOVX genomic DNA include Southern hybridizations. Furthermore, in vivo techniques for detection of NOVX protein include introducing into a subject a labeled anti-NOVX antibody. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject. A preferred biological sample is a peripheral blood leukocyte sample isolated by conventional means from a subject.

In another embodiment, the methods further involve obtaining a control biological sample from a control subject, contacting the control sample with a compound or agent capable 20 of detecting NOVX protein, mRNA, or genomic DNA, such that the presence of NOVX protein, mRNA or genomic DNA is detected in the biological sample, and comparing the presence of NOVX protein, mRNA or genomic DNA in the control sample with the presence of NOVX protein, mRNA or genomic DNA in the test sample.

The invention also encompasses kits for detecting the presence of NOVX in a biological sample. For example, the kit can comprise: a labeled compound or agent capable of detecting NOVX protein or mRNA in a biological sample; means for determining the amount of NOVX in the sample; and means for comparing the amount of NOVX in the sample with a standard. The compound or agent can be packaged in a suitable container. The kit can further comprise instructions for using the kit to detect NOVX protein or nucleic acid.

Prognostic Assays

The diagnostic methods described herein can furthermore be utilized to identify subjects having or at risk of developing a disease or disorder associated with aberrant NOVX expression or activity. For example, the assays described herein, such as the preceding diagnostic assays or the following assays, can be utilized to identify a subject having or at risk of developing a disorder associated with NOVX protein, nucleic acid expression or activity. Alternatively, the prognostic assays can be utilized to identify a subject having or at risk for developing a disease or disorder. Thus, the invention provides a method for identifying a disease or disorder associated with aberrant NOVX expression or activity in which a test sample is obtained from a subject and NOVX protein or nucleic acid (e.g., mRNA, genomic DNA) is detected, wherein the presence of NOVX protein or nucleic acid is diagnostic for a subject having or at risk of developing a disease or disorder associated with aberrant NOVX expression or activity. As used herein, a “test sample” refers to a biological sample obtained from a subject of interest. For example, a test sample can be a biological fluid (e.g., serum), cell sample, or tissue.

Furthermore, the prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant NOVX expression or activity. For example, such methods can be used to determine whether a subject can be effectively treated with an agent for a disorder. Thus, the invention provides methods for determining whether a subject can be effectively treated with an agent for a disorder associated with aberrant NOVX expression or activity in which a test sample is obtained and NOVX protein or nucleic acid is detected (e.g., wherein the presence of NOVX protein or nucleic acid is diagnostic for a subject that can be administered the agent to treat a disorder associated with aberrant NOVX expression or activity).

The methods of the invention can also be used to detect genetic lesions in an NOVX gene, thereby determining if a subject with the lesioned gene is at risk for a disorder characterized by aberrant cell proliferation and/or differentiation. In various embodiments, the methods include detecting, in a sample of cells from the subject, the presence or absence of a genetic lesion characterized by at least one of an alteration affecting the integrity of a gene encoding an NOVX-protein, or the misexpression of the NOVX gene. For example, such genetic lesions can be detected by ascertaining the existence of at least one of: (i) a deletion of one or more nucleotides from an NOVX gene; (ii) an addition of one or more nucleotides to an NOVX gene; (iii) a substitution of one or more nucleotides of an NOVX gene, (iv) a chromosomal rearrangement of an NOVX gene; (v) an alteration in the level of a messenger RNA transcript of an NOVX gene, (vi) aberrant modification of an NOVX gene, such as of the methylation pattern of the genomic DNA, (vii) the presence of a non-wild-type splicing pattern of a messenger RNA transcript of an NOVX gene, (viii) a non-wild-type level of an NOVX protein, (ix) allelic loss of an NOVX gene, and (x) inappropriate post-translational modification of an NOVX protein. As described herein, there are a large number of assay techniques known in the art which can be used for detecting lesions in an NOVX gene. A preferred biological sample is a peripheral blood leukocyte sample isolated by conventional means from a subject. However, any biological sample containing nucleated cells may be used, including, for example, buccal mucosal cells.

In certain embodiments, detection of the lesion involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran, et al., 1988. Science 241: 1077-1080; and Nakazawa, et al., 1994. Proc. Natl. Acad. Sci. USA 91: 360-364), the latter of which can be particularly useful for detecting point mutations in the NOVX-gene (see, Abravaya, et al., 1995. Nucl. Acids Res. 23: 675-682). This method can include the steps of collecting a sample of cells from a patient, isolating nucleic acid (e.g., genomic, mRNA or both) from the cells of the sample, contacting the nucleic acid sample with one or more primers that specifically hybridize to an NOVX gene under conditions such that hybridization and amplification of the NOVX gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (see, Guatelli, et al., 1990. Proc. Natl Acad. Sci. USA 87: 1874-1878), transcriptional amplification system (see, Kwoh, et al., 1989. Proc. Natl Acad. Sci. USA 86: 1173-1177); Qβ Replicase (see, Lizardi, et al, 1988. BioTechnology 6: 1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in an NOVX gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, e.g., U.S. Pat. No. 5,493,531) can be used to score for the presence of specific mutations by development or loss of a ribozyme cleavage site.

In other embodiments, genetic mutations in NOVX can be identified by hybridizing a sample and control nucleic acids, e.g., DNA or RNA, to high-density arrays containing hundreds or thousands of oligonucleotides probes. See, e.g., Cronin, et at., 1996. Human Mutation 7:244-255; Kozal, et al., 1996. Nat. Med. 2: 753-759. For example, genetic mutations in NOVX can be identified in two dimensional arrays containing light-generated DNA probes as described in Cronin, et al., supra. Briefly, a first hybridization array of probes can be used to scan through long stretches of DNA in a sample and control to identify base changes between the sequences by making linear arrays of sequential overlapping probes. This step allows the identification of point mutations. This is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the NOVX gene and detect mutations by comparing the sequence of the sample NOVX with the corresponding wild-type (control) sequence. Examples of sequencing reactions include those based on techniques developed by Maxim and Gilbert, 1977. Proc. Natl. Acad. Sci. USA 74: 560 or Sanger, 1977. Proc. Natl. Acad. Sci. USA 74: 5463. It is also contemplated that any of a variety of automated sequencing procedures can be utilized when performing the diagnostic assays (see, e.g., Naeve, et al., 1995. Biotechniques 19: 448), including sequencing by mass spectrometry (see, e.g., PCT International Publication No. WO 94/16101; Cohen, et al., 1996.Adv. Chromatography 36: 127-162; and Griffin, et al., 1993. Appl. Biochem. Biotechnol. 38: 147-159).

Other methods for detecting mutations in the NOVX gene include methods in which protection from cleavage agents is used to detect mismatched bases in RNA/RNA or RNA/DNA heteroduplexes. See, e.g., Myers, et at., 1985. Science 230: 1242. In general, the art technique of “mismatch cleavage” starts by providing heteroduplexes of formed by hybridizing (labeled) RNA or DNA containing the wild-type NOVX sequence with potentially mutant RNA or DNA obtained from a tissue sample. The double-stranded duplexes are treated with an agent that cleaves single-stranded regions of the duplex such as which will exist due to basepair mismatches between the control and sample strands. For instance, RNA/DNA duplexes can be treated with RNase and DNA/DNA hybrids treated with S₁ nuclease to enzymatically digesting the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, e.g., Cotton, et al, 1988. Proc. Natl. Acad. Sci. USA 85: 4397; Saleeba, et al, 1992. Methods Enzymol. 217: 286-295. In an embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in NOVX cDNAs obtained from samples of cells. For example, the mutY enzyme of E. coli cleaves A at G/A mismatches and the thymidine DNA glycosylase from HeLa cells cleaves T at G/T mismatches. See, e.g., Hsu, et al., 1994. Carcinogenesis 15: 1657-1662. According to an exemplary embodiment, a probe based on an NOVX sequence, e.g., a wild-type NOVX sequence, is hybridized to a cDNA or other DNA product from a test cell(s). The duplex is treated with a DNA mismatch repair enzyme, and the cleavage products, if any, can be detected from electrophoresis protocols or the like. See, e.g., U.S. Pat. No. 5,459,039.

In other embodiments, alterations in electrophoretic mobility will be used to identify mutations in NOVX genes. For example, single strand conformation polymorphism (SSCP) may be used to detect differences in electrophoretic mobility between mutant and wild type nucleic acids. See, e.g., Orita, et al., 1989. Proc. Natl. Acad. Sci. USA: 86: 2766; Cotton, 1993. Mutat. Res. 285: 125-144; Hayashi, 1992. Genet. Anal. Tech. Appl. 9: 73-79. Single-stranded DNA fragments of sample and control NOVX nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In one embodiment, the subject method utilizes heteroduplex analysis to separate double stranded heteroduplex molecules on the basis of changes in electrophoretic mobility. See, e.g., Keen, et al., 1991. Trends Genet. 7: 5.

In yet another embodiment, the movement of mutant or wild-type fragments in polyacrylamide gels containing a gradient of denaturant is assayed using denaturing gradient gel electrophoresis (DGGE). See, e.g., Myers, et al., 1985. Nature 313: 495. When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA. See, e.g., Rosenbaum and Reissner, 1987. Biophys. Chem. 265: 12753.

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions that permit hybridization only if a perfect match is found. See, e.g., Saiki, et al., 1986. Nature 324: 163; Saiki, et al., 1989. Proc. Natl. Acad. Sci. USA 86: 6230. Such allele specific oligonucleotides are hybridized to PCR amplified target DNA or a number of different mutations when the oligonucleotides are attached to the hybridizing membrane and hybridized with labeled target DNA.

Alternatively, allele specific amplification technology that depends on selective PCR amplification may be used in conjunction with the instant invention. Oligonucleotides used as primers for specific amplification may carry the mutation of interest in the center of the molecule (so that amplification depends on differential hybridization; see, e.g., Gibbs, et al., 1989. Nucl. Acids Res. 17: 2437-2448) or at the extreme 3′-terminus of one primer where, under appropriate conditions, mismatch can prevent, or reduce polymerase extension (see, e.g., Prossner, 1993. Tibtech. 11: 238). In addition it may be desirable to introduce a novel restriction site in the region of the mutation to create cleavage-based detection. See, e.g., Gasparini, et al., 1992. Mol. Cell Probes 6: 1. It is anticipated that in certain embodiments amplification may also be performed using Taq ligase for amplification. See, e.g., Barany, 1991. Proc. Natl. Acad. Sci. USA 88: 189. In such cases, ligation will occur only if there is a perfect match at the 3′-terminus of the 5′ sequence, making it possible to detect the presence of a known mutation at a specific site by looking for the presence or absence of amplification.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving an NOVX gene.

Furthermore, any cell type or tissue, preferably peripheral blood leukocytes, in which NOVX is expressed may be utilized in the prognostic assays described herein. However, any biological sample containing nucleated cells may be used, including, for example, buccal mucosal cells.

Pharmacogenomics

Agents, or modulators that have a stimulatory or inhibitory effect on NOVX activity (e.g., NOVX gene expression), as identified by a screening assay described herein can be administered to individuals to treat (prophylactically or therapeutically) disorders (The disorders include metabolic disorders, diabetes, obesity, infectious disease, anorexia, cancer-associated cachexia, cancer, neurodegenerative disorders, Alzheimer's Disease, Parkinson's Disorder, immune disorders, and hematopoietic disorders, and the various dyslipidemias, metabolic disturbances associated with obesity, the metabolic syndrome X and wasting disorders associated with chronic diseases and various cancers.) In conjunction with such treatment, the pharmacogenomics (i.e., the study of the relationship between an individual's genotype and that individual's response to a foreign compound or drug) of the individual may be considered. Differences in metabolism of therapeutics can lead to severe toxicity or therapeutic failure by altering the relation between dose and blood concentration of the pharmacologically active drug. Thus, the pharmacogenomics of the individual permits the selection of effective agents (e.g., drugs) for prophylactic or therapeutic treatments based on a consideration of the individual's genotype. Such pharmacogenomics can further be used to determine appropriate dosages and therapeutic regimens. Accordingly, the activity of NOVX protein, expression of NOVX nucleic acid, or mutation content of NOVX genes in an individual can be determined to thereby select appropriate agent(s) for therapeutic or prophylactic treatment of the individual.

Pharmacogenomics deals with clinically significant hereditary variations in the response to drugs due to altered drug disposition and abnormal action in affected persons. See e.g., Eichelbaum, 1996. Clin. Exp. Pharmacol. Physiol., 23: 983-985; Linder, 1997. Clin. Chem., 43: 254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare defects or as polymorphisms. For example, glucose-6-phosphate dehydrogenase (G6PD) deficiency is a common inherited enzymopathy in which the main clinical complication is hemolysis after ingestion of oxidant drugs (anti-malarials, sulfonamides, analgesics, nitrofurans) and consumption of fava beans.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, PM show no therapeutic response, as demonstrated for the analgesic effect of codeine mediated by its CYP2D6-formed metabolite morphine. At the other extreme are the so called ultra-rapid metabolizers who do not respond to standard doses. Recently, the molecular basis of ultra-rapid metabolism has been identified to be due to CYP2D6 gene amplification.

Thus, the activity of NOVX protein, expression of NOVX nucleic acid, or mutation content of NOVX genes in an individual can be determined to thereby select appropriate agent(s) for therapeutic or prophylactic treatment of the individual. In addition, pharmacogenetic studies can be used to apply genotyping of polymorphic alleles encoding drug-metabolizing enzymes to the identification of an individual's drug responsiveness phenotype. This knowledge, when applied to dosing or drug selection, can avoid adverse reactions or therapeutic failure and thus enhance therapeutic or prophylactic efficiency when treating a subject with an NOVX modulator, such as a modulator identified by one of the exemplary screening assays described herein.

Monitoring of Effects During Clinical Trials

Monitoring the influence of agents (e.g., drugs, compounds) on the expression or activity of NOVX (e.g., the ability to modulate aberrant cell proliferation and/or differentiation) can be applied not only in basic drug screening, but also in clinical trials. For example, the effectiveness of an agent determined by a screening assay as described herein to increase NOVX gene expression, protein levels, or upregulate NOVX activity, can be monitored in clinical trails of subjects exhibiting decreased NOVX gene expression, protein levels, or downregulated NOVX activity. Alternatively, the effectiveness of an agent determined by a screening assay to decrease NOVX gene expression, protein levels, or downregulate NOVX activity, can be monitored in clinical trails of subjects exhibiting increased NOVX gene expression, protein levels, or upregulated NOVX activity. In such clinical trials, the expression or activity of NOVX and, preferably, other genes that have been implicated in, for example, a cellular proliferation or immune disorder can be used as a “read out” or markers of the immune responsiveness of a particular cell.

By way of example, and not of limitation, genes, including NOVX, that are modulated in cells by treatment with an agent (e.g., compound, drug or small molecule) that modulates NOVX activity (e.g., identified in a screening assay as described herein) can be identified. Thus, to study the effect of agents on cellular proliferation disorders, for example, in a clinical trial, cells can be isolated and RNA prepared and analyzed for the levels of expression of NOVX and other genes implicated in the disorder. The levels of gene expression (i.e., a gene expression pattern) can be quantified by Northern blot analysis or RT-PCR, as described herein, or alternatively by measuring the amount of protein produced, by one of the methods as described herein, or by measuring the levels of activity of NOVX or other genes. In this manner, the gene expression pattern can serve as a marker, indicative of the physiological response of the cells to the agent. Accordingly, this response state may be determined before, and at various points during, treatment of the individual with the agent.

In one embodiment, the invention provides a method for monitoring the effectiveness of treatment of a subject with an agent (e.g., an agonist., antagonist, protein, peptide, peptidomimetic, nucleic acid, small molecule, or other drug candidate identified by the screening assays described herein) comprising the steps of (i) obtaining a pre-administration sample from a subject prior to administration of the agent; (ii) detecting the level of expression of an NOVX protein, mRNA, or genomic DNA in the preadministration sample; (iii) obtaining one or more post-administration samples from the subject; (iv) detecting the level of expression or activity of the NOVX protein, mRNA, or genomic DNA in the post-administration samples; (v) comparing the level of expression or activity of the NOVX protein, mRNA, or genomic DNA in the pre-administration sample with the NOVX protein, mRNA, or genomic DNA in the post administration sample or samples; and (vi) altering the administration of the agent to the subject accordingly. For example, increased administration of the agent may be desirable to increase the expression or activity of NOVX to higher levels than detected, i.e., to increase the effectiveness of the agent. Alternatively, decreased administration of the agent may be desirable to decrease expression or activity of NOVX to lower levels than detected, i.e., to decrease the effectiveness of the agent.

Methods of Treatment

The invention provides for both prophylactic and therapeutic methods of treating a subject at risk of (or susceptible to) a disorder or having a disorder associated with aberrant NOVX expression or activity. The disorders include cardiomyopathy, atherosclerosis, hypertension, congenital heart defects, aortic stenosis, atrial septal defect (ASD), atrioventricular (A-V) canal defect, ductus arteriosus, pulmonary stenosis, subaortic stenosis, ventricular septal defect (VSD), valve diseases, tuberous sclerosis, scleroderma, obesity, transplantation, adrenoleukodystrophy, congenital adrenal hyperplasia, prostate cancer, neoplasm; adenocarcinoma, lymphoma, uterus cancer, fertility, hemophilia, hypercoagulation, idiopathic thrombocytopenic purpura, immunodeficiencies, graft versus host disease, AIDS, bronchial asthma, Crohn's disease; multiple sclerosis, treatment of Albright Hereditary Ostoeodystrophy, and other diseases, disorders and conditions of the like.

These methods of treatment will be discussed more fully, below.

Disease and Disorders

Diseases and disorders that are characterized by increased (relative to a subject not suffering from the disease or disorder) levels or biological activity may be treated with Therapeutics that antagonize (i.e., reduce or inhibit) activity. Therapeutics that antagonize activity may be administered in a therapeutic or prophylactic manner. Therapeutics that may be utilized include, but are not limited to: (i) an aforementioned peptide, or analogs, derivatives, fragments or homologs thereof; (ii) antibodies to an aforementioned peptide; (iii) nucleic acids encoding an aforementioned peptide; (iv) administration of antisense nucleic acid and nucleic acids that are “dysfunctional” (i.e., due to a heterologous insertion within the coding sequences of coding sequences to an aforementioned peptide) that are utilized to “knockout” endogenous function of an aforementioned peptide by homologous recombination (see, e.g., Capecchi, 1989. Science 244: 1288-1292); or (v) modulators ( i.e., inhibitors, agonists and antagonists, including additional peptide mimetic of the invention or antibodies specific to a peptide of the invention) that alter the interaction between an aforementioned peptide and its binding partner.

Diseases and disorders that are characterized by decreased (relative to a subject not suffering from the disease or disorder) levels or biological activity may be treated with Therapeutics that increase (i.e., are agonists to) activity. Therapeutics that upregulate activity may be administered in a therapeutic or prophylactic manner. Therapeutics that may be utilized include, but are not limited to, an aforementioned peptide, or analogs, derivatives, fragments or homologs thereof; or an agonist that increases bioavailability.

Increased or decreased levels can be readily detected by quantifying peptide and/or RNA, by obtaining a patient tissue sample (e.g., from biopsy tissue) and assaying it in vitro for RNA or peptide levels, structure and/or activity of the expressed peptides (or mRNAs of an aforementioned peptide). Methods that are well-known within the art include, but are not limited to, immunoassays (e.g., by Western blot analysis, immunoprecipitation followed by sodium dodecyl sulfate (SDS) polyacrylamide gel electrophoresis, immunocytochemistry, etc.) and/or hybridization assays to detect expression of mRNAs (e.g., Northern assays, dot blots, in situ hybridization, and the like).

Prophylactic Methods

In one aspect, the invention provides a method for preventing, in a subject, a disease or condition associated with an aberrant NOVX expression or activity, by administering to the subject an agent that modulates NOVX expression or at least one NOVX activity. Subjects at risk for a disease that is caused or contributed to by aberrant NOVX expression or activity can be identified by, for example, any or a combination of diagnostic or prognostic assays as described herein. Administration of a prophylactic agent can occur prior to the manifestation of symptoms characteristic of the NOVX aberrancy, such that a disease or disorder is prevented or, alternatively, delayed in its progression. Depending upon the type of NOVX aberrancy, for example, an NOVX agonist or NOVX antagonist agent can be used for treating the subject. The appropriate agent can be determined based on screening assays described herein. The prophylactic methods of the invention are further discussed in the following subsections.

Therapeutic Methods

Another aspect of the invention pertains to methods of modulating NOVX expression or activity for therapeutic purposes. The modulatory method of the invention involves contacting a cell with an agent that modulates one or more of the activities of NOVX protein activity associated with the cell. An agent that modulates NOVX protein activity can be an agent as described herein, such as a nucleic acid or a protein, a naturally-occurring cognate ligand of an NOVX protein, a peptide, an NOVX peptidomimetic, or other small molecule. In one embodiment, the agent stimulates one or more NOVX protein activity. Examples of such stimulatory agents include active NOVX protein and a nucleic acid molecule encoding NOVX that has been introduced into the cell. In another embodiment, the agent inhibits one or more NOVX protein activity. Examples of such inhibitory agents include antisense NOVX nucleic acid molecules and anti-NOVX antibodies. These modulatory methods can be performed in vitro (e.g., by culturing the cell with the agent) or, alternatively, in vivo (e.g., by administering the agent to a subject). As such, the invention provides methods of treating an individual afflicted with a disease or disorder characterized by aberrant expression or activity of an NOVX protein or nucleic acid molecule. In one embodiment, the method involves administering an agent (e.g., an agent identified by a screening assay described herein), or combination of agents that modulates (e.g., up-regulates or down-regulates) NOVX expression or activity. In another embodiment, the method involves administering an NOVX protein or nucleic acid molecule as therapy to compensate for reduced or aberrant NOVX expression or activity.

Stimulation of NOVX activity is desirable in situations in which NOVX is abnormally downregulated and/or in which increased NOVX activity is likely to have a beneficial effect. One example of such a situation is where a subject has a disorder characterized by aberrant cell proliferation and/or differentiation (e.g., cancer or immune associated disorders). Another example of such a situation is where the subject has a gestational disease (e.g., preclampsia).

Determination of the Biological Effect of the Therapeutic

In various embodiments of the invention, suitable in vitro or in vivo assays are performed to determine the effect of a specific Therapeutic and whether its administration is indicated for treatment of the affected tissue.

In various specific embodiments, in vitro assays may be performed with representative cells of the type(s) involved in the patient's disorder, to determine if a given Therapeutic exerts the desired effect upon the cell type(s). Compounds for use in therapy may be tested in suitable animal model systems including, but not limited to rats, mice, chicken, cows, monkeys, rabbits, and the like, prior to testing in human subjects. Similarly, for in vivo testing, any of the animal model system known in the art may be used prior to administration to human subjects.

Prophylactic and Therapeutic Uses of the Compositions of the Invention

The NOVX nucleic acids and proteins of the invention are useful in potential prophylactic and therapeutic applications implicated in a variety of disorders including, but not limited to: metabolic disorders, diabetes, obesity, infectious disease, anorexia, cancer-associated cancer, neurodegenerative disorders, Alzheimer's Disease, Parkinson's Disorder, immune disorders, hematopoietic disorders, and the various dyslipidemias, metabolic disturbances associated with obesity, the metabolic syndrome X and wasting disorders associated with chronic diseases and various cancers.

As an example, a cDNA encoding the NOVX protein of the invention may be useful in gene therapy, and the protein may be useful when administered to a subject in need thereof. By way of non-limiting example, the compositions of the invention will have efficacy for treatment of patients suffering from: metabolic disorders, diabetes, obesity, infectious disease, anorexia, cancer-associated cachexia, cancer, neurodegenerative disorders, Alzheimer's Disease, Parkinson's Disorder, immune disorders, hematopoietic disorders, and the various dyslipidemias.

Both the novel nucleic acid encoding the NOVX protein, and the NOVX protein of the invention, or fragments thereof, may also be useful in diagnostic applications, wherein the presence or amount of the nucleic acid or the protein are to be assessed. A further use could be as an anti-bacterial molecule (i.e., some peptides have been found to possess anti-bacterial properties). These materials are further useful in the generation of antibodies, which immunospecifically-bind to the novel substances of the invention for use in therapeutic or diagnostic methods.

The invention will be further described in the following examples, which do not limit the scope of the invention described in the claims.

### EXAMPLE 1.

### Quantitative Expression Analysis of Clones in Various Cells and Tissues

The quantitative expression of various clones was assessed using microtiter plates containing RNA samples from a variety of normal and pathology-derived cells, cell lines and tissues using real time quantitative PCR (RTQ PCR; TAQMAN®). RTQ PCR was performed on a Perkin-Elmer Biosystems ABI PRISM® 7700 Sequence Detection System. Various collections of samples are assembled on the plates, and referred to as Panel 1 (containing cells and cell lines from normal and cancer sources), Panel 2 (containing samples derived from tissues, in particular from surgical samples, from normal and cancer sources), Panel 3 (containing samples derived from a wide variety of cancer sources) and Panel 4 (containing cells and cell lines from normal cells and cells related to inflammatory conditions).

First, the RNA samples were normalized to constitutively expressed genes such as β-actin and GAPDH. RNA (˜50 ng total or ˜1 ng polyA+) was converted to cDNA using the TAQMAN® Reverse Transcription Reagents Kit (PE Biosystems, Foster City, Calif.; Catalog No. N808-0234) and random hexamers according to the manufacturer's protocol. Reactions were performed in 20 ul and incubated for 30 min. at 48° C. cDNA (5 ul) was then transferred to a separate plate for the TAQMAN® reaction using β-actin and GAPDH TAQMAN® Assay Reagents (PE Biosystems; Catalog Nos. 4310881 E and 4310884E, respectively) and TAQMAN® universal PCR Master Mix (PE Biosystems; Catalog No. 4304447) according to the manufacturer's protocol. Reactions were performed in 25 ul using the following parameters: 2 min. at 50° C.; 10 min. at 95° C.; 15 sec. at 95° C./1 min. at 60° C. (40 cycles). Results were recorded as CT values (cycle at which a given sample crosses a threshold level of fluorescence) using a log scale, with the difference in RNA concentration between a given sample and the sample with the lowest CT value being represented as 2 to the power of delta CT. The percent relative expression is then obtained by taking the reciprocal of this RNA difference and multiplying by 100. The average CT values obtained for β-actin and GAPDH were used to normalize RNA samples. The RNA sample generating the highest CT value required no further diluting, while all other samples were diluted relative to this sample according to their β-actin/GAPDH average CT values.

Normalized RNA (5 ul) was converted to cDNA and analyzed via TAQMAN® using One Step RT-PCR Master Mix Reagents (PE Biosystems; Catalog No. 4309169) and gene-specific primers according to the manufacturer's instructions. Probes and primers were designed for each assay according to Perkin Elmer Biosystem's Primer Express Software package (version I for Apple Computer's Macintosh Power PC) or a similar algorithm using the target sequence as input. Default settings were used for reaction conditions and the following parameters were set before selecting primers: primer concentration=250 nM, primer melting temperature (Tₘ) range=58°-60° C., primer optimal Tm=59° C., maximum primer difference =2° C., probe does not have 5′ G, probe Tₘ must be 10° C. greater than primer Tₘ, amplicon size 75 bp to 100 bp. The probes and primers selected (see below) were synthesized by Synthegen (Houston, Tex., USA). Probes were double purified by HPLC to remove uncoupled dye and evaluated by mass spectroscopy to verify coupling of reporter and quencher dyes to the 5′ and 3′ ends of the probe, respectively. Their final concentrations were: forward and reverse primers, 900 nM each, and probe, 200 nM.

PCR conditions: Normalized RNA from each tissue and each cell line was spotted in each well of a 96 well PCR plate (Perkin Elmer Biosystems). PCR cocktails including two probes (a probe specific for the target clone and another gene-specific probe multiplexed with the target probe) were set up using 1× TaqMan™ PCR Master Mix for the PE Biosystems 7700, with 5 mM MgC12, dNTPs (dA, G, C, U at 1:1:1:2 ratios), 0.25 U/ml AmpliTaq Gold™ (PE Biosystems), and 0.4 U/μl RNase inhibitor, and 0.25 U/μl reverse transcriptase. Reverse transcription was performed at 48° C. for 30 minutes followed by amplification/PCR cycles as follows: 95° C. 10 min, then 40 cycles of 95° C. for 15 seconds, 60° C. for 1 minute.

In the results for Panel 1, the following abbreviations are used:

ca.=carcinoma,

*=established from metastasis,

met=metastasis,

s cell var=small cell variant,

non-s=non-sm=non-small,

squam=squamous,

p1 eff=p1 effusion=pleural effusion,

glio=glioma,

astro=astrocytoma, and

neuro=neuroblastoma.

Panel 2

The plates for Panel 2 generally include 2 control wells and 94 test samples composed of RNA or cDNA isolated from human tissue procured by surgeons working in close cooperation with the National Cancer Institute's Cooperative Human Tissue Network (CHTN) or the National Disease Research Initiative (NDRI). The tissues are derived from human malignancies and in cases where indicated many malignant tissues have “matched margins” obtained from noncancerous tissue just adjacent to the tumor. These are termed normal adjacent tissues and are denoted “NAT” in the results below. The tumor tissue and the “matched margins” are evaluated by two independent pathologists (the surgical pathologists and again by a pathologists at NDRI or CHTN). This analysis provides a gross histopathological assessment of tumor differentiation grade. Moreover, most samples include the original surgical pathology report that provides information regarding the clinical stage of the patient. These matched margins are taken from the tissue surrounding (i.e. immediately proximal) to the zone of surgery (designated “NAT”, for normal adjacent tissue, in Table RR). In addition, RNA and cDNA samples were obtained from various human tissues derived from autopsies performed on elderly people or sudden death victims (accidents, etc.). These tissue were ascertained to be free of disease and were purchased from various commercial sources such as Clontech (Palo Alto, Calif.), Research Genetics, and Invitrogen.

RNA integrity from all samples is controlled for quality by visual assessment of agarose gel electropherograms using 28S and 18S ribosomal RNA staining intensity ratio as a guide (2:1 to 2.5:1 28s:18s) and the absence of low molecular weight RNAs that would be indicative of degradation products. Samples are controlled against genomic DNA contamination by RTQ PCR reactions run in the absence of reverse transcriptase using probe and primer sets designed to amplify across the span of a single exon.

Panel 4

Panel 4 includes samples on a 96 well plate (2 control wells, 94 test samples) composed of RNA (Panel 4r) or cDNA (Panel 4d) isolated from various human cell lines or tissues related to inflammatory conditions. Total RNA from control normal tissues such as colon and lung (Stratagene ,La Jolla, Calif.) and thymus and kidney (Clontech) were employed. Total RNA from liver tissue from cirrhosis patients and kidney from lupus patients was obtained from BioChain (Biochain Institute, Inc., Hayward, Calif.). Intestinal tissue for RNA preparation from patients diagnosed as having Crohn's disease and ulcerative colitis was obtained from the National Disease Research Interchange (NDRI) (Philadelphia, Pa.).

Astrocytes, lung fibroblasts, dermal fibroblasts, coronary artery smooth muscle cells, small airway epithelium, bronchial epithelium, microvascular dermal endothelial cells, microvascular lung endothelial cells, human pulmonary aortic endothelial cells, human umbilical vein endothelial cells were all purchased from Clonetics (Walkersville, Md.) and grown in the media supplied for these cell types by Clonetics. These primary cell types were activated with various cytokines or combinations of cytokines for 6 and/or 12-14 hours, as indicated. The following cytokines were used; IL-1 beta at approximately 1-5 ng/ml, TNF alpha at approximately 5-10 ng/ml, IFN gamma at approximately 20-50 ng/ml, IL-4 at approximately 5-10 ng/ml, IL-9 at approximately 5-10 ng/ml, IL-13 at approximately 5-10 ng/ml. Endothelial cells were sometimes starved for various times by culture in the basal media from Clonetics with 0.1% serum.

Mononuclear cells were prepared from blood of employees at CuraGen Corporation, using Ficoll. LAK cells were prepared from these cells by culture in DMEM 5% FCS (Hyclone), 100 μM non essential amino acids (Gibco/Life Technologies, Rockville, Md.), I mM sodium pyruvate (Gibco), mercaptoethanol 5.5×10₋₅ M (Gibco), and 10 mM Hepes (Gibco) and Interleukin 2 for 4-6 days. Cells were then either activated with 10-20 ng/ml PMA and 1-2 μg/ml ionomycin, IL-12 at 5-10 ng/ml, IFN gamma at 20-50 ng/ml and IL-18 at 5-10 ng/ml for 6 hours. In some cases, mononuclear cells were cultured for 4-5 days in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco) with PHA (phytohemagglutinin) or PWM (pokeweed mitogen) at approximately 5 μg/ml. Samples were taken at 24, 48 and 72 hours for RNA preparation. MLR (mixed lymphocyte reaction) samples were obtained by taking blood from two donors, isolating the mononuclear cells using Ficoll and mixing the isolated mononuclear cells 1:1 at a final concentration of approximately 2×10⁶ cells/ml in DMEM 5% FCS (Hyclone), 100 μM non essential amino acids (Gibco), 1 mM sodium pyruvate (Gibco), mercaptoethanol (5.5×10⁻⁵ M) (Gibco), and 10 mM Hepes (Gibco). The MLR was cultured and samples taken at various time points ranging from 1- 7 days for RNA preparation.

Monocytes were isolated from mononuclear cells using CD14 Miltenyi Beads, +ve VS selection columns and a Vario Magnet according to the manufacturer's instructions. Monocytes were differentiated into dendritic cells by culture in DMEM 5% fetal calf serum (FCS) (Hyclone, Logan, Utah), 100 μM non essential amino acids (Gibco), 1 mM sodium pyruvate (Gibco), mercaptoethanol 5.5×10⁻⁵ M (Gibco), and 10 mM Hepes (Gibco), 50 ng/ml GMCSF and 5 ng/ml IL-4 for 5-7 days. Macrophages were prepared by culture of monocytes for 5-7 days in DMEM 5% FCS (Hyclone), 100 μM non essential amino acids (Gibco), 1 mM sodium pyruvate (Gibco), mercaptoethanol 5.5×10⁻⁵ M (Gibco), 10 mM Hepes (Gibco) and 10% AB Human Serum or MCSF at approximately 50 ng/ml. Monocytes, macrophages and dendritic cells were stimulated for 6 and 12-14 hours with lipopolysaccharide (LPS) at 100 ng/ml. Dendritic cells were also stimulated with anti-CD40 monoclonal antibody (Pharmingen) at 10 μg/ml for 6 and 12-14 hours.

CD4 lymphocytes, CD8 lymphocytes and NK cells were also isolated from mononuclear cells using CD4, CD8 and CD56 Miltenyi beads, positive VS selection columns and a Vario Magnet according to the manufacturer's instructions. CD45RA and CD45RO CD4 lymphocytes were isolated by depleting mononuclear cells of CD8, CD56, CD14 and CD19 cells using CD8, CD56, CD14 and CD19 Miltenyi beads and positive selection. Then CD45RO beads were used to isolate the CD45RO CD4 lymphocytes with the remaining cells being CD45RA CD4 lymphocytes. CD45RA CD4, CD45RO CD4 and CD8 lymphocytes were placed in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco) and plated at 10⁶ cells/ml onto Falcon 6 well tissue culture plates that had been coated overnight with 0.5 μg/ml anti-CD28 (Pharmingen) and 3 ug/ml anti-CD3 (OKT3, ATCC) in PBS. After 6 and 24 hours, the cells were harvested for RNA preparation. To prepare chronically activated CD8 lymphocytes, we activated the isolated CD8 lymphocytes for 4 days on anti-CD28 and anti-CD3 coated plates and then harvested the cells and expanded them in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco) and IL-2. The expanded CD8 cells were then activated again with plate bound anti-CD3 and anti-CD28 for 4 days and expanded as before. RNA was isolated 6 and 24 hours after the second activation and after 4 days of the second expansion culture. The isolated NK cells were cultured in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco) and IL-2 for 4-6 days before RNA was prepared.

To obtain B cells, tonsils were procured from NDRI. The tonsil was cut up with sterile dissecting scissors and then passed through a sieve. Tonsil cells were then spun down and resupended at 10⁶ cells/ml in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco). To activate the cells, we used PWM at 5 pg/ml or anti-CD40 (Pharmingen) at approximately 10 μg/ml and IL-4 at 5-10 ng/ml. Cells were harvested for RNA preparation at 24,48 and 72 hours.

To prepare the primary and secondary Th1/Th2 and Trl cells, six-well Falcon plates were coated overnight with 10 μg/ml anti-CD28 (Pharmingen) and 2 μg/ml OKT3 (ATCC), and then washed twice with PBS. Umbilical cord blood CD4 lymphocytes (Poietic Systems, German Town, Md.) were cultured at 10⁵-10⁶ cells/ml in DMEM 5% FCS (Hyclone), 100 μM non essential amino acids (Gibco), 1 mM sodium pyruvate (Gibco), mercaptoethanol 5.5×10⁻⁵ M (Gibco), 10 mM Hepes (Gibco) and IL-2 (4 ng/ml). IL-12 (5 ng/ml) and anti-IL4 (1 □g/ml) were used to direct to Th1, while IL-4 (5 ng/ml) and anti-IFN gamma (1 □g/ml) were used to direct to Th2 and IL-10 at 5 ng/ml was used to direct to Tr1. After 4-5 days, the activated Th1, Th2 and Tr1 lymphocytes were washed once in DMEM and expanded for 4-7 days in DMEM 5% FCS (Hyclone), 100 μM non essential amino acids (Gibco), 1 mM sodium pyruvate (Gibco), mercaptoethanol 5.5×10⁻⁵ M (Gibco), 10 mM Hepes (Gibco) and IL-2 (1 ng/ml). Following this, the activated Th1, Th2 and Tr1 lymphocytes were re-stimulated for 5 days with anti-CD28/OKT3 and cytokines as described above, but with the addition of anti-CD95L (1 □g/ml) to prevent apoptosis. After 4-5 days, the Th1, Th2 and Tr1 lymphocytes were washed and then expanded again with IL-2 for 4-7 days. Activated Th1 and Th2 lymphocytes were maintained in this way for a maximum of three cycles. RNA was prepared from primary and secondary Th1, Th2 and Tr1 after 6 and 24 hours following the second and third activations with plate bound anti-CD3 and anti-CD28 mAbs and 4 days into the second and third expansion cultures in Interleukin 2.

The following leukocyte cells lines were obtained from the ATCC: Ramos, EOL-1, KU-812. EOL cells were further differentiated by culture in 0.1 mM dbcAMP at 5×10⁵ cells/ml for 8 days, changing the media every 3 days and adjusting the cell concentration to 5×10⁵ cells/ml. For the culture of these cells, we used DMEM or RPMI (as recommended by the ATCC), with the addition of 5% FCS (Hyclone), 100 μM non essential amino acids (Gibco), 1 mM sodium pyruvate (Gibco), mercaptoethanol 5.5×10⁻⁵ M (Gibco), 10 mM Hepes (Gibco). RNA was either prepared from resting cells or cells activated with PMA at 10 ng/ml and ionomycin at 1 μg/ml for 6 and 14 hours. Keratinocyte line CCD106 and an airway epithelial tumor line NCI-H292 were also obtained from the ATCC. Both were cultured in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco). CCD1106 cells were activated for 6 and 14 hours with approximately 5 ng/ml TNF alpha and 1 ng/ml IL-1 beta, while NCI-H292 cells were activated for 6 and 14 hours with the following cytokines: 5 ng/ml IL-4, 5 ng/ml IL-9, 5 ng/ml IL-13 and 25 ng/ml IFN gamma.

For these cell lines and blood cells, RNA was prepared by lysing approximately 10⁷ cells/ml using Trizol (Gibco BRL). Briefly, 1/10volume of bromochloropropane (Molecular Research Corporation) was added to the RNA sample, vortexed and after 10 minutes at room temperature, the tubes were spun at 14,000 rpm in a Sorvall SS34 rotor. The aqueous phase was removed and placed in a 15 ml Falcon Tube. An equal volume of isopropanol was added and left at −20 degrees C. overnight. The precipitated RNA was spun down at 9,000 rpm for 15 min in a Sorvall SS34 rotor and washed in 70% ethanol. The pellet was redissolved in 300 μl of RNAsefree water and 35 μl buffer (Promega) 5 μl DTT, 7 μl RNAsin and 8 Al DNAse were added. The tube was incubated at 37 degrees C. for 30 minutes to remove contaminating genomic DNA, extracted once with phenol chloroform and re-precipitated with 1/10volume of 3 M sodium acetate and 2 volumes of 100% ethanol. The RNA was spun down and placed in RNAse free water. RNA was stored at −80 degrees C.

Central Nervous System (CNS) Panel

The CNS panel (e.g., CNS.D01) was run on 96 well plates. The plates generally include 2 control wells and 94 test samples composed of RNA or cDNA isolated from human tissue procured by the Harvard Brain Bank. These are samples of human brain tissue collected at autopsy from patients diagnosed as having various CNS disease such as schizophrenia, depression, progressive supranuclear palsy, Alzheimer's disease, Huntington's disease, and Parkinson's disease, as well as neurologically normal controls. The samples were obtained from different brain regions.

RNA integrity from all samples is controlled for quality by visual assessment of agarose gel electropherograms using (1) the ratio of staining intensity for 28S and 18S ribosomal RNA as a guide (2:1 to 2.5:1 for the ratio of 28S RNA: I 8S RNA) and (2) the absence of low molecular weight RNAs that would be indicative of degradation products. Samples are controlled against genomic DNA contamination by RTQ PCR reactions run in the absence of reverse transcriptase using probe and primer sets designed to amplify across the span of a single exon.

In the labels employed to identify tissues in the CNS panel the following abbreviations are used:

PSP: Progressive supranuclear palsy

Sub Nigra: Substantia nigra

Glob Palladus:Globus pallidus

Temp Pole: Temporal pole

Cing Gyr: Cingulate gyrus

NOV1a

Expression of gene NOV1a was assessed using the primer-probe set Ag1219, described in Table B.

TABLE B
Probe Name: Ag1219

| | | | | | SEQ |
|---|---|---|---|---|---|
| | | | | Start | ID |
| Primers | Sequences | TM | Length | Position | # |
| Forward | 5′-GGAGGGCCTGAAAGTACTCA-3′ | 59.3 | 20 | 74 | 60 |
| Probe | FAM-5′-AAAAGACCCTCTGGCTGCAGACATAA-3′- | 67.2 | 26 | 128 | 61 |
| | TAMRA | | | | |
| Reverse | 5′-CTGGTCCATGTCCTTCATTC-3′ | 58.9 | 20 | 173 | 62 |

Expression of this gene in panels 1.2 and 4D was low/undetectable (Ct values>35) in all samples.

NOV2

Expression of gene NOV2 was assessed using the primer-probe sets Ag1702 and Ag1209 (identical sequences), described in Table C. Results of the RTQ-PCR runs are shown in Tables D and E.

TABLE C
Probe Name: Ag1702/Ag1209

| | | | | | SEQ |
|---|---|---|---|---|---|
| | | | | Start | ID |
| Primers | Sequences | TM | Length | Position | # |
| Forward | 5′-TGGAATGACACAAGCCAATC-3′ | 59.5 | 20 | 1 | 63 |
| Probe | FAM-5′-AGACCTAGAGCATCCCTGCAGTGCCT-3′- | 70.3 | 26 | 23 | 64 |
| | TAMRA | | | | |
| Reverse | 5′-TGGGTGCTACAAGAGGAGAA-3′ | 58.4 | 20 | 58 | 65 |

TABLE D
Panel 1.2

| | Relative | | Relative |
|---|---|---|---|
| | Expression (%) | | Expression (%) |
| | 1.2tm1401t_ag1 | | 1.2tm1401t_ag |
| Tissue Name | 209 | Tissue Name | 1209 |
| Endothelial cells | 0.0 | Kidney (fetal) | 0.0 |
| Endothelial cells (treated) | 0.0 | Renal ca. 786-0 | 0.0 |
| Pancreas | 0.0 | Renal ca. A498 | 0.0 |
| Pancreatic ca. CAPAN 2 | 0.0 | Renal ca. RXF 393 | 0.0 |
| Adrenal Gland (new lot*) | 0.0 | Renal ca. ACHN | 0.0 |
| Thyroid | 0.0 | Renal ca. UO-31 | 0.0 |
| Salivary gland | 0.0 | Renal ca. TK-10 | 0.0 |
| Pituitary gland | 0.0 | Liver | 0.0 |
| Brain (fetal) | 0.0 | Liver (fetal) | 0.0 |
| Brain (whole) | 0.0 | Liver ca. (hepatoblast) HepG2 | 0.0 |
| Brain (amygdala) | 0.0 | Lung | 0.0 |
| Brain (cerebellum) | 0.0 | Lung (fetal) | 0.0 |
| Brain (hippocampus) | 0.0 | Lung ca. (small cell) LX-1 | 0.0 |
| Brain (thalamus) | 0.0 | Lung ca. (small cell) NCI-H69 | 21.2 |
| Cerebral Cortex | 0.0 | Lung ca. (s. cell var.) SHP-77 | 0.0 |
| Spinal cord | 0.0 | Lung ca. (large cell) NCI-H460 | 0.0 |
| CNS ca. (glio/astro) U87-MG | 0.0 | Lung ca. (non-sm. cell) A549 | 19.5 |
| CNS ca. (glio/astro) U-118-MG | 0.0 | Lung ca. (non-s. cell) NCI-H23 | 0.0 |
| CNS ca. (astro) SW1783 | 0.0 | Lung ca (non-s. cell) HOP-62 | 0.0 |
| CNS ca.* (neuro; met) SK-N-AS | 0.0 | Lung ca. (non-s. cl) NCI-H522 | 0.0 |
| CNS ca. (astro) SF-539 | 0.0 | Lung ca. (squam.) SW 900 | 0.0 |
| CNS ca. (astro) SNB-75 | 0.0 | Lung ca. (squam.) NCI-H596 | 0.0 |
| CNS ca. (glio) SNB-19 | 0.0 | Mammary gland | 0.0 |
| CNS ca. (glio) U251 | 0.0 | Breast ca.* (pl. effusion) MCF-7 | 0.0 |
| CNS ca. (glio) SF-295 | 0.0 | Breast ca.* (pl. ef) MDA-MB-231 | 0.0 |
| Heart | 0.0 | Breast ca.* (pl. effusion) T47D | 0.0 |
| Skeletal Muscle (new lot*) | 0.0 | Breast ca. BT-549 | 0.0 |
| Bone marrow | 0.0 | Breast ca. MDA-N | 0.0 |
| Thymus | 0.0 | Ovary | 0.0 |
| Spleen | 0.0 | Ovarian ca. OVCAR-3 | 0.0 |
| Lymph node | 0.0 | Ovarian ca. OVCAR-4 | 0.0 |
| Colorectal | 0.0 | Ovarian ca. OVCAR-5 | 2.8 |
| Stomach | 0.0 | Ovarian ca. OVCAR-8 | 0.0 |
| Small intestine | 0.0 | Ovarian ca. IGROV-1 | 0.0 |
| Colon ca. SW480 | 0.0 | Ovarian ca.* (ascites) SK-OV-3 | 0.0 |
| Colon ca.* (SW480 met) SW620 | 0.0 | Uterus | 0.0 |
| Colon ca. HT29 | 0.0 | Placenta | 0.0 |
| Colon ca. HCT-116 | 0.0 | Prostate | 0.0 |
| Colon ca. CaCo-2 | 0.0 | Prostate ca.* (bone met) PC-3 | 0.0 |
| 83219 CC Well to Mod Diff (ODO3866) | 100.0 | Testis | 0.0 |
| Colon ca. HCC-2998 | 0.0 | Melanoma Hs688(A).T | 0.0 |
| Gastric ca.* (liver met) NCI-N87 | 0.0 | Melanoma* (met) Hs688(B).T | 3.9 |
| Bladder | 0.0 | Melanoma UACC-62 | 0.0 |
| Trachea | 0.0 | Melanoma M14 | 29.3 |
| Kidney | 0.0 | Melanoma LOX IMVI | 0.0 |
| | | Melanoma* (met) SK-MEL-5 | 0.0 |

TABLE E
Panel 4D

| | Relative | Relative |
|---|---|---|
| | Expression | Expression |
| | (%) | (%) |
| | 4dx4tm5105f_ | 4dtm2065t_ag |
| Tissue Name | ag1702_a2 | 1209 |
| 93768_Secondary Th1_anti-CD28/anti-CD3 | 0.0 | 0.0 |
| 93769_Secondary Th2_anti-CD28/anti-CD3 | 0.0 | 0.0 |
| 93770_Secondary Tr1_anti-CD28/anti-CD3 | 0.0 | 0.0 |
| 93573_Secondary Th1_resting day 4-6 in IL − 2 | 0.0 | 0.0 |
| 93572_Secondary Th2_resting day 4-6 in IL − 2 | 0.0 | 0.0 |
| 93571_Secondary Tr1_resting day 4-6 in IL − 2 | 0.0 | 0.0 |
| 93568_primary Th1_anti-CD28/anti-CD3 | 0.0 | 0.0 |
| 93569_primary Th2_anti-CD28/anti-CD3 | 0.0 | 0.0 |
| 93570_primary Tr1_anti-CD28/anti-CD3 | 0.0 | 0.0 |
| 93565_primary Th1_resting dy 4-6 in IL − 2 | 0.0 | 0.0 |
| 93566_primary Th2_resting dy 4-6 in IL − 2 | 0.0 | 0.0 |
| 93567_primary Tr1_resting dy 4-6 in IL − 2 | 0.0 | 0.0 |
| 93351_CD45RA CD4 lymphocyte_anti-CD28/anti-CD3 | 0.0 | 0.0 |
| 93352_CD45RO CD4 lymphocyte_anti-CD28/anti-CD3 | 0.0 | 0.0 |
| 93251_CD8 Lymphocytes_anti-CD28/anti-CD3 | 0.0 | 0.0 |
| 93353_chronic CD8 Lymphocytes 2ry_resting dy 4-6 in IL − 2 | 0.0 | 0.0 |
| 93574_chronic CD8 Lymphocytes 2ry_activated CD3/CD28 | 0.0 | 0.0 |
| 93354_CD4_none | 0.0 | 0.0 |
| 93252_Secondary Th1/Th2/Tr1_anti-CD95 CH11 | 0.0 | 0.0 |
| 93103_LAK cells_resting | 0.0 | 0.0 |
| 93788_LAK cells_IL − 2 | 0.0 | 0.0 |
| 93787_LAK cells_IL − 2 + IL − 12 | 92.7 | 0.0 |
| 93789_LAK cells_IL − 2 + IFN gamma | 0.0 | 0.0 |
| 93790_LAK cells_IL − 2 + IL − 18 | 0.0 | 0.0 |
| 93104_LAK cells_PMA/ionomycin and IL − 18 | 0.0 | 0.0 |
| 93578_NK Cells IL − 2_resting | 0.0 | 0.0 |
| 93109_Mixed Lymphocyte Reaction_Two Way MLR | 0.0 | 0.0 |
| 93110_Mixed Lymphocyte Reaction_Two Way MLR | 0.0 | 0.0 |
| 93111_Mixed Lymphocyte Reaction_Two Way MLR | 0.0 | 0.0 |
| 93112_Mononuclear Cells (PBMCs)_resting | 0.0 | 0.0 |
| 93113_Mononuclear Cells (PBMCs)_PWM | 0.0 | 0.0 |
| 93114_Mononuclear Cells (PBMCs)_PHA-L | 0.0 | 0.0 |
| 93249_Ramos (B cell)_none | 0.0 | 0.0 |
| 93250_Ramos (B cell)_ionomycin | 0.0 | 0.0 |
| 93349_B lymphocytes_PWM | 0.0 | 0.0 |
| 93350_B lymphoytes_CD40L and IL − 4 | 100.0 | 0.0 |
| 92665_EOL-1 (Eosinophil)_dbcAMP differentiated | 0.0 | 0.0 |
| 93248_EOL-1 (Eosinophil)_dbcAMP/PMA ionomycin | 0.0 | 0.0 |
| 93356_Dendritic Cells_none | 0.0 | 0.0 |
| 93355_Dendritic Cells_LPS 100 ng/ml | 0.0 | 0.0 |
| 93775_Dendritic Cells_anti-CD40 | 0.0 | 0.0 |
| 93774_Monocytes_resting | 0.0 | 0.0 |
| 93776_Monocytes_LPS 50 ng/ml | 0.0 | 0.0 |
| 93581_Macrophages_resting | 0.0 | 0.0 |
| 93582_Macrophages_LPS 100 ng/ml | 0.0 | 0.0 |
| 93098_HUVEC (Endothelial)_none | 0.0 | 0.0 |
| 93099_HUVEC (Endothelial)_starved | 0.0 | 0.0 |
| 93100_HUVEC (Endothelial)_IL − 1b | 0.0 | 0.0 |
| 93779_HUVEC (Endothelial)_IFN gamma | 0.0 | 0.0 |
| 93102_HUVEC (Endothelial)_TNF alpha + IFN gamma | 0.0 | 0.0 |
| 93101_HUVEC (Endothelial)_TNF alpha + IL4 | 0.0 | 0.0 |
| 93781_HUVEC (Endothelial)_IL − 11 | 0.0 | 0.0 |
| 93583_Lung Microvascular Endothelial Cells_none | 0.0 | 0.0 |
| 93584_Lung Microvascular Endothelial Cells_TNFa (4 ng/ml) | 0.0 | 0.0 |
| and IL1b (1 ng/ml) | | |
| 92662_Microvascular Dermal endothelium_none | 0.0 | 0.0 |
| 92663_Microsvasular Dermal endothelium_TNFa (4 ng/ml) and | 0.0 | 0.0 |
| IL1b (1 ng/ml) | | |
| 93773_Bronchial epithelium_TNFa (4 ng/ml) and IL1b | 0.0 | 0.0 |
| (1 ng/ml)** | | |
| 93347_Small Airway Epithelium_none | 0.0 | 0.0 |
| 93348_Small Airway Epithelium_TNFa (4 ng/ml) and IL1b | 0.0 | 0.0 |
| (1 ng/ml) | | |
| 92668_Coronery Artery SMC_resting | 0.0 | 0.0 |
| 92669_Coronery Artery SMC_TNFa (4 ng/ml) and IL1b | 0.0 | 0.0 |
| (1 ng/ml) | | |
| 93107_astrocytes_resting | 0.0 | 0.0 |
| 93108_astrocytes_TNFa (4 ng/ml) and IL1b (1 ng/ml) | 0.0 | 0.0 |
| 92666_KU-812 (Basophil)_resting | 0.0 | 0.0 |
| 92667_KU-812 (Basophil)_PMA/ionomycin | 0.0 | 0.0 |
| 93579_CCD1106 (Keratinocytes)_none | 0.0 | 0.0 |
| 93580_CCD1106 (Keratinocytes)_TNFa and IFNg** | 0.0 | 0.0 |
| 93791_Liver Cirrhosis | 33.3 | 88.3 |
| 93792_Lupus Kidney | 0.0 | 0.0 |
| 93577_NCI-H292 | 0.0 | 0.0 |
| 93358_NCI-H292_IL − 4 | 42.4 | 0.0 |
| 93360_NCI-H292_IL − 9 | 2.1 | 0.0 |
| 93359_NCI-H292_IL − 13 | 0.0 | 0.0 |
| 93357_NCI-H292_IFN gamma | 0.0 | 0.0 |
| 93777_HPAEC_ − | 0.0 | 0.0 |
| 93778_HPAEC_IL − 1 beta/TNA alpha | 0.0 | 0.0 |
| 93254_Normal Human Lung Fibroblast_none | 0.0 | 0.0 |
| 93253_Normal Human Lung Fibroblast_TNFa (4 ng/ml) and | 0.0 | 0.0 |
| IL − 1b (1 ng/ml) | | |
| 93257_Normal Human Lung Fibroblast_IL − 4 | 0.0 | 0.0 |
| 93256_Normal Human Lung Fibroblast_IL − 9 | 0.0 | 0.0 |
| 93255_Normal Human Lung Fibroblast_IL − 13 | 0.0 | 0.0 |
| 93258_Normal Human Lung Fibroblast_IFN gamma | 0.0 | 0.0 |
| 93106_Dermal Fibroblasts CCD1070_resting | 0.0 | 0.0 |
| 93361_Dermal Fibroblasts CCD1070_TNF alpha 4 ng/ml | 0.0 | 0.0 |
| 93105_Dermal Fibroblasts CCD1070_IL − 1 beta 1 ng/ml | 0.0 | 0.0 |
| 93772_dermal fibroblast_IFN gamma | 0.0 | 0.0 |
| 93771_dermal fibroblast_IL − 4 | 0.0 | 0.0 |
| 93259_IBD Colitis 1 ** | 0.0 | 100.0 |
| 93260_IBD Colitis 2 | 33.6 | 0.0 |
| 93261_IBD Crohn's | 0.0 | 6.7 |
| 735010_Colon_normal | 0.0 | 0.0 |
| 735019_Lung_none | 0.0 | 0.0 |
| 64028-1_Thymus_none | 0.0 | 0.0 |
| 64030-1_Kidney_none | 0.0 | 0.0 |

Panel 1.2 Summary: Expression of gene NOV2 in this panel is skewed by genomic DNA contamination in the adipose. Excluding this, the only sample that shows modest levels of expression of this gene is colon cancer. Other tissues show low/undetectable (Ct values>35) levels of expression. However expression in colon cancer is undetectable in the same sample of panel 1.2D, run with Ag1702.

Panel 4D Summary: Ag 1209—The level of expression of gene NOV2 is very low with the highest CT value at 34.5. Ag 1702—Expression of the NOV2 appears to be regulated by IL-12 in LAK cells, with IL-4 and CD40L (CD154) treatment in B cells and by IL-4 in NCI-H292 cells. This gene (NOV2) is also upregulated in colitis. The role of the protein encoded for by this transcript in inflammation may be in B cell interactions with T cells and the subsequent signaling events that eventually result in B cell proliferation and isotype switching. Similarly, the protein encoded for by this molecule may also be involved in the activation of LAK cells and goblet cells. Antibody or small molecule therapeutics designed with the protein encoded for by this molecule could reduce or inhibit inflammation by preventing T and B cell interactions that result in the production of IgE. Increased IgE levels correlate with allergy and asthma suggesting that these therapies may be effective treatments for these diseases. Antagonistic antibody or small molecule therapeutic approaches may also reduce or eliminate inflammation in colitis and prevent organ transplant rejection by blocking LAK activity.

NOV3

Expression of gene NOV3 was assessed using the primer-probe set Ag1213, described in Table F. Results of the RTQ-PCR runs are shown in Table G and H.

TABLE F
Probe Name: Ag1213

| | | | | | SEQ |
|---|---|---|---|---|---|
| | | | | Start | ID |
| Primers | Sequences | TM | Length | Position | # |
| Forward | 5′-TGACAGATGCGTTCAGTAACTG-3′ | 59 | 22 | 1103 | 66 |
| Probe | FAM-5′-CAAAATTGTTTTGTACATTCAGCCCGG-3′-TAMRA | 68.6 | 27 | 1141 | 67 |
| Reverse | 5′-GGAGCAAGTGTTTTGAAAACAC-3′ | 58.8 | 22 | 1169 | 68 |

TABLE G
Panel 1.2

| | Relative | | Relative |
|---|---|---|---|
| | Expression (%) | | Expression (%) |
| | 1.2tm1402f_ag | | 1.2tm1402f_ag |
| Tissue Name | 1213 | Tissue Name | 1213 |
| Endothelial cells | 0.0 | Renal ca. 786-0 | 0.0 |
| Endothelial cells (treated) | 0.0 | Renal ca. A498 | 0.0 |
| Pancreas | 0.0 | Renal ca. RXF 393 | 0.0 |
| Pancreatic ca. CAPAN 2 | 0.0 | Renal ca. ACHN | |
| Adrenal Gland (new lot*) | 0.0 | Renal ca. UO-31 | 0.0 |
| Thyroid | 0.0 | Renal ca. TK-10 | 0.0 |
| Salivary gland | 0.0 | Liver | 0.0 |
| Pituitary gland | 0.0 | Liver (fetal) | 0.0 |
| Brain (fetal) | 3.5 | Liver ca. (hepatoblast) HepG2 | 0.0 |
| Brain (whole) | 0.0 | Lung | 0.0 |
| Brain (amygdala) | 0.0 | Lung (fetal) | 0.0 |
| Brain (cerebellum) | 1.5 | Lung ca. (small cell) LX-1 | 0.0 |
| Brain (hippocampus) | 0.0 | Lung ca. (small cell) NCI-H69 | 5.6 |
| Brain (thalamus) | 0.0 | Lung ca. (s. cell var.) SHP-77 | 0.0 |
| Cerebral Cortex | 0.0 | Lung ca. (large cell) NCI-H460 | 30.1 |
| Spinal cord | 0.0 | Lung ca. (non-sm. cell) A549 | 0.0 |
| CNS ca. (glio/astro) U87-MG | 0.0 | Lung ca. (non-s. cell) NCI-H23 | 0.0 |
| CNS ca. (glio/astro) U-118-MG | 0.0 | Lung ca (non-s. cell) HOP-62 | 0.0 |
| CNS ca. (astro) SW1783 | 0.0 | Lung ca. (non-s. cl) NCI-H522 | 0.0 |
| CNS ca.* (neuro; met) SK-N-AS | 4.6 | Lung ca. (squam.) SW 900 | 0.0 |
| CNS ca. (astro) SF-539 | 0.0 | Lung ca. (squam.) NCI-H596 | 25.9 |
| CNS ca. (astro) SNB-75 | 0.0 | Mammary gland | 0.0 |
| CNS ca. (glio) SNB-19 | 0.0 | Breast ca.* (pl. effusion) MCF-7 | 0.0 |
| CNS ca. (glio) U251 | 0.0 | Breast ca.* (pl. ef) MDA-MB- | 0.0 |
| | | 231 | |
| CNS ca. (glio) SF-295 | 0.0 | Breast ca.* (pl. effusion) T47D | 0.0 |
| Heart | 0.0 | Breast ca. BT-549 | 3.8 |
| Skeletal Muscle (new lot*) | 0.0 | Breast ca. MDA-N | 0.0 |
| Bone marrow | 0.0 | Ovary | 0.0 |
| Thymus | 0.0 | Ovarian ca. OVCAR-3 | 0.0 |
| Spleen | 0.0 | Ovarian ca. OVCAR-4 | 0.0 |
| Lymph node | 0.0 | Ovarian ca. OVCAR-5 | 16.2 |
| Colorectal | 2.0 | Ovarian ca. OVCAR-8 | 0.0 |
| Stomach | 0.0 | Ovarian ca. IGROV-1 | 0.0 |
| Small intestine | 0.0 | Ovarian ca.* (ascites) SK-OV-3 | 0.0 |
| Colon ca. SW480 | 0.0 | Uterus | 0.0 |
| Colon ca.* (SW480 met) SW620 | 0.0 | Placenta | 0.0 |
| Colon ca. HT29 | 0.0 | Prostate | 0.0 |
| Colon ca. HCT-116 | 0.0 | Prostate ca.* (bone met) PC-3 | 0.0 |
| Colon ca. CaCo-2 | 0.0 | Testis | 0.0 |
| 83219 CC Well to Mod Diff | 100.0 | Melanoma Hs688(A).T | 0.0 |
| (ODO3866) | | | |
| Colon ca. HCC-2998 | 0.0 | Melanoma* (met) Hs688(B).T | 0.0 |
| Gastric ca.* (liver met) | 0.0 | Melanoma UACC-62 | 0.0 |
| NCI-N87 | | | |
| Bladder | 0.0 | Melanoma M14 | 0.0 |
| Trachea | 0.0 | Melanoma LOX IMVI | 0.0 |
| Kidney | 0.0 | Melanoma* (met) SK-MEL-5 | 0.0 |
| Kidney (fetal) | 0.0 | | |

TABLE H
Panel 2.1

| | Rel. Expr., (%) | | Rel. Expr., (%) |
|---|---|---|---|
| | 2.1tm6099f_ag | | 2.1tm6099f_ag |
| Tissue Name | 1213 | Tissue Name | 1213 |
| Normal Colon GENPAK | 0.0 | Kidney Cancer Clontech | 0.0 |
| 061003 | | 9010320 | |
| 97759 Colon cancer (OD06064) | 0.0 | Kidney NAT Clontech 9010321 | 0.0 |
| 97760 Colon cancer NAT | 0.0 | Kidney Cancer Clontech | 0.0 |
| (OD06064) | | 8120607 | |
| 97778 Colon cancer (OD06159) | 0.0 | Kidney NAT Clontech 8120608 | 0.0 |
| 97779 Colon cancer NAT | 0.0 | Normal Uterus GENPAK | 0.0 |
| (OD06159) | | 061018 | |
| 98859 Colon cancer | 0.0 | Uterus Cancer GENPAK | 0.0 |
| (OD06298-08) | | 064011 | |
| 98860 Colon cancer NAT | 0.0 | Normal Thyroid Clontech A+ | 0.0 |
| (OD06298-018) | | 6570-1 (7080817) | |
| 83237 CC Gr.2 ascend colon | 0.0 | Thyroid Cancer GENPAK | 0.0 |
| (ODO3921) | | 064010 | |
| | 0.0 | Thyroid Cancer INVITROGEN | 0.0 |
| 83238 CC NAT (ODO3921) | | A302152 | |
| 97766 Colon cancer metastasis | 0.0 | Thyroid NAT INVITROGEN | 0.0 |
| (OD06104) | | A302153 | |
| | 0.0 | Normal Breast GENPAK | 0.0 |
| 97767 Lung NAT (OD06104) | | 061019 | |
| 87472 Colon mets to lung | 0.0 | 84877 Breast Cancer | 0.0 |
| (OD04451-01) | | (OD04566) | |
| 87473 Lung NAT | 0.0 | | 0.0 |
| (OD04451-02) | | Breast Cancer Res. Gen. 1024 | |
| Normal Prostate Clontech A+ | 0.0 | 85975 Breast Cancer | 0.0 |
| 6546-1 (8090438) | | (OD04590-01) | |
| 84140 Prostate Cancer | 0.0 | 85976 Breast Cancer Mets | 0.0 |
| (OD04410) | | (OD04590-03) | |
| 84141 Prostate NAT | 0.0 | 87070 Breast Cancer Metastasis | 0.0 |
| (OD04410) | | (OD04655-05) | |
| | 0.0 | GENPAK Breast Cancer | 0.0 |
| Normal Lung GENPAK 061010 | | 064006 | |
| 92337 Invasive poor diff. lung | 0.0 | Breast Cancer Clontech | 0.0 |
| adeno (ODO4945-01 | | 9100266 | |
| 92338 Lung NAT | 0.0 | | 0.0 |
| (ODO4945-03) | | Breast NAT Clontech 9100265 | |
| 84136 Lung Malignant Cancer | 0.0 | Breast Cancer INVITROGEN | 0.0 |
| (OD03126) | | A209073 | |
| | 0.0 | Breast NAT INVITROGEN | 0.0 |
| 84137 Lung NAT (OD03126) | | A2090734 | |
| 90372 Lung Cancer | 0.0 | Normal Liver GENPAK | 0.4 |
| (OD05014A) | | 061009 | |
| | 0.0 | Liver Cancer Research Genetics | 0.0 |
| 90373 Lung NAT (OD05014B) | | RNA 1026 | |
| 85950 Lung Cancer | 0.0 | Liver Cancer Research Genetics | 0.0 |
| (OD04237-01) | | RNA 1025 | |
| | 0.0 | Paired Liver Cancer Tissue | 0.0 |
| 85970 Lung NAT | | Research Genetics RNA 6004-T | |
| (OD04237-02) | | | |
| 83255 Ocular Mel Met to Liver | 0.0 | Paired Liver Tissue Research | 0.0 |
| (ODO4310) | | Genetics RNA 6004-N | |
| | 0.0 | Paired Liver Cancer Tissue | 0.0 |
| | | Research Genetics RNA 6005-T | |
| 83256 Liver NAT (ODO4310) | | | |
| 84139 Melanoma Mets to Lung | 0.0 | Paired Liver Tissue Research | 0.0 |
| (OD04321) | | Genetics RNA 6005-N | |
| 84138 Lung NAT (OD04321) | 0.0 | Liver Cancer GENPAK 064003 | 0.0 |
| Normal Kidney GENPAK | 0.0 | Normal Bladder GENPAK | 0.0 |
| 061008 | | 061001 | |
| 83786 Kidney Ca, Nuclear | 0.0 | Bladder Cancer Research | 0.0 |
| grade 2 (OD04338) | | Genetics RNA 1023 | |
| 83787 Kidney NAT | 0.0 | Bladder Cancer INVITROGEN | 0.0 |
| (OD04338) | | A302173 | |
| 83788 Kidney Ca Nuclear grade | 0.0 | | 0.0 |
| 1/2 (OD04339) | | Normal Ovary Res. Gen. | |
| | 0.0 | Ovarian Cancer GENPAK | 100.0 |
| 83789 Kidney NAT (OD04339) | | 064008 | |
| 83790 Kidney Ca, Clear cell | 0.0 | 97773 Ovarian cancer | 0.0 |
| type (OD04340) | | (OD06145) | |
| | 0.0 | 97775 Ovarian cancer NAT | 0.0 |
| 83791 Kidney NAT (OD04340) | | (OD06145) | |
| 83792 Kidney Ca, Nuclear | 0.0 | Normal Stomach GENPAK | 0.0 |
| grade 3 (OD04348) | | 061017 | |
| | 0.0 | Gastric Cancer Clontech | 0.0 |
| 83793 Kidney NAT (OD04348) | | 9060397 | |
| 85973 Kidney Cancer | 0.0 | NAT Stomach Clontech | 0.0 |
| (OD04450-01) | | 9060396 | |
| 85974 Kidney NAT | 0.0 | Gastric Cancer Clontech | 0.0 |
| (OD04450-03) | | 9060395 | |
| Kidney Cancer Clontech | 0.0 | NAT Stomach Clontech | 0.0 |
| 8120613 | | 9060394 | |
| | 0.0 | Gastric Cancer GENPAK | 0.0 |
| Kidney NAT Clontech 8120614 | | 064005 | |

Panel 1.2 Summary: Expression of gene NOV3 is skewed by expression in adipose, likely due to genomic DNA contamination. Disregarding this, highest expression of this gene is seen in a sample of colon cancer, with lower levels of expression in two lung cancer cell lines and an ovarian cancer cell line. Thus expression of this gene may be a characteristic of these kinds of cancers and be of utility in the diagnosis or as a marker in these diseases. Antibody therapeutics to this protein may be used in the treatment of certain kinds of cancer.

Panel 2.1 Summary: The expression of this gene, NOV3 is restricted exclusively to one ovarian cancer sample. Thus, the expression of this gene may be a characteristic of ovarian cancer, or a subset thereof and may therefore be of utility in the diagnosis of ovarian cancer.

NOV4

Expression of gene NOV4 was assessed using the primer-probe set Ag1252, described in Table I. Results of the RTQ-PCR runs are shown in Tables J, K, L and M.

TABLE I
Probe Name: Ag1252

| | | | | | SEQ |
|---|---|---|---|---|---|
| | | | | Start | ID |
| Primers | Sequences | TM | Length | Position | # |
| Forward | 5′-CCAGTGCCAGTACCAGAATATC-3′ | 58.6 | 22 | 1210 | 69 |
| Probe | FAM-5′-AACCAGACACTCTCAGCTGCAGGCAT-3′-TAMRA | 70.4 | 26 | 1232 | 70 |
| Reverse | 5′-CTCTCTGGCCTCTCAGCAA-3′ | 59.4 | 19 | 1275 | 71 |

TABLE J
Panel 1.2

| | Relative | | Relative |
|---|---|---|---|
| | Expression (%) | | Expression (%) |
| | 1.2tm1422f_ag | | 1.2tm1422f_ag |
| Tissue Name | 1252 | Tissue Name | 1252 |
| Endothelial cells | 27.7 | Renal ca. 786-0 | 3.7 |
| Endothelial cells (treated) | 1.5 | Renal ca. A498 | 1.1 |
| Pancreas | 7.9 | Renal ca. RXF 393 | 0.9 |
| Pancreatic ca. CAPAN 2 | 2.9 | Renal ca. ACHN | 10.4 |
| Adrenal Gland (new lot*) | 8.3 | Renal ca. UO-31 | 11.0 |
| Thyroid | 15.7 | Renal ca. TK-10 | 3.9 |
| Salivary gland | 4.7 | Liver | 7.2 |
| Pituitary gland | 8.5 | Liver (fetal) | 3.7 |
| Brain (fetal) | 6.0 | Liver ca. (hepatoblast) HepG2 | 3.4 |
| Brain (whole) | 17.2 | Lung | 2.7 |
| Brain (amygdala) | 10.0 | Lung (fetal) | 3.1 |
| Brain (cerebellum) | 4.8 | Lung ca. (small cell) LX-1 | 4.3 |
| Brain (hippocampus) | 12.0 | Lung ca. (small cell) NCI-H69 | 1.8 |
| Brain (thalamus) | 12.2 | Lung ca. (s. cell var.) SHP-77 | 0.5 |
| Cerebral Cortex | 15.7 | Lung ca. (large cell) NCI-H460 | 27.4 |
| Spinal cord | 2.8 | Lung ca. (non-sm. cell) A549 | 6.4 |
| CNS ca. (glio/astro) U87-MG | 12.2 | Lung ca. (non-s. cell) NCI-H23 | 5.9 |
| CNS ca. (glio/astro) U-118-MG | 4.4 | Lung ca (non-s. cell) HOP-62 | 26.4 |
| CNS ca. (astro) SW1783 | 1.9 | Lung ca. (non-s. cl) NCI-H522 | 100.0 |
| CNS ca.* (neuro; met) SK-N-AS | 4.2 | Lung ca. (squam.) SW 900 | 7.6 |
| CNS ca. (astro) SF-539 | 2.8 | Lung ca. (squam.) NCI-H596 | 2.4 |
| CNS ca. (astro) SNB-75 | 0.8 | Mammary gland | 11.2 |
| CNS ca. (glio) SNB-19 | 11.0 | Breast ca.* (pl. effusion) MCF-7 | 15.6 |
| CNS ca. (glio) U251 | 6.9 | Breast ca.* (pl. ef) MDA-MB- | 2.3 |
| | | 231 | |
| CNS ca. (glio) SF-295 | 10.0 | Breast ca.* (pl. effusion) T47D | 2.1 |
| Heart | 5.9 | Breast ca. BT-549 | 6.5 |
| Skeletal Muscle (new lot*) | 2.9 | Breast ca. MDA-N | 0.0 |
| Bone marrow | 2.4 | Ovary | 6.0 |
| Thymus | 1.5 | Ovarian ca. OVCAR-3 | 7.3 |
| Spleen | 2.9 | Ovarian ca. OVCAR-4 | 3.0 |
| Lymph node | 4.3 | Ovarian ca. OVCAR-5 | 14.1 |
| Colorectal | 1.1 | Ovarian ca. OVCAR-8 | 3.6 |
| Stomach | 3.3 | Ovarian ca. IGROV-1 | 10.4 |
| Small intestine | 3.7 | Ovarian ca.* (ascites) SK-OV-3 | 6.7 |
| Colon ca. SW480 | 4.5 | Uterus | 3.5 |
| Colon ca.* (SW480 met) SW620 | 5.2 | Placenta | 13.4 |
| Colon ca. HT29 | 0.2 | Prostate | 5.9 |
| Colon ca. HCT-116 | 8.0 | Prostate ca.* (bone met) PC-3 | 12.8 |
| Colon ca. CaCo-2 | 4.1 | Testis | 7.4 |
| 83219 CC Well to Mod Diff | 1.2 | Melanoma Hs688(A).T | 4.3 |
| (ODO3866) | | | |
| Colon ca. HCC-2998 | 8.1 | Melanoma* (met) Hs688(B).T | 3.8 |
| Gastric ca.* (liver met) | 6.9 | Melanoma UACC-62 | 11.7 |
| NCI-N87 | | | |
| Bladder | 12.0 | Melanoma M14 | 3.6 |
| Trachea | 2.6 | Melanoma LOX IMVI | 6.0 |
| Kidney | 8.4 | Melanoma* (met) SK-MEL-5 | 4.4 |
| Kidney (fetal) | 9.2 | | |

TABLE K
Panel 2D

| | Relative | | Relative |
|---|---|---|---|
| | Expression (%) | | Expression (%) |
| | 2dtm4588f_ag | | 2dtm4588f_ag |
| Tissue Name | 1252 | Tissue Name | 1252 |
| Normal Colon GENPAK | 21.0 | Kidney NAT Clontech | 2.2 |
| 061003 | | 8120608 | |
| 83219 CC Well to Mod Diff | 2.6 | Kidney Cancer Clontech | 10.7 |
| (ODO3866) | | 8120613 | |
| 83220 CC NAT | 2.3 | Kidney NAT Clontech | 2.9 |
| (ODO3866) | | 8120614 | |
| 83221 CC Gr.2 | 2.3 | Kidney Cancer Clontech | 16.0 |
| rectosigmoid (ODO3868) | | 9010320 | |
| 83222 CC NAT | 0.2 | Kidney NAT Clontech | 8.5 |
| (ODO3868) | | 9010321 | |
| 83235 CC Mod Diff | 7.7 | Normal Uterus GENPAK | 3.0 |
| (ODO3920) | | 061018 | |
| 83236 CC NAT | 4.2 | Uterus Cancer GENPAK | 14.5 |
| (ODO3920) | | 064011 | |
| 83237 CC Gr.2 ascend | 7.3 | Normal Thyroid Clontech | 20.2 |
| colon (ODO3921) | | A+ 6570-1 | |
| 83238 CC NAT | 3.6 | Thyroid Cancer GENPAK | 8.9 |
| (ODO3921) | | 064010 | |
| 83241 CC from Partial | 9.1 | Thyroid Cancer | 11.0 |
| Hepatectomy (ODO4309) | | INVITROGEN A302152 | |
| 83242 Liver NAT | 9.4 | Thyroid NAT | 21.0 |
| (ODO4309) | | INVITROGEN A302153 | |
| 87472 Colon mets to Lung | 7.0 | Normal Breast GENPAK | 22.5 |
| (OD04451-01) | | 061019 | |
| 87473 Lung NAT | 7.5 | 84877 Breast Cancer | 40.1 |
| (OD04451-02) | | (OD04566) | |
| Normal Prostate Clontech | 44.1 | 85975 Breast Cancer | 20.4 |
| A+ 6546-1 | | (OD04590-01) | |
| 84140 Prostate Cancer | 32.5 | 85976 Breast Cancer Mets | 26.1 |
| (OD04410) | | (OD04590-03) | |
| 84141 Prostate NAT | 18.7 | 87070 Breast Cancer | 100.0 |
| (OD04410) | | Metastasis (OD04655-05) | |
| 87073 Prostate Cancer | 21.8 | GENPAK Breast Cancer | 10.7 |
| (OD04720-01) | | 064006 | |
| 87074 Prostate NAT | 31.2 | Breast Cancer Res. Gen. | 15.7 |
| (OD04720-02) | | 1024 | |
| Normal Lung GENPAK | 9.9 | Breast Cancer Clontech | 14.8 |
| 061010 | | 9100266 | |
| 83239 Lung Met to Muscle | 8.1 | Breast NAT Clontech | 7.4 |
| (ODO4286) | | 9100265 | |
| 83240 Muscle NAT | 7.3 | Breast Cancer | 20.4 |
| (ODO4286) | | INVITROGEN A209073 | |
| 84136 Lung Malignant | 9.2 | Breast NAT INVITROGEN | 11.4 |
| Cancer (OD03126) | | A2090734 | |
| 84137 Lung NAT | 14.5 | Normal Liver GENPAK | 11.1 |
| (OD03126) | | 061009 | |
| 84871 Lung Cancer | 14.5 | Liver Cancer GENPAK | 2.9 |
| (OD04404) | | 064003 | |
| 84872 Lung NAT | 7.9 | Liver Cancer Research | 4.9 |
| (OD04404) | | Genetics RNA 1025 | |
| 84875 Lung Cancer | 13.5 | Liver Cancer Research | 5.8 |
| (OD04565) | | Genetics RNA 1026 | |
| 84876 Lung NAT | 7.9 | Paired Liver Cancer Tissue | 5.8 |
| (OD04565) | | Research Genetics RNA | |
| | | 6004-T | |
| 85950 Lung Cancer | 15.8 | Paired Liver Tissue | 6.1 |
| (OD04237-01) | | Research Genetics RNA | |
| | | 6004-N | |
| 85970 Lung NAT | 11.3 | Paired Liver Cancer Tissue | 8.5 |
| (OD04237-02) | | Research Genetics RNA | |
| | | 6005-T | |
| 83255 Ocular Mel Met to | 6.7 | Paired Liver Tissue | 1.3 |
| Liver (ODO4310) | | Research Genetics RNA | |
| | | 6005-N | |
| 83256 Liver NAT | 6.5 | Normal Bladder GENPAK | 15.3 |
| (ODO4310) | | 061001 | |
| 84139 Melanoma Mets to | 4.1 | Bladder Cancer Research | 2.9 |
| Lung (OD04321) | | Genetics RNA 1023 | |
| 84138 Lung NAT | 8.1 | Bladder Cancer | 8.5 |
| (OD04321) | | INVITROGEN A302173 | |
| Normal Kidney GENPAK | 27.5 | 87071 Bladder Cancer | 14.1 |
| 061008 | | (OD04718-01) | |
| 83786 Kidney Ca, Nuclear | 17.9 | 87072 Bladder Normal | 18.9 |
| grade 2 (OD04338) | | Adjacent (OD04718-03) | |
| 83787 Kidney NAT | 9.9 | Normal Ovary Res. Gen. | 6.7 |
| (OD04338) | | | |
| 83788 Kidney Ca Nuclear | 13.3 | Ovarian Cancer GENPAK | 32.3 |
| grade 1/2 (OD04339) | | 064008 | |
| 83789 Kidney NAT | 11.8 | 87492 Ovary Cancer | 6.1 |
| (OD04339) | | (OD04768-07) | |
| 83790 Kidney Ca, Clear | 23.2 | 87493 Ovary NAT | 4.6 |
| cell type (OD04340) | | (OD04768-08) | |
| 83791 Kidney NAT | 14.8 | Normal Stomach GENPAK | 5.6 |
| (OD04340) | | 061017 | |
| 83792 Kidney Ca, Nuclear | 24.3 | Gastric Cancer Clontech | 3.6 |
| grade 3 (OD04348) | | 9060358 | |
| 83793 Kidney NAT | 13.7 | NAT Stomach Clontech | 5.3 |
| (OD04348) | | 9060359 | |
| 87474 Kidney Cancer | 9.8 | Gastric Cancer Clontech | 8.1 |
| (OD04622-01) | | 9060395 | |
| 87475 Kidney NAT | 2.0 | NAT Stomach Clontech | 4.5 |
| (OD04622-03) | | 9060394 | |
| 85973 Kidney Cancer | 11.7 | Gastric Cancer Clontech | 11.2 |
| (OD04450-01) | | 9060397 | |
| 85974 Kidney NAT | 20.6 | NAT Stomach Clontech | 2.0 |
| (OD04450-03) | | 9060396 | |
| Kidney Cancer Clontech | 1.1 | Gastric Cancer GENPAK | 19.2 |
| 8120607 | | 064005 | |

TABLE L
Panel 4D

| | Relative Expression (%) |
|---|---|
| Tissue Name | 4Dtm2111f_ag1252 |
| 93768_Secondary Th1_anti- | 1.0 |
| CD28/anti-CD3 | |
| 93769_Secondary Th2_anti- | 4.6 |
| CD28/anti-CD3 | |
| 93770_Secondary Tr1_anti- | 1.6 |
| CD28/anti-CD3 | |
| 93573_Secondary Th1_resting | 1.7 |
| day 4-6 in IL-2 | |
| 93572_Secondary Th2_resting | 4.1 |
| day 4-6 in IL-2 | |
| 93571_Secondary Tr1_resting | 2.3 |
| day 4-6 in IL-2 | |
| 93568_primary Th1_anti- | 4.6 |
| CD28/anti-CD3 | |
| 93569_primary Th2_anti- | 4.5 |
| CD28/anti-CD3 | |
| 93570_primary Tr1_anti- | 9.5 |
| CD28/anti-CD3 | |
| 93565_primary Th1_resting dy | 12.9 |
| 4-6 in IL-2 | |
| 93566_primary Th2_resting dy | 8.5 |
| 4-6 in IL-2 | |
| 93567_primary Tr1_resting dy | 15.8 |
| 4-6 in IL-2 | |
| 93351_CD45RA CD4 | 12.6 |
| lymphocyte_anti-CD28/anti- | |
| CD3 | |
| 93352_CD45RO CD4 | 5.6 |
| lymphocyte_anti-CD28/anti- | |
| CD3 | |
| 93251_CD8 Lymphocytes_anti- | 5.4 |
| CD28/anti-CD3 | |
| 93353_chronic CD8 | 3.2 |
| Lymphocytes 2ry_resting dy 4- | |
| 6 in IL-2 | |
| 93574_chronic CD8 | 1.9 |
| Lymphocytes 2ry_activated | |
| CD3/CD28 | |
| 93354_CD4_none | 4.9 |
| 93252_Secondary | 11.9 |
| Th1/Th2/Tr1_anti-CD95 CH11 | |
| 93103_LAK cells_resting | 47.3 |
| 93788_LAK cells_IL-2 | 1.9 |
| 93787_LAK cells_IL-2 + IL-12 | 5.8 |
| 93789_LAK cells_IL-2 + IFN | 6.0 |
| gamma | |
| 93790_LAK cells_IL-2 + IL-18 | 5.5 |
| 93104_LAK | 15.2 |
| cells_PMA/ionomycin and IL- | |
| 18 | |
| 93578_NK Cells IL-2_resting | 1.1 |
| 93109_Mixed Lymphocyte | 20.9 |
| Reaction_Two Way MLR | |
| 93110_Mixed Lymphocyte | 8.7 |
| Reaction_Two Way MLR | |
| 93111_Mixed Lymphocyte | 1.1 |
| Reaction_Two Way MLR | |
| 93112_Mononuclear Cells | 14.6 |
| (PBMCs)_resting | |
| 93113_Mononuclear Cells | 15.2 |
| (PBMCs)_PWM | |
| 93114_Mononuclear Cells | 7.0 |
| (PBMCs)_PHA-L | |
| 93249_Ramos (B cell)_none | 4.0 |
| 93250_Ramos (B | 7.6 |
| cell)_ionomycin | |
| 93349_B lymphocytes_PWM | 8.2 |
| 93350_B lymphoytes_CD40L | 8.5 |
| and IL-4 | |
| 92665_EOL-1 | 33.4 |
| (Eosinophil)_dbcAMP | |
| differentiated | |
| 93248_EOL-1 | 82.9 |
| (Eosinophil)_dbcAMP/PMAion | |
| omycin | |
| 93356_Dendritic Cells_none | 28.1 |
| 93355_Dendritic Cells_LPS | 12.8 |
| 100 ng/ml | |
| 93775_Dendritic Cells_anti- | 35.1 |
| CD40 | |
| 93774_Monocytes_resting | 35.8 |
| 93776_Monocytes_LPS 50 | 54.0 |
| ng/ml | |
| 93581_Macrophages_resting | 100.0 |
| 93582_Macrophages_LPS 100 | 23.3 |
| ng/ml | |
| 93098_HUVEC | 37.6 |
| (Endothelial)_none | |
| 93099_HUVEC | 70.2 |
| (Endothelial)_starved | |
| 93100_HUVEC | 23.3 |
| (Endothelial)_IL-1b | |
| 93779_HUVEC | 55.9 |
| (Endothelial)_IFN gamma | |
| 93102_HUVEC | 8.6 |
| (Endothelial)_TNF alpha + IFN | |
| gamma | |
| 93101_HUVEC | 21.5 |
| (Endothelial)_TNF alpha + IL4 | |
| 93781_HUVEC | 31.9 |
| (Endothelial)_IL-11 | |
| 93583_Lung Microvascular | 58.6 |
| Endothelial Cells_none | |
| 93584_Lung Microvascular | 32.1 |
| Endothelial Cells_TNFa (4 | |
| ng/ml) and IL1b (1 ng/ml) | |
| 92662_Microvascular Dermal | 84.7 |
| endothelium_none | |
| 92663_Microsvasular Dermal | 41.8 |
| endothelium_TNFa (4 ng/ml) | |
| and IL1b (1 ng/ml) | |
| 93773_Bronchial | 27.4 |
| epithelium_TNFa (4 ng/ml) and | |
| IL1b (1 ng/ml)** | |
| 93347_Small Airway | 13.3 |
| Epithelium_none | |
| 93348_Small Airway | 61.6 |
| Epithelium_TNFa (4 ng/ml) | |
| and IL1b (1 ng/ml) | |
| 92668_Coronery Artery | 50.7 |
| SMC_resting | |
| 92669_Coronery Artery | 20.3 |
| SMC_TNFa (4 ng/ml) and IL1b | |
| (1 ng/ml) | |
| 93107_astrocytes_resting | 7.0 |
| 93108_astrocytes_TNFa (4 | 13.2 |
| ng/ml) and IL1b (1 ng/ml) | |
| 92666_KU-812 | 9.2 |
| (Basophil)_resting | |
| 92667_KU-812 | 5.3 |
| (Basophil)_PMA/ionomycin | |
| 93579_CCD1106 | 15.3 |
| (Keratinocytes)_none | |
| 93580_CCD1106 | 53.2 |
| (Keratinocytes)_TNFa and | |
| IFNg** | |
| 93791_Liver Cirrhosis | 8.9 |
| 93792_Lupus Kidney | 10.9 |
| 93577_NCI-H292 | 42.3 |
| 93358_NCI-H292_IL-4 | 34.2 |
| 93360_NCI-H292_IL-9 | 43.8 |
| 93359_NCI-H292_IL-13 | 22.7 |
| 93357_NCI-H292_IFN gamma | 17.8 |
| 93777_HPAEC_- | 46.7 |
| 93778_HPAEC_IL-1 beta/TNA | 59.9 |
| alpha | |
| 93254_Normal Human Lung | 52.5 |
| Fibroblast_none | |
| 93253_Normal Human Lung | 17.2 |
| Fibroblast_TNFa (4 ng/ml) and | |
| IL-1b (1 ng/ml) | |
| 93257_Normal Human Lung | 71.7 |
| Fibroblast_IL-4 | |
| 93256_Normal Human Lung | 54.7 |
| Fibroblast_IL-9 | |
| 93255_Normal Human Lung | 81.8 |
| Fibroblast_IL-13 | |
| 93258_Normal Human Lung | 73.7 |
| Fibroblast_IFN gamma | |
| 93106_Dermal Fibroblasts | 69.3 |
| CCD1070_resting | |
| 93361_Dermal Fibroblasts | 39.8 |
| CCD1070_TNF alpha 4 ng/ml | |
| 93105_Dermal Fibroblasts | 36.6 |
| CCD1070_IL-1 beta 1 ng/ml | |
| 93772_dermal fibroblast_IFN | 15.7 |
| gamma | |
| 93771_dermal fibroblast_IL-4 | 36.3 |
| 93259_IBD Colitis 1** | 12.6 |
| 93260_IBD Colitis 2 | 0.6 |
| 93261_IBD Crohn's | 2.6 |
| 735010_Colon_normal | 20.2 |
| 735019_Lung_none | 13.9 |
| 64028-1_Thymus_none | 40.3 |
| 64030-1_Kidney_none | 8.2 |

TABLE M
CNS Panel

| | Rel. Expr., % |
|---|---|
| Tissue Name | cns1x4tm6178f_ag1252_a1 |
| 102633_BA4 Control | 46.5 |
| 102641_BA4 Control2 | 78.1 |
| 102625_BA4 Alzheimer's2 | 4.7 |
| 102649_BA4 Parkinson's | 64.1 |
| 102656_BA4 Parkinson's2 | 80.0 |
| 102664_BA4 Huntington's | 37.5 |
| 102671_BA4 Huntington's2 | 12.1 |
| 102603_BA4 PSP | 22.6 |
| 102610_BA4 PSP2 | 39.7 |
| 102588_BA4 Depression | 29.0 |
| 102596_BA4 Depression2 | 4.2 |
| 102634_BA7 Control | 62.2 |
| 102642_BA7 Control2 | 95.0 |
| 102626_BA7 Alzheimer's2 | 5.1 |
| 102650_BA7 Parkinson's | 23.0 |
| 102657_BA7 Parkinson's2 | 55.0 |
| 102665_BA7 Huntington's | 56.4 |
| 102672_BA7 Huntington's2 | 44.2 |
| 102604_BA7 PSP | 32.6 |
| 102611_BA7 PSP2 | 44.3 |
| 102589_BA7 Depression | 8.1 |
| 102632_BA9 Control | 33.9 |
| 102640_BA9 Control2 | 78.0 |
| 102617_BA9 Alzheimer's | 0.0 |
| 102624_BA9 Alzheimer's2 | 26.8 |
| 102648_BA9 Parkinson's | 16.2 |
| 102655_BA9 Parkinson's2 | 37.2 |
| 102663_BA9 Huntington's | 100.0 |
| 102670_BA9 Huntington's2 | 35.6 |
| 102602_BA9 PSP | 13.7 |
| 102609_BA9 PSP2 | 9.4 |
| 102587_BA9 Depression | 7.1 |
| 102595_BA9 Depression2 | 18.3 |
| 102635_BA17 Control | 39.2 |
| 102643_BA17 Control2 | 26.0 |
| 102627_BA17 Alzheimer's2 | 5.2 |
| 102651_BA17 Parkinson's | 65.2 |
| 102658_BA17 Parkinson's2 | 51.9 |
| 102666_BA17 Huntington's | 43.3 |
| 102673_BA17 Huntington's2 | 12.4 |
| 102590_BA17 Depression | 16.7 |
| 102597_BA17 Depression2 | 5.8 |
| 102605_BA17 PSP | 12.2 |
| 102612_BA17 PSP2 | 1.4 |
| 102637_Sub Nigra Control | 29.0 |
| 102645_Sub Nigra Control2 | 44.1 |
| 102629_Sub Nigra | 25.0 |
| Alzheimer's2 | |
| 102660_Sub Nigra Parkinson's2 | 34.8 |
| 102667_Sub Nigra | 67.5 |
| Huntington's | |
| 102674_Sub Nigra | 32.8 |
| Huntington's2 | |
| 102614_Sub Nigra PSP2 | 9.8 |
| 102592_Sub Nigra Depression | 0.0 |
| 102599_Sub Nigra Depression2 | 1.1 |
| 102636_Glob Palladus Control | 14.4 |
| 102644_Glob Palladus Control2 | 31.3 |
| 102620_Glob Palladus | 12.3 |
| Alzheimer's | |
| 102628_Glob Palladus | 0.0 |
| Alzheimer's2 | |
| 102652_Glob Palladus | 71.6 |
| Parkinson's | |
| 102659_Glob Palladus | 19.8 |
| Parkinson's2 | |
| 102606_Glob Palladus PSP | 8.4 |
| 102613_Glob Palladus PSP2 | 12.8 |
| 102591_Glob Palladus | 0.9 |
| Depression | |
| 102638_Temp Pole Control | 14.6 |
| 102646_Temp Pole Control2 | 46.4 |
| 102622_Temp Pole Alzheimer's | 17.5 |
| 102630_Temp Pole | 0.0 |
| Alzheimer's2 | |
| 102653_Temp Pole Parkinson's | 26.4 |
| 102661_Temp Pole | 36.3 |
| Parkinson's2 | |
| 102668_Temp Pole | 83.0 |
| Huntington's | |
| 102607_Temp Pole PSP | 5.9 |
| 102615_Temp Pole PSP2 | 5.2 |
| 102600_Temp Pole | 5.7 |
| Depression2 | |
| 102639_Cing Gyr Control | 63.5 |
| 102647_Cing Gyr Control2 | 32.1 |
| 102623_Cing Gyr Alzheimer's | 22.1 |
| 102631_Cing Gyr Alzheimer's2 | 1.2 |
| 102654_Cing Gyr Parkinson's | 15.7 |
| 102662_Cing Gyr Parkinson's2 | 54.0 |
| 102669_Cing Gyr Huntington's | 58.2 |
| 102676_Cing Gyr | 25.0 |
| Huntington's2 | |
| 102608_Cing Gyr PSP | 26.8 |
| 102616_Cing Gyr PSP2 | 16.9 |
| 102594_Cing Gyr Depression | 3.4 |
| 102601_Cing Gyr Depression2 | 1.4 |

Panel 1.2 Summary: The expression of NOV4 shows a basal level of expression across the majority of samples in panel 1.2. Of interest is the observation that lung cancer cell lines highly express NOV4 than the other samples. This might indicate that this gene may play a role in lung cancer.

Panel 2D Summary: NOV4 is expressed at basal levels across the majority of the panel. There is small, but consistent, dysregulation between a number of samples derived from cancers when compared to normal adjacent controls. In the context of the data derived from panel 1.2, this is particularly interesting in the case of lung cancers. In addition there is apparent interest in gastric and breast cancers. Thus, therapies targeted toward this gene may be beneficial to the treatment of lung, breast or gastric cancers.

Panel 4D Summary: This gene, NOV4, has a wide tissue distribution including fibroblasts, endothelial cells and macrophages. There is no apparent link between activation or inflammation and the induction of this transcript.

CNS Panel Summary: Serotonin receptors have been implicated in neuropsychiatric disorders including schizophrenia, bipolar disorder, depression, and Alzheimer's disease. This gene is downregulated in the cingulate gyrus, parietal cortex, and substantia nigra in depression as measured via RTQ-PCR analysis in postmortem brain tissue. Because many antidepressants are serotonin reuptake inhibitors (e.g., fluvoxamine, hypericum perforatum, clomipramine, milnacipran, etc) the downregulation of a serotonin receptor in the brain of patients suffering from chronic depression suggests that this molecule may have a critical role in the etiology of this disease and be an excellent small molecule target for the treatment of psychiatric disease.

NOV5

Expression of gene NOV5 was assessed using the primer-probe sets Ag1641 and Ag1210 (identical sequences), described in Table N. Results of the RTQ-PCR runs are shown in Table O.

TABLE N
Probe Name: Ag1641/Ag1210

| | | | | | SEQ |
|---|---|---|---|---|---|
| | | | | Start | ID |
| Primers | Sequences | TM | Length | Position | # |
| Forward | 5′-GGCCTGTCCTGTGAAGTGA-3′ | 59.8 | 19 | 543 | 72 |
| Probe | TET-5′-CCTACCCCAGTCATCACGTGGAGAAA-3′-TAMRA | 70 | 26 | 570 | 73 |
| Reverse | 5′-CTCAGGGGACTTCGTGACTAC-3′ | 58.8 | 21 | 597 | 74 |

TABLE O
Panel 1.2

| | Relative Expression (%) |
|---|---|
| Tissue Name | 1.2tm1402t_ag1210 |
| Endothelial cells | 0.0 |
| Endothelial cells (treated) | 0.4 |
| Pancreas | 0.0 |
| Pancreatic ca. CAPAN 2 | 0.0 |
| Adrenal Gland (new lot*) | 1.2 |
| Thyroid | 10.2 |
| Salivary gland | 3.3 |
| Pituitary gland | 4.8 |
| Brain (fetal) | 12.1 |
| Brain (whole) | 3.6 |
| Brain (amygdala) | 3.7 |
| Brain (cerebellum) | 9.2 |
| Brain (hippocampus) | 6.9 |
| Brain (thalamus) | 1.5 |
| Cerebral Cortex | 6.8 |
| Spinal cord | 0.5 |
| CNS ca. (glio/astro) U87-MG | 0.0 |
| CNS ca. (glio/astro) U-118-MG | 0.0 |
| CNS ca. (astro) SW1783 | 0.0 |
| CNS ca.* (neuro; met) SK-N- | 24.8 |
| AS | |
| CNS ca. (astro) SF-539 | 0.0 |
| CNS ca. (astro) SNB-75 | 0.0 |
| CNS ca. (glio) SNB-19 | 0.0 |
| CNS ca. (glio) U251 | 0.0 |
| CNS ca. (glio) SF-295 | 0.0 |
| Heart | 0.4 |
| Skeletal Muscle (new lot*) | 0.0 |
| Bone marrow | 0.0 |
| Thymus | 0.5 |
| Spleen | 0.0 |
| Lymph node | 0.0 |
| Colorectal | 0.0 |
| Stomach | 3.7 |
| Small intestine | 2.0 |
| Colon ca. SW480 | 0.0 |
| Colon ca.* (SW480 met) SW620 | 1.3 |
| Colon ca. HT29 | 0.0 |
| Colon ca. HCT-116 | 0.0 |
| Colon ca. CaCo-2 | 0.0 |
| 83219 CC Well to Mod Diff | 0.0 |
| (ODO3866) | |
| Colon ca. HCC-2998 | 0.0 |
| Gastric ca.* (liver met) NCI- | 0.0 |
| N87 | |
| Bladder | 0.4 |
| Trachea | 1.8 |
| Kidney | 0.0 |
| Kidney (fetal) | 0.9 |
| Renal ca. 786-0 | 0.0 |
| Renal ca. A498 | 0.0 |
| Renal ca. RXF 393 | 0.0 |
| Renal ca. ACHN | 0.0 |
| Renal ca. UO-31 | 0.2 |
| Renal ca. TK-10 | 0.0 |
| Liver | 0.0 |
| Liver (fetal) | 0.0 |
| Liver ca. (hepatoblast) HepG2 | 0.2 |
| Lung | 0.0 |
| Lung (fetal) | 0.6 |
| Lung ca. (small cell) LX-1 | 0.9 |
| Lung ca. (small cell) NCI-H69 | 14.5 |
| Lung ca. (s. cell var.) SHP-77 | 0.0 |
| Lung ca. (large cell) NCI-H460 | 3.5 |
| Lung ca. (non-sm. cell) A549 | 1.8 |
| Lung ca. (non-s. cell) NCI-H23 | 3.3 |
| Lung ca (non-s. cell) HOP-62 | 0.7 |
| Lung ca. (non-s.cl) NCI-H522 | 4.2 |
| Lung ca. (squam.) SW 900 | 4.5 |
| Lung ca. (squam.) NCI-H596 | 100.0 |
| Mammary gland | 0.0 |
| Breast ca.* (pl. effusion) MCF- | 0.5 |
| 7 | |
| Breast ca.* (pl. ef) MDA-MB- | 2.6 |
| 231 | |
| Breast ca.* (pl. effusion) T47D | 2.9 |
| Breast ca. BT-549 | 0.7 |
| Breast ca. MDA-N | 0.0 |
| Ovary | 0.3 |
| Ovarian ca. OVCAR-3 | 0.5 |
| Ovarian ca. OVCAR-4 | 0.0 |
| Ovarian ca. OVCAR-5 | 1.1 |
| Ovarian ca. OVCAR-8 | 4.3 |
| Ovarian ca. IGROV-1 | 0.0 |
| Ovarian ca.* (ascites) SK-OV-3 | 2.4 |
| Uterus | 0.9 |
| Placenta | 1.0 |
| Prostate | 0.8 |
| Prostate ca.* (bone met) PC-3 | 0.0 |
| Testis | 4.3 |
| Melanoma Hs688(A).T | 0.9 |
| Melanoma* (met) Hs688(B).T | 0.0 |
| Melanoma UACC-62 | 0.0 |
| Melanoma M14 | 0.0 |
| Melanoma LOX IMVI | 0.0 |
| Melanoma* (met) SK-MEL-5 | 2.9 |

Panel 1.2 Summary: The NOV5 gene shows highest expression in a sample of lung cancer, followed by CNS cancer. Expression is also seen in other lung cancer, breast cancer, ovarian cancer, melanoma and colon cancer. Therapeutics designed against the protein encoded by this transcript may be used in the treatment of certain types of cancer. In normal tissues, expression is highest in various regions of the brain, with lower levels in the thyroid, testis, pituitary and salivary glands, stomach and intestine. Still lower levels are seen in the prostate, placenta, uterus and adrenal gland. However, expression in panel 1.3 with Ag 1641 is not consistent with these results, probably due to probe failure.

NOV6

Expression of gene NOV6 was assessed using the primer-probe set Ag1254, described in Table P. Results of the RTQ-PCR runs are shown in Tables Q and R.

TABLE P
Probe Name: Ag1254

| | | | | | SEQ |
|---|---|---|---|---|---|
| | | | | Start | ID |
| Primers | Sequences | TM | Length | Position | # |
| Forward | 5′-CTGAGATGGAGGTCCAGGAT-3′ | 59 | 20 | 1076 | 75 |
| Probe | TET-5′-CAGAGCTAAAGGCCCTCCTCCAGAGT-3′-TAMRA | 68.9 | 26 | 1097 | 76 |
| Reverse | 5′-TGGAGGCTTCTTTTCTTTTCTT-3′ | 58.7 | 22 | 1147 | 77 |

TABLE Q
Panels 1.2 and 2D

| | Relative Expression (%) | | Relative Expression (%) |
|---|---|---|---|
| Tissue Name | 1.2tm1420t_ag1254 | Tissue Name | 2Dtm2362t_ag1254 |
| Endothelial cells | 21.2 | Normal Colon GENPAK | 82.4 |
| Endothelial cells (treated) | 22.1 | 061003 | |
| Pancreas | 6.3 | 83219 CC Well to Mod Diff | 42 |
| Pancreatic ca. CAPAN 2 | 9.9 | (ODO3866) | |
| Adrenal Gland (new lot*) | 56.3 | 83220 CC NAT (ODO3866) | 39.2 |
| Thyroid | 7.6 | 83221 CC Gr.2 rectosigmoid | 42.9 |
| Salivary gland | 42.3 | (ODO3868) | |
| Pituitary gland | 12.8 | 83222 CC NAT (ODO3868) | 0 |
| Brain (fetal) | 9.4 | 83235 CC Mod Diff | 46 |
| Brain (whole) | 17 | (ODO3920) | |
| Brain (amygdala) | 18.3 | 83236 CC NAT (ODO3920) | 17.3 |
| Brain (cerebellum) | 15.1 | 83237 CC Gr.2 ascend colon | 47.3 |
| Brain (hippocampus) | 27.4 | (ODO3921) | |
| Brain (thalamus) | 11.7 | 83238 CC NAT (ODO3921) | 10.5 |
| Cerebral Cortex | 56.3 | 83241 CC from Partial | 14 |
| Spinal cord | 18.3 | Hepatectomy (ODO4309) | |
| CNS ca. (glio/astro) U87-MG | 48.6 | 83242 Liver NAT (ODO4309) | 3.5 |
| CNS ca. (glio/astro) U-118-MG | 25.9 | 87472 Colon mets to lung | 5.9 |
| CNS ca. (astro) SW1783 | 11 | (OD04451-01) | |
| CNS ca.* (neuro; met) SK-N-AS | 71.7 | 87473 Lung NAT (OD04451-02) | 19.8 |
| CNS ca. (astro) SF-539 | 5.4 | Normal Prostate Clontech A + | 52.8 |
| CNS ca. (astro) SNB-75 | 5.9 | 6546-1 | |
| CNS ca. (glio) SNB-19 | 11.1 | 84140 Prostate Cancer | 28.3 |
| CNS ca. (glio) U251 | 3.3 | OD04410 | |
| CNS ca. (glio) SF-295 | 8.3 | 84141 Prostate NAT | 20.6 |
| Heart | 89.5 | (OD04410) | |
| Skeletal Muscle (new lot*) | 64.2 | 87073 Prostate Cancer | 16.6 |
| Bone marrow | 13.1 | OD04720-01 | |
| Thymus | 13.2 | 87074 Prostate NAT | 27.9 |
| Spleen | 5 | OD04720-02 | |
| Lymph node | 17.7 | Normal Lung GENPAK | 22.5 |
| Colorectal | 5.9 | 061010 | |
| Stomach | 33.9 | 83239 Lung Met to Muscle | 19.3 |
| Small intestine | 56.6 | (ODO4286) | |
| Colon ca. SW480 | 11.7 | 83240 Muscle NAT | 11.8 |
| Colon ca.* (SW480 met) SW620 | 32.8 | (ODO4286) | |
| Colon ca. HT29 | 19.6 | 84136 Lung Malignant Cancer | 12.2 |
| Colon ca. HCT-116 | 60.7 | (OD03126) | |
| Colon ca. CaCo-2 | 16.7 | 84137 Lung NAT (OD03126) | 5.1 |
| 83219 CC Well to Mod Diff (ODO3866) | 22.7 | 84871 Lung Cancer (OD04404) | 12.4 |
| Colon ca. HCC-2998 | 66.9 | 84872 Lung NAT (OD04404) | 16 |
| Gastric ca.* (liver met) NCI- | 30.4 | 84875 Lung Cancer (OD04565) | 17 |
| N87 | | 84876 Lung NAT (OD04565) | 7.3 |
| Bladder | 33.9 | 85950 Lung Cancer (OD04237- | 39 |
| Trachea | 12.9 | 01) | |
| Kidney | 26.2 | 85970 Lung NAT (OD04237- | 21.3 |
| Kidney (fetal) | 32.8 | 02) | |
| Renal ca. 786-0 | 19.3 | 83255 Ocular Mel Met to Liver | 39.2 |
| Renal ca. A498 | 25.2 | (ODO4310) | |
| Renal ca. RXF 393 | 7.7 | 83256 Liver NAT (ODO4310) | 11.4 |
| Renal ca. ACHN | 19.3 | 84139 Melanoma Mets to Lung | 53.6 |
| Renal ca. UO-31 | 9.3 | (OD04321) | |
| Renal ca. TK-10 | 31 | 84138 Lung NAT (OD04321) | 15.5 |
| Liver | 26.2 | Normal Kidney GENPAK | 17.6 |
| Liver (fetal) | 37.4 | 061008 | |
| Liver ca. (hepatoblast) HepG2 | 36.1 | 83786 Kidney Ca. Nuclear | 20 |
| Lung | 14.7 | grade 2 (OD04338) | |
| Lung (fetal) | 10.4 | 83787 Kidney NAT (OD04338) | 3.9 |
| Lung ca. (small cell) LX-1 | 35.8 | 83788 Kidney Ca Nuclear grade | 16.3 |
| Lung ca. (small cell) NCI-H69 | 30.6 | 1/2 (OD04339) | |
| Lung ca. (s. cell var.) SHP-77 | 12.2 | 83789 Kidney NAT (OD04339) | 16.7 |
| Lung ca. (large cell) NCI-H460 | 100 | 83790 Kidney Ca, Clear cell | 40.6 |
| Lung ca. (non-sm. cell) A549 | 31 | type (OD04340) | |
| Lung ca. (non-s. cell) NCI-H23 | 14 | 83791 Kidney NAT (OD04340) | 18.9 |
| Lung ca (non-s. cell) HOP-62 | 12.6 | 83792 Kidney Ca, Nuclear | 18.7 |
| Lung ca. (non-s.cl) NCI-H522 | 47 | grade 3 (OD04348) | |
| Lung ca. (squam.) SW 900 | 12.9 | 83793 Kidney NAT (OD04348) | 14.7 |
| Lung ca. (squam.) NCI-H596 | 46 | 87474 Kidney Cancer | 12.4 |
| Mammary gland | 9.7 | (OD04622-01) | |
| Breast ca.* (pl. effusion) MCF- | 25.9 | 87475 Kidney NAT (OD04622- | 4.9 |
| 7 | | 03) | |
| Breast ca.* (pl. ef) MDA-MB- | 13.6 | 85973 Kidney Cancer | 18.3 |
| 231 | | (OD04450-01) | |
| Breast ca.* (pl. effusion) T47D | 20 | 85974 Kidney NAT (OD04450- | 18 |
| Breast ca. BT-549 | 41.8 | 03) | |
| Breast ca. MDA-N | 27.4 | Kidney Cancer Clontech | 6.4 |
| Ovary | 19.1 | 8120613 | |
| Ovarian ca. OVCAR-3 | 23 | Kidney NAT Clontech 8120614 | 0 |
| Ovarian ca. OVCAR-4 | 39.8 | Kidney Cancer Clontech | 13.7 |
| Ovarian ca. OVCAR-5 | 44.4 | 9010320 | |
| Ovarian ca. OVCAR-8 | 11.3 | Kidney NAT Clontech 9010321 | 7.3 |
| Ovarian ca. IGROV-1 | 22.8 | Kidney Cancer Clontech | 8.5 |
| Ovarian ca.* (ascites) SK-OV-3 | 29.3 | 8120607 | |
| Uterus | 10.7 | Kidney NAT Clontech 8120608 | 9.5 |
| Placenta | 32.1 | Normal Uterus GENPAK | 5.7 |
| Prostate | 30.4 | 061018 | |
| Prostate ca.* (bone met)PC-3 | 77.4 | Uterus Cancer GENPAK | 12.5 |
| Testis | 29.1 | 064011 | |
| Melanoma Hs688(A).T | 7.7 | Normal Thyroid Clontech A + | 42 |
| Melanoma* (met) Hs688(B).T | 3.2 | 6570-1 | |
| Melanoma UACC-62 | 44.4 | Thyroid Cancer GENPAK | 27.4 |
| Melanoma M14 | 13.7 | 064010 | |
| Melanoma LOX IMVI | 34.6 | Thyroid Cancer INVITROGEN | 10.3 |
| Melanoma* (met) SK-MEL-5 | 46.3 | A302152 | |
| Adipose | 15.3 | Thyroid NAT INVITROGEN | 9.3 |
| | | A302153 | |
| | | Normal Breast GENPAK | 9.3 |
| | | 061019 | |
| | | 84877 Breast Cancer | 26.4 |
| | | (OD04566) | |
| | | Breast Cancer Res. Gen. 1024 | 18.2 |
| | | 85975 Breast Cancer | 18.2 |
| | | (OD04590-01) | |
| | | 85976 Breast Cancer Mets | 17.7 |
| | | (OD04590-03) | |
| | | 87070 Breast Cancer Metastasis | 21.3 |
| | | (OD04655-05) | |
| | | GENPAK Breast Cancer | 16.8 |
| | | 064006 | |
| | | Breast Cancer Clontech | 40.9 |
| | | 9100266 | |
| | | Breast NAT Clontech 9100265 | 19.6 |
| | | Breast Cancer INVITROGEN | 24.8 |
| | | A209073 | |
| | | Breast NAT INVITROGEN | 18.4 |
| | | A2090734 | |
| | | Normal Liver GENPAK | 7.6 |
| | | 061009 | |
| | | Liver Cancer Research Genetics | 7.1 |
| | | RNA 1026 | |
| | | Liver Cancer Research Genetics | 5.5 |
| | | RNA 1025 | |
| | | Paired Liver Cancer Tissue | 7 |
| | | Research Genetics RNA 6004- | |
| | | T | |
| | | Paired Liver Tissue Research | 21.6 |
| | | Genetics RNA 6004-N | |
| | | Paired Liver Cancer Tissue | 5 |
| | | Research Genetics RNA 6005- | |
| | | T | |
| | | Paired Liver Tissue Research | 0 |
| | | Genetics RNA 6005-N | |
| | | Liver Cancer GENPAK 064003 | 25.7 |
| | | Normal Bladder GENPAK | 31.6 |
| | | 061001 | |
| | | Bladder Cancer Research | 20.3 |
| | | Genetics RNA 1023 | |
| | | 87071 Bladder Cancer | 48.6 |
| | | OD04718-01 | |
| | | 87072 Bladder Normal | 25.3 |
| | | Adjacent (OD04718-03) | |
| | | Bladder Cancer INVITROGEN | 41.2 |
| | | A302173 | |
| | | Normal Ovary Res. Gen. | 11.1 |
| | | Ovarian Cancer GENPAK | 17.9 |
| | | 064008 | |
| | | 87492 Ovary Cancer | 41.5 |
| | | OD04768-07 | |
| | | 87493 Ovary NAT (OD04768- | 3.8 |
| | | 08) | |
| | | Normal Stomach GENPAK | 11.6 |
| | | 061017 | |
| | | Gastric Cancer Clontech | 8.4 |
| | | 9060358 | |
| | | NAT Stomach Clontech | 27.9 |
| | | 9060359 | |
| | | Gastric Cancer Clontech | 100 |
| | | 9060397 | |
| | | NAT Stomach Clontech | 10.9 |
| | | 9060396 | |
| | | Gastric Cancer Clontech | 27.2 |
| | | 9060395 | |
| | | NAT Stomach Clontech | 33.4 |
| | | 9060394 | |
| | | Gastric Cancer GENPAK | 45.1 |
| | | 064005 | |

TABLE R
Panel 4D

| | Relative Expression (%) |
|---|---|
| Tissue Name | 4Dtm2160t_ag1254 |
| 93768_Secondary Th1_anti- | 27.2 |
| CD28/anti-CD3 | |
| 93769_Secondary Th2_anti- | 22.2 |
| CD28/anti-CD3 | |
| 93770_Secondary Tr1_anti- | 31.6 |
| CD28/anti-CD3 | |
| 93573_Secondary Th1_resting | 4.3 |
| day 4-6 in IL-2 | |
| 93572_Secondary Th2_resting | 5.3 |
| day 4-6 in IL-2 | |
| 93571_Secondary Tr1_resting | 2.4 |
| day 4-6 in IL-2 | |
| 93568_primary Th1_anti- | 87.7 |
| CD28/anti-CD3 | |
| 93569_primary Th2_anti- | 55.5 |
| CD28/anti-CD3 | |
| 93570_primary Tr1_anti- | 41.8 |
| CD28/anti-CD3 | |
| 93565_primary Th1_resting dy | 13.7 |
| 4-6 in IL-2 | |
| 93566_primary Th2_resting dy | 4.5 |
| 4-6 in IL-2 | |
| 93567_primary Tr1_resting dy | 5.6 |
| 4-6 in IL-2 | |
| 93351_CD45RA CD4 | 7.3 |
| lymphocyte_anti-CD28/anti- | |
| CD3 | |
| 93352_CD45RO CD4 | 31.6 |
| lymphocyte_anti-CD28/anti- | |
| CD3 | |
| 93251_CD8 Lymphocytes_anti- | 16.7 |
| CD28/anti-CD3 | |
| 93353_chronic CD8 | 20.4 |
| Lymphocytes 2ry_resting dy 4- | |
| 6 in IL-2 | |
| 93574_chronic CD8 | 14.3 |
| Lymphocytes 2ry_activated | |
| CD3/CD28 | |
| 93354_CD4_none | 1.3 |
| 93252_Secondary | 2.8 |
| Th1/Th2/Tr1_anti-CD95 CH11 | |
| 93103_LAK cells_resting | 4.2 |
| 93788_LAK cells_IL-2 | 9.3 |
| 93787_LAK cells_IL-2 + IL-12 | 7.5 |
| 93789_LAK cells_IL-2 + IFN | 15.2 |
| gamma | |
| 93790_LAK cells_IL-2 + IL-18 | 5.4 |
| 93104_LAK | 1.9 |
| cells_PMA/ionomycin and IL- | |
| 18 | |
| 93578_NK Cells IL-2_resting | 2.4 |
| 93109_Mixed Lymphocyte | 3 |
| Reaction_Two Way MLR | |
| 93110_Mixed Lymphocyte | 4 |
| Reaction_Two Way MLR | |
| 93111_Mixed Lymphocyte | 3.4 |
| Reaction_Two Way MLR | |
| 93112_Mononuclear Cells | 0.9 |
| (PBMCs)_resting | |
| 93113_Mononuclear Cells | 45.4 |
| (PBMCs)_PWM | |
| 93114_Mononuclear Cells | 21.6 |
| (PBMCs)_PHA-L | |
| 93249_Ramos (B cell)_none | 20.2 |
| 93250_Ramos (B | 100 |
| cell)_ionomycin | |
| 93349_B lymphocytes_PWM | 44.1 |
| 93350_B lymphoytes_CD40L | 3.2 |
| and IL-4 | |
| 92665_EOL-1 | 2.6 |
| (Eosinophil)_dbcAMP | |
| differentiated | |
| 93248_EOL-1 | 2.1 |
| (Eosinophil)_dbcAMP/PMAion | |
| omycin | |
| 93356_Dendritic Cells_none | 1.1 |
| 93355_Dendritic Cells_LPS | 0.9 |
| 100 ng/ml | |
| 93775_Dendritic Cells_anti- | 1.2 |
| CD40 | |
| 93774_Monocytes_resting | 0.8 |
| 93776_Monocytes_LPS 50 | 0.9 |
| ng/ml | |
| 93581_Macrophages_resting | 3.5 |
| 93582_Macrophages_LPS 100 | 1.5 |
| ng/ml | |
| 93098_HUVEC | 3.1 |
| (Endothelial)_none | |
| 93099_HUVEC | 10.6 |
| (Endothelial)_starved | |
| 93100_HUVEC | 37.6 |
| (Endothelial)_IL-1b | |
| 93779_HUVEC | 1.9 |
| (Endothelial)_IFN gamma | |
| 93102_HUVEC | 1.3 |
| (Endothelial)_TNF alpha + IFN | |
| gamma | |
| 93101_HUVEC | 2 |
| (Endothelial)_TNF alpha + IL4 | |
| 93781_HUVEC | 2.4 |
| (Endothelial)_IL-11 | |
| 93583_Lung Microvascular | 2 |
| Endothelial Cells_none | |
| 93584_Lung Microvascular | 2.6 |
| Endothelial Cells_TNFa (4 | |
| ng/ml) and IL1b (1 ng/ml) | |
| 92662_Microvascular Dermal | 5.3 |
| endothelium_none | |
| 92663_Microsvasular Dermal | 4.7 |
| endothelium_TNFa (4 ng/ml) | |
| and IL1b (1 ng/ml) | |
| 93773_Bronchial | 2.9 |
| epithelium_TNFa (4 ng/ml) and | |
| IL1b (1 ng/ml)** | |
| 93347_Small Airway | 2 |
| Epithelium_none | |
| 93348_Small Airway | 21.5 |
| Epithelium_TNFa (4 ng/ml) | |
| and IL1b (1 ng/ml) | |
| 92668_Coronery Artery | 2.6 |
| SMC_resting | |
| 92669_Coronery Artery | 0.6 |
| SMC_TNFa (4 ng/ml) and IL1b | |
| (1 ng/ml) | |
| 93107_astrocytes_resting | 0.7 |
| 93108_astrocytes_TNFa (4 | 0.9 |
| ng/ml) and IL1b (1 ng/ml) | |
| 92666_KU-812 | 3.8 |
| (Basophil)_resting | |
| 92667_KU-812 | 12.1 |
| (Basophil)_PMA/ionomycin | |
| 93579_CCD1106 | 4 |
| (Keratinocytes)_none | |
| 93580_CCD1106 | 66.4 |
| (Keratinocytes)_TNFa and | |
| IFNg** | |
| 93791_Liver Cirrhosis | 0.3 |
| 93792_Lupus Kidney | 0.6 |
| 93577_NCI-H292 | 7.3 |
| 93358_NCI-H292_IL-4 | 12.1 |
| 93360_NCI-H292_IL-9 | 9 |
| 93359_NCI-H292_IL-13 | 2.7 |
| 93357_NCI-H292_IFN gamma | 3.1 |
| 93777_HPAEC_- | 1.5 |
| 93778_HPAEC_IL-1 beta/TNA | 2.9 |
| alpha | |
| 93254_Normal Human Lung | 1.5 |
| Fibroblast_none | |
| 93253_Normal Human Lung | 1.3 |
| Fibroblast_TNFa (4 ng/ml) and | |
| IL-1b (1 ng/ml) | |
| 93257_Normal Human Lung | 5.4 |
| Fibroblast_IL-4 | |
| 93256_Normal Human Lung | 3.4 |
| Fibroblast_IL-9 | |
| 93255_Normal Human Lung | 11.4 |
| Fibroblast_IL-13 | |
| 93258_Normal Human Lung | 10.6 |
| Fibroblast_IFN gamma | |
| 93106_Dermal Fibroblasts | 11 |
| CCD1070_resting | |
| 93361_Dermal Fibroblasts | 15.2 |
| CCD1070_TNF alpha 4 ng/ml | |
| 93105_Dermal Fibroblasts | 2.5 |
| CCD1070_IL-1 beta 1 ng/ml | |
| 93772_dermal fibroblast_IFN | 0.7 |
| gamma | |
| 93771_dermal fibroblast_IL-4 | 2.8 |
| 93259_IBD Colitis 1** | 0.5 |
| 93260_IBD Colitis 2 | 0.6 |
| 93261_IBD Crohn's | 0.5 |
| 735010_Colon_normal | 2.6 |
| 735019_Lung_none | 3.5 |
| 64028-1_Thymus_none | 10 |
| 64030-1_Kidney_none | 7.9 |

Panel 1.2 Summary: NOV6 is widely expressed across the majority of panel 1.2. This is particularly true of the cell lines, which are characteristically proliferating cells, but is also true of the samples derived from heart and skeletal muscle. Thus, this gene may play a role in muscle homeostasis and/or cell proliferation.

Panel 2D Summary: Consistent with the results in panel 1.2, the expression of NOV6 seems to be widespread across the samples in panel 2D. Of interest is the fact that there appears to be significant dysregulation of this gene in colon and gastric cancers when compared to their normal adjacent controls. Thus, therapeutic targeting of this gene may be useful for the treatment of gastric or colon cancers.

Panel 4D Summary: The transcript is expressed highly in activated keratinocytes and in B cells but not in the untreated controls for both of these cell types. Lower level induction of the transcript is observed in small airway epithelium after treatment with TNF alpha and IL-1 beta and in activated T cells. Role in inflammation: The protein encoded for by this transcript could be important for the proliferation, differentiation, activation and signal transduction of cells in response to pro-inflammatory mediators and polarizing cytokines. Therapeutic utilization: Antagonistic therapies designed against the protein encoded for by this transcript could reduce or inhibit inflammation in psoriasis and delayed type hypersensitivity, asthma, and emphysema.

NOV7B

Expression of gene NOV7B was assessed using the primer-probe set Ag1207, described in Table S. Results of the RTQ-PCR runs are shown in Table T.

TABLE S
Probe Name: Ag1207

| | | | | | SEQ |
|---|---|---|---|---|---|
| | | | | Start | ID |
| Primers | Sequences | TM | Length | Position | # |
| Forward | 5′-TTGGATGAAGTGCAGTGGAT-3′ | 59.1 | 22 | 43 | 75 |
| Probe | TET-5′-CAAGTGGCGGCTCTATTATCAAGTCC-3′-TAMRA | 66.9 | 26 | 66 | 76 |
| Reverse | 5′-AACCCCTTCCAGATCATGAG-3′ | 58.9 | 20 | 140 | 77 |

TABLE T
Panel 4D

| | Rel. Expr., % |
|---|---|
| Tissue Name | 4Dtm2064t_ag1207 |
| 93768_Secondary Th1_anti- | 0.0 |
| CD28/anti-CD3 | |
| 93769_Secondary Th2_anti- | 0.0 |
| CD28/anti-CD3 | |
| 93770_Secondary Tr1_anti- | 0.0 |
| CD28/anti-CD3 | |
| 93573_Secondary Th1_resting | 0.0 |
| day 4-6 in IL-2 | |
| 93572_Secondary Th2_resting | 0.0 |
| day 4-6 in IL-2 | |
| 93571_Secondary Tr1_resting | 0.0 |
| day 4-6 in IL-2 | |
| 93568_primary Th1_anti- | 0.0 |
| CD28/anti-CD3 | |
| 93569_primary Th2_anti- | 0.0 |
| CD28/anti-CD3 | |
| 93570_primary Tr1_anti- | 0.0 |
| CD28/anti-CD3 | |
| 93565_primary Th1_resting dy | 0.0 |
| 4-6 in IL-2 | |
| 93566_primary Th2_resting dy | 0.0 |
| 4-6 in IL-2 | |
| 93567_primary Tr1_resting dy | 0.0 |
| 4-6 in IL-2 | |
| 93351_CD45RA CD4 | 0.0 |
| lymphocyte_anti-CD28/anti- | |
| CD3 | |
| 93352_CD45RO CD4 | 0.0 |
| lymphocyte_anti-CD28/anti- | |
| CD3 | |
| 93251_CD8 Lymphocytes_anti- | 0.0 |
| CD28/anti-CD3 | |
| 93353_chronic CD8 | 0.0 |
| Lymphocytes 2ry_resting dy 4- | |
| 6 in IL-2 | |
| 93574_chronic CD8 | 0.0 |
| Lymphocytes 2ry_activated | |
| CD3/CD28 | |
| 93354_CD4_none | 0.0 |
| 93252_Secondary | 0.0 |
| Th1/Th2/Tr1_anti-CD95 CH11 | |
| 93103_LAK cells_resting | 0.0 |
| 93788_LAK cells_IL-2 | 0.0 |
| 93787_LAK cells_IL-2 + IL-12 | 0.0 |
| 93789_LAK cells_IL-2 + IFN | 0.0 |
| gamma | |
| 93790_LAK cells_IL-2 + IL-18 | 0.0 |
| 93104_LAK | 0.0 |
| cells_PMA/ionomycin and IL- | |
| 18 | |
| 93578_NK Cells IL-2_resting | 0.0 |
| 93109_Mixed Lymphocyte | 0.0 |
| Reaction_Two Way MLR | |
| 93110_Mixed Lymphocyte | 0.0 |
| Reaction_Two Way MLR | |
| 93111_Mixed Lymphocyte | 0.0 |
| Reaction_Two Way MLR | |
| 93112_Mononuclear Cells | 0.0 |
| (PBMCs)_resting | |
| 93113_Mononuclear Cells | 0.0 |
| (PBMCs)_PWM | |
| 93114_Mononuclear Cells | 0.0 |
| (PBMCs)_PHA-L | |
| 93249_Ramos (B cell)_none | 0.0 |
| 93250_Ramos (B | 0.0 |
| cell)_ionomycin | |
| 93349_B lymphocytes_PWM | 0.0 |
| 93350_B lymphoytes_CD40L | 0.0 |
| and IL-4 | |
| 92665_EOL-1 | 0.0 |
| (Eosinophil)_dbcAMP | |
| differentiated | |
| 93248_EOL-1 | 0.0 |
| (Eosinophil)_dbcAMP/PMAion | |
| omycin | |
| 93356_Dendritic Cells_none | 0.0 |
| 93355_Dendritic Cells_LPS | 0.0 |
| 100 ng/ml | |
| 93775_Dendritic Cells_anti- | 0.0 |
| CD40 | |
| 93774_Monocytes_resting | 0.0 |
| 93776_Monocytes_LPS 50 | 0.0 |
| ng/ml | |
| 93581_Macrophages_resting | 0.0 |
| 93582_Macrophages_LPS 100 | 0.0 |
| ng/ml | |
| 93098_HUVEC | 0.0 |
| (Endothelial)_none | |
| 93099_HUVEC | 0.0 |
| (Endothelial)_starved | |
| 93100_HUVEC | 0.0 |
| (Endothelial)_IL-1b | |
| 93779_HUVEC | 0.0 |
| (Endothelial)_IFN gamma | |
| 93102_HUVEC | 0.0 |
| (Endothelial)_TNF alpha + IFN | |
| gamma | |
| 93101_HUVEC | 0.0 |
| (Endothelial)_TNF alpha + IL4 | |
| 93781_HUVEC | 0.0 |
| (Endothelial)_IL-11 | |
| 93583_Lung Microvascular | 0.0 |
| Endothelial Cells_none | |
| 93584_Lung Microvascular | 0.0 |
| Endothelial Cells_TNFa (4 | |
| ng/ml) and IL1b (1 ng/ml) | |
| 92662_Microvascular Dermal | 0.0 |
| endothelium_none | |
| 92663_Microsvasular Dermal | 0.0 |
| endothelium_TNFa (4 ng/ml) | |
| and IL1b (1 ng/ml) | |
| 93773_Bronchial | 0.0 |
| epithelium_TNFa (4 ng/ml) and | |
| IL1b (1 ng/ml)** | |
| 93347_Small Airway | 0.0 |
| Epithelium_none | |
| 93348_Small Airway | 0.0 |
| Epithelium_TNFa (4 ng/ml) | |
| and IL1b (1 ng/ml) | |
| 92668_Coronery Artery | 0.0 |
| SMC_resting | |
| 92669_Coronery Artery | 0.0 |
| SMC_TNFa (4 ng/ml) and IL1b | |
| (1 ng/ml) | |
| 93107_astrocytes_resting | 0.0 |
| 93108_astrocytes_TNFa (4 | 0.0 |
| ng/ml) and IL1b (1 ng/ml) | |
| 92666_KU-812 | 0.0 |
| (Basophil)_resting | |
| 92667_KU-812 | 0.0 |
| (Basophil)_PMA/ionomycin | |
| 93579_CCD1106 | 0.0 |
| (Keratinocytes)_none | |
| 93580_CCD1106 | 0.0 |
| (Keratinocytes)_TNFa and | |
| IFNg** | |
| 93791_Liver Cirrhosis | 0.0 |
| 93792_Lupus Kidney | 0.0 |
| 93577_NCI-H292 | 0.0 |
| 93358_NCI-H292_IL-4 | 0.0 |
| 93360_NCI-H292_IL-9 | 0.0 |
| 93359_NCI-H292_IL-13 | 0.0 |
| 93357_NCI-H292_IFN gamma | 0.0 |
| 93777_HPAEC _- | 0.0 |
| 93778_HPAEC_IL-1 beta/TNA | 0.0 |
| alpha | |
| 93254_Normal Human Lung | 0.0 |
| Fibroblast_none | |
| 93253_Normal Human Lung | 0.0 |
| Fibroblast_TNFa (4 ng/ml) and | |
| IL-1b (1 ng/ml) | |
| 93257_Normal Human Lung | 0.0 |
| Fibroblast_IL-4 | |
| 93256_Normal Human Lung | 0.0 |
| Fibroblast_IL-9 | |
| 93255_Normal Human Lung | 0.0 |
| Fibroblast_IL-13 | |
| 93258_Normal Human Lung | 0.0 |
| Fibroblast_IFN gamma | |
| 93106_Dermal Fibroblasts | 0.0 |
| CCD1070_resting | |
| 93361_Dermal Fibroblasts | 0.0 |
| CCD1070_TNF alpha 4 ng/ml | |
| 93105_Dermal Fibroblasts | 0.0 |
| CCD1070_IL-1 beta 1 ng/ml | |
| 93772_dermal fibroblast_IFN | 0.0 |
| gamma | |
| 93771_dermal fibroblast_IL-4 | 0.0 |
| 93259_IBD Colitis 1** | 100.0 |
| 93260_IBD Colitis 2 | 0.0 |
| 93261_IBD Crohn's | 3.9 |
| 735010_Colon_normal | 7.3 |
| 735019_Lung_none | 0.0 |
| 64028-1_Thymus_none | 16.7 |
| 64030-1_Kidney_none | 3.0 |

Panel 4D Summary: Expression of this gene is high in IBD colitis 1, probably due to genomic DNA contamination. Expression in other tissues and cell lines is low/undetectable (Ct values>35).

NOV8

Expression of gene NOV8 was assessed using the primer-probe sets Ag1290 and Ag2655, described in Tables U and V. Results of the RTQ-PCR runs are shown in Table W, X and Y.

TABLE U
Probe Name: Ag1290

| | | | | | SEQ |
|---|---|---|---|---|---|
| | | | | Start | ID |
| Primers | Sequences | TM | Length | Position | # |
| Forward | 5′-AAATTCTTCCATTGGAAGGAAA-3′ | 59 | 22 | 1639 | 78 |
| Probe | TET-5′-CAGTCTCCTCCTTTAAGCCTCGAGAT-3′-TAMRA | 65.4 | 26 | 1661 | 79 |
| Reverse | 5′-ATCTTTCGCGGAGATAATGTTT-3′ | 59.2 | 22 | 1692 | 80 |

TABLE V
Probe Name: Ag2655

| | | | | | SEQ |
|---|---|---|---|---|---|
| | | | | Start | ID |
| Primers | Sequences | TM | Length | Position | # |
| Forward | 5′-AAATTCTTCCATTGGAAGGAAA-3′ | 59 | 22 | 1639 | 81 |
| Probe | TET-5′-CAGTCTCCTCCTTTAAGCCTCGAGAT-3′-TAMRA | 65.4 | 26 | 1661 | 82 |
| Reverse | 5′-ATCTTTCGCGGAGATAATGTTT-3′ | 59.2 | 22 | 1692 | 83 |

TABLE W
Panel 1.3D

| | | |
|---|---|---|
| | Relative | Relative |
| | Expression (%) | Expression (%) |
| Tissue Name | 1.3Dtm2776t_ag1290 | 1.3dtm3908f_ag2655 |

| | | |
|---|---|---|
| Liver adenocarcinoma | 12.2 | 7.1 |
| Pancreas | 15.2 | 6.1 |
| Pancreatic ca. CAPAN 2 | 4.8 | 3.9 |
| Adrenal gland | 15.3 | 4.3 |
| Thyroid | 10.7 | 8.6 |
| Salivary gland | 12.8 | 6.3 |
| Pituitary gland | 25.5 | 19.5 |
| Brain (fetal) | 13.8 | 8.0 |
| Brain (whole) | 19.9 | 16.8 |
| Brain (amygdala) | 52.5 | 22.7 |
| Brain (cerebellum) | 12.3 | 11.5 |
| Brain (hippocampus) | 70.2 | 100.0 |
| Brain (substantia nigra) | 5.7 | 6.1 |
| Brain (thalamus) | 16.2 | 11.9 |
| Cerebral Cortex | 66.0 | 33.0 |
| Spinal cord | 15.3 | 9.5 |
| CNS ca. (glio/astro) U87-MG | 11.5 | 7.4 |
| CNS ca. (glio/astro) U-118-MG | 40.3 | 48.0 |
| CNS ca. (astro) SW1783 | 8.3 | 6.7 |
| CNS ca.* (neuro; met) SK-N-AS | 42.9 | 67.4 |
| CNS ca. (astro) SF-539 | 5.6 | 5.1 |
| CNS ca. (astro) SNB-75 | 43.2 | 21.3 |
| CNS ca. (glio) SNB-19 | 9.3 | 5.6 |
| CNS ca. (glio) U251 | 5.2 | 2.7 |
| CNS ca. (glio) SF-295 | 13.6 | 9.7 |
| Heart (fetal) | 7.3 | 1.7 |
| Heart | 4.2 | 2.8 |
| Fetal Skeletal | 24.0 | 15.3 |
| Skeletal muscle | 5.9 | 2.3 |
| Bone marrow | 14.4 | 5.5 |
| Thymus | 31.9 | 13.4 |
| Spleen | 23.0 | 28.1 |
| Lymph node | 31.6 | 14.2 |
| Colorectal | 13.4 | 7.9 |
| Stomach | 21.2 | 8.8 |
| Small intestine | 20.3 | 18.0 |
| Colon ca. SW480 | 12.5 | 7.2 |
| Colon ca.* (SW480 met) SW620 | 13.8 | 7.4 |
| Colon ca. HT29 | 18.7 | 13.8 |
| Colon ca. HCT-116 | 12.8 | 12.3 |
| Colon ca. CaCo-2 | 11.8 | 5.7 |
| 83219 CC Well to Mod Diff (ODO3866) | 11.0 | 6.4 |
| Colon ca. HCC-2998 | 18.3 | 29.1 |
| Gastric ca.* (liver met) NCI-N87 | 100.0 | 70.7 |
| Bladder | 12.9 | 4.7 |
| Trachea | 61.1 | 62.4 |
| Kidney | 5.6 | 4.2 |
| Kidney (fetal) | 9.1 | 6.4 |
| Renal ca. 786-0 | 4.9 | 4.8 |
| Renal ca. A498 | 23.3 | 14.3 |
| Renal ca. RXF 393 | 0.9 | 0.5 |
| Renal ca. ACHN | 5.3 | 0.9 |
| Renal ca. UO-31 | 12.5 | 4.9 |
| Renal ca. TK-10 | 2.5 | 2.6 |
| Liver | 5.1 | 3.7 |
| Liver (fetal) | 7.0 | 4.2 |
| Liver ca. (hepatoblast) HepG2 | 18.8 | 8.4 |
| Lung | 21.8 | 0.0 |
| Lung (fetal) | 40.3 | 15.2 |
| Lung ca. (small cell) LX-1 | 29.9 | 9.0 |
| Lung ca. (small cell) NCI-H69 | 19.6 | 11.5 |
| Lung ca. (s. cell var.) SHP-77 | 15.7 | 8.1 |
| Lung ca. (large cell) NCI-H460 | 3.4 | 2.4 |
| Lung ca. (non-sm. cell) A549 | 7.9 | 7.2 |
| Lung ca. (non-s. cell) NCI-H23 | 23.2 | 14.4 |
| Lung ca (non-s. cell) HOP-62 | 6.4 | 2.0 |
| Lung ca. (non-s.cl) NCI-H522 | 10.5 | 2.9 |
| Lung ca. (squam.) SW 900 | 24.3 | 13.2 |
| Lung ca. (squam.) NCI-H596 | 11.8 | 5.3 |
| Mammary gland | 28.5 | 0.0 |
| Breast ca.* (pl. effusion) MCF-7 | 10.0 | 6.5 |
| Breast ca. (pl. ef) MDA-MB-231 | 24.8 | 27.7 |
| Breast ca.* (pl. effusion) T47D | 3.1 | 2.1 |
| Breast ca. BT-549 | 25.7 | 27.7 |
| Breast ca. MDA-N | 15.0 | 12.7 |
| Ovary | 37.9 | 27.0 |
| Ovarian ca. OVCAR-3 | 6.6 | 4.2 |
| Ovarian ca. OVCAR-4 | 0.7 | 0.5 |
| Ovarian ca. OVCAR-5 | 17.7 | 11.5 |
| Ovarian ca. OVCAR-8 | 10.2 | 6.2 |
| Ovarian ca. IGROV-1 | 3.9 | 3.6 |
| Ovarian ca.* (ascites) SK-OV-3 | 19.6 | 11.3 |
| Uterus | 20.7 | 10.7 |
| Placenta | 17.1 | 9.2 |
| Prostate | 11.7 | 11.5 |
| Prostate ca.* (bone met) PC-3 | 8.2 | 5.3 |
| Testis | 21.6 | 9.5 |
| Melanoma Hs688(A).T | 16.6 | 2.0 |
| Melanoma* (met) Hs688(B).T | 30.8 | 1.1 |
| Melanoma UACC-62 | 0.1 | 0.8 |
| Melanoma M14 | 2.5 | 1.1 |
| Melanoma LOX IMVI | 0.7 | 1.9 |
| Melanoma* (met) SK-MEL-5 | 2.4 | 2.1 |
| Adipose | 21.2 | 7.5 |

TABLE X
Panel 2D

| | Rel. Expr., % | Rel. Expr., % |
|---|---|---|
| | 2dx4tm4810t_{—} | 2dtm3909f_ag |
| Tissue Name | ag1290_b1 | 2655 |
| Normal Colon GENPAK 061003 | 31.1 | 81.8 |
| 83219 CC Well to Mod Diff (ODO3866) | 0.3 | 10.6 |
| 83220 CC NAT (ODO3866) | 2.2 | 15.1 |
| 83221 CC Gr.2 rectosigmoid (ODO3868) | 4.1 | 7.7 |
| 83222 CC NAT (ODO3868) | 0.2 | 6.4 |
| 83235 CC Mod Diff (ODO3920) | 1.7 | 8.4 |
| 83236 CC NAT (ODO3920) | 3.4 | 11.0 |
| 83237 CC Gr.2 ascend colon (ODO3921) | 11.2 | 40.3 |
| 83238 CC NAT (ODO3921) | 4.5 | 10.5 |
| 83241 CC from Partial Hepatectomy (ODO4309) | 12.9 | 22.5 |
| 83242 Liver NAT (ODO4309) | 7.5 | 18.7 |
| 87472 Colon mets to lung (OD04451-01) | 4.1 | 11.0 |
| 87473 Lung NAT (OD04451-02) | 7.8 | 20.4 |
| Normal Prostate Clontech A + 6546-1 | 100.0 | 33.9 |
| 84140 Prostate Cancer (OD04410) | 17.7 | 54.7 |
| 84141 Prostate NAT (OD04410) | 22.9 | 46.0 |
| 87073 Prostate Cancer (OD04720-01) | 20.1 | 38.2 |
| 87074 Prostate NAT (OD04720-02) | 23.9 | 62.8 |
| Normal Lung GENPAK 061010 | 35.7 | 98.6 |
| 83239 Lung Met to Muscle (ODO4286) | 10.8 | 23.2 |
| 83240 Muscle NAT (ODO4286) | 6.6 | 13.9 |
| 84136 Lung Malignant Cancer (OD03126) | 34.1 | 100.0 |
| 84137 Lung NAT (OD03126) | 26.4 | 60.3 |
| 84871 Lung Cancer (OD04404) | 27.9 | 68.8 |
| 84872 Lung NAT (OD04404) | 12.9 | 0.0 |
| 84875 Lung Cancer (OD04565) | 5.6 | 11.6 |
| 84876 Lung NAT (OD04565) | 7.3 | 15.7 |
| 85950 Lung Cancer (OD04237-01) | 16.9 | 49.3 |
| 85970 Lung NAT (OD04237-02) | 16.8 | 32.8 |
| 83255 Ocular Mel Met to Liver (ODO4310) | 2.7 | 7.1 |
| 83256 Liver NAT (ODO4310) | 5.8 | 18.7 |
| 84139 Melanoma Mets to Lung (OD04321) | 17.2 | 29.9 |
| 84138 Lung NAT (OD04321) | 22.4 | 58.6 |
| Normal Kidney GENPAK 061008 | 25.9 | 47.3 |
| 83786 Kidney Ca, Nuclear grade 2 (OD04338) | 22.3 | 31.2 |
| 83787 Kidney NAT (OD04338) | 16.3 | 4.3 |
| 83788 Kidney Ca Nuclear grade 1/2 (OD04339) | 8.2 | 15.6 |
| 83789 Kidney NAT (OD04339) | 9.6 | 25.2 |
| 83790 Kidney Ca, Clear cell type (OD04340) | 22.7 | 55.1 |
| 83791 Kidney NAT (OD04340) | 15.7 | 37.1 |
| 83792 Kidney Ca, Nuclear grade 3 (OD04348) | 3.7 | 9.2 |
| 83793 Kidney NAT (OD04348) | 8.6 | 13.3 |
| 87474 Kidney Cancer (OD04622-01) | 4.3 | 11.9 |
| 87475 Kidney NAT (OD04622-03) | 1.1 | 2.0 |
| 85973 Kidney Cancer (OD04450-01) | 6.0 | 6.2 |
| 85974 Kidney NAT (OD04450-03) | 11.8 | 27.9 |
| Kidney Cancer Clontech 8120607 | 1.2 | 3.3 |
| Kidney NAT Clontech 8120608 | 2.2 | 2.5 |
| Kidney Cancer Clontech 8120613 | 2.6 | 0.0 |
| Kidney NAT Clontech 8120614 | 0.7 | 2.1 |
| Kidney Cancer Clontech 9010320 | 3.2 | 7.4 |
| Kidney NAT Clontech 9010321 | 1.9 | 5.7 |
| Normal Uterus GENPAK 061018 | 4.6 | 8.3 |
| Uterus Cancer GENPAK 064011 | 19.1 | 35.6 |
| Normal Thyroid Clontech A+ 6570-1 | 11.5 | 24.8 |
| Thyroid Cancer GENPAK 064010 | 9.0 | 15.2 |
| Thyroid Cancer INVITROGEN A302152 | 8.2 | 11.7 |
| Thyroid NAT INVITROGEN A302153 | 10.6 | 20.0 |
| Normal Breast GENPAK 061019 | 14.3 | 29.7 |
| 84877 Breast Cancer (OD04566) | 3.7 | 6.0 |
| 85975 Breast Cancer (OD04590-01) | 18.3 | 30.4 |
| 85976 Breast Cancer Mets (OD04590-03) | 27.0 | 39.5 |
| 87070 Breast Cancer Metastasis (OD04655-05) | 37.4 | 49.0 |
| GENPAK Breast Cancer 064006 | 5.3 | 13.7 |
| Breast Cancer Res. Gen. 1024 | 11.6 | 12.9 |
| Breast Cancer Clontech 9100266 | 6.8 | 17.7 |
| Breast NAT Clontech 9100265 | 6.0 | 11.1 |
| Breast Cancer INVITROGEN A209073 | 28.6 | 43.8 |
| Breast NAT INVITROGEN A2090734 | 7.6 | 21.8 |
| Normal Liver GENPAK 061009 | 7.1 | 16.8 |
| Liver Cancer GENPAK 064003 | 5.1 | 10.4 |
| Liver Cancer Research Genetics RNA 1025 | 2.1 | 4.4 |
| Liver Cancer Research Genetics RNA 1026 | 1.6 | 3.7 |
| Paired Liver Cancer Tissue Research Genetics RNA 6004-T | 2.2 | 7.6 |
| Paired Liver Tissue Research Genetics RNA 6004-N | 1.9 | 3.9 |
| Paired Liver Cancer Tissue Research Genetics RNA 6005-T | 2.1 | 5.0 |
| Paired Liver Tissue Research Genetics RNA 6005-N | 1.1 | 2.3 |
| Normal Bladder GENPAK 061001 | 11.0 | 35.1 |
| Bladder Cancer Research Genetics RNA 1023 | 2.2 | 7.9 |
| Bladder Cancer INVITROGEN A302173 | 20.0 | 41.2 |
| 87071 Bladder Cancer (OD04718-01) | 8.4 | 27.2 |
| 87072 Bladder Normal Adjacent (OD04718-03) | 17.8 | 23.0 |
| Normal Ovary Res. Gen. | 7.5 | 15.8 |
| Ovarian Cancer GENPAK 064008 | 33.7 | 51.0 |
| 87492 Ovary Cancer (OD04768-07) | 21.4 | 31.2 |
| 87493 Ovary NAT (OD04768-08) | 3.3 | 7.4 |
| Normal Stomach GENPAK 061017 | 16.5 | 33.2 |
| Gastric Cancer Clontech 9060358 | 3.9 | 7.4 |
| NAT Stomach Clontech 9060359 | 0.0 | 10.7 |
| Gastric Cancer Clontech 9060395 | 12.3 | 16.2 |
| NAT Stomach Clontech 9060394 | 9.6 | 17.2 |
| Gastric Cancer Clontech 9060397 | 13.8 | 35.1 |
| NAT Stomach Clontech 9060396 | 2.9 | 12.0 |
| Gastric Cancer GENPAK 064005 | 33.3 | 60.3 |

TABLE Y
Panel 4D

| | Rel Exp (%) | Rel Exp (%) |
|---|---|---|
| Tissue Name | 4dtm2467t_ag1290 | 4dtm3910f_ag2655 |
| 93768_Secondary Th1_anti-CD28/anti-CD3 | 12.3 | 12.9 |
| 93769_Secondary Th2_anti-CD28/anti-CD3 | 9.9 | 10.9 |
| 93770_Secondary Tr1_anti-CD28/anti-CD3 | 11.9 | 10.0 |
| 93573_Secondary Th1_resting day 4-6 in IL-2 | 4.2 | 3.3 |
| 93572_Secondary Th2_resting day 4-6 in IL-2 | 3.3 | 4.7 |
| 93571_Secondary Tr1_resting day 4-6 in IL-2 | 5.3 | 5.6 |
| 93568_primary Th1_anti-CD28/anti-CD3 | 14.5 | 12.9 |
| 93569_primary Th2_anti-CD28/anti-CD3 | 9.7 | 9.5 |
| 93570_primary Tr2_anti-CD28/anti-CD3 | 17.6 | 19.1 |
| 93565_primary Th1_resting dy 4-6 in IL-2 | 32.5 | 32.3 |
| 93566_primary Th2_resting dy 4-6 in IL-2 | 12.0 | 14.8 |
| 93567_primary Tr1_resting dy 4-6 in IL-2 | 3.6 | 0.0 |
| 93351_CD45RA CD4 lymphocyte_anti-CD28/anti-CD3 | 4.8 | 6.5 |
| 93352_CD45RO CD4 lymphocyte_anti-CD28/anti-CD3 | 13.8 | 16.3 |
| 93251_CD8 Lymphocytes_anti-CD28/anti-CD3 | 8.7 | 7.5 |
| 93353_chronic CD8 Lymphocytes 2ry_resting dy 4-6 in IL-2 | 6.3 | 5.1 |
| 93574_chronic CD8 Lymphocytes 2ry_activated CD3/CD28 | 5.1 | 6.6 |
| 93354_CD4_none | 6.3 | 6.7 |
| 93252_Secondary Th1/Th2/Tr1_anti-CD95 CH11 | 18.7 | 8.1 |
| 93103_LAK cells_resting | 6.7 | 7.4 |
| 93788_LAK cells_IL-2 | 6.8 | 11.5 |
| 93787_LAK cells_IL-2 + IL-12 | 9.0 | 9.2 |
| 93789_LAK cells_IL-2 + IFN gamma | 18.0 | 16.0 |
| 93790_LAK cells_IL-2 + IL-18 | 10.2 | 20.0 |
| 93104_LAK cells_PMA/ionomycin and IL-18 | 3.0 | 4.7 |
| 93578_NK Cells IL-2_resting | 10.3 | 8.7 |
| 93109_Mixed Lymphocyte Reaction_Two Way MLR | 12.1 | 9.0 |
| 93110_Mixed Lymphocyte Reaction_Two Way MLR | 4.4 | 4.3 |
| 93111_Mixed Lymphocyte Reaction_Two Way MLR | 4.9 | 2.6 |
| 93112_Mononuclear Cells (PBMCs)_resting | 5.2 | 4.6 |
| 93113_Mononuclear Cells (PBMCs)_PWM | 26.8 | 17.1 |
| 93114_Mononuclear Cells (PBMCs)_PHA-L | 10.7 | 8.9 |
| 93249_Ramos (B cell)_none | 21.9 | 16.5 |
| 93250_Ramos (B cell)_ionomycin | 100.0 | 100.0 |
| 93349_B lymphocytes_PWM | 34.4 | 31.0 |
| 93350_B lymphoytes_CD40L and IL-4 | 14.2 | 14.2 |
| 92665_EOL-1 (Eosinophil)_dbcAMP differentiated | 2.1 | 2.5 |
| 93248_EOL-1 (Eosinophil)_dbcAMP/PMAionomycin | 6.2 | 8.4 |
| 93356_Dendritic Cells_none | 6.3 | 5.0 |
| 93355_Dendritic Cells_LPS 100 ng/ml | 4.5 | 4.3 |
| 93775_Dendritic Cells_anti-CD40 | 7.1 | 5.3 |
| 93774_Monocytes_resting | 4.1 | 5.8 |
| 93776_Monocytes_LPS 50 ng/ml | 1.8 | 1.8 |
| 93581_Macrophages_resting | 5.8 | 4.4 |
| 93582_Macrophages_LPS 100 ng/ml | 2.1 | 2.6 |
| 93098_HUVEC (Endothelial)_none | 3.7 | 3.4 |
| 93099_HUVEC (Endothelial)_starved | 6.7 | 7.1 |
| 93100_HUVEC (Endothelial)_IL-1b | 3.2 | 4.9 |
| 93779_HUVEC (Endothelial)_IFN gamma | 4.4 | 7.2 |
| 93102_HUVEC (Endothelial)_TNF alpha + IFN gamma | 2.6 | 2.6 |
| 93101_HUVEC (Endothelial)_TNF alpha + IL4 | 2.0 | 2.7 |
| 93781_HUVEC (Endothelial)_IL-11 | 1.6 | 1.8 |
| 93583_Lung Microvascular Endothelial Cells_none | 2.9 | 2.6 |
| 93584_Lung Microvascular Endothelial Cells_TNFa (4 | 2.7 | 2.6 |
| ng/ml) and IL1b (1 ng/ml) | | |
| 92662_Microvascular Dermal endothelium_none | 5.7 | 5.1 |
| 92663_Microsvasular Dermal endothelium_TNFa (4 ng/ml) | 2.6 | 1.7 |
| and IL1b (1 ng/ml) | | |
| 93773_Bronchial epithelium_TNFa (4 ng/ml) and IL1b (1 | 2.4 | 0.0 |
| ng/ml)** | | |
| 93347_Small Airway Epithelium_none | 1.8 | 0.8 |
| 93348_Small Airway Epithelium_TNFa (4 ng/ml) and IL1b | 8.8 | 13.1 |
| (1 ng/ml) | | |
| 92668_Coronery Artery SMC_resting | 1.9 | 2.7 |
| 92669_Coronery Artery SMC_TNFa (4 ng/ml) and IL1b (1 | 0.8 | 1.5 |
| ng/ml) | | |
| 93107_astrocytes_resting | 1.6 | 2.8 |
| 93108_astrocytes_TNFa (4 ng/ml) and IL1b (1 ng/ml) | 0.8 | 1.1 |
| 92666_KU-812 (Basophil)_resting | 2.0 | 3.0 |
| 92667_KU-812 (Basophil)_PMA/ionomycin | 16.0 | 19.3 |
| 93579_CCD1106 (Keratinocytes)_none | 3.0 | 3.1 |
| 93580_CCD1106 (Keratinocytes)_TNFa and IFNg** | 0.7 | 0.4 |
| 93791_Liver Cirrhosis | 0.8 | 0.7 |
| 93792_Lupus Kidney | 1.0 | 1.2 |
| 93577_NCI-H292 | 5.8 | 6.2 |
| 93358_NCI-H292_IL-4 | 9.7 | 10.0 |
| 93360_NCI-H292_IL-9 | 7.9 | 8.7 |
| 93359_NCI-H292_IL-13 | 2.5 | 4.8 |
| 93357_NCI-H292_IFN gamma | 3.8 | 5.2 |
| 93777_HPAEC_- | 1.3 | 1.9 |
| 93778_HPAEC_IL-1 beta/TNA alpha | 2.9 | 2.3 |
| 93254_Normal Human Lung Fibroblast_none | 8.3 | 6.0 |
| 93253_Normal Human Lung Fibroblast_TNFa (4 ng/ml) and | 2.4 | 2.8 |
| IL-1b (1 ng/ml) | | |
| 93257_Normal Human Lung Fibroblast_IL-4 | 30.1 | 24.0 |
| 93256_Normal Human Lung Fibroblast_IL-9 | 9.5 | 9.7 |
| 93255_Normal Human Lung Fibroblast_IL-13 | 16.6 | 14.1 |
| 93258_Normal Human Lung Fibroblast_IFN gamma | 15.2 | 18.4 |
| 93106_Dermal Fibroblasts CCD1070_resting | 10.0 | 8.7 |
| 93361_Dermal Fibroblasts CCD1070_TNF alpha 4 ng/ml | 22.7 | 24.1 |
| 93105_Dermal Fibroblasts CCD1070_IL-1 beta 1 ng/ml | 2.7 | 4.2 |
| 93772_dermal fibroblast_IFN gamma | 1.7 | 2.0 |
| 93771_dermal fibroblast_IL-4 | 2.7 | 2.5 |
| 93259_IBD Colitis 1** | 0.3 | 0.0 |
| 93260_IBD Colitis 2 | 1.0 | 1.0 |
| 93261_IBD Crohn's | 0.5 | 0.8 |
| 735010_Colon_normal | 7.2 | 9.3 |
| 735019_Lung_none | 5.5 | 7.5 |
| 64028-1_Thymus_none | 5.8 | 8.4 |
| 64030-1_Kidney_none | 16.7 | 13.2 |

Panel 1.3D Summary: This gene appears to be expressed across the majority of samples in panel 1.3D. In addition, specific regions of the brain, some brain cancers and a sample of metastatic gastric cancer expressed this gene highly.

Panel 2D Summary: In some tissues this gene is expressed very highly. In particular, one sample each of ovarian, liver, breast, lung, colon and two of kidney cancer all express this gene highly. Thus, this gene may play an important role in these cancers and therefore therapeutic targeting of this gene may be of use for disease intervention.

Panel 4D Summary: This transcript is induced in the Ramos B cell line after activation with PMA and ionomycin. Induction of the transcript is also seen in PWM stimulated PBMC (which includes normal B cells). This protein encoded for by this transcript may be important in B cell signal transduction and/or the interaction of B cells with other cell types as an adhesion molecule. Antibody or small molecule therapies designed against the protein encoded for by this transcript could reduce or inhibit inflammation by controlling B cell activation and/or Ig production. Antagonistic therapies could be important in the treatment of autoimmune diseases such as asthma, allergy, arthritis, psoriasis, delayed type hypersensitivity, emphysema and lupus. Agonistic protein therapeutics could have adjuvant like properties and could be coadministered with vaccines.

### OTHER EMBODIMENTS

Although particular embodiments have been disclosed herein in detail, this has been done by way of example for purposes of illustration only, and is not intended to be limiting with respect to the scope of the appended claims, which follow. In particular, it is contemplated by the inventors that various substitutions, alterations, and modifications may be made to the invention without departing from the spirit and scope of the invention as defined by the claims. The choice of nucleic acid starting material, clone of interest, or library type is believed to be a matter of routine for a person of ordinary skill in the art with knowledge of the embodiments described herein. Other aspects, advantages, and modifications considered to be within the scope of the following claims.

What is claimed is:

1. An isolated polypeptide comprising an amino acid sequence selected from the group consisting of:
(a) a mature form of an amino acid sequence selected from the group consisting of SEQ ID NOS:2, 4, 6, 8, 10, 12, 14, 16, 18, 20, 22, and 24;
(b) a variant of a mature form of an amino acid sequence selected from the group consisting of SEQ ID NOS:2, 4, 6, 8, 10, 12, 14, 16, 18, 20, 22, and 24, wherein one or more amino acid residues in said variant differs from the amino acid sequence of said mature form, provided that said variant differs in no more than 15% of the amino acid residues from the amino acid sequence of said mature form;
(c) an amino acid sequence selected from the group consisting SEQ ID NOS:2, 4, 6, 8, 10, 12, 14, 16, 18,20,22,and24;and
(d) a variant of an amino acid sequence selected from the group consisting of SEQ ID NOS:2, 4, 6, 8, 10, 12, 14, 16, 18, 20, 22, and 24, wherein one or more amino acid residues in said variant differs from the amino acid sequence of said mature form, provided that said variant differs in no more than 15% of amino acid residues from said amino acid sequence.

2. The polypeptide of claim 1, wherein said polypeptide comprises the amino acid sequence of a naturally-occurring allelic variant of an amino acid sequence selected from the group consisting SEQ ID NOS:2, 4, 6, 8, 10, 12, 14, 16, 18, 20, 22, and 24.

3. The polypeptide of claim 2, wherein said allelic variant comprises an amino acid sequence that is the translation of a nucleic acid sequence differing by a single nucleotide from a nucleic acid sequence selected from the group consisting of SEQ ID NOS: 1, 3, 5, 7, 9,11,13,15, 17, 19, 21, and 23.

4. The polypeptide of claim 1, wherein the amino acid sequence of said variant comprises a conservative amino acid substitution.

5. An isolated nucleic acid molecule comprising a nucleic acid sequence encoding a polypeptide comprising an amino acid sequence selected from the group consisting of:
(a) a mature form of an amino acid sequence selected from the group consisting of SEQ ID NOS: 2, 4, 6, 8, 10, 12, 14, 16, 18, 20, 22, and 24;
(b) a variant of a mature form of an amino acid sequence selected from the group consisting of SEQ ID NOS: 2, 4, 6, 8, 10, 12, 14, 16, 18, 20, 22, and 24, wherein one or more amino acid residues in said variant differs from the amino acid sequence of said mature form, provided that said variant differs in no more than 15% of the amino acid residues from the amino acid sequence of said mature form;
(c) an amino acid sequence selected from the group consisting of SEQ ID NOS:2, 4, 6, 8, 10, 12, 14, 16, 18, 20, 22, and 24;
(d) a variant of an amino acid sequence selected from the group consisting SEQ ID NOS:2, 4, 6, 8, 10, 12, 14, 16, 18, 20, 22, and 24, wherein one or more amino acid residues in said variant differs from the amino acid sequence of said mature form, provided that said variant differs in no more than 15% of amino acid residues from said amino acid sequence;
(e) a nucleic acid fragment encoding at least a portion of a polypeptide comprising an amino acid sequence chosen from the group consisting of SEQ ID NOS:2, 4, 6, 8, 10, 12, 14, 16, 18, 20, 22, and 24, ora variant of said polypeptide, wherein one or more amino acid residues in said variant differs from the amino acid sequence of said mature form, provided that said variant differs in no more than 15% of amino acid residues from said amino acid sequence; and
(f) a nucleic acid molecule comprising the complement of (a), (b), (c), (d) or (e).

6. The nucleic acid molecule of claim 5, wherein the nucleic acid molecule comprises the nucleotide sequence of a naturally-occurring allelic nucleic acid variant.

7. The nucleic acid molecule of claim 5, wherein the nucleic acid molecule encodes a polypeptide comprising the amino acid sequence of a naturally-occurring polypeptide variant.

8. The nucleic acid molecule of claim 5, wherein the nucleic acid molecule differs by a single nucleotide from a nucleic acid sequence selected from the group consisting of SEQ ID NOS:1, 3, 5, 7, 9, 11, 13, 15, 17, 19, 21, and 23.

9. The nucleic acid molecule of claim 5, wherein said nucleic acid molecule comprises a nucleotide sequence selected from the group consisting of:
(a) a nucleotide sequence selected from the group consisting of SEQ ID NOS: 1, 3, 5, 7, 9, 11, 13, 15, 17, 19, 21, and 23;
(b) a nucleotide sequence differing by one or more nucleotides from a nucleotide sequence selected from the group consisting of SEQ ID NOS: 1, 3, 5, 7, 9, 11, 13, 15, 17, 19, 21, and 23, provided that no more than 20% of the nucleotides differ from said nucleotide sequence;
(c) a nucleic acid fragment of (a); and
(d) a nucleic acid fragment of (b).

10. The nucleic acid molecule of claim 5, wherein said nucleic acid molecule hybridizes under stringent conditions to a nucleotide sequence chosen from the group consisting SEQ ID NOS: 1, 3, 5, 7, 9, 11, 13, 15, 17, 19, 21, and 23, or a complement of said nucleotide sequence.

11. The nucleic acid molecule of claim 5, wherein the nucleic acid molecule comprises a nucleotide sequence selected from the group consisting of:
(a) a first nucleotide sequence comprising a coding sequence differing by one or more nucleotide sequences from a coding sequence encoding said amino acid sequence, provided that no more than 20% of the nucleotides in the coding sequence in said first nucleotide sequence differ from said coding sequence;
(b) an isolated second polynucleotide that is a complement of the first polynucleotide; and
(c) a nucleic acid fragment of (a) or (b).

12. A vector comprising the nucleic acid molecule of claim 11.

13. The vector of claim 12, further comprising a promoter operably-linked to said nucleic acid molecule.

14. A cell comprising the vector of claim 12.

15. An antibody that binds immunospecifically to the polypeptide of claim 1.

16. The antibody of claim 15, wherein said antibody is a monoclonal antibody.

17. The antibody of claim 15, wherein the antibody is a humanized antibody.

18. A method for determining the presence or amount of the polypeptide of claim 1 in a sample, the method comprising:
(a) providing the sample;
(b) contacting the sample with an antibody that binds immunospecifically to the polypeptide; and
(c) determining the presence or amount of antibody bound to said polypeptide, thereby determining the presence or amount of polypeptide in said sample.

19. A method for determining the presence or amount of the nucleic acid molecule of claim 5 in a sample, the method comprising:
(a) providing the sample;
(b) contacting the sample with a probe that binds to said nucleic acid molecule; and
(c) determining the presence or amount of the probe bound to said nucleic acid molecule, thereby determining the presence or amount of the nucleic acid molecule in said sample.

20. The method of claim 19 wherein presence or amount of the nucleic acid molecule is used as a marker for cell or tissue type.

21. The method of claim 20 wherein the cell or tissue type is cancerous.

22. A method of identifying an agent that binds to a polypeptide of claim 1, the method comprising:
(a) contacting said polypeptide with said agent; and
(b) determining whether said agent binds to said polypeptide.

23. The method of claim 22 wherein the agent is a cellular receptor or a downstream effector.

24. A method for identifying an agent that modulates the expression or activity of the polypeptide of claim 1, the method comprising:
(a) providing a cell expressing said polypeptide;
(b) contacting the cell with said agent, and (c) determining whether the agent modulates expression or activity of said polypeptide,
whereby an alteration in expression or activity of said peptide indicates said agent modulates expression or activity of said polypeptide.

25. A method for modulating the activity of the polypeptide of claim 1, the method comprising contacting a cell sample expressing the polypeptide of said claim with a compound that binds to said polypeptide in an amount sufficient to modulate the activity of the polypeptide.

26. A method of treating or preventing a NOVX-associated disorder, said method comprising administering to a subject in which such treatment or prevention is desired the polypeptide of claim 1 in an amount sufficient to treat or prevent said NOVX-associated disorder in said subject.

27. The method of claim 26 wherein the disorder is selected from the group consisting of cardiomyopathy and atherosclerosis.

28. The method of claim 26 wherein the disorder is related to cell signal processing and metabolic pathway modulation.

29. The method of claim 26, wherein said subject is a human.

30. A method of treating or preventing a NOVX-associated disorder, said method comprising administering to a subject in which such treatment or prevention is desired the nucleic acid of claim 5 in an amount sufficient to treat or prevent said NOVX-associated disorder in said subject.

31. The method of claim 30 wherein the disorder is selected from the group consisting of cardiomyopathy and atherosclerosis.

32. The method of claim 30 wherein the disorder is related to cell signal processing and metabolic pathway modulation.

33. The method of claim 30, wherein said subject is a human.

34. A method of treating or preventing a NOVX-associated disorder, said method comprising administering to a subject in which such treatment or prevention is desired the antibody of claim 15 in an amount sufficient to treat or prevent said NOVX-associated disorder in said subject.

35. The method of claim 34 wherein the disorder is diabetes.

36. The method of claim 34 wherein the disorder is related to cell signal processing and metabolic pathway modulation.

37. The method of claim 34, wherein the subject is a human.

38. A pharmaceutical composition comprising the polypeptide of claim 1 and a pharmaceutically-acceptable carrier.

39. A pharmaceutical composition comprising the nucleic acid molecule of claim 5 and a pharmaceutically-acceptable carrier.

40. A pharmaceutical composition comprising the antibody of claim 15 and a pharmaceutically-acceptable carrier.

41. A kit comprising in one or more containers, the pharmaceutical composition of claim 38.

42. A kit comprising in one or more containers, the pharmaceutical composition of claim 39.

43. A kit comprising in one or more containers, the pharmaceutical composition of claim 40.

44. A method for determining the presence of or predisposition to a disease associated with altered levels of the polypeptide of claim 1 in a first mammalian subject, the method comprising:
(a) measuring the level of expression of the polypeptide in a sample from the first mammalian subject; and
(b) comparing the amount of said polypeptide in the sample of step (a) to the amount of the polypeptide present in a control sample from a second mammalian subject known not to have, or not to be predisposed to, said disease;
wherein an alteration in the expression level of the polypeptide in the first subject as compared to the control sample indicates the presence of or predisposition to said disease.

45. The method of claim 44 wherein the predisposition is to cancers.

46. A method for determining the presence of or predisposition to a disease associated with altered levels of the nucleic acid molecule of claim 5 in a first mammalian subject, the method comprising:
(a) measuring the amount of the nucleic acid in a sample from the first mammalian subject; and
(b) comparing the amount of said nucleic acid in the sample of step (a) to the amount of the nucleic acid present in a control sample from a second mammalian subject known not to have or not be predisposed to, the disease;
wherein an alteration in the level of the nucleic acid in the first subject as compared to the control sample indicates the presence of or predisposition to the disease.

47. The method of claim 46 wherein the predisposition is to a cancer.

48. A method of treating a pathological state in a mammal, the method comprising administering to the mammal a polypeptide in an amount that is sufficient to alleviate the pathological state, wherein the polypeptide is a polypeptide having an amino acid sequence at least 95% identical to a polypeptide comprising an amino acid sequence of at least one of SEQ ID NOS:2, 4, 6, 8, 10, 12, 14, 16, 18, 20, 22, and 24, or a biologically active fragment thereof.

49. A method of treating a pathological state in a mammal, the method comprising administering to the mammal the antibody of claim 15 in an amount sufficient to alleviate the pathological state.
